SEQUENCE LISTING (167 sequences)

SEQ ID NO: 1 - protein, 6 aa, Homo sapiens
NSASDY

SEQ ID NO: 2 - protein, 7 aa, Homo sapiens
IRSNMDK

SEQ ID NO: 3 - protein, 11 aa, Homo sapiens
AEYIQGAQKLV

SEQ ID NO: 4 - protein, 5 aa, Homo sapiens
LGHNA

SEQ ID NO: 5 - protein, 6 aa, Homo sapiens
YSLEER

SEQ ID NO: 6 - protein, 10 aa, Homo sapiens
ASSQEDNEQF

SEQ ID NO: 7 - protein, 132 aa, Homo sapiens
MAGIRALFMYLWLQLDWVSRGESVGLHLPTLSVQEGDNSIINCAYSNSASDYFIWYKQESGKGPQFIIDIRSNMDKRQGQRVTVLLNKTVKHLSLQIAATQPGDSAVYFCAEYIQGAQKLVFGQGTRLTINP

SEQ ID NO: 8 - protein, 133 aa, Artificial Sequence
  Synthetic
MACRLLCCAVLCLLGAGELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQEDNEQFFGPGTRLTVL

SEQ ID NO: 9 - protein, 189 aa, Homo sapiens [sequence omitted]

SEQ ID NO: 10 - protein, 188 aa, Homo sapiens [sequence omitted]

SEQ ID NO: 11 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 12 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 13 - protein, 189 aa, Homo sapiens
MTEYKLVVVGAVGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQRVEDAFYTLVREIRQYRLKKISKEEKTPGCVKIKKCIIM

SEQ ID NO: 14 - protein, 188 aa, Homo sapiens
MTEYKLVVVGAVGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEKMSKDGKKKKKKSKTKCVIM

SEQ ID NO: 15 - protein, 189 aa, Homo sapiens [sequence omitted]

SEQ ID NO: 16 - protein, 189 aa, Homo sapiens
MTEYKLVVVGAVGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 17 - protein, 137 aa, Artificial Sequence
  Synthetic
  MISC_FEATURE (48)..(48): Xaa is Thr or Cys
  MISC_FEATURE (112)..(112): Xaa is Ser, Ala, Val, Leu, Ile, Pro, Phe, Met, or Trp
  MISC_FEATURE (114)..(114): Xaa is Met, Ala, Val, Leu, Ile, Pro, Phe, or Trp
  MISC_FEATURE (115)..(115): Xaa is Gly, Ala, Val, Leu, Ile, Pro, Phe, Met, or Trp
NIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 18 - protein, 173 aa, Artificial Sequence
  Synthetic
  MISC_FEATURE (57)..(57): Xaa is Ser or Cys
EDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 19 - protein, 137 aa, Mus musculus
NIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 20 - protein, 173 aa, Mus musculus
EDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 21 - protein, 269 aa, Artificial Sequence
  Synthetic
  MISC_FEATURE (180)..(180): Xaa is Thr or Cys
  MISC_FEATURE (244)..(244): Xaa is Ser, Ala, Val, Leu, Ile, Pro, Phe, Met, or Trp
  MISC_FEATURE (246)..(246): Xaa is Met, Ala, Val, Leu, Ile, Pro, Phe, or Trp
  MISC_FEATURE (247)..(247): Xaa is Gly, Ala, Val, Leu, Ile, Pro, Phe, Met, or Trp
MAGIRALFMYLWLQLDWVSRGESVGLHLPTLSVQEGDNSIINCAYSNSASDYFIWYKQESGKGPQFIIDIRSNMDKRQGQRVTVLLNKTVKHLSLQIAATQPGDSAVYFCAEYIQGAQKLVFGQGTRLTINPNIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 22 - protein, 306 aa, Artificial Sequence
  Synthetic
  MISC_FEATURE (190)..(190): Xaa is Ser or Cys
MACRLLCCAVLCLLGAGELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQEDNEQFFGPGTRLTVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 23 - protein, 269 aa, Artificial Sequence
  Synthetic
MAGIRALFMYLWLQLDWVSRGESVGLHLPTLSVQEGDNSIINCAYSNSASDYFIWYKQESGKGPQFIIDIRSNMDKRQGQRVTVLLNKTVKHLSLQIAATQPGDSAVYFCAEYIQGAQKLVFGQGTRLTINPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 24 - protein, 306 aa, Artificial Sequence
  Synthetic
MACRLLCCAVLCLLGAGELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQEDNEQFFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 25 - protein, 27 aa, Artificial Sequence
  Synthetic
RAKRSGSGATNFSLLKQAGDVEENPGP

SEQ ID NO: 26 - protein, 9 aa, Homo sapiens
AGGVGKSAL

SEQ ID NO: 27 - protein, 24 aa, Homo sapiens
MTEYKLVVVGAGGVGKSALTIQLI

SEQ ID NO: 28 - protein, 23 aa, Homo sapiens
TEYKLVVVGAGGVGKSALTIQLI

SEQ ID NO: 29 - protein, 9 aa, Homo sapiens
AVGVGKSAL

SEQ ID NO: 30 - protein, 24 aa, Homo sapiens
MTEYKLVVVGAVGVGKSALTIQLI

SEQ ID NO: 31 - protein, 6 aa, Homo sapiens
NSASQS

SEQ ID NO: 32 - protein, 6 aa, Homo sapiens
VYSSGN

SEQ ID NO: 33 - protein, 13 aa, Homo sapiens
AVNPKYTGGFKTI

SEQ ID NO: 34 - protein, 5 aa, Homo sapiens
LGHNA

SEQ ID NO: 35 - protein, 6 aa, Homo sapiens
YSLEER

SEQ ID NO: 36 - protein, 15 aa, Homo sapiens
ASSQDVTSEWVDTIY

SEQ ID NO: 37 - protein, 133 aa, Homo sapiens
MISLRVLLVILWLQLSWVWSQRKEVEQDPGPFNVPEGATVAFNCTYSNSASQSFFWYRQDCRKEPKLLMSVYSSGNEDGRFTAHLNRASQYISLLIRDSKLSDSATYLCAVNPKYTGGFKTIFGAGTRLFVKA

SEQ ID NO: 38 - protein, 138 aa, Artificial Sequence
  Synthetic
MACRLLCCAVLCLLGAGELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQDVTSEWVDTIYFGEGSWLTVV

SEQ ID NO: 39 - protein, 270 aa, Artificial Sequence
  Synthetic
MISLRVLLVILWLQLSWVWSQRKEVEQDPGPFNVPEGATVAFNCTYSNSASQSFFWYRQDCRKEPKLLMSVYSSGNEDGRFTAHLNRASQYISLLIRDSKLSDSATYLCAVNPKYTGGFKTIFGAGTRLFVKANIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 40 - protein, 311 aa, Artificial Sequence
  Synthetic
MACRLLCCAVLCLLGAGELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQDVTSEWVDTIYFGEGSWLTVVEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 41 - protein, 270 aa, Artificial Sequence
  Synthetic
  MISC_FEATURE (181)..(181): Xaa is Thr or Cys
  MISC_FEATURE (245)..(245): Xaa is Ser, Ala, Val, Leu, Ile, Pro, Phe, Met, or Trp
  MISC_FEATURE (247)..(247): Xaa is Met, Ala, Val, Leu, Ile, Pro, Phe, or Trp
  MISC_FEATURE (248)..(248): Xaa is Gly, Ala, Val, Leu, Ile, Pro, Phe, Met, or Trp
MISLRVLLVILWLQLSWVWSQRKEVEQDPGPFNVPEGATVAFNCTYSNSASQSFFWYRQDCRKEPKLLMSVYSSGNEDGRFTAHLNRASQYISLLIRDSKLSDSATYLCAVNPKYTGGFKTIFGAGTRLFVKANIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 42 - protein, 311 aa, Artificial Sequence
  Synthetic
  MISC_FEATURE (195)..(195): Xaa is Ser or Cys
MACRLLCCAVLCLLGAGELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQDVTSEWVDTIYFGEGSWLTVVEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 43 - DNA, 396 bp, Homo sapiens
atggccggcatccgggccctgtttatgtacctgtggctgcagctggactgggtgagccggggagagagcgtgggcctgcacctgcctacactgtctgtgcaggagggcgataacagcatcatcaattgcgcctattctaacagcgcctccgactacttcatctggtataagcaggagagcggcaagggcccacagtttatcatcgatatccggtccaacatggacaagaggcagggacagagggtgaccgtgctgctgaataagacagtgaagcacctgtctctgcagatcgcagcaacccagcctggcgacagcgccgtgtacttctgtgccgagtatatccagggcgcccagaagctggtgtttggccagggcacccggctgacaatcaaccca

SEQ ID NO: 44 - DNA, 399 bp, Homo sapiens
atggcctgcaggctgctgtgctgtgccgtgctgtgcctgctgggagcaggagagctggtgcctatggagacaggcgtgacccagacacctcgccacctggtcatgggcatgacaaacaagaagagcctgaagtgcgagcagcacctgggccacaatgccatgtactggtataagcagtccgccaagaagcctctggagctgatgttcgtgtactctctggaggagagggtggagaacaattccgtgccatctcgcttcagcccagagtgccccaatagctcccacctgtttctgcacctgcacaccctgcagccagaggattccgccctgtacctgtgcgcctctagccaggaggacaacgagcagttctttggaccaggaaccaggctgacagtgctg

SEQ ID NO: 45 - DNA, 399 bp, Homo sapiens
atgatctccctgagggtgctgctggtcatcctgtggctgcagctgtcctgggtgtggtctcagaggaaggaggtggagcaggacccaggacctttcaatgtgcctgagggcgccaccgtggcctttaactgcacatactccaattctgccagccagtccttcttttggtataggcaggattgtcgcaaggagccaaagctgctgatgagcgtgtacagcagcggaaacgaggacggccggtttaccgcacacctgaatagagccagccagtatatctccctgctgatccgggacagcaagctgtctgatagcgccacctacctgtgcgccgtgaaccccaagtataccggcggcttcaagacaatctttggagcaggaacaaggctgttcgtgaaggca

SEQ ID NO: 46 - DNA, 414 bp, Homo sapiens
atggcctgcaggctgctgtgctgtgccgtgctgtgcctgctgggagcaggagagctggtgccaatggagaccggagtgacccagacaccaaggcacctggtcatgggcatgacaaacaagaagtccctgaagtgcgagcagcacctgggccacaatgccatgtactggtataagcagtccgccaagaagcctctggagctgatgttcgtgtactctctggaggagagggtggagaacaatagcgtgccatcccgcttctctcctgagtgcccaaacagctcccacctgtttctgcacctgcacaccctgcagcctgaggatagcgccctgtacctgtgcgcctctagccaggatgtgaccagcgagtgggtggacacaatctatttcggagagggatcctggctgaccgtggtg

SEQ ID NO: 47 - protein, 112 aa, Homo sapiens
GESVGLHLPTLSVQEGDNSIINCAYSNSASDYFIWYKQESGKGPQFIIDIRSNMDKRQGQRVTVLLNKTVKHLSLQIAATQPGDSAVYFCAEYIQGAQKLVFGQGTRLTINP

SEQ ID NO: 48 - protein, 111 aa, Homo sapiens
ETGVTQTPRHLVMGMTNKKSLKCEQHLGHNAMYWYKQSAKKPLELMFVYSLEERVENNSVPSRFSPECPNSSHLFLHLHTLQPEDSALYLCASSQEDNEQFFGPGTRLTVL

SEQ ID NO: 49 - protein, 112 aa, Homo sapiens
RKEVEQDPGPFNVPEGATVAFNCTYSNSASQSFFWYRQDCRKEPKLLMSVYSSGNEDGRFTAHLNRASQYISLLIRDSKLSDSATYLCAVNPKYTGGFKTIFGAGTRLFVKA

SEQ ID NO: 50 - protein, 116 aa, Homo sapiens
ETGVTQTPRHLVMGMTNKKSLKCEQHLGHNAMYWYKQSAKKPLELMFVYSLEERVENNSVPSRFSPECPNSSHLFLHLHTLQPEDSALYLCASSQDVTSEWVDTIYFGEGSWLTVV

SEQ ID NO: 51 - protein, 249 aa, Artificial Sequence
  Synthetic
GESVGLHLPTLSVQEGDNSIINCAYSNSASDYFIWYKQESGKGPQFIIDIRSNMDKRQGQRVTVLLNKTVKHLSLQIAATQPGDSAVYFCAEYIQGAQKLVFGQGTRLTINPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 52 - protein, 284 aa, Artificial Sequence
  Synthetic
ETGVTQTPRHLVMGMTNKKSLKCEQHLGHNAMYWYKQSAKKPLELMFVYSLEERVENNSVPSRFSPECPNSSHLFLHLHTLQPEDSALYLCASSQEDNEQFFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 53 - protein, 249 aa, Artificial Sequence
  Synthetic
RKEVEQDPGPFNVPEGATVAFNCTYSNSASQSFFWYRQDCRKEPKLLMSVYSSGNEDGRFTAHLNRASQYISLLIRDSKLSDSATYLCAVNPKYTGGFKTIFGAGTRLFVKANIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 54 - protein, 289 aa, Artificial Sequence
  Synthetic
ETGVTQTPRHLVMGMTNKKSLKCEQHLGHNAMYWYKQSAKKPLELMFVYSLEERVENNSVPSRFSPECPNSSHLFLHLHTLQPEDSALYLCASSQDVTSEWVDTIYFGEGSWLTVVEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 55 - protein, 249 aa, Artificial Sequence
  Synthetic
  MISC_FEATURE (160)..(160): Xaa is Thr or Cys
  MISC_FEATURE (224)..(224): Xaa is Ser, Ala, Val, Leu, Ile, Pro, Phe, Met, or Trp
  MISC_FEATURE (226)..(226): Xaa is Met, Ala, Val, Leu, Ile, Pro, Phe, or Trp
  MISC_FEATURE (227)..(227): Xaa is Gly, Ala, Val, Leu, Ile, Pro, Phe, Met, or Trp
GESVGLHLPTLSVQEGDNSIINCAYSNSASDYFIWYKQESGKGPQFIIDIRSNMDKRQGQRVTVLLNKTVKHLSLQIAATQPGDSAVYFCAEYIQGAQKLVFGQGTRLTINPNIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 56 - protein, 284 aa, Artificial Sequence
  Synthetic
  MISC_FEATURE (168)..(168): Xaa is Ser or Cys
ETGVTQTPRHLVMGMTNKKSLKCEQHLGHNAMYWYKQSAKKPLELMFVYSLEERVENNSVPSRFSPECPNSSHLFLHLHTLQPEDSALYLCASSQEDNEQFFGPGTRLTVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 57 - protein, 249 aa, Artificial Sequence
  Synthetic
  MISC_FEATURE (160)..(160): Xaa is Thr or Cys
  MISC_FEATURE (224)..(224): Xaa is Ser, Ala, Val, Leu, Ile, Pro, Phe, Met, or Trp
  MISC_FEATURE (226)..(226): Xaa is Met, Ala, Val, Leu, Ile, Pro, Phe, or Trp
  MISC_FEATURE (227)..(227): Xaa is Gly, Ala, Val, Leu, Ile, Pro, Phe, Met, or Trp
RKEVEQDPGPFNVPEGATVAFNCTYSNSASQSFFWYRQDCRKEPKLLMSVYSSGNEDGRFTAHLNRASQYISLLIRDSKLSDSATYLCAVNPKYTGGFKTIFGAGTRLFVKANIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 58 - protein, 289 aa, Artificial Sequence
  Synthetic
  MISC_FEATURE (173)..(173): Xaa is Ser or Cys
ETGVTQTPRHLVMGMTNKKSLKCEQHLGHNAMYWYKQSAKKPLELMFVYSLEERVENNSVPSRFSPECPNSSHLFLHLHTLQPEDSALYLCASSQDVTSEWVDTIYFGEGSWLTVVEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 59 - protein, 10 aa, Homo sapiens
KLVVVGAVGV

SEQ ID NO: 60 - protein, 9 aa, Homo sapiens
VVGAVGVGK

SEQ ID NO: 61 - protein, 10 aa, Homo sapiens
VVVGAVGVGK

SEQ ID NO: 62 - protein, 9 aa, Homo sapiens
VVVGAVGVG

SEQ ID NO: 63 - protein, 9 aa, Homo sapiens
AVGVGKSAL

SEQ ID NO: 64 - protein, 6 aa, Homo sapiens
NSASQS

SEQ ID NO: 65 - protein, 6 aa, Homo sapiens
VYSSGN

SEQ ID NO: 66 - protein, 12 aa, Homo sapiens
VVDDQTGANNLF

SEQ ID NO: 67 - protein, 5 aa, Homo sapiens
MNHEY

SEQ ID NO: 68 - protein, 6 aa, Homo sapiens
SVGAGI

SEQ ID NO: 69 - protein, 10 aa, Homo sapiens
ASRNLGDTQY

SEQ ID NO: 70 - protein, 132 aa, Homo sapiens
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVDDQTGANNLFFGTGTRLTVIP

SEQ ID NO: 71 - protein, 130 aa, Artificial Sequence
  Synthetic
MAIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASRNLGDTQYFGPGTRLTVL

SEQ ID NO: 72 - protein, 111 aa, Homo sapiens
RKEVEQDPGPFNVPEGATVAFNCTYSNSASQSFFWYRQDCRKEPKLLMSVYSSGNEDGRFTAQLNRASQYISLLIRDSKLSDSATYLCVVDDQTGANNLFFGTGTRLTVIP

SEQ ID NO: 73 - protein, 111 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASRNLGDTQYFGPGTRLTVL

SEQ ID NO: 74 - protein, 269 aa, Artificial Sequence
  Synthetic
  MISC_FEATURE (180)..(180): Xaa is Thr or Cys
  MISC_FEATURE (244)..(244): Xaa is Ser, Ala, Val, Leu, Ile, Pro, Phe, Met, or Trp
  MISC_FEATURE (246)..(246): Xaa is Met, Ala, Val, Leu, Ile, Pro, Phe, or Trp
  MISC_FEATURE (247)..(247): Xaa is Gly, Ala, Val, Leu, Ile, Pro, Phe, Met, or Trp
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVDDQTGANNLFFGTGTRLTVIPNIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 75 - protein, 303 aa, Artificial Sequence
  Synthetic
  MISC_FEATURE (187)..(187): Xaa is Ser or Cys
MAIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASRNLGDTQYFGPGTRLTVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 76 - protein, 248 aa, Artificial Sequence
  Synthetic
  MISC_FEATURE (159)..(159): Xaa is Thr or Cys
  MISC_FEATURE (223)..(223): Xaa is Ser, Ala, Val, Leu, Ile, Pro, Phe, Met, or Trp
  MISC_FEATURE (225)..(225): Xaa is Met, Ala, Val, Leu, Ile, Pro, Phe, or Trp
  MISC_FEATURE (226)..(226): Xaa is Gly, Ala, Val, Leu, Ile, Pro, Phe, Met, or Trp
RKEVEQDPGPFNVPEGATVAFNCTYSNSASQSFFWYRQDCRKEPKLLMSVYSSGNEDGRFTAQLNRASQYISLLIRDSKLSDSATYLCVVDDQTGANNLFFGTGTRLTVIPNIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 77 - protein, 284 aa, Artificial Sequence
  Synthetic
  MISC_FEATURE (168)..(168): Xaa is Ser or Cys
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASRNLGDTQYFGPGTRLTVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 78 - protein, 269 aa, Artificial Sequence
  Synthetic
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVDDQTGANNLFFGTGTRLTVIPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 79 - protein, 303 aa, Artificial Sequence
  Synthetic
MAIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASRNLGDTQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 80 - protein, 248 aa, Artificial Sequence
  Synthetic
RKEVEQDPGPFNVPEGATVAFNCTYSNSASQSFFWYRQDCRKEPKLLMSVYSSGNEDGRFTAQLNRASQYISLLIRDSKLSDSATYLCVVDDQTGANNLFFGTGTRLTVIPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 81 - protein, 284 aa, Artificial Sequence
  Synthetic
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASRNLGDTQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 82 - DNA, 396 bp, Homo sapiens
atgatcagcctgagggtgctgctggtcatcctgtggctgcagctgtcctgggtgtggtctcagaggaaggaggtggagcaggatccaggacctttcaatgtgcctgagggcgccaccgtggcctttaactgcacatactctaatagcgcctcccagtctttcttttggtataggcaggattgtcgcaaggagccaaagctgctgatgagcgtgtactctagcggaaacgaggacggccggtttaccgcacagctgaatagagccagccagtacatctccctgctgatcagggactccaagctgagcgattccgccacctacctgtgcgtggtggacgatcagacaggcgccaacaatctgttctttggcaccggcacacggctgaccgtgatccct

SEQ ID NO: 83 - DNA, 390 bp, Homo sapiens
atggccatcggactgctgtgctgtgccgccctgagcctgctgtgggcaggaccagtgaacgcaggagtgacccagacacccaagttccaggtgctgaagaccggccagagcatgacactgcagtgcgcccaggatatgaatcacgagtacatgtcctggtataggcaggacccaggaatgggactgaggctgatccactactccgtgggagcaggaatcaccgatcagggagaggtgcccaacggctataacgtgagcaggagcaccacagaggacttcccactgaggctgctgtccgccgcaccttcccagaccagcgtgtacttctgcgcctctcggaacctgggcgatacacagtattttggacctggaaccaggctgacagtgctg

SEQ ID NO: 84 - protein, 111 aa, Homo sapiens
ESVGLHLPTLSVQEGDNSIINCAYSNSASDYFIWYKQESGKGPQFIIDIRSNMDKRQGQRVTVLLNKTVKHLSLQIAATQPGDSAVYFCAEYIQGAQKLVFGQGTRLTINP

SEQ ID NO: 85 - protein, 109 aa, Homo sapiens
GVTQTPRHLVMGMTNKKSLKCEQHLGHNAMYWYKQSAKKPLELMFVYSLEERVENNSVPSRFSPECPNSSHLFLHLHTLQPEDSALYLCASSQEDNEQFFGPGTRLTVL

SEQ ID NO: 86 - protein, 113 aa, Homo sapiens
QRKEVEQDPGPFNVPEGATVAFNCTYSNSASQSFFWYRQDCRKEPKLLMSVYSSGNEDGRFTAHLNRASQYISLLIRDSKLSDSATYLCAVNPKYTGGFKTIFGAGTRLFVKA

SEQ ID NO: 87 - protein, 114 aa, Homo sapiens
GVTQTPRHLVMGMTNKKSLKCEQHLGHNAMYWYKQSAKKPLELMFVYSLEERVENNSVPSRFSPECPNSSHLFLHLHTLQPEDSALYLCASSQDVTSEWVDTIYFGEGSWLTVV

SEQ ID NO: 88 - protein, 112 aa, Homo sapiens
QRKEVEQDPGPFNVPEGATVAFNCTYSNSASQSFFWYRQDCRKEPKLLMSVYSSGNEDGRFTAQLNRASQYISLLIRDSKLSDSATYLCVVDDQTGANNLFFGTGTRLTVIP

SEQ ID NO: 89 - protein, 109 aa, Homo sapiens
GVTQTPKFQVLKTGQSMTLQCAQDMNHEYMSWYRQDPGMGLRLIHYSVGAGITDQGEVPNGYNVSRSTTEDFPLRLLSAAPSQTSVYFCASRNLGDTQYFGPGTRLTVL

SEQ ID NO: 90 - protein, 132 aa, Homo sapiens; identical to SEQ ID NO: 7

SEQ ID NO: 91 - protein, 133 aa, Homo sapiens
MGCRLLCCAVLCLLGAGELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQEDNEQFFGPGTRLTVL

SEQ ID NO: 92 - protein, 133 aa, Artificial Sequence
  Synthetic
MASLRVLLVILWLQLSWVWSQRKEVEQDPGPFNVPEGATVAFNCTYSNSASQSFFWYRQDCRKEPKLLMSVYSSGNEDGRFTAHLNRASQYISLLIRDSKLSDSATYLCAVNPKYTGGFKTIFGAGTRLFVKA

SEQ ID NO: 93 - protein, 138 aa, Homo sapiens
MGCRLLCCAVLCLLGAGELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQDVTSEWVDTIYFGEGSWLTVV

SEQ ID NO: 94 - protein, 111 aa, Homo sapiens; identical to SEQ ID NO: 84

SEQ ID NO: 95 - protein, 116 aa, Homo sapiens
ELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQEDNEQFFGPGTRLTVL

SEQ ID NO: 96 - protein, 113 aa, Homo sapiens; identical to SEQ ID NO: 86

SEQ ID NO: 97 - protein, 121 aa, Homo sapiens
ELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQDVTSEWVDTIYFGEGSWLTVV

SEQ ID NO: 98 - protein, 137 aa, Artificial Sequence
  Synthetic
NIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 99 - protein, 173 aa, Artificial Sequence
  Synthetic
EDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 100 - protein, 269 aa, Artificial Sequence; identical to SEQ ID NO: 21
  Synthetic
  MISC_FEATURE (180)..(180): Xaa is Thr or Cys
  MISC_FEATURE (244)..(244): Xaa is Ser, Ala, Val, Leu, Ile, Pro, Phe, Met, or Trp
  MISC_FEATURE (246)..(246): Xaa is Met, Ala, Val, Leu, Ile, Pro, Phe, or Trp
  MISC_FEATURE (247)..(247): Xaa is Gly, Ala, Val, Leu, Ile, Pro, Phe, Met, or Trp

SEQ ID NO: 101 - protein, 306 aa, Artificial Sequence
  Synthetic
  MISC_FEATURE (190)..(190): Xaa is Ser or Cys
MGCRLLCCAVLCLLGAGELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQEDNEQFFGPGTRLTVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 102 - protein, 269 aa, Artificial Sequence; identical to SEQ ID NO: 23
  Synthetic

SEQ ID NO: 103 - protein, 306 aa, Artificial Sequence
  Synthetic
MGCRLLCCAVLCLLGAGELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQEDNEQFFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 104 - protein, 269 aa, Artificial Sequence
  Synthetic
MAGIRALFMYLWLQLDWVSRGESVGLHLPTLSVQEGDNSIINCAYSNSASDYFIWYKQESGKGPQFIIDIRSNMDKRQGQRVTVLLNKTVKHLSLQIAATQPGDSAVYFCAEYIQGAQKLVFGQGTRLTINPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 105 - protein, 306 aa, Artificial Sequence
  Synthetic
MGCRLLCCAVLCLLGAGELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQEDNEQFFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 106 - protein, 270 aa, Artificial Sequence
  Synthetic
  MISC_FEATURE (181)..(181): Xaa is Thr or Cys
  MISC_FEATURE (245)..(245): Xaa is Ser, Ala, Val, Leu, Ile, Pro, Phe, Met, or Trp
  MISC_FEATURE (247)..(247): Xaa is Met, Ala, Val, Leu, Ile, Pro, Phe, or Trp
  MISC_FEATURE (248)..(248): Xaa is Gly, Ala, Val, Leu, Ile, Pro, Phe, Met, or Trp
MASLRVLLVILWLQLSWVWSQRKEVEQDPGPFNVPEGATVAFNCTYSNSASQSFFWYRQDCRKEPKLLMSVYSSGNEDGRFTAHLNRASQYISLLIRDSKLSDSATYLCAVNPKYTGGFKTIFGAGTRLFVKANIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 107 - protein, 311 aa, Artificial Sequence
  Synthetic
  MISC_FEATURE (195)..(195): Xaa is Ser or Cys
MGCRLLCCAVLCLLGAGELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQDVTSEWVDTIYFGEGSWLTVVEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 108 - protein, 270 aa, Artificial Sequence
  Synthetic
MASLRVLLVILWLQLSWVWSQRKEVEQDPGPFNVPEGATVAFNCTYSNSASQSFFWYRQDCRKEPKLLMSVYSSGNEDGRFTAHLNRASQYISLLIRDSKLSDSATYLCAVNPKYTGGFKTIFGAGTRLFVKANIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 109 - protein, 311 aa, Artificial Sequence
  Synthetic
MGCRLLCCAVLCLLGAGELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQDVTSEWVDTIYFGEGSWLTVVEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 110 - protein, 270 aa, Artificial Sequence
  Synthetic
MASLRVLLVILWLQLSWVWSQRKEVEQDPGPFNVPEGATVAFNCTYSNSASQSFFWYRQDCRKEPKLLMSVYSSGNEDGRFTAHLNRASQYISLLIRDSKLSDSATYLCAVNPKYTGGFKTIFGAGTRLFVKANIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 111 - protein, 311 aa, Artificial Sequence
  Synthetic
MGCRLLCCAVLCLLGAGELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQDVTSEWVDTIYFGEGSWLTVVEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 112 - protein, 248 aa, Artificial Sequence
  Synthetic
  MISC_FEATURE (159)..(159): Xaa is Thr or Cys
  MISC_FEATURE (223)..(223): Xaa is Ser, Ala, Val, Leu, Ile, Pro, Phe, Met, or Trp
  MISC_FEATURE (225)..(225): Xaa is Met, Ala, Val, Leu, Ile, Pro, Phe, or Trp
  MISC_FEATURE (226)..(226): Xaa is Gly, Ala, Val, Leu, Ile, Pro, Phe, Met, or Trp
ESVGLHLPTLSVQEGDNSIINCAYSNSASDYFIWYKQESGKGPQFIIDIRSNMDKRQGQRVTVLLNKTVKHLSLQIAATQPGDSAVYFCAEYIQGAQKLVFGQGTRLTINPNIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 113 - protein, 289 aa, Artificial Sequence
  Synthetic
  MISC_FEATURE (173)..(173): Xaa is Ser or Cys
ELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQEDNEQFFGPGTRLTVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 114 - protein, 248 aa, Artificial Sequence
  Synthetic
ESVGLHLPTLSVQEGDNSIINCAYSNSASDYFIWYKQESGKGPQFIIDIRSNMDKRQGQRVTVLLNKTVKHLSLQIAATQPGDSAVYFCAEYIQGAQKLVFGQGTRLTINPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 115 - protein, 289 aa, Artificial Sequence
  Synthetic
ELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQEDNEQFFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 116 - protein, 248 aa, Artificial Sequence
  Synthetic
ESVGLHLPTLSVQEGDNSIINCAYSNSASDYFIWYKQESGKGPQFIIDIRSNMDKRQGQRVTVLLNKTVKHLSLQIAATQPGDSAVYFCAEYIQGAQKLVFGQGTRLTINPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 117 - protein, 289 aa, Artificial Sequence
  Synthetic
ELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQEDNEQFFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 118 - protein, 250 aa, Artificial Sequence
  Synthetic
  MISC_FEATURE (161)..(161): Xaa is Thr or Cys
  MISC_FEATURE (225)..(225): Xaa is Ser, Ala, Val, Leu, Ile, Pro, Phe, Met, or Trp
  MISC_FEATURE (227)..(227): Xaa is Met, Ala, Val, Leu, Ile, Pro, Phe, or Trp
  MISC_FEATURE (228)..(228): Xaa is Gly, Ala, Val, Leu, Ile, Pro, Phe, Met, or Trp
QRKEVEQDPGPFNVPEGATVAFNCTYSNSASQSFFWYRQDCRKEPKLLMSVYSSGNEDGRFTAHLNRASQYISLLIRDSKLSDSATYLCAVNPKYTGGFKTIFGAGTRLFVKANIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 119 - protein, 294 aa, Artificial Sequence
  Synthetic
  MISC_FEATURE (178)..(178): Xaa is Ser or Cys
ELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQDVTSEWVDTIYFGEGSWLTVVEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 120 - protein, 250 aa, Artificial Sequence
  Synthetic
QRKEVEQDPGPFNVPEGATVAFNCTYSNSASQSFFWYRQDCRKEPKLLMSVYSSGNEDGRFTAHLNRASQYISLLIRDSKLSDSATYLCAVNPKYTGGFKTIFGAGTRLFVKANIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 121 - protein, 294 aa, Artificial Sequence
  Synthetic
ELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQDVTSEWVDTIYFGEGSWLTVVEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 122 - protein, 250 aa, Artificial Sequence
  Synthetic
QRKEVEQDPGPFNVPEGATVAFNCTYSNSASQSFFWYRQDCRKEPKLLMSVYSSGNEDGRFTAHLNRASQYISLLIRDSKLSDSATYLCAVNPKYTGGFKTIFGAGTRLFVKANIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 123 - protein, 294 aa, Artificial Sequence
  Synthetic
ELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQDVTSEWVDTIYFGEGSWLTVVEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 124 - protein, 269 aa, Artificial Sequence; identical to SEQ ID NO: 104
  Synthetic

SEQ ID NO: 125 - protein, 306 aa, Artificial Sequence
  Synthetic
MACRLLCCAVLCLLGAGELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQEDNEQFFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 126 - protein, 270 aa, Artificial Sequence
  Synthetic
MISLRVLLVILWLQLSWVWSQRKEVEQDPGPFNVPEGATVAFNCTYSNSASQSFFWYRQDCRKEPKLLMSVYSSGNEDGRFTAHLNRASQYISLLIRDSKLSDSATYLCAVNPKYTGGFKTIFGAGTRLFVKANIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 127 - protein, 311 aa, Artificial Sequence
  Synthetic
MACRLLCCAVLCLLGAGELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQDVTSEWVDTIYFGEGSWLTVVEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 128 - protein, 249 aa, Artificial Sequence
  Synthetic
GESVGLHLPTLSVQEGDNSIINCAYSNSASDYFIWYKQESGKGPQFIIDIRSNMDKRQGQRVTVLLNKTVKHLSLQIAATQPGDSAVYFCAEYIQGAQKLVFGQGTRLTINPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 129 - protein, 284 aa, Artificial Sequence
  Synthetic
ETGVTQTPRHLVMGMTNKKSLKCEQHLGHNAMYWYKQSAKKPLELMFVYSLEERVENNSVPSRFSPECPNSSHLFLHLHTLQPEDSALYLCASSQEDNEQFFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 130 - protein, 249 aa, Artificial Sequence
  Synthetic
RKEVEQDPGPFNVPEGATVAFNCTYSNSASQSFFWYRQDCRKEPKLLMSVYSSGNEDGRFTAHLNRASQYISLLIRDSKLSDSATYLCAVNPKYTGGFKTIFGAGTRLFVKANIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 131 - protein, 289 aa, Artificial Sequence
  Synthetic
ETGVTQTPRHLVMGMTNKKSLKCEQHLGHNAMYWYKQSAKKPLELMFVYSLEERVENNSVPSRFSPECPNSSHLFLHLHTLQPEDSALYLCASSQDVTSEWVDTIYFGEGSWLTVVEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 132 - protein, 132 aa, Artificial Sequence
  Synthetic
MASLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVDDQTGANNLFFGTGTRLTVIP

SEQ ID NO: 133 - protein, 130 aa, Homo sapiens
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASRNLGDTQYFGPGTRLTVL

SEQ ID NO: 134 - protein, 269 aa, Artificial Sequence
  Synthetic
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVDDQTGANNLFFGTGTRLTVIPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 135 - protein, 303 aa, Artificial Sequence
  Synthetic
MAIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASRNLGDTQYFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 136 - protein, 269 aa, Artificial Sequence
  Synthetic
  MISC_FEATURE (180)..(180): Xaa is Thr or Cys
  MISC_FEATURE (244)..(244): Xaa is Ser, Ala, Val, Leu, Ile, Pro, Phe, Met, or Trp
  MISC_FEATURE (246)..(246): Xaa is Met, Ala, Val, Leu, Ile, Pro, Phe, or Trp
  MISC_FEATURE (247)..(247): Xaa is Gly, Ala, Val, Leu, Ile, Pro, Phe, Met, or Trp
MASLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVDDQTGANNLFFGTGTRLTVIPNIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 137 - protein, 303 aa, Artificial Sequence
  Synthetic
  MISC_FEATURE (187)..(187): Xaa is Ser or Cys
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASRNLGDTQYFGPGTRLTVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 138 - protein, 269 aa, Artificial Sequence
  Synthetic
MASLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVDDQTGANNLFFGTGTRLTVIPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 139 - protein, 303 aa, Artificial Sequence
  Synthetic
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASRNLGDTQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 140 - protein, 269 aa, Artificial Sequence
  Synthetic
MASLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVDDQTGANNLFFGTGTRLTVIPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 141 - protein, 303 aa, Artificial Sequence
  Synthetic
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASRNLGDTQYFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 142 - protein, 248 aa, Artificial Sequence
  Synthetic
RKEVEQDPGPFNVPEGATVAFNCTYSNSASQSFFWYRQDCRKEPKLLMSVYSSGNEDGRFTAQLNRASQYISLLIRDSKLSDSATYLCVVDDQTGANNLFFGTGTRLTVIPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 143 - protein, 284 aa, Artificial Sequence
  Synthetic
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASRNLGDTQYFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 144 - protein, 249 aa, Artificial Sequence
  Synthetic
  MISC_FEATURE (160)..(160): Xaa is Thr or Cys
  MISC_FEATURE (224)..(224): Xaa is Ser, Ala, Val, Leu, Ile, Pro, Phe, Met, or Trp
  MISC_FEATURE (226)..(226): Xaa is Met, Ala, Val, Leu, Ile, Pro, Phe, or Trp
  MISC_FEATURE (227)..(227): Xaa is Gly, Ala, Val, Leu, Ile, Pro, Phe, Met, or Trp
QRKEVEQDPGPFNVPEGATVAFNCTYSNSASQSFFWYRQDCRKEPKLLMSVYSSGNEDGRFTAQLNRASQYISLLIRDSKLSDSATYLCVVDDQTGANNLFFGTGTRLTVIPNIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 145 - protein, 282 aa, Artificial Sequence
  Synthetic
  MISC_FEATURE (166)..(166): Xaa is Ser or Cys
GVTQTPKFQVLKTGQSMTLQCAQDMNHEYMSWYRQDPGMGLRLIHYSVGAGITDQGEVPNGYNVSRSTTEDFPLRLLSAAPSQTSVYFCASRNLGDTQYFGPGTRLTVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 146 - protein, 249 aa, Artificial Sequence
  Synthetic
QRKEVEQDPGPFNVPEGATVAFNCTYSNSASQSFFWYRQDCRKEPKLLMSVYSSGNEDGRFTAQLNRASQYISLLIRDSKLSDSATYLCVVDDQTGANNLFFGTGTRLTVIPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 147 - protein, 282 aa, Artificial Sequence
  Synthetic
GVTQTPKFQVLKTGQSMTLQCAQDMNHEYMSWYRQDPGMGLRLIHYSVGAGITDQGEVPNGYNVSRSTTEDFPLRLLSAAPSQTSVYFCASRNLGDTQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 148 - protein, 249 aa, Artificial Sequence
  Synthetic
QRKEVEQDPGPFNVPEGATVAFNCTYSNSASQSFFWYRQDCRKEPKLLMSVYSSGNEDGRFTAQLNRASQYISLLIRDSKLSDSATYLCVVDDQTGANNLFFGTGTRLTVIPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 149 - protein, 282 aa, Artificial Sequence
  Synthetic
GVTQTPKFQVLKTGQSMTLQCAQDMNHEYMSWYRQDPGMGLRLIHYSVGAGITDQGEVPNGYNVSRSTTEDFPLRLLSAAPSQTSVYFCASRNLGDTQYFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 150 - protein, 248 aa, Artificial Sequence; identical to SEQ ID NO: 112
  Synthetic
  MISC_FEATURE (159)..(159): Xaa is Thr or Cys
  MISC_FEATURE (223)..(223): Xaa is Ser, Ala, Val, Leu, Ile, Pro, Phe, Met, or Trp
  MISC_FEATURE (225)..(225): Xaa is Met, Ala, Val, Leu, Ile, Pro, Phe, or Trp
  MISC_FEATURE (226)..(226): Xaa is Gly, Ala, Val, Leu, Ile, Pro, Phe, Met, or Trp

SEQ ID NO: 151 - protein, 282 aa, Artificial Sequence
  Synthetic
  MISC_FEATURE (166)..(166): Xaa is Ser or Cys
GVTQTPRHLVMGMTNKKSLKCEQHLGHNAMYWYKQSAKKPLELMFVYSLEERVENNSVPSRFSPECPNSSHLFLHLHTLQPEDSALYLCASSQEDNEQFFGPGTRLTVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 152 - protein, 248 aa, Artificial Sequence; identical to SEQ ID NO: 114
  Synthetic

SEQ ID NO: 153 - protein, 282 aa, Artificial Sequence
  Synthetic
GVTQTPRHLVMGMTNKKSLKCEQHLGHNAMYWYKQSAKKPLELMFVYSLEERVENNSVPSRFSPECPNSSHLFLHLHTLQPEDSALYLCASSQEDNEQFFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 154 - protein, 248 aa, Artificial Sequence; identical to SEQ ID NO: 116
  Synthetic

SEQ ID NO: 155 - protein, 282 aa, Artificial Sequence
  Synthetic
GVTQTPRHLVMGMTNKKSLKCEQHLGHNAMYWYKQSAKKPLELMFVYSLEERVENNSVPSRFSPECPNSSHLFLHLHTLQPEDSALYLCASSQEDNEQFFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 156 - protein, 250 aa, Artificial Sequence; identical to SEQ ID NO: 118
  Synthetic
  MISC_FEATURE (161)..(161): Xaa is Thr or Cys
  MISC_FEATURE (225)..(225): Xaa is Ser, Ala, Val, Leu, Ile, Pro, Phe, Met, or Trp
  MISC_FEATURE (227)..(227): Xaa is Met, Ala, Val, Leu, Ile, Pro, Phe, or Trp
  MISC_FEATURE (228)..(228): Xaa is Gly, Ala, Val, Leu, Ile, Pro, Phe, Met, or Trp

SEQ ID NO: 157 - protein, 287 aa, Artificial Sequence
  Synthetic
  MISC_FEATURE (171)..(171): Xaa is Ser or Cys
GVTQTPRHLVMGMTNKKSLKCEQHLGHNAMYWYKQSAKKPLELMFVYSLEERVENNSVPSRFSPECPNSSHLFLHLHTLQPEDSALYLCASSQDVTSEWVDTIYFGEGSWLTVVEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 158 - protein, 250 aa, Artificial Sequence; identical to SEQ ID NO: 120
  Synthetic

SEQ ID NO: 159 - protein, 287 aa, Artificial Sequence
  Synthetic
GVTQTPRHLVMGMTNKKSLKCEQHLGHNAMYWYKQSAKKPLELMFVYSLEERVENNSVPSRFSPECPNSSHLFLHLHTLQPEDSALYLCASSQDVTSEWVDTIYFGEGSWLTVVEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 160 - protein, 250 aa, Artificial Sequence; identical to SEQ ID NO: 122
  Synthetic

SEQ ID NO: 161 - protein, 287 aa, Artificial Sequence
  Synthetic
GVTQTPRHLVMGMTNKKSLKCEQHLGHNAMYWYKQSAKKPLELMFVYSLEERVENNSVPSRFSPECPNSSHLFLHLHTLQPEDSALYLCASSQDVTSEWVDTIYFGEGSWLTVVEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 162 - protein, 602 aa, Artificial Sequence
  Synthetic
MACRLLCCAVLCLLGAGELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQEDNEQFFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMAGIRALFMYLWLQLDWVSRGESVGLHLPTLSVQEGDNSIINCAYSNSASDYFIWYKQESGKGPQFIIDIRSNMDKRQGQRVTVLLNKTVKHLSLQIAATQPGDSAVYFCAEYIQGAQKLVFGQGTRLTINPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 163 - protein, 602 aa, Artificial Sequence
  Synthetic
MAGIRALFMYLWLQLDWVSRGESVGLHLPTLSVQEGDNSIINCAYSNSASDYFIWYKQESGKGPQFIIDIRSNMDKRQGQRVTVLLNKTVKHLSLQIAATQPGDSAVYFCAEYIQGAQKLVFGQGTRLTINPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSRAKRSGSGATNFSLLKQAGDVEENPGPMGCRLLCCAVLCLLGAGELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQEDNEQFFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 164 - protein, 608 aa, Artificial Sequence
  Synthetic
MACRLLCCAVLCLLGAGELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQDVTSEWVDTIYFGEGSWLTVVEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMISLRVLLVILWLQLSWVWSQRKEVEQDPGPFNVPEGATVAFNCTYSNSASQSFFWYRQDCRKEPKLLMSVYSSGNEDGRFTAHLNRASQYISLLIRDSKLSDSATYLCAVNPKYTGGFKTIFGAGTRLFVKANIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 165 - protein, 608 aa, Artificial Sequence
  Synthetic
MASLRVLLVILWLQLSWVWSQRKEVEQDPGPFNVPEGATVAFNCTYSNSASQSFFWYRQDCRKEPKLLMSVYSSGNEDGRFTAHLNRASQYISLLIRDSKLSDSATYLCAVNPKYTGGFKTIFGAGTRLFVKANIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSRAKRSGSGATNFSLLKQAGDVEENPGPMGCRLLCCAVLCLLGAGELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQDVTSEWVDTIYFGEGSWLTVVEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 166 - protein, 599 aa, Artificial Sequence
  Synthetic
MAIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASRNLGDTQYFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVDDQTGANNLFFGTGTRLTVIPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 167 - protein, 599 aa, Artificial Sequence
  Synthetic
MASLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVDDQTGANNLFFGTGTRLTVIPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSRAKRSGSGATNFSLLKQAGDVEENPGPMSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASRNLGDTQYFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

(19) United States
(12) Patent Application Publication
(10) Pub. No.: US 2023/0082787 A1
(43) Pub. Date: Mar. 16, 2023

(54) HLA CLASS I-RESTRICTED T CELL RECEPTORS AGAINST RAS WITH G12V MUTATION

(71) Applicant: The United States of America,as represented by the Secretary,Department of Health and Human Services, Bethesda, MD (US)

(72) Inventors: Noam Levin, Rockville, MD (US); Maria R. Parkhurst, Ellicott City, MD (US); Frank J. Lowery, III, Clarksburg, MD (US); Steven A. Rosenberg, Potomac, MD (US)

(73) Assignee: The United States of America,as represented by the Secretary,Department of Health and Human Services, Bethesda, MD (US)

(21) Appl. No.: 17/799,193

(22) PCT Filed: Feb. 12, 2021
(86) PCT No.: PCT/US2021/017852

§ 371 Date: Aug. 11, 2022

Related U.S. Application Data
(60) Provisional application No. 63/060,340, filed on Aug. 3, 2020.
(60) Provisional application No. 62/976,655, filed on Feb. 14, 2020.

(51) Int. Cl. C07K 14/725; A61P 35/00; A61P 37/04; A61K 35/17; A61P 1/00

(52) U.S. Cl. CPC C07K 14/7051; A61P 35/00; A61P 37/04; A61K 35/17; A61P 1/00; A61K 38/00

(57) ABSTRACT

Disclosed are isolated or purified T cell receptors (TCRs), wherein the TCRs have antigenic specificity for a mutated RAS amino acid sequence presented by a human leukocyte antigen (HLA) Class I molecule. Related polypeptides and proteins, as well as related nucleic acids, recombinant expression vectors, host cells, populations of cells, and pharmaceutical compositions are also provided. Also disclosed are methods of detecting the presence of cancer in a mammal and methods of treating or preventing cancer in a mammal

### CROSS REFERENCE TO RELATED APPLICATIONS

This patent application claims the benefit of U.S. Provisional Patent Application No. 62/976,655, filed Feb. 14, 2020, and U.S. Provisional Patent Application No. 63/060,340, filed Aug. 3, 2020, each of which is incorporated by reference in its entirety herein.

### STATEMENT REGARDING FEDERALLY SPONSORED RESEARCH OR DEVELOPMENT

This invention was made with Government support under project number ZIABC010984 by the National Institutes of Health, National Cancer Institute. The Government has certain rights in the invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 307,206 Byte ASCII (Text) file named “751508 ST25.txt” dated Feb. 4, 2021.

### BACKGROUND OF THE INVENTION

Some cancers may have very limited treatment options, particularly when the cancer becomes metastatic and unresectable. Despite advances in treatments such as, for example, surgery, chemotherapy, and radiation therapy, the prognosis for many cancers, such as, for example, pancreatic, colorectal, lung, endometrial, ovarian, and prostate cancers, may be poor. Accordingly, there exists an unmet need for additional treatments for cancer.

### BRIEF SUMMARY OF THE INVENTION

An embodiment of the invention provides an isolated or purified T-cell receptor (TCR) comprising the amino acid sequences of (a) SEQ ID NOs: 1-3, (b) SEQ ID NOs: 4-6, (c) SEQ ID NOs: 31-33, (d) SEQ ID NOs: 34-36, (e) SEQ ID NOs: 64-66, (f) SEQ ID NOs: 67-69, (g) SEQ ID NOs: 1-6, (h) SEQ ID NOs: 31-36, or (i) SEQ ID NOs: 64-69 wherein the TCR has antigenic specificity for a mutated human RAS amino acid sequence with a substitution of glycine at position 12 with valine, presented by a human leukocyte antigen (HLA) Class I molecule, and wherein the mutated human RAS amino acid sequence is a mutated human Kirsten rat sarcoma viral oncogene homolog (KRAS), a mutated human Harvey rat sarcoma viral oncogene homolog (HRAS), or a mutated human Neuroblastoma rat sarcoma viral oncogene homolog (NRAS) amino acid sequence, and wherein position 12 is defined by reference to the wild-type human KRAS, wild-type human HRAS, or wild-type human NRAS protein, respectively.

Another embodiment of the invention provides an isolated or purified polypeptide comprising a functional portion of the inventive TCR, wherein the functional portion comprises the amino acid sequences of: (a) all of SEQ ID NOs: 1-3, (b) all of SEQ ID NOs: 4-6, (c) all of SEQ ID NOs: 31-33, (d) all of SEQ ID NOs: 34-36, (e) all of SEQ ID NOs: 64-66, (0 all of SEQ ID NOs: 67-69, (g) all of SEQ ID NOs: 1-6, (h) all of SEQ ID NOs: 31-36, or (i) all of SEQ ID NOs: 64-69.

Still another embodiment of the invention provides an isolated or purified protein comprising at least one of the inventive polypeptides.

Further embodiments of the invention provide nucleic acids, recombinant expression vectors, host cells, populations of cells, and pharmaceutical compositions relating to the inventive TCRs, polypeptides, and proteins.

An embodiment of the invention provides an isolated or purified nucleic acid comprising, from 5′ to 3′, a first nucleic acid sequence and a second nucleotide sequence, wherein the first and second nucleotide sequence, respectively, encode the amino sequences of SEQ ID NOs: 7 and 8; 7 and 91; 90 and 8; 90 and 91; 8 and 7; 91 and 7; 8 and 90; 91 and 90; 37 and 38; 37 and 93; 92 and 38; 92 and 93; 38 and 37; 93 and 37; 38 and 92; 93 and 92; 70 and 71; 70 and 133; 132 and 71; 132 and 133; 71 and 70; 133 and 70; 71 and 132; 133 and 132; 23 and 24; 23 and 103; 102 and 24; 102 and 103; 24 and 23; 103 and 23; 24 and 102; 103 and 102; 39 and 40; 39 and 109; 108 and 40; 108 and 109; 40 and 39; 109 and 39; 40 and 108; 109 and 108; 78 and 79; 78 and 139; 138 and 79; 138 and 139; 79 and 78; 139 and 78; 79 and 138; 139 and 138; 21 and 22; 21 and 101; 100 and 22; 100 and 101; 22 and 21; 101 and 21; 22 and 100; 101 and 100; 41 and 42; 41 and 107; 106 and 42; 106 and 107; 42 and 41; 107 and 41; 42 and 106; 107 and 106; 74 and 75; 74 and 101; 100 and 75, 100 and 101; 75 and 74; 101 and 74; 75 and 100; 101 and 100; 124 and 125; 124 and 105; 104 and 125; 104 and 105; 125 and 124; 105 and 124; 125 and 104; 105 and 104; 126 and 127; 126 and 111; 110 and 127; 110 and 111; 127 and 126; 111 and 126; 127 and 110; 111 and 110; 134 and 135; 140 and 135; 134 and 141; 140 and 141; 135 and 134; 135 and 140; 141 and 134; 141 and 140; 47 and 48; 48 and 47; 49 and 50; 50 and 49; 72 and 73; 73 and 72; 94 and 95; 95 and 94; 94 and 85; 85 and 94; 96 and 97; 97 and 96; 96 and 87; 87 and 96; 88 and 89; 89 and 88; 51 and 52; 52 and 51; 53 and 54; 54 and 53; 80 and 81; 81 and 80; 55 and 56; 56 and 55; 57 and 58; 58 and 57; 76 and 77; 77 and 76; 128 and 129; 129 and 128; 130 and 131; 131 and 130; 142 and 143; 143 and 142; 112 and 113; 113 and 112; 118 and 119; 119 and 118; 144 and 145; 145 and 144; 114 and 115; 115 and 114; 120 and 121; 121 and 120; 146 and 147; 147 and 146; 116 and 117; 117 and 116; 122 and 123; 123 and 122; 148 and 149; 149 and 148; 150 and 151; 151 and 150; 154 and 155; 155 and 154; 152 and 153; 153 and 152; 156 and 157; 157 and 156; 160 and 161; 161 and 160; 158 and 159; or 159 and 158.

Methods of detecting the presence of cancer in a mammal, methods of treating or preventing cancer in a mammal, methods of inducing an immune response against a cancer in a mammal, methods of producing a host cell expressing a TCR that has antigenic specificity for the peptide of SEQ ID NO: 29 or 30, and methods of producing the inventive TCRs, polypeptides, and proteins, are further provided by embodiments of the invention.

Additional embodiments are as described herein.

### BRIEF DESCRIPTION OF THE DRAWINGS

FIGS. 1A-1D show the reactivity of tumor infiltrating lymphocytes (TIL) to autologous DC target cells transfected with a tandem minigene (TMG) mRNA encoding wild-type (WT) RAS (FIG. 1A), with an mRNA TMG encoding mutated (Mut) RAS (FIG. 1B), pulsed with a WT RAS long peptide (LP) (FIG. 1C), or pulsed with a RAS

G12V LP (FIG. 1D). The TIL were isolated from tumor fragments Fl, F13, and or a combination of tumor fragments F2, F3, and F5 from Patient 4391. The TIL were gated on live/CD3+/CD8+. Reactivity was measured by detecting upregulation of OX40 and 4-1BB by FACS.

FIGS. 2A and 2B shows the reactivity of TIL from tumor fragment F1 from Patient 4391 to target cells pulsed with one of RAS G12V minimal epitopes — ME 4-7 (FIG. 2A), WT LP, or RAS G12V LP (FIG. 2B). Target cells pulsed with DMSO served as a control (FIG. 2B). The target cells were 4391 autologous DC, COS-A03 (COS7 cells stably express HLA-A*03:01), or COS-A02 (COS7 cells stably express HLA-A*02:01) cell lines. The TIL were gated on live/CD3+/CD8+. Reactivity was measured by detecting upregulation of OX40 and 4-1BB by FACS.

FIG. 3 is a graph showing ELISPOT measurement of IFN-γ secretion (spots/3e4 cells) by TIL from tumor fragment F1 of Patient 4391 in response to co-culture with autologous DC pulsed with WT LP, G12V LP, or G12V ME. TIL cultured alone served as a negative control. TIL non-specifically stimulated by co-culture with anti-CD3/anti-CD28 Dynabeads material served as a positive control.

FIGS. 4A-4D are graphs showing ELISPOT measurement of IFN-γ secreted (spots/3e4 cells) by TIL from tumor fragment F1 of Patient 4391 in response to co-culture with target COS7 cells which were not DNA transfected (COS7) or DNA transfected with one of four different HLA alleles expressed by Patient 4391 (HLA-A*03:01, HLA-A*11:01, HLA-B*55:01, or HLA-C*01:02). The target cells were pulsed with the indicated concentrations of WT LP (FIG. 4C), G12V LP (FIG. 4B), or G12V ME8 (FIG. 4A). COS7 cells DNA transfected with HLA-C*01:02, pulsed with the indicated concentrations of G12V ME8, G12V LP, or WT LP (FIG. 4D).

FIGS. 5A-5C are graphs showing ELISPOT measurement of IFN-γ secreted (spots/3e4 cells) (FIG. 5A) or the percentage of 4-1BB/OX40+ cells (FIGS. 5B and 5C) in response to co-culture of PBL transduced with the TCR with target cells pulsed with the indicated concentrations of G12V ME8 or RAS ME WT4 (WT sequence of ME8). Transduced cells co-cultured with anti-CD3/anti-CD28 Dynabeads material served as a positive control. CD8+ gated cells are shown in FIG. 5B. CD4+ gated cells are shown in FIG. 5C.

FIG. 6 presents ELISPOT measurement of IFN-γ secreted by 4385 TIL fragment 11 screened for new-antigen reactivity against different peptides pools (PP) or different TMGs, PMA/Io material served as a positive control.

FIGS. 7A-7C are graphs showing the percentage of 4-1BB/OX40+ cells in CD8+ gated cells (FIG. 7A) and CD4+ gated cells (7 FIG. B) or ELISPOT measurement of IFN-γ secreted (spots/3e4 cells) (FIG. 7C) in response to co-culture of 4385 PBL transduced with TCR 4 or TIL fragment 11 (F11) with 4385 autologous DC target cells pulsed with one of RAS minimal epitopes (ME) 4-8, RAS WT LP, RAS G12V LP or with DC mRNA transfected with RAS WT FL, G12V FL or, TMG2 (the TMG from FIG. 6 and containing RAS G12V). Target cells pulsed with DMSO or T cell only served as a negative control. T cell co-culture with anti-CD3/anti-CD28 Dynabeads material served as a positive control. Labels are used to distinguish markers, where appropriate.

FIG. 8A presents dot plots showing TCR transduction efficacy into 4385 PBLs and the CD8/CD4 population distribution of the cells used in this experiment. FIG. 8B presents the IFN-γ ELISPOT picture.

FIGS. 9A-9C are graphs showing ELISPOT measurement of IFN-γ secreted (spots/3e4 cells) (FIG. 9A) or the percentage of 4-1BB/OX40+ cells in CD8+ gated cells (FIG. 9B) or CD4+ gated cells (FIG. 9C) as a response to co-culture of 4385 TIL Fll with autologous DC target cells pulsed with the indicated concentrations of RAS WT LP, RAS G12V LP or, RAS G12V ME8. TIL F11 cells co-cultured with anti-CD3/anti-CD28 Dynabeads material served as a positive control. T cell only (without target cells) served as a negative control.

FIGS. 10A-10C are graphs showing ELISPOT measurement of IFN-γ secreted (spots/3e4 cells) (FIG. 10A) or the percentage of 4-1BB/OX40+ cells in CD8+ gated cells (FIG. 10B) or CD4+ gated cells (FIG. 10C) as a response to co-culture of PBL transduced with 4385 anti-RAS TCR with autologous DC target cells pulsed with the indicated concentrations of RAS WT LP, RAS G12V LP, or RAS G12V ME8. Transduced cells co-cultured with anti-CD3/anti-CD28 Dynabeads material served as a positive control. T cell only (without target cells) served as a negative control.

FIGS. 11A-11C are graphs showing ELISPOT measurement of IFN-γ secreted (spots/3e4 cells) (FIG. 11A) or the percentage of 4-1BB/OX40+ cells in CD8+ gated cells (FIG. 11B) or CD4+ gated cells (FIG. 11C) as a response to co-culture of PBL transduced with 4385 anti-RAS G12V TCR4 with autologous DC target cells pulsed with the indicated concentrations of RAS G12V ME8 or RAS G12V ME WT4 (WT sequence of ME8). Transduced cells co-cultured with anti-CD3/anti-CD28 Dynabeads material served as a positive control.

FIGS. 12A-12C are graphs showing reactivity tested by IFNγ ELISPOT (FIG. 12A) and using 41BB/OX40 flow cytometry assay gated to CD8 (FIG. 12B) and CD4 (FIG. 12C) of TIL fragments co-culture with autologous DC transfected with RAS G12V FL, RAS WT FL, loaded with RAS WT LP, RAS G12V LP, RAS G12V MEs (without ME8) or ME8. DC treated with DMSO or anti-CD3/anti-CD28 Dynabeads served as a negative or positive control (respectively).

FIGS. 13A-13B are graphs showing reactivity tested using IFNγ ELISPOT (FIG. 13A) and 41BB/OX40 flow cytometry assay (FIG. 13B) gated to CD8+ after sort enrichment. TILs from tumor fragment F12 of Patient 4394 were co-cultured with autologous DC loaded with RAS G12V ME8 or RAS ME WT4 (WT sequence of ME 8) in different concentrations, after enrichment of the RAS reactive TIL population.

FIGS. 14A-14C are graphs showing reactivity tested by IFNγ ELISPOT (FIG. 14A) and 41BB/OX40 flow cytometry with gating to CD4 (FIG. 14B) and gating to CD8 (FIG. 14C) for 4394 TCRA- and 4394 TCRB-transduced PBLs co-cultured with autologous DC loaded with RAS G12V ME8 or RAS WT4 ME (WT sequence of ME8), RAS WT LP, RAS G12V LP or transfected with an mRNA of RAS G12V FL or RAS WT FL gene. co-culture with DC treated with DMSO served as negative controls. Non-specifically stimulated by co-culture with anti-CD3/anti-CD28 Dynabeads material served as a positive control.

FIGS. 15A-15C are graphs showing reactivity tested by IFNγ ELISPOT (FIG. 15A), and 41BB/OX40 flow cytometry with gating to CD4 (FIG. 15B) and gating to CD8 (FIG. 15C) for 4394 TCRA-transduced PBL co-cultured with 4394 DC loaded with RAS G12V ME8 or RAS ME WT4 (WT sequence of ME8) in different concentrations. TCR transduced PBL cultured alone served as negative controls. TCR transduced PBL non-specifically stimulated by co-culture with anti-CD3/anti-CD28 Dynabeads material served as a positive control.

### DETAILED DESCRIPTION OF THE INVENTION

RAS family proteins belong to the large family of small GTPases. Without being bound to a particular theory or mechanism, it is believed that, when mutated, RAS proteins may be involved in signal transduction early in the oncogenesis of many human cancers. A single amino acid substitution may activate the protein. The mutated RAS protein product may be constitutively activated. Mutated RAS proteins may be expressed in any of a variety of human cancers such as, for example, pancreatic (e.g., pancreatic carcinoma), colorectal, lung (e.g., lung adenocarcinoma), endometrial, ovarian (e.g., epithelial ovarian cancer), and prostate cancers. The human RAS family proteins include Kirsten rat sarcoma viral oncogene homolog (KRAS), Harvey rat sarcoma viral oncogene homolog (HRAS), and Neuroblastoma rat sarcoma viral oncogene homolog (NRAS).

KRAS is also referred to as GTPase KRas, V-Ki-Ras2 Kirsten rat sarcoma viral oncogene, or KRAS2. There are two transcript variants of KRAS: KRAS variant A and KRAS variant B. Wild-type (WT) KRAS variant A has the amino acid sequence of SEQ ID NO: 9. Wild-type (WT) KRAS variant B has the amino acid sequence of SEQ ID NO: 10. Hereinafter, references to “KRAS” (mutated or unmutated (WT)) refer to both variant A and variant B, unless specified otherwise. When activated, mutated KRAS binds to guanosine-5′-triphosphate (GTP) and converts GTP to guanosine 5′-diphosphate (GDP).

HRAS is another member of the RAS protein family. HRAS is also referred to as Harvey Rat Sarcoma Viral Oncoprotein, V-Ha-Ras Harvey Rat Sarcoma Viral Oncogene Homolog, or Ras Family Small GTP Binding Protein H-Ras. WT HRAS has the amino acid sequence of SEQ ID NO: 11.

NRAS is still another member of the RAS protein family. NRAS is also referred to as GTPase NRas, V-Ras Neuroblastoma RAS Viral Oncogene Homolog, or NRAS1. WT NRAS has the amino acid sequence of SEQ ID NO: 12.

An embodiment of the invention provides an isolated or purified TCR having antigenic specificity for a mutated human RAS amino acid sequence with a substitution of glycine at position 12 with valine (hereinafter, “mutated RAS”) presented by a human leukocyte antigen (HLA) Class I molecule, wherein the mutated human RAS amino acid sequence is a mutated human KRAS, a mutated human HRAS, or a mutated human NRAS amino acid sequence, and wherein position 12 is defined by reference to the WT human KRAS, WT human HRAS, or WT human NRAS protein, respectively. Hereinafter, references to a “TCR” also refer to functional portions and functional variants of the TCR, unless specified otherwise.

The inventive TCR may have antigenic specificity for any mutated human RAS protein, polypeptide or peptide amino acid sequence. In embodiments of the invention, the mutated human RAS amino acid sequence is a mutated human KRAS amino acid sequence, a mutated human HRAS amino acid sequence, or a mutated human NRAS amino acid sequence. The amino acid sequences of WT human KRAS, NRAS, and HRAS protein each have a length of 188-189 amino acid residues and have a high degree of identity to one another. For example, the amino acid sequence of the WT human NRAS protein is 86.8% identical to that of the WT human KRAS protein. Amino acid residues 1-86 of the WT human NRAS protein and the WT human KRAS protein are 100% identical. The amino acid sequence of the WT human HRAS protein is 86.3% identical to that of the WT human KRAS protein. Amino acid residues 1-94 of the WT human HRAS protein and the WT human KRAS protein are 100% identical. Hereinafter, references to “RAS” (mutated or unmutated (WT)) collectively refer to KRAS, HRAS, and NRAS, unless specified otherwise.

In embodiments of the invention, the mutated human RAS amino acid sequence comprises a WT RAS amino acid sequence with a substitution of glycine at position 12, wherein position 12 is defined by reference to the WT RAS protein, respectively. The WT RAS protein may be any of WT KRAS protein (SEQ ID NO: 9 or 10), WT HRAS protein (SEQ ID NO: 11), or WT NRAS protein (SEQ ID NO: 12) because, as explained above, amino acid residues 1-86 of the WT human NRAS protein and the WT human KRAS protein are 100% identical, and amino acid residues 1-94 of the WT human HRAS protein and the WT human KRAS protein are 100% identical. Accordingly, the amino acid residue at position 12 of each of WT KRAS, WT HRAS, and WT NRAS protein is the same, namely, glycine.

The glycine at position 12 of the WT RAS amino acid sequence may be substituted with any amino acid residue other than glycine. In embodiments of the invention, the substitution is a substitution of glycine at position 12 of the WT RAS amino acid sequence with valine. In this regard, embodiments of the invention provide TCRs with antigenic specificity for any WT RAS protein, polypeptide or peptide amino acid sequence with a G12V mutation.

Mutations and substitutions of RAS are defined herein by reference to the amino acid sequence of WT RAS protein. Thus, mutations and substitutions of RAS are described herein by reference to the amino acid residue present at a particular position in WT RAS protein, followed by the position number, followed by the amino acid residue with which that residue has been replaced in the particular mutation or substitution under discussion. A RAS amino acid sequence (e.g., a RAS peptide) may comprise fewer than all of the amino acid residues of the full-length, WT RAS protein. Accordingly, position 12 is defined herein by reference to the WT full-length RAS protein (namely, any one of SEQ ID NOs: 9-12) with the understanding that the actual position of the corresponding residue in a particular example of a RAS amino acid sequence may be different. When the positions are as defined by any one of SEQ ID NOs: 9-12, the term “G12” refers to the glycine normally present at position 12 of any one of SEQ ID NOs: 9-12, and “G12V” indicates that the glycine normally present at position 12 of any one of SEQ ID NOs: 9-12 is replaced by a valine. For example, when a particular example of a RAS amino acid sequence is, e.g., TEYKLVVVGAGGVGKSALTIQLI (SEQ ID NO: 28) (an exemplary WT RAS peptide corresponding to contiguous amino acid residues 2 to 24 of SEQ ID NO: 9), “G12V” refers to a substitution of the underlined glycine in SEQ ID NO: 28 with valine, even though the actual position of the underlined glycine in SEQ ID NO: 28 is 11.

Examples of full-length RAS proteins with the G12V mutation are set forth in Table 1 below.

TABLE 1

| Mutated Full-Length RAS Protein | SEQ ID NO: |
|---|---|
| G12V KRAS variant A | 13 |
| G12V KRAS variant B | 14 |
| G12V HRAS | 15 |
| G12V NRAS | 16 |

In embodiments of the invention, the TCR has antigenic specificity for a RAS peptide with the G12V mutation described above, wherein the mutated RAS peptide has any length. In embodiments of the invention, the mutated RAS peptide has any length suitable for binding to any of the HLA Class I molecules described herein. For example, the TCR may have antigenic specificity for a RAS peptide with the G12V mutation, the RAS peptide having a length of about 9 to about 10 amino acid residues. The mutated RAS peptide may comprise any contiguous amino acid residues of mutated RAS protein which include the G12V mutation. In embodiments of the invention, the TCR may have antigenic specificity for a RAS peptide with the G12V mutation, the mutated RAS peptide having a length of about 9 amino acid residues or about 10 amino acid residues. An example of a specific peptide with the G12V which may be recognized by the inventive G12V TCR is 9-mer AVGVGKSAL (SEQ ID NO: 29) within, e.g., the 24-mer MTEYKLVVVGAVGVGKSALTIQLI (SEQ ID NO: 30), of which SEQ ID NOs: 26 and 27 are the WT versions of the peptides, respectively. In an embodiment of the invention, the TCR has antigenic specificity for the mutated human RAS amino acid sequence of SEQ ID NO: 29 or 30. In an embodiment of the invention, the TCR does not have antigenic specificity for the wild-type human RAS amino acid sequence of SEQ ID NO: 26 or SEQ ID NO: 27. Without wishing to be bound by theory, the 24-mer of SEQ ID NO: 30 may be processed and presented in smaller segments, e.g., such as SEQ ID NO: 29.

In embodiments of the invention, the inventive TCRs are able to recognize mutated RAS presented by an HLA Class I molecule. In this regard, the TCR may elicit an immune response upon binding to mutated RAS within the context of an HLA Class I molecule. The inventive TCRs may bind to the HLA Class I molecule in addition to mutated RAS.

In embodiments of the invention, the HLA Class I molecule is an HLA-C molecule. The HLA-C molecule is a heterodimer of an α chain and (32 microglobulin. The HLA-C α chain may be encoded by an HLA-C gene. (32 microglobulin binds non-covalently to the alphal, alpha2 and alpha3 domains of the alphα chain to build the HLA-C complex. The HLA-C molecule may be any HLA-C molecule. In embodiments of the invention, the HLA Class I molecule is an HLA-001 molecule. The HLA-001 molecule may be any HLA-C01 molecule. Examples of HLA-001 molecules may include, but are not limited to, HLA-C*01:02.

The TCRs of the invention may provide any one or more of a variety of advantages, including when expressed by cells used for adoptive cell transfer. Mutated RAS is expressed by cancer cells and is not expressed by normal, noncancerous cells. Without being bound to a particular theory or mechanism, it is believed that the inventive TCRs advantageously target the destruction of cancer cells while minimizing or eliminating the destruction of normal, non-cancerous cells, thereby reducing, toxicity. Moreover, the inventive TCRs may, advantageously, successfully treat or prevent mutated RAS-positive cancers that do not respond to other types of treatment such as, for example, chemotherapy, surgery, or radiation. The RASG¹² mutations are among the most common hotspot mutations found in many cancer types. For example, the KRAS G12V mutation is expressed in about 27% and about 9% of patients with pancreatic and colorectal cancers, respectively. Moreover, RAS family members share the G12 hotspot mutation in different cancer types (e.g. NRAS in melanoma). Additionally, the inventive TCRs may provide highly avid recognition of mutated RAS, which may provide the ability to recognize unmanipulated tumor cells (e.g., tumor cells that have not been treated with interferon (IFN)-y, transfected with a vector encoding one or both of mutated RAS and HLA-C*01:02, pulsed with a RAS peptide with the G12V mutation, or a combination thereof). Moreover, the HLA-C*01:02 allele is expressed in approximately 6% and 10% in Caucasian and Hispanic ethnicities respectively and can get up to 40% in the Asian ethnicity in the United States. Accordingly, the inventive TCRs may increase the number of immunotherapy-eligible cancer patients to include those patients that express the HLA-C*01:02 allele who may not be eligible for immunotherapy using TCRs that recognize RAS presented by other MHC molecules. Moreover, the inventive TCRs, polypeptides and proteins comprise human amino acid sequences, which may reduce the risk of rejection by the human immune system as compared to, e.g., TCRs, polypeptides and proteins comprising mouse amino acid sequences.

The phrase “antigenic specificity,” as used herein, means that the TCR can specifically bind to and immunologically recognize mutated RAS with high avidity. For example, a TCR may be considered to have “antigenic specificity” for mutated RAS if about 1×10⁴ to about 1×10⁵ T cells expressing the TCR secrete at least about 200 pg/mL or more (e.g., 200 pg/mL or more, 300 pg/mL or more, 400 pg/mL or more, 500 pg/mL or more, 600 pg/mL or more, 700 pg/mL or more, 1000 pg/mL or more, 5,000 pg/mL or more, 7,000 pg/mL or more, 10,000 pg/mL or more, 20,000 pg/mL or more, or a range defined by any two of the foregoing values) of IFN-γ upon co-culture with (a) antigen-negative, HLA Class I molecule positive target cells pulsed with a low concentration of mutated RAS peptide (e.g., about 0.05 ng/mL to about 10 ng/mL, 1 ng/mL, 2 ng/mL, 5 ng/mL, 8 ng/mL, 10 ng/mL, or a range defined by any two of the foregoing values) or (b) antigen-negative, HLA Class I molecule positive target cells into which a nucleotide sequence encoding mutated RAS has been introduced such that the target cell expresses mutated RAS. Cells expressing the inventive TCRs may also secrete IFN-γ upon co-culture with antigen-negative, HLA Class I molecule positive target cells pulsed with higher concentrations of mutated RAS peptide. The HLA Class I molecule may be any of the HLA Class I molecules described herein (e.g., an HLA-C*01:02 molecule).

Alternatively or additionally, a TCR may be considered to have “antigenic specificity” for mutated RAS if T cells expressing the TCR secrete at least twice as much IFN-γ upon co-culture with (a) antigen-negative, HLA Class I molecule positive target cells pulsed with a low concentration of mutated RAS peptide or (b) antigen-negative, HLA Class I molecule positive target cells into which a nucleotide sequence encoding mutated RAS has been introduced such that the target cell expresses mutated RAS as compared to the amount of IFN-γ expressed by a negative control. The negative control may be, for example, (i) T cells expressing the TCR, co-cultured with (a) antigen-negative, HLA Class I molecule positive target cells pulsed with the same concentration of an irrelevant peptide (e.g., some other peptide with a different sequence from the mutated RAS peptide) or (b) antigen-negative, HLA Class I molecule positive target cells into which a nucleotide sequence encoding an irrelevant peptide has been introduced such that the target cell expresses the irrelevant peptide, or (ii) untransduced T cells (e.g., derived from PBMC, which do not express the TCR) co-cultured with (a) antigen-negative, HLA Class I molecule positive target cells pulsed with the same concentration of mutated RAS peptide or (b) antigen-negative, HLA Class I molecule positive target cells into which a nucleotide sequence encoding mutated RAS has been introduced such that the target cell expresses mutated RAS. The HLA Class I molecule expressed by the target cells of the negative control would be the same HLA Class I molecule expressed by the target cells that are co-cultured with the T cells being tested. The HLA Class I molecule may be any of the HLA Class I molecules described herein (e.g., an HLA-C*01:02 molecule). IFN-γ secretion may be measured by methods known in the art such as, for example, enzyme-linked immunosorbent assay (ELISA).

Alternatively or additionally, a TCR may be considered to have “antigenic specificity” for mutated RAS if at least twice as many of the numbers of T cells expressing the TCR secrete IFN-γ upon co-culture with (a) antigen-negative, HLA Class I molecule positive target cells pulsed with a low concentration of mutated RAS peptide or (b) antigen-negative, HLA Class I molecule positive target cells into which a nucleotide sequence encoding mutated RAS has been introduced such that the target cell expresses mutated RAS as compared to the numbers of negative control T cells that secrete IFN-γ. The HLA Class I molecule, concentration of peptide, and the negative control may be as described herein with respect to other aspects of the invention. The numbers of cells secreting IFN-γ may be measured by methods known in the art such as, for example, ELISPOT.

Alternatively or additionally, a TCR may be considered to have “antigenic specificity” for mutated RAS if T cells expressing the TCR upregulate expression of one or more T-cell activation markers as measured by, for example, flow cytometry after stimulation with target cells expressing mutated RAS. Examples of T-cell activation markers include 4-1BB, OX40, CD107a, CD69, and cytokines that are upregulated upon antigen stimulation (e.g., tumor necrosis factor (TNF), interleukin (IL)-2, etc.).

An embodiment of the invention provides a TCR comprising two polypeptides (i.e., polypeptide chains), such as an alpha (a) chain of a TCR, a beta (β) chain of a TCR, a gamma (y) chain of a TCR, a delta (6) chain of a TCR, or a combination thereof. The polypeptides of the inventive TCR can comprise any amino acid sequence, provided that the TCR has antigenic specificity for mutated RAS. In some embodiments, the TCR is non-naturally occurring.

In an embodiment of the invention, the TCR comprises two polypeptide chains, each of which comprises a variable region comprising a complementarity determining region (CDR)1, a CDR2, and a CDR3 of a TCR. In an embodiment of the invention, the TCR comprises a first polypeptide chain comprising a CDR1 comprising the amino acid sequence of SEQ ID NO: 1 (CDR1 of α chain), a CDR2 comprising the amino acid sequence of SEQ ID NO: 2 (CDR2 of α chain), and a CDR3 comprising the amino acid sequence of SEQ ID NO: 3 (CDR3 of α chain), and a second polypeptide chain comprising a CDR1 comprising the amino acid sequence of SEQ ID NO: 4 (CDR1 of β chain), a CDR2 comprising the amino acid sequence of SEQ ID NO: 5 (CDR2 of β chain), and a CDR3 comprising the amino acid sequence of SEQ ID NO: 6 (CDR3 of β chain).

In another embodiment of the invention, the TCR comprises a first polypeptide chain comprising a CDR1 comprising the amino acid sequence of SEQ ID NO: 31 (CDR1 of α chain), a CDR2 comprising the amino acid sequence of SEQ ID NO: 32 (CDR2 of α chain), and a CDR3 comprising the amino acid sequence of SEQ ID NO: 33 (CDR3 of α chain), and a second polypeptide chain comprising a CDR1 comprising the amino acid sequence of SEQ ID NO: 34 (CDR1 of β chain), a CDR2 comprising the amino acid sequence of SEQ ID NO: 35 (CDR2 of β chain), and a CDR3 comprising the amino acid sequence of SEQ ID NO: 36 (CDR3 of β chain).

In another embodiment of the invention, the TCR comprises a first polypeptide chain comprising a CDR1 comprising the amino acid sequence of SEQ ID NO: 64 (CDR1 of α chain), a CDR2 comprising the amino acid sequence of SEQ ID NO: 65 (CDR2 of α chain), and a CDR3 comprising the amino acid sequence of SEQ ID NO: 66 (CDR3 of α chain), and a second polypeptide chain comprising a CDR1 comprising the amino acid sequence of SEQ ID NO: 67 (CDR1 of β chain), a CDR2 comprising the amino acid sequence of SEQ ID NO: 68 (CDR2 of β chain), and a CDR3 comprising the amino acid sequence of SEQ ID NO: 69 (CDR3 of β chain).

In this regard, the inventive TCR can comprise any one or more of the amino acid sequences selected from SEQ ID NOs: 1-6, 31-36, and 64-69. In an embodiment of the invention, the TCR comprises the amino acid sequences of: (a) all of SEQ ID NOs: 1-3, (b) all of SEQ ID NOs: 4-6, (c) all of SEQ ID NOs: 31-33, (d) all of SEQ ID NOs: 34-36, (e) all of SEQ ID NOs: 64-66, (0 all of SEQ ID NOs: 67-69, (g) all of SEQ ID NOs: 1-6, (h) all of SEQ ID NOs: 31-36, or (i) all of SEQ ID NOs: 64-69. In an especially preferred embodiment, the TCR comprises the amino acid sequences of: (i) all of SEQ ID NOs: 1-6, (ii) all of SEQ ID NOs: 31-36, or (iii) all of SEQ ID NOs: 64-69.

The CDR3 of any one or more of SEQ ID NOS: 3, 6, 33, 36, 66, and 69, i.e., of the α chain or 13 chain or both, may further comprise a cysteine immediately N-terminal to the first amino acid of the CDR or a phenylalanine immediately C-terminal to the final amino acid or both.

In embodiments of the invention, the TCR comprises an amino acid sequence of a variable region of a TCR comprising the CDRs set forth above. The TCR may comprise a human variable region, e.g., a human α chain variable region and a human 13 chain variable region. In this regard, the TCR can, e.g., comprise the amino acid sequence of: SEQ ID NO: 7 (variable region of α chain of 4391 TCR with wild type N-terminal signal peptide); SEQ ID NO: 90 (variable region of α chain of 4391 TCR with variant N-terminal signal peptide); SEQ ID NO: 8 (variable region of β chain of 4391 TCR with variant N-terminal signal peptide); SEQ ID NO: 91 (variable region of β chain of 4391 TCR with wild type N-terminal signal peptide); SEQ ID NO: 37 (variable region of α chain of 4385 TCR with wild type N-terminal signal peptide); SEQ ID NO: 92 (variable region of α chain of 4385 TCR with variant N-terminal signal peptide); SEQ ID NO: 38 (variable region of β chain of 4385 TCR with variant N-terminal signal peptide); SEQ ID NO: 93 (variable region of β chain of 4385 TCR with wild type N-terminal signal peptide); SEQ ID NO: 70 (variable region of α chain of 4394 TCR with wild type N-terminal signal peptide); SEQ ID NO: 132 (variable region of α chain of 4394 TCR with variant N-terminal signal peptide); SEQ ID NO: 71 (variable region of β chain of 4394 TCR with variant N-terminal signal peptide); SEQ ID NO: 133 (variable region of β chain of 4394 TCR with wild type N-terminal signal peptide); SEQ ID NO: 47 (variable region of α chain of 4391 TCR without N-terminal signal peptide predicted with IMGT); SEQ ID NO: 94 (variable region of α chain of 4391 TCR without N-terminal signal peptide predicted with SignalP); SEQ ID NO: 48 (variable region of β chain of 4391 TCR without N-terminal signal peptide predicted with IMGT); SEQ ID NO: 95 (variable region of β chain of 4391 TCR without N-terminal signal sequence predicted with SignalP); SEQ ID NO: 49 (variable region of α chain of 4385 TCR without N-terminal signal peptide predicted with IMGT); SEQ ID NO: 96 (variable region of α chain of 4385 TCR without N-terminal signal peptide predicted with SignalP); SEQ ID NO: 50 (variable region of β chain of 4385 TCR without N-terminal signal peptide predicted with IMGT); SEQ ID NO: 97 (variable region of β chain of 4385 TCR without N-terminal signal peptide predicted with SignalP); SEQ ID NO: 72 (variable region of α chain of 4394 TCR without N-terminal signal peptide predicted with IMGT); SEQ ID NO: 88 (variable region of α chain of 4394 TCR without N-terminal signal peptide predicted with SignalP); SEQ ID NO: 73 (variable region of β chain of 4394 TCR without N-terminal signal peptide predicted with IMGT);

SEQ ID NO: 89 (variable region of β chain of 4394 TCR without N-terminal signal sequence predicted with SignalP); both of SEQ ID NOs: 7 and 8; both of SEQ ID NOs: 7 and 91; both of SEQ ID NOs: 90 and 8; both of SEQ ID NOs: 90 and 91; both of SEQ ID NOs: 37 and 38; both of SEQ ID NOs: 37 and 93; both of SEQ ID NOs: 92 and 38; both of SEQ ID NOs: 92 and 93; both of SEQ ID NOs: 70 and 71; both of SEQ ID NOs: 70 and 133; both of SEQ ID NOs: 132 and 71; both of SEQ ID NOs: 132 and 133; both of SEQ ID NOs: 47 and 48; both of SEQ ID NOs: 94 and 95; both of SEQ ID NOs: 49 and 50; both of SEQ ID NOs: 96 and 97; both of SEQ ID NOs: 72 and 73; or both of SEQ ID NOs: 88 and 89. Preferably, the TCR comprises the amino acid sequences of (i) both of SEQ ID NOs: 7 and 8; (ii) both of SEQ ID NOs: 90 and 91; (iii) both of SEQ ID NOs: 37 and 38; (iv) both of SEQ ID NOs: 92 and 93; (v) both of SEQ ID NOs: 70 and 71; (vi) both of SEQ ID NOs: 132 and 133; (vii) both of SEQ ID NOs: 47 and 48; (viii) both of SEQ ID NOs: 94 and 95; (ix) both of SEQ ID NOs: 49 and 50; (x) both of SEQ ID NOs: 96 and 97; (xi) both of SEQ ID NOs: 72 and 73; or (xii) both of SEQ ID NOs: 88 and 89.

The inventive TCRs may further comprise an α chain constant region and a (3 chain constant region. The constant region may be derived from any suitable species such as, e.g., human or mouse. In embodiments of the invention, the TCRs further comprise murine a and 13 chain constant regions or human a and 13 chain constant regions. As used herein, the term “murine” or “human,” when referring to a TCR or any component of a TCR described herein (e.g., complementarity determining region (CDR), variable region, constant region, a chain, and/or β chain), means a TCR (or component thereof) which is derived from a mouse or a human, respectively, i.e., a TCR (or component thereof) that originated from or was, at one time, expressed by a mouse T cell or a human T cell, respectively.

An embodiment of the invention provides a chimeric TCR comprising a human variable region and a murine constant region, wherein the TCR has antigenic specificity for a mutated human RAS amino acid sequence presented by an HLA Class I molecule. The murine constant region may provide any one or more advantages. For example, the murine constant region may diminish mispairing of the inventive TCR with endogenous TCRs of the host cell into which the inventive TCR is introduced. Alternatively or additionally, the murine constant region may increase expression of the inventive TCR as compared to the same TCR with a human constant region. The chimeric TCR may comprise the amino acid sequence of SEQ ID NO: 19 (wild-type (WT) murine α chain constant region), SEQ ID NO: 20 (WT murine β chain constant region), or both SEQ ID NOs: 19 and 20. Preferably, the inventive TCR comprises the amino acid sequences of both of SEQ ID NOs: 19 and 20. The chimeric TCR may comprise any of the murine constant regions described herein in combination with any of the CDR regions as described herein with respect to other aspects of the invention. In this regard, the TCR, e.g., may comprise the amino acid sequences of: (a) all of SEQ ID NOs: 1-3 and 19; (b) all of SEQ ID NOs: 4-6 and 20; (c) all of SEQ ID NOs: 31-33 and 19; (d) all of SEQ ID NOs: 34-36 and 20; (e) all of SEQ ID NOs: 64-66 and 19; (0 all of SEQ ID NOs: 67-69 and 20; (g) all of SEQ ID NOs: 1-6 and 19-20; (h) all of SEQ ID NOs: 31-36 and 19-20; or all of SEQ ID NOs: 64-69. In another embodiment of the invention, the chimeric TCR, e.g., may comprise any of the murine constant regions described herein in combination with any of the variable regions described herein with respect to other aspects of the invention. In this regard, the TCR may comprise the amino acid sequences of: (i) both of SEQ ID NOs: 7 and 19; (ii) both of SEQ ID NOs: 90 and 19; (iii) both of SEQ ID NOs: 8 and 20; (iv) both of SEQ ID NOs: 91 and 20; (v) both of SEQ ID NOs: 37 and 19; (vi) both of SEQ ID NOs: 92 and 19; (vii) both of SEQ ID NOs: 38 and 20; (viii) both of SEQ ID NOs: 93 and 20; (ix) both of SEQ ID NOs: 70 and 19; (x) both of SEQ ID NOs: 132 and 19; (xi) both of SEQ ID NOs: 71 and 20; (xii) both of SEQ ID NOs: 133 and 20; (xiii) all of SEQ ID NOs: 7-8 and 19-20; (xiv) all of SEQ ID NOs: 90-91 and 19-20; (xv) all of SEQ ID NOs: 37-38 and 19-20; (xvi) all of SEQ ID NOs: 92-93 and 19-20; or (xvii) all of SEQ ID NOs: 70-71 and 19-20; (xviii) all of SEQ ID NOs: 132-133 and 19-20;.

In another embodiment of the invention, the TCR comprises the amino acid sequence(s) of: SEQ ID NO: 23 (α chain of 4391 TCR with WT murine constant region and WT N-terminal signal peptide), SEQ ID NO: 102 (α chain of 4391 TCR with WT murine constant region and variant N-terminal signal peptide), SEQ ID NO: 24 (β chain of 4391 TCR with WT murine constant region and variant N-terminal signal peptide), SEQ ID NO: 103 03 chain of 4391 TCR with WT murine constant region and WT N-terminal signal peptide), SEQ ID NO: 39 (α chain of 4385 TCR with WT murine constant region and WT N-terminal signal peptide), SEQ ID NO: 108 (α chain of 4385 TCR with WT murine constant region and variant N-terminal signal peptide), SEQ ID NO: 40 (β chain of 4385 TCR with WT murine constant region and variant N-terminal signal peptide), SEQ ID NO: 109 (β chain of 4385 TCR with WT murine constant region and WT N-terminal signal peptide), SEQ ID NO: 78 (α chain of 4394 TCR with WT murine constant region and WT N-terminal signal peptide), SEQ ID NO: 138 (α chain of 4394 TCR with WT murine constant region and variant N-terminal signal peptide), SEQ ID NO: 79 (β chain of 4394 TCR with WT murine constant region and variant N-terminal signal peptide), SEQ ID NO: 139 (β chain of 4394 TCR with WT murine constant region and WT N-terminal signal peptide), SEQ ID NO: 51 (α chain of 4391 TCR with WT murine constant region and without N-terminal signal peptide predicted by IMGT), SEQ ID NO: 114 (α chain of 4391 TCR with WT murine constant region and without N-terminal signal peptide predicted by SignalP), SEQ ID NO: 52 (α chain of 4391 TCR with WT murine constant region and without N-terminal signal peptide predicted by IMGT), SEQ ID NO: 115 (β chain of 4391 TCR with WT murine constant region and without N-terminal signal peptide predicted by SignalP), SEQ ID NO: 53 (α chain of 4385 TCR with WT murine constant region and without N-terminal signal peptide predicted by IMGT), SEQ ID NO: 120 (α chain of 4385 TCR with WT murine constant region and without N-terminal signal peptide predicted by SignalP), SEQ ID NO: 54 (α chain of 4385 TCR with WT murine constant region and without N-terminal signal peptide predicted by IMGT), SEQ ID NO: 121 (β chain of 4385 TCR with WT murine constant region and without N-terminal signal peptide predicted by SignalP), SEQ ID NO: 80 (α chain of 4391 TCR with WT murine constant region and without N-terminal signal peptide predicted by IMGT), SEQ ID NO: 146 (α chain of 4391 TCR with WT murine constant region and without N-terminal signal peptide predicted by SignalP), SEQ ID NO: 81 (α chain of 4391 TCR with WT murine constant region and without N-terminal signal peptide predicted by IMGT), SEQ ID NO: 147 (β chain of 4391 TCR with WT murine constant region and without N-terminal signal peptide predicted by SignalP), both of SEQ ID NO: 23 and 24, both of SEQ ID NO: 23 and 103, both of SEQ ID NO: 102 and 24, both of SEQ ID NO: 102 and 103, both of SEQ ID NO: 39 and 40, both of SEQ ID NO: 39 and 109, both of SEQ ID NO: 108 and 40, both of SEQ ID NO: 108 and 109, both of SEQ ID NO: 78 and 79, both of SEQ ID NO: 78 and 139, both of SEQ ID NO: 138 and 79, both of SEQ ID NO: 138 and 139, both of SEQ ID NO: 51 and 52, both of SEQ ID NO: 114 and 115, both of SEQ ID NO: 53-54, both of SEQ ID NO: 120 and 121; both of SEQ ID NO: 80 and 81, or both of SEQ ID NO: 146 and 147.

In embodiments of the invention, the TCR comprises an α chain comprising a variable region and a constant region and a β chain comprising a variable region and a constant region. In this regard, the TCR, e.g., may comprise (a) an α chain comprising the amino acid sequence of SEQ ID NO: 21 (α chain of 4391 TCR with a wild type N-terminal signal peptide), wherein: (i) X at position 180 of SEQ ID NO: 21 is Thr or Cys; (ii) X at position 244 of SEQ ID NO: 21 is Ser, Ala, Val, Leu, Ile, Pro, Phe, Met, or Trp; (iii) X at position 246 of SEQ ID NO: 21 is Met, Ala, Val, Leu, Ile, Pro, Phe, or Trp; and (iv) X at position 247 of SEQ ID NO: 21 is Gly, Ala, Val, Leu, Ile, Pro, Phe, Met, or Trp; (b) an a chain comprising the amino acid sequence of SEQ ID NO: 100 (α chain of 4391 TCR with a variant N-terminal signal peptide), werein: (i) X at position 180 of SEQ ID NO: 100 is Thr or Cys; (ii) X at position 244 of SEQ ID NO: 100 is Ser, Ala, Val, Leu, Ile, Pro, Phe, Met, or Trp; (iii) X at position 246 of SEQ ID NO: 100 is Met, Ala, Val, Leu, Ile, Pro, Phe, or Trp; and (iv) X at position 247 of SEQ ID NO: 100 is Gly, Ala, Val, Leu, Ile, Pro, Phe, Met, or Trp; (c) a β chain comprising the amino acid sequence of SEQ ID NO: 22 (β chain of 4391 TCR with a variant N-terminal signal peptide), wherein X at position 190 of SEQ ID NO: 22 is Ser or Cys; (d) a β chain comprising the amino acid sequence of SEQ ID NO: 101 (β chain of 4391 TCR with a wild type N-terminal signal peptide), wherein X at position 190 of SEQ ID NO: 101 is Ser or Cys; (e) an α chain comprising the amino acid sequence of SEQ ID NO: 41 (α chain of 4385 TCR with a wild type N-terminal signal peptide), wherein: (i) X at position 181 of SEQ ID NO: 41 is Thr or Cys; (ii) X at position 245 of SEQ ID NO: 41 is Ser, Ala, Val, Leu, Ile, Pro, Phe, Met, or Trp; (iii) X at position 247 of SEQ ID NO: 41 is Met, Ala, Val, Leu, Ile, Pro, Phe, or Trp; and (iv) X at position 248 of SEQ ID NO: 41 is Gly, Ala, Val, Leu, Ile, Pro, Phe, Met, or Trp; (0 an α chain comprising the amino acid sequence of SEQ ID NO: 106 (α chain of 4385 TCR with a variant N-terminal signal peptide), wherein: (i) X at position 181 of SEQ ID NO: 106 is Thr or Cys; (ii) X at position 245 of SEQ ID NO: 106 is Ser, Ala, Val, Leu, Ile, Pro, Phe, Met, or Trp; (iii) X at position 247 of SEQ ID NO: 106 is Met, Ala, Val, Leu, Ile, Pro, Phe, or Trp; and (iv) X at position 248 of SEQ ID NO: 106 is Gly, Ala, Val, Leu, Ile, Pro, Phe, Met, or Trp; (g) a β chain comprising the amino acid sequence of SEQ ID NO: 42 (β chain of 4385 TCR with a variant N-terminal signal peptide), wherein X at position 195 of SEQ ID NO: 42 is Ser or Cys; (h) a β chain comprising the amino acid sequence of SEQ ID NO: 107 (β chain of 4385 TCR with a wild type N-terminal signal peptide), wherein X at position 195 of SEQ ID NO: 107 is Ser or Cys; (I) an α chain comprising the amino acid sequence of SEQ ID NO: 74 (α chain of 4394 TCR with a wild type N-terminal signal peptide), wherein: (i) X at position 180 of SEQ ID NO: 74 is Thr or Cys; (ii) X at position 244 of SEQ ID NO: 74 is Ser, Ala, Val, Leu, Ile, Pro, Phe, Met, or Trp; (iii) X at position 246 of SEQ ID NO: 74 is Met, Ala, Val, Leu, Ile, Pro, Phe, or Trp; and (iv) X at position 247 of SEQ ID NO: 74 is Gly, Ala, Val, Leu, Ile, Pro, Phe, Met, or Trp; (j) an α chain comprising the amino acid sequence of SEQ ID NO: 136 (α chain of 4394 TCR with a variant N-terminal signal peptide), werein: (i) X at position 180 of SEQ ID NO: 136 is Thr or Cys; (ii) X at position 244 of SEQ ID NO: 136 is Ser, Ala, Val, Leu, Ile, Pro, Phe, Met, or Trp; (iii) X at position 246 of SEQ ID NO: 136 is Met, Ala, Val, Leu, Ile, Pro, Phe, or Trp; and (iv) X at position 247 of SEQ ID NO: 136 is Gly, Ala, Val, Leu, Ile, Pro, Phe, Met, or Trp; (k) a β chain comprising the amino acid sequence of SEQ ID NO: 75 (β chain of 4394 TCR with a variant N-terminal signal peptide), wherein X at position 187 of SEQ ID NO: 75 is Ser or Cys; (1) a β chain comprising the amino acid sequence of SEQ ID NO: 137 (α chain of 4394 TCR with a wild type N-terminal signal peptide), wherein X at position 187 of SEQ ID NO: 137 is Ser or Cys; (m) both (a) and (c); (n) both (b) and (d); (o) both (e) and (g); (p) both (0 and (h); (q) both (i) and (k); (r) both (j) and (1); (s) an α chain comprising the amino acid sequence of SEQ ID NO: 55 (α chain of 4391 TCR without N-terminal signal peptide predicted by IMGT), wherein: (i) X at position 160 of SEQ ID NO: 55 is Thr or Cys; (ii) X at position 224 of SEQ ID NO: 55 is Ser, Ala, Val, Leu, Ile, Pro, Phe, Met, or Trp; (iii) X at position 226 of SEQ ID NO: 55 is Met, Ala, Val, Leu, Ile, Pro, Phe, or Trp; and (iv) X at position 227 of SEQ ID NO: 55 is Gly, Ala, Val, Leu, Ile, Pro, Phe, Met, or Trp; (t) an a chain comprising the amino acid sequence of SEQ ID NO: 112 (α chain of 4391 TCR without N-terminal signal peptide predicted by SignalP), wherein: (i) X at position 159 of SEQ ID NO: 112 is Thr or Cys; (ii) X at position 223 of SEQ ID NO: 112 is Ser, Ala, Val, Leu, Ile, Pro, Phe, Met, or Trp; (iii) X at position 225 of SEQ ID NO: 112 is Met, Ala, Val, Leu, Ile, Pro, Phe, or Trp; and (iv) X at position 226 of SEQ ID NO: 112 is Gly, Ala, Val, Leu, Ile, Pro, Phe, Met, or Trp; (u) a β chain comprising the amino acid sequence of SEQ ID NO: 56 03 chain of 4391 TCR without N-terminal signal peptide predicted by IMGT), wherein X at position 168 of SEQ ID NO: 56 is Ser or Cys; (v) a β chain comprising the amino acid sequence of SEQ ID NO: 113 (β chain of 4391 TCR without N-terminal signal peptide predicted by SignalP), wherein X at position 173 of SEQ ID NO: 113 is Ser or Cys; (w) an a chain comprising the amino acid sequence of SEQ ID NO: 57 (α chain of 4385 TCR without N-terminal signal peptide predicted by IMGT), wherein: (i) X at position 160 of SEQ ID NO: 57 is Thr or Cys; (ii) X at position 224 of SEQ ID NO: 57 is Ser, Ala, Val, Leu, Ile, Pro, Phe, Met, or Trp; (iii) X at position 226 of SEQ ID NO: 57 is Met, Ala, Val, Leu, Ile, Pro, Phe, or Trp; and (iv) X at position 227 of SEQ ID NO: 57 is Gly, Ala, Val, Leu, Ile, Pro, Phe, Met, or Trp; (x) an α chain comprising the amino acid sequence of SEQ ID NO: 118 (α chain of 4385 TCR without N-terminal signal peptide predicted by SignalP), wherein: (i) X at position 161 of SEQ ID NO: 118 is Thr or Cys; (ii) X at position 225 of SEQ ID NO: 118 is Ser, Ala, Val, Leu, Ile, Pro, Phe, Met, or Trp; (iii) X at position 227 of SEQ ID NO: 118 is Met, Ala, Val, Leu, Ile, Pro, Phe, or Trp; and (iv) X at position 228 of SEQ ID NO: 118 is Gly, Ala, Val, Leu, Ile, Pro, Phe, Met, or Trp; (y) a β chain comprising the amino acid sequence of SEQ ID NO: 58 (β chain of 4385 TCR without N-terminal signal peptide predicted by IMGT), wherein X at position 173 of SEQ ID NO: 58 is Ser or Cys; (z) a β chain comprising the amino acid sequence of SEQ ID NO: 119 (β chain of 4385 TCR without N-terminal signal peptide predicted by SignalP), wherein X at position 178 of SEQ ID NO: 119 is Ser or Cys; (aa) an α chain comprising the amino acid sequence of SEQ ID NO: 76 (α chain of 4394 TCR without N-terminal signal peptide predicted by IMGT), wherein: (i) X at position 159 of SEQ ID NO: 76 is Thr or Cys; (ii) X at position 223 of SEQ ID NO: 76 is Ser, Ala, Val, Leu, Ile, Pro, Phe, Met, or Trp; (iii) X at position 225 of SEQ ID NO: 76 is Met, Ala, Val, Leu, Ile, Pro, Phe, or Trp; and (iv) X at position 226 of SEQ ID NO: 76 is Gly, Ala, Val, Leu, Ile, Pro, Phe, Met, or Trp; (bb) an α chain comprising the amino acid sequence of SEQ ID NO: 144 (a chain of 4394 TCR without N-terminal signal peptide predicted by SignalP), wherein: (i) X at position 160 of SEQ ID NO: 144 is Thr or Cys; (ii) X at position 224 of SEQ ID NO: 144 is Ser, Ala, Val, Leu, Ile, Pro, Phe, Met, or Trp; (iii) X at position 226 of SEQ ID NO: 144 is Met, Ala, Val, Leu, Ile, Pro, Phe, or Trp; and (iv) X at position 227 of SEQ ID NO: 144 is Gly, Ala, Val, Leu, Ile, Pro, Phe, Met, or Trp; (cc) a β chain comprising the amino acid sequence of SEQ ID NO: 77 (β chain of 4394 TCR without N-terminal signal peptide predicted by IMGT), wherein X at position 168 of SEQ ID NO: 77 is Ser or Cys; (dd) a (3 chain comprising the amino acid sequence of SEQ ID NO: 145 (β chain of 4394 TCR without N-terminal signal peptide predicted by SignalP), wherein X at position 166 of SEQ ID NO: 145 is Ser or Cys; (ee) both (s) and (u); (ff) both (t) and (v); (gg) both (w) and (y); (hh) both (x)) and (z); (hh) both (aa) and (cc); or (ii) both (bb) and (dd). In embodiments of the invention, the TCR comprising SEQ ID NO: 21 does not comprise SEQ ID NO: 23 (unsubstituted α chain of 4391 TCR). In embodiments of the invention, the TCR comprising SEQ ID NO: 100 does not comprise SEQ ID NO: 102 (unsubstituted α chain of 4391 TCR). In embodiments of the invention, the TCR comprising SEQ ID NO: 22 does not comprise SEQ ID NO: 24 (unsubstituted β chain of 4391 TCR). In embodiments of the invention, the TCR comprising SEQ ID NO: 101 does not comprise SEQ ID NO: 103 (unsubstituted β chain of 4391 TCR). In embodiments of the invention, the TCR comprising SEQ ID NO: 41 does not comprise SEQ ID NO: 39 (unsubstituted α chain of 4385 TCR). In embodiments of the invention, the TCR comprising SEQ ID NO: 106 does not comprise SEQ ID NO: 108 (unsubstituted α chain of 4385 TCR). In embodiments of the invention, the TCR comprising SEQ ID NO: 42 does not comprise SEQ ID NO: 40 (unsubstituted β chain of 4385 TCR). In embodiments of the invention, the TCR comprising SEQ ID NO: 107 does not comprise SEQ ID NO: 109 (unsubstituted β chain of 4385 TCR). In embodiments of the invention, the TCR comprising SEQ ID NO: 74 does not comprise SEQ ID NO: 78 (unsubstituted α chain of 4394 TCR). In embodiments of the invention, the TCR comprising SEQ ID NO: 136 does not comprise SEQ ID NO: 138 (unsubstituted α chain of 4394 TCR). In embodiments of the invention, the TCR comprising SEQ ID NO: 75 does not comprise SEQ ID NO: 79 (unsubstituted β chain of 4394 TCR). In embodiments of the invention, the TCR comprising SEQ ID NO: 137 does not comprise SEQ ID NO: 139 (unsubstituted β chain of 4394 TCR).

In embodiments of the invention, the TCR comprises a substituted constant region. In this regard, the TCR, e.g., may comprise the amino acid sequence of any of the TCRs described herein with one, two, three, or four amino acid substitution(s) in the constant region of one or both of the a and β chain. Preferably, the TCR comprises a murine constant region with one, two, three, or four amino acid substitution(s) in the murine constant region of one or both of the a and β chains. In an especially preferred embodiment, the TCR comprises a murine constant region with one, two, three, or four amino acid substitution(s) in the murine constant region of the α chain and one amino acid substitution in the murine constant region of the 13 chain. In some embodiments, the TCRs comprising the substituted constant region advantageously provide one or more of increased recognition of mutated RAS⁺ targets, increased expression by a host cell, diminished mispairing with endogenous TCRs, and increased anti-tumor activity as compared to the parent TCR comprising an unsubstituted (wild-type) constant region. In general, the substituted amino acid sequences of the murine constant regions of the TCR a and β chains, SEQ ID NOs: 17 and 18, respectively, correspond with all or portions of the unsubstituted murine constant region amino acid sequences SEQ ID NOs: 19 and 20, respectively, with SEQ ID NO: 17 having one, two, three, or four amino acid substitution(s) when compared to SEQ ID NO: 19 and SEQ ID NO: 18 having one amino acid substitution when compared to SEQ ID NO: 20. In this regard, an embodiment of the invention provides a TCR comprising the amino acid sequence of (a) SEQ ID NO: 17 (constant region of α chain), wherein (i) X at position 48 is Thr or Cys; (ii) X at position 112 is Ser, Ala, Val, Leu, Ile, Pro, Phe, Met, or Trp; (iii) X at position 114 is Met, Ala, Val, Leu, Ile, Pro, Phe, or Trp; and (iv) X at position 115 is Gly, Ala, Val, Leu, Ile, Pro, Phe, Met, or Trp; (b) SEQ ID NO: 18 (constant region of β chain), wherein X at position 57 is Ser or Cys; or (c) both of SEQ ID NOs: 17 and 18. In embodiments of the invention, the TCR comprising SEQ ID NO: 17 does not comprise SEQ ID NO: 19 (unsubstituted murine constant region of α chain). In embodiments of the invention, the TCR comprising SEQ ID NO: 18 does not comprise SEQ ID NO: 20 (unsubstituted murine constant region of β chain).

The first amino acid of any of the mouse alpha constant regions described herein may be different than N as provided in SEQ ID NOS: 17, 19 and 98. For example, in any TCR construct, polypeptide, protein, etc., as described herein, this first amino acid can be encoded by a split codon (having nucleotides from both a variable region and a constant region) such that any of the murine alpha constant regions may have a different amino acid at that position. For example, SEQ ID NOS: 21, 23, 39, 41, 51, 53, 55, 57, 74, 78, 100, 102, 104, 106, 108, 110, 126, 134, 136, 138 or 140 may each have an H at the position corresponding to the first amino acid in the constant region. Similarly, first amino acid of any of the mouse beta constant regions described herein may be different than E as provided in SEQ ID NOS: 18, 20 and 99, e.g., this first amino acid can be encoded by a split codon.

In embodiments of the invention, the substituted constant region includes cysteine substitutions in the constant region of one or both of the a and 13 chains to provide a cysteine-substituted TCR. Opposing cysteines in the a and the 13 chains provide a disulfide bond that links the constant regions of the a and the 13 chains of the substituted TCR to one another and which is not present in a TCR comprising the unsubstituted murine constant regions. In this regard, the TCR, e.g., may be a cysteine-substituted TCR in which one or both of the native Thr at position 48 (Thr48) of SEQ ID NO: 19 and the native Ser at position 57 (Ser57) of SEQ ID NO: 20 may be substituted with Cys. Preferably, both of the native Thr48 of SEQ ID NO: 19 and the native Ser57 of SEQ ID NO: 20 are substituted with Cys. Examples of cysteine-substituted TCR constant regions sequences are set forth in Table 2. In embodiments of the invention, the cysteine-substituted TCR comprises (i) SEQ ID NO: 17, (ii) SEQ ID NO: 18, or (iii) both of SEQ ID NOs: 17 and 18, wherein both of SEQ ID NOs: 17 and 18 are as defined in Table 2. The cysteine-substituted TCRs of the invention may include the substituted constant region in addition to any of the CDRs or variable regions described herein.

In embodiments of the invention, the cysteine-substituted, chimeric TCR comprises a full length alphα chain and a full-length betα chain. Examples of cysteine-substituted, chimeric TCR alphα chain and betα chain sequences are set forth in Table 2. In embodiments of the invention, the TCR comprises (i) SEQ ID NO: 21, (ii) SEQ ID NO: 22, (iii) SEQ ID NO: 100; (iv) SEQ ID NO: 101; (v) SEQ ID NO: 41, (vi) SEQ ID NO: 42; (vii) SEQ ID NO: 106; (viii) SEQ ID NO: 107; (i) SEQ ID NO: 74, (ii) SEQ ID NO: 75, (iii) SEQ ID NO: 136; (iv) SEQ ID NO: 137; (ix) both of SEQ ID NOs: 21 and 22; (x) both of SEQ ID NOs: 100 and 101; (xi) both of SEQ ID NOs: 41 and 42; (xii) both of SEQ ID NOs: 106 and 107; (xi) both of SEQ ID NOs: 74 and 75; (xi) both of SEQ ID NOs: 136 and 137; ((xiii) SEQ ID NO: 55; (xiv) SEQ ID NO: 56; (xv) SEQ ID NO: 112; (xvi) SEQ ID NO: 113; (xvii) SEQ ID NO: 57; (xviii) SEQ ID NO: 58; (xix) SEQ ID NO: 118; (xx) SEQ ID NO: 119; (xiii) SEQ ID NO: 76; (xiv) SEQ ID NO: 77; (xv) SEQ ID NO: 144; (xvi) SEQ ID NO: 145; (xxi) both of SEQ ID NOs: 55 and 56; (xxii) both of SEQ ID NOs: 112 and 113; (xxiii) both of SEQ ID NOs: 57 and 58; (xxiv) both of SEQ ID NOs: 118 and 119, (xxi) both of SEQ ID NOs: 76 and 77; (xxii) both of SEQ ID NOs: 144 and 145;wherein all of SEQ ID NOs: 21, 22, 41, 42, 55-58, 74-77, 100, 101, 106, 107, 112, 113, 118, 119, 136, 137, 144 and 145 are as defined in Table 2.

TABLE 2

| SEQ ID NO: | Definitions of “X” in some embodiments |
|---|---|
| SEQ ID NO: 17 | X at position 48 is Cys, |
| (constant region α chain) | X at position 112 is Ser, |
| | X at position 114 is Met, and |
| | X at position 115 is Gly. |
| SEQ ID NO: 18 | X at position 57 is Cys |
| (constant region β chain) | |
| SEQ ID NO: 21 | X at position 180 is Cys, |
| (4391 α chain with wild | X at position 244 is Ser, |
| type N-terminal signal | X at position 246 is Met, and |
| peptide) | X at position 247 is Gly. |
| SEQ ID NO: 22 | X at position 190 is Cys |
| (4391 β chain with variant | |
| N-terminal signal peptide) | |
| SEQ ID NO: 100 | X at position 180 is Cys, |
| (4391 α chain with variant | X at position 244 is Ser, |
| N-terminal signal peptide) | X at position 246 is Met, and |
| | X at position 247 is Gly. |
| SEQ ID NO: 101 | X at position 190 is Cys |
| (4391 β chain with wild | |
| type N-terminal signal | |
| peptide) | |
| SEQ ID NO: 41 | X at position 181 is Cys, |
| (4385 α chain with wild | X at position 245 is Ser, |
| type N-terminal signal | X at position 247 is Met, and |
| peptide) | X at position 248 is Gly. |
| SEQ ID NO:42 | X at position 195 is Cys |
| (4385 β chain with variant | |
| N-terminal signal peptide) | |
| SEQ ID NO: 106 | X at position 181 is Cys, |
| (4385 α chain with variant | X at position 245 is Ser, |
| N-terminal signal peptide) | X at position 247 is Met, and |
| | X at position 248 is Gly. |
| SEQ ID NO: 107 | X at position 195 is Cys |
| (4385 β chain with wild | |
| type N-terminal signal | |
| peptide) | |
| SEQ ID NO: 74 | X at position 180 is Cys, |
| (4394 α chain with wild | X at position 244 is Ser, |
| type N-terminal signal | X at position 246 is Met, and |
| peptide) | X at position 247 is Gly. |
| SEQ ID NO: 75 | X at position 187 is Cys |
| (4394 β chain with variant | |
| N-terminal signal peptide) | |
| SEQ ID NO: 136 | X at position 180 is Cys, |
| (4394 α chain with variant | X at position 244 is Ser, |
| N-terminal signal peptide) | X at position 246 is Met, and |
| | X at position 247 is Gly. |
| SEQ ID NO: 137 | X at position 187 is Cys |
| (4394 β chain with wild | |
| type N-terminal signal | |
| peptide) | |
| SEQ ID NO: 55 | X at position 160 is Cys, |
| (4391 α chain predicted | X at position 224 is Ser, |
| sequence using IMGT | X at position 226 is Met, and |
| without N-terminal signal | X at position 227 is Gly. |
| peptide) | |
| SEQ ID NO: 56 | X at position 168 is Cys |
| (4391 β chain predicted | |
| sequence using IMGT | |
| without N-terminal signal | |
| peptide) | |
| SEQ ID NO: 112 | X at position 159 is Cys, |
| (4391 α chain predicted | X at position 223 is Ser, |
| sequence using SignalP | X at position 225 is Met, and |
| without N-terminal signal | X at position 226 is Gly. |
| peptide) | |
| SEQ ID NO: 113 | X at position 173 is Cys |
| (4391 β chain predicted | |
| sequence using SignalP | |
| without N-terminal signal | |
| peptide) | |
| SEQ ID NO: 57 | X at position 160 is Cys, |
| (4385 α chain predicted | X at position 224 is Ser, |
| sequence using IMGT | X at position 226 is Met, and |
| without N-terminal signal | X at position 227 is Gly. |
| peptide) | |
| SEQ ID NO: 58 | X at position 173 is Cys |
| (4385 β chain predicted | |
| sequence using IMGT | |
| without N-terminal signal | |
| peptide) | |
| SEQ ID NO: 118 | X at position 161 is Cys, |
| (4385 α chain predicted | X at position 225 is Ser, |
| sequence using SignalP | X at position 227 is Met, and |
| without N-terminal signal | X at position 228 is Gly. |
| peptide) | |
| SEQ ID NO: 119 | X at position 178 is Cys |
| (4385 β chain predicted | |
| sequence using SignalP | |
| without N-terminal signal | |
| peptide) | |
| SEQ ID NO: 76 | X at position 159 is Cys, |
| (4394 α chain predicted | X at position 223 is Ser, |
| sequence using IMGT | X at position 225 is Met, and |
| without N-terminal signal | X at position 226 is Gly. |
| peptide) | |
| SEQ ID NO: 77 | X at position 168 is Cys |
| (4394 β chain predicted | |
| sequence using IMGT | |
| without N-terminal signal | |
| peptide) | |
| SEQ ID NO: 144 | X at position 160 is Cys, |
| (4394 α chain predicted | X at position 224 is Ser, |
| sequence using SignalP | X at position 226 is Met, and |
| without N-terminal signal | X at position 227 is Gly. |
| peptide) | |
| SEQ ID NO: 145 | X at position 166 is Cys |
| (4394 β chain predicted | |
| sequence using SignalP | |
| without N-terminal signal | |
| peptide) | |

In embodiments of the invention, the substituted amino acid sequence includes substitutions of one, two, or three amino acids in the transmembrane (TM) domain of the constant region of one or both of the a and 13 chains with a hydrophobic amino acid to provide a hydrophobic amino acid-substituted TCR (also referred to herein as an “LVL-modified TCR”). The hydrophobic amino acid substitution(s) in the TM domain of the TCR may increase the hydrophobicity of the TM domain of the TCR as compared to a TCR that lacks the hydrophobic amino acid substitution(s) in the TM domain. In this regard, the TCR is an LVL-modified TCR in which one, two, or three of the native Ser112, Met114, and Glyl15 of SEQ ID NO: 19 may, independently, be substituted with Ala, Val, Leu, Ile, Pro, Phe, Met, or Trp; preferably with Leu, Ile, or Val; and the native Ser57 of SEQ ID NO: 20 may be substituted with Cys. Preferably, all three of the native Ser112, Met114, and Glyl15 of SEQ ID NO: 19 may, independently, be substituted with Ala, Val, Leu, Ile, Pro, Phe, Met, or Trp; preferably with Leu, Ile, or Val. In embodiments of the invention, the LVL-modified TCR comprises (i) SEQ ID NO: 17, (ii) SEQ ID NO: 18, or (iii) both of SEQ ID NOs: 17 and 18, wherein both of SEQ ID NOs: 17 and 18 are as defined in Table 3. The LVL-modified TCRs of the invention may include the substituted constant region in addition to any of the CDRs or variable regions described herein.

In embodiments of the invention, the LVL-modified TCR comprises a full length alphα chain and a full-length betα chain. Examples of LVL-modified TCR alphα chain and betα chain sequences are set forth in Table 3. In embodiments of the invention, the TCR comprises (i) SEQ ID NO: 21, (ii) SEQ ID NO: 22, (iii) SEQ ID NO: 100; (iv) SEQ ID NO: 101; (v) SEQ ID NO: 41, (vi) SEQ ID NO: 42; (vii) SEQ ID NO: 106; (viii) SEQ ID NO: 107; (i) SEQ ID NO: 74, (ii) SEQ ID NO: 75, (iii) SEQ ID NO: 136; (iv) SEQ ID NO: 137; (ix) both of SEQ ID NOs: 21 and 22; (x) both of SEQ ID NOs: 100 and 101; (xi) both of SEQ ID NOs: 41 and 42; (xii) both of SEQ ID NOs: 106 and 107; (xi) both of SEQ ID NOs: 74 and 75; (xi) both of SEQ ID NOs: 136 and 137; ((xiii) SEQ ID NO: 55; (xiv) SEQ ID NO: 56; (xv) SEQ ID NO: 112; (xvi) SEQ ID NO: 113; (xvii) SEQ ID NO: 57; (xviii) SEQ ID NO: 58; (xix) SEQ ID NO: 118; (xx) SEQ ID NO: 119; (xiii) SEQ ID NO: 76; (xiv) SEQ ID NO: 77; (xv) SEQ ID NO: 144; (xvi) SEQ ID NO: 145; (xxi) both of SEQ ID NOs: 55 and 56; (xxii) both of SEQ ID NOs: 112 and 113; (xxiii) both of SEQ ID NOs: 57 and 58; (xxiv) both of SEQ ID NOs: 118 and 119, (xxi) both of SEQ ID NOs: 76 and 77; (xxii) both of SEQ ID NOs: 144 and 145;wherein all of SEQ ID NOs: 21, 22, 41, 42, 55-58, 74-77, 100, 101, 106, 107, 112, 113, 118, 119, 136, 137, 144 and 145 are as defined in Table 3.

TABLE 3

| SEQ ID NO: | Definitions of “X” in some embodiments |
|---|---|
| SEQ ID NO: 17 | X at position 48 is Thr; |
| (constant region α | X at position 112 is Ser, Ala, Val, Leu, Ile, Pro, Phe, Met, or Trp; |
| chain) | preferably wherein X at position 112 is Leu, Ile, or Val; |
| | especially preferably wherein X at position 112 is Leu; |
| | X at position 114 is Met, Ala, Val, Leu, Ile, Pro, Phe, or Trp; |
| | preferably wherein X at position 114 is Leu, Ile, or Val; |
| | especially preferably wherein X at position 114 is Ile; and |
| | X at position 115 is Gly, Ala, Val, Leu, Ile, Pro, Phe, Met, or Trp; |
| | preferably wherein X at position 115 is Leu, Ile, or Val; |
| | especially preferably wherein X at position 115 is Val; |
| | Wherein SEQ ID NO: 17 does not comprise SEQ ID NO: 19 (unsubstituted |
| | constant region of alpha chain) |
| SEQ ID NO: 18 | X at position 57 is Ser |
| (constant region β | |
| chain) | |
| SEQ ID NO: 21 | X at position 180 is Thr; |
| (4391 α chain with | X at position 244 is Ser, Ala, Val, Leu, Ile, Pro, Phe, Met, or Trp; |
| wild type N-terminal | preferably wherein X at position 244 is Leu, Ile, or Val; |
| signal peptide) | especially preferably wherein X at position 244 is Leu; |
| | X at position 246 is Met, Ala, Val, Leu, Ile, Pro, Phe, or Trp; |
| | preferably wherein X at position 246 is Leu, Ile, or Val; |
| | especially preferably wherein X at position 246 is Ile; and |
| | X at position 247 is Gly, Ala, Val, Leu, Ile, Pro, Phe, Met, or Trp; |
| | preferably wherein X at position 247 is Leu, Ile, or Val; |
| | especially preferably wherein X at position 247 is Val, |
| | Wherein SEQ ID NO: 21 does not comprise SEQ ID NO: 23 (unsubstituted |
| | alpha chain) |
| SEQ ID NO: 22 | X at position 190 is Ser |
| (4391 β chain with | |
| variant N-terminal | |
| signal peptide) | |
| SEQ ID NO: 100 | X at position 180 is Thr; |
| (4391 α chain with | X at position 244 is Ser, Ala, Val, Leu, Ile, Pro, Phe, Met, or Trp; |
| variant N-terminal | preferably wherein X at position 244 is Leu, Ile, or Val; |
| signal peptide) | especially preferably wherein X at position 244 is Leu; |
| | X at position 246 is Met, Ala, Val, Leu, Ile, Pro, Phe, or Trp; |
| | preferably wherein X at position 246 is Leu, Ile, or Val; |
| | especially preferably wherein X at position 246 is Ile; and |
| | X at position 247 is Gly, Ala, Val, Leu, Ile, Pro, Phe, Met, or Trp; |
| | preferably wherein X at position 247 is Leu, Ile, or Val; |
| | especially preferably wherein X at position 247 is Val, |
| | Wherein SEQ ID NO: 100 does not comprise SEQ ID NO: 102 |
| | (unsubstituted alpha chain) |
| SEQ ID NO: 101 | X at position 190 is Ser |
| (4391 β chain with | |
| wild type N-terminal | |
| signal peptide) | |
| SEQ ID NO: 41 | X at position 181 is Thr; |
| (4385 α chain with | X at position 245 is Ser, Ala, Val, Leu, Ile, Pro, Phe, Met, or Trp; |
| wild type N-terminal | preferably wherein X at position 245 is Leu, Ile, or Val; |
| signal peptide) | especially preferably wherein X at position 245 is Leu; |
| | X at position 247 is Met, Ala, Val, Leu, Ile, Pro, Phe, or Trp; |
| | preferably wherein X at position 247 is Leu, Ile, or Val; |
| | especially preferably wherein X at position 247 is Ile; and |
| | X at position 248 is Gly, Ala, Val, Leu, Ile, Pro, Phe, Met, or Trp; |
| | preferably wherein X at position 248 is Leu, Ile, or Val; |
| | especially preferably wherein X at position 248 is Val, |
| | Wherein SEQ ID NO: 41 does not comprise SEQ ID NO: 39 (unsubstituted |
| | alpha chain) |
| SEQ ID NO: 42 | X at position 195 is Ser |
| (4385 β chain with | |
| variant N-terminal | |
| signal peptide) | |
| SEQ ID NO: 106 | X at position 181 is Thr; |
| (4385 α chain with | X at position 245 is Ser, Ala, Val, Leu, Ile, Pro, Phe, Met, or Trp; |
| variant N-terminal | preferably wherein X at position 245 is Leu, Ile, or Val; |
| signal peptide) | especially preferably wherein X at position 245 is Leu; |
| | X at position 247 is Met, Ala, Val, Leu, Ile, Pro, Phe, or Trp; |
| | preferably wherein X at position 247 is Leu, Ile, or Val; |
| | especially preferably wherein X at position 247 is Ile; and |
| | X at position 248 is Gly, Ala, Val, Leu, Ile, Pro, Phe, Met, or Trp; |
| | preferably wherein X at position 248 is Leu, Ile, or Val; |
| | especially preferably wherein X at position 248 is Val, |
| | Wherein SEQ ID NO: 106 does not comprise SEQ ID NO: 108 |
| | (unsubstituted alpha chain) |
| SEQ ID NO: 107 | X at position 195 is Ser |
| (4385 β chain with | |
| wild type N-terminal | |
| signal peptide) | |
| SEQ ID NO: 74 | X at position 180 is Thr; |
| (4394 α chain with | X at position 244 is Ser, Ala, Val, Leu, Ile, Pro, Phe, Met, or Trp; |
| wild type N-terminal | preferably wherein X at position 244 is Leu, Ile, or Val; |
| signal peptide) | especially preferably wherein X at position 244 is Leu; |
| | X at position 246 is Met, Ala, Val, Leu, Ile, Pro, Phe, or Trp; |
| | preferably wherein X at position 246 is Leu, Ile, or Val; |
| | especially preferably wherein X at position 246 is Ile; and |
| | X at position 247 is Gly, Ala, Val, Leu, Ile, Pro, Phe, Met, or Trp; |
| | preferably wherein X at position 247 is Leu, Ile, or Val; |
| | especially preferably wherein X at position 247 is Val, |
| | Wherein SEQ ID NO: 74 does not comprise SEQ ID NO: 78 (unsubstituted |
| | alpha chain) |
| SEQ ID NO: 75 | X at position 187 is Ser |
| (4394 β chain with | |
| variant N-terminal | |
| signal peptide) | |
| SEQ ID NO: 136 | X at position 180 is Thr; |
| (4394 α chain with | X at position 244 is Ser, Ala, Val, Leu, Ile, Pro, Phe, Met, or Trp; |
| variant N-terminal | preferably wherein X at position 244 is Leu, Ile, or Val; |
| signal peptide) | especially preferably wherein X at position 244 is Leu; |
| | X at position 246 is Met, Ala, Val, Leu, Ile, Pro, Phe, or Trp; |
| | preferably wherein X at position 246 is Leu, Ile, or Val; |
| | especially preferably wherein X at position 246 is Ile; and |
| | X at position 247 is Gly, Ala, Val, Leu, Ile, Pro, Phe, Met, or Trp; |
| | preferably wherein X at position 247 is Leu, Ile, or Val; |
| | especially preferably wherein X at position 247 is Val, |
| | Wherein SEQ ID NO: 74 does not comprise SEQ ID NO: 78 (unsubstituted |
| | alpha chain) |
| SEQ ID NO: 137 | X at position 187 is Ser |
| (4394 β chain with | |
| wild type N-terminal | |
| signal peptide) | |
| SEQ ID NO: 55 | X at position 160 is Thr; |
| (4391 α chain | X at position 224 is Ser, Ala, Val, Leu, Ile, Pro, Phe, Met, or Trp; |
| predicted sequence | preferably wherein X at position 224 is Leu, Ile, or Val; |
| using IMGT without | especially preferably wherein X at position 224 is Leu; |
| N-terminal signal | X at position 226 is Met, Ala, Val, Leu, Ile, Pro, Phe, or Trp; |
| peptide) | preferably wherein X at position 226 is Leu, Ile, or Val; |
| | especially preferably wherein X at position 226 is Ile; and |
| | X at position 227 is Gly, Ala, Val, Leu, Ile, Pro, Phe, Met, or Trp; |
| | preferably wherein X at position 227 is Leu, Ile, or Val; |
| | especially preferably wherein X at position 227 is Val, |
| | Wherein SEQ ID NO: 55 does not comprise SEQ ID NO: 51 (unsubstituted |
| | alpha chain) |
| SEQ ID NO: 56 | X at position 168 is Ser |
| (4391 β chain | |
| predicted sequence | |
| using IMGT without | |
| N-terminal signal | |
| peptide) | |
| SEQ ID NO: 112 | X at position 159 is Thr; |
| (4391 α chain | X at position 223 is Ser, Ala, Val, Leu, Ile, Pro, Phe, Met, or Trp; |
| predicted sequence | preferably wherein X at position 224 is Leu, Ile, or Val; |
| using SignalP without | especially preferably wherein X at position 224 is Leu; |
| N-terminal signal | X at position 225 is Met, Ala, Val, Leu, Ile, Pro, Phe, or Trp; |
| peptide) | preferably wherein X at position 226 is Leu, Ile, or Val; |
| | especially preferably wherein X at position 226 is Ile; and |
| | X at position 226 is Gly, Ala, Val, Leu, Ile, Pro, Phe, Met, or Trp; |
| | preferably wherein X at position 227 is Leu, Ile, or Val; |
| | especially preferably wherein X at position 227 is Val, |
| | Wherein SEQ ID NO: 112 does not comprise SEQ ID NO: 114 |
| | (unsubstituted alpha chain) |
| SEQ ID NO: 113 | X at position 173 is Ser |
| (4391 β chain | |
| predicted sequence | |
| using SignalP without | |
| N-terminal signal | |
| peptide) | |
| SEQ ID NO: 57 | X at position 160 is Thr; |
| (4385 α chain | X at position 224 is Ser, Ala, Val, Leu, Ile, Pro, Phe, Met, or Trp; |
| predicted sequence | preferably wherein X at position 224 is Leu, Ile, or Val; |
| using IMGT without | especially preferably wherein X at position 224 is Leu; |
| N-terminal signal | X at position 226 is Met, Ala, Val, Leu, Ile, Pro, Phe, or Trp; |
| peptide) | preferably wherein X at position 226 is Leu, Ile, or Val; |
| | especially preferably wherein X at position 226 is Ile; and |
| | X at position 227 is Gly, Ala, Val, Leu, Ile, Pro, Phe, Met, or Trp; |
| | preferably wherein X at position 227 is Leu, Ile, or Val; |
| | especially preferably wherein X at position 227 is Val, |
| | Wherein SEQ ID NO: 57 does not comprise SEQ ID NO: 53 (unsubstituted |
| | alpha chain) |
| SEQ ID NO: 58 | X at position 173 is Ser |
| (4385 β chain | |
| predicted sequence | |
| using IMGT without | |
| N-terminal signal | |
| peptide) | |
| SEQ ID NO: 118 | X at position 161 is Thr; |
| (4385 α chain | X at position 225 is Ser, Ala, Val, Leu, Ile, Pro, Phe, Met, or Trp; |
| predicted sequence | preferably wherein X at position 224 is Leu, Ile, or Val; |
| using SignalP without | especially preferably wherein X at position 224 is Leu; |
| N-terminal signal | X at position 227 is Met, Ala, Val, Leu, Ile, Pro, Phe, or Trp; |
| peptide) | preferably wherein X at position 226 is Leu, Ile, or Val; |
| | especially preferably wherein X at position 226 is Ile; and |
| | X at position 228 is Gly, Ala, Val, Leu, Ile, Pro, Phe, Met, or Trp; |
| | preferably wherein X at position 227 is Leu, Ile, or Val; |
| | especially preferably wherein X at position 227 is Val, |
| | Wherein SEQ ID NO: 118 does not comprise SEQ ID NO: 120 |
| | (unsubstituted alpha chain) |
| SEQ ID NO: 119 | X at position 178 is Ser |
| (4385 β chain | |
| predicted sequence | |
| using SignalP without | |
| N-terminal signal | |
| peptide) | |
| SEQ ID NO: 76 | X at position 159 is Thr; |
| (4394 α chain | X at position 223 is Ser, Ala, Val, Leu, Ile, Pro, Phe, Met, or Trp; |
| predicted sequence | preferably wherein X at position 223 is Leu, Ile, or Val; |
| using IMGT without | especially preferably wherein X at position 223 is Leu; |
| N-terminal signal | X at position 225 is Met, Ala, Val, Leu, Ile, Pro, Phe, or Trp; |
| peptide) | preferably wherein X at position 225 is Leu, Ile, or Val; |
| | especially preferably wherein X at position 225 is Ile; and |
| | X at position 226 is Gly, Ala, Val, Leu, Ile, Pro, Phe, Met, or Trp; |
| | preferably wherein X at position 226 is Leu, Ile, or Val; |
| | especially preferably wherein X at position 226 is Val, |
| | Wherein SEQ ID NO: 76 does not comprise SEQ ID NO: 80 (unsubstituted |
| | alpha chain) |
| SEQ ID NO: 77 | X at position 168 is Ser |
| (4394 β chain | |
| predicted sequence | |
| using IMGT without | |
| N-terminal signal | |
| peptide) | |
| SEQ ID NO: 144 | X at position 160 is Thr; |
| (4394 α chain | X at position 224 is Ser, Ala, Val, Leu, Ile, Pro, Phe, Met, or Trp; |
| predicted sequence | preferably wherein X at position 223 is Leu, Ile, or Val; |
| using SignalP without | especially preferably wherein X at position 223 is Leu; |
| N-terminal signal | X at position 226 is Met, Ala, Val, Leu, Ile, Pro, Phe, or Trp; |
| peptide) | preferably wherein X at position 225 is Leu, Ile, or Val; |
| | especially preferably wherein X at position 225 is Ile; and |
| | X at position 227 is Gly, Ala, Val, Leu, Ile, Pro, Phe, Met, or Trp; |
| | preferably wherein X at position 226 is Leu, Ile, or Val; |
| | especially preferably wherein X at position 226 is Val, |
| | Wherein SEQ ID NO: 144 does not comprise SEQ ID NO: 146 |
| | (unsubstituted alpha chain) |
| SEQ ID NO: 145 | X at position 166 is Ser |
| (4394 β chain | |
| predicted sequence | |
| using SignalP without | |
| N-terminal signal | |
| peptide) | |

In embodiments of the invention, the substituted amino acid sequence includes the cysteine substitutions in the constant region of one or both of the a and β chains in combination with the substitution(s) of one, two, or three amino acids in the transmembrane (TM) domain of the constant region of one or both of the a and β chains with a hydrophobic amino acid (also referred to herein as “cysteine-substituted, LVL-modified TCR”). In this regard, the TCR is a cysteine-substituted, LVL-modified, chimeric TCR in which the native Thr48 of SEQ ID NO: 19 is substituted with Cys; one, two, or three of the native Ser112, Met114, and Glyl15 of SEQ ID NO: 19 are, independently, substituted with Ala, Val, Leu, Ile, Pro, Phe, Met, or Trp; preferably with Leu, Ile, or Val; and the native Ser57 of SEQ ID NO: 20 is substituted with Cys. Preferably, all three of the native Ser112, Met114, and Glyl15 of SEQ ID NO: 19 may, independently, be substituted with Ala, Val, Leu, Ile, Pro, Phe, Met, or Trp; preferably with Leu, Ile, or Val. In embodiments of the invention, the cysteine-substituted, LVL-modified TCR comprises (i) SEQ ID NO: 17, (ii) SEQ ID NO: 18, or (iii) both of SEQ ID NOs: 17 and 18, wherein both of SEQ ID NOs: 17 and 18 are as defined in Table 4. The cysteine-substituted, LVL-modified TCRs of the invention may include the substituted constant region in addition to any of the CDRs or variable regions described herein.

In embodiments, the cysteine-substituted, LVL-modified TCR comprises a full-length alphα chain and a full-length betα chain. In embodiments of the invention, the cysteine-substituted, LVL-modified TCR comprises (i) SEQ ID NO: 21, (ii) SEQ ID NO: 22, (iii) SEQ ID NO: 100; (iv) SEQ ID NO: 101; (v) SEQ ID NO: 41, (vi) SEQ ID NO: 42; (vii) SEQ ID NO: 106; (viii) SEQ ID NO: 107; (i) SEQ ID NO: 74, (ii) SEQ ID NO: 75, (iii) SEQ ID NO: 136; (iv) SEQ ID NO: 137; (ix) both of SEQ ID NOs: 21 and 22; (x) both of SEQ ID NOs: 100 and 101; (xi) both of SEQ ID NOs: 41 and 42; (xii) both of SEQ ID NOs: 106 and 107; (xi) both of SEQ ID NOs: 74 and 75; (xi) both of SEQ ID NOs: 136 and 137; ((xiii) SEQ ID NO: 55; (xiv) SEQ ID NO: 56; (xv) SEQ ID NO: 112; (xvi) SEQ ID NO: 113; (xvii) SEQ ID NO: 57; (xviii) SEQ ID NO: 58; (xix) SEQ ID NO: 118; (xx) SEQ ID NO: 119; (xiii) SEQ ID NO: 76; (xiv) SEQ ID NO: 77; (xv) SEQ ID NO: 144; (xvi) SEQ ID NO: 145; (xxi) both of SEQ ID NOs: 55 and 56; (xxii) both of SEQ ID NOs: 112 and 113; (xxiii) both of SEQ ID NOs: 57 and 58; (xxiv) both of SEQ ID NOs: 118 and 119, (xxi) both of SEQ ID NOs: 76 and 77; (xxii) both of SEQ ID NOs: 144 and 145;wherein all of SEQ ID NOs: 21, 22, 41, 42, 55-58, 74-77, 100, 101, 106, 107, 112, 113, 118, 119, 136, 137, 144 and 145 are as defined in Table 4.

TABLE 4

| SEQ ID NO: | Definitions of “X” in some embodiments |
|---|---|
| SEQ ID NO: 17 | X at position 48 is Cys; |
| (constant region α | X at position 112 is Ser, Ala, Val, Leu, Ile, Pro, Phe, Met, or Trp; |
| chain) | preferably wherein X at position 112 is Leu, Ile, or Val; |
| | especially preferably wherein X at position 112 is Leu; |
| | X at position 114 is Met, Ala, Val, Leu, Ile, Pro, Phe, or Trp; |
| | preferably wherein X at position 114 is Leu, Ile, or Val; |
| | especially preferably wherein X at position 114 is Ile; and |
| | X at position 115 is Gly, Ala, Val, Leu, Ile, Pro, Phe, Met, or Trp; |
| | preferably wherein X at position 115 is Leu, Ile, or Val; and |
| | especially preferably wherein X at position 115 is Val, |
| | wherein SEQ ID NO: 17 does not simultaneously comprise all of Ser at |
| | position 112, Met at position 114, and Gly at position 115. |
| SEQ ID NO: 18 | X at position 57 is Cys |
| (constant region β | |
| chain) | |
| SEQ ID NO: 21 | X at position 180 is Cys; |
| (4391 α chain with wild | X at position 244 is Ser, Ala, Val, Leu, Ile, Pro, Phe, Met, or Trp; |
| type N-terminal signal | preferably wherein X at position 244 is Leu, Ile, or Val; |
| peptide) | especially preferably wherein X at position 244 is Leu; |
| | X at position 246 is Met, Ala, Val, Leu, Ile, Pro, Phe, or Trp; |
| | preferably wherein X at position 246 is Leu, Ile, or Val; |
| | especially preferably wherein X at position 246 is Ile; and |
| | X at position 247 is Gly, Ala, Val, Leu, Ile, Pro, Phe, Met, or Trp; |
| | preferably wherein X at position 247 is Leu, Ile, or Val; and |
| | especially preferably wherein X at position 247 is Val, |
| | wherein SEQ ID NO: 21 does not simultaneously comprise all of Ser at |
| | position 244, Met at position 246, and Gly at position 247. |
| SEQ ID NO: 22 | X at position 190 is Cys |
| (4391 β chain with | |
| variant N-terminal signal | |
| peptide) | |
| SEQ ID NO: 100 | X at position 180 is Cys; |
| (4391 α chain with | X at position 244 is Ser, Ala, Val, Leu, Ile, Pro, Phe, Met, or Trp; |
| variant N-terminal signal | preferably wherein X at position 244 is Leu, Ile, or Val; |
| peptide) | especially preferably wherein X at position 244 is Leu; |
| | X at position 246 is Met, Ala, Val, Leu, Ile, Pro, Phe, or Trp; |
| | preferably wherein X at position 246 is Leu, Ile, or Val; |
| | especially preferably wherein X at position 246 is Ile; and |
| | X at position 247 is Gly, Ala, Val, Leu, Ile, Pro, Phe, Met, or Trp; |
| | preferably wherein X at position 247 is Leu, Ile, or Val; and |
| | especially preferably wherein X at position 247 is Val, |
| | wherein SEQ ID NO: 100 does not simultaneously comprise all of Ser at |
| | position 244, Met at position 246, and Gly at position 247. |
| SEQ ID NO: 101 | X at position 190 is Cys |
| (4391 β chain with wild | |
| type N-terminal signal | |
| peptide) | |
| SEQ ID NO: 41 | X at position 181 is Cys; |
| (4385 α chain with wild | X at position 245 is Ser, Ala, Val, Leu, Ile, Pro, Phe, Met, or Trp; |
| type N-terminal signal | preferably wherein X at position 245 is Leu, Ile, or Val; |
| peptide) | especially preferably wherein X at position 245 is Leu; |
| | X at position 247 is Met, Ala, Val, Leu, Ile, Pro, Phe, or Trp; |
| | preferably wherein X at position 247 is Leu, Ile, or Val; |
| | especially preferably wherein X at position 247 is Ile; and |
| | X at position 248 is Gly, Ala, Val, Leu, Ile, Pro, Phe, Met, or Trp; |
| | preferably wherein X at position 248 is Leu, Ile, or Val; and |
| | especially preferably wherein X at position 248 is Val, |
| | wherein SEQ ID NO: 41 does not simultaneously comprise all of Ser at |
| | position 245, Met at position 247, and Gly at position 248. |
| SEQ ID NO: 42 | X at position 195 is Cys |
| (4385 β chain with | |
| variant N-terminal signal | |
| peptide) | |
| SEQ ID NO: 106 | X at position 181 is Cys; |
| (4385 α chain with | X at position 245 is Ser, Ala, Val, Leu, Ile, Pro, Phe, Met, or Trp; |
| variant N-terminal signal | preferably wherein X at position 245 is Leu, Ile, or Val; |
| peptide) | especially preferably wherein X at position 245 is Leu; |
| | X at position 247 is Met, Ala, Val, Leu, Ile, Pro, Phe, or Trp; |
| | preferably wherein X at position 247 is Leu, Ile, or Val; |
| | especially preferably wherein X at position 247 is Ile; and |
| | X at position 248 is Gly, Ala, Val, Leu, Ile, Pro, Phe, Met, or Trp; |
| | preferably wherein X at position 248 is Leu, Ile, or Val; and |
| | especially preferably wherein X at position 248 is Val, |
| | wherein SEQ ID NO: 106 does not simultaneously comprise all of Ser at |
| | position 245, Met at position 247, and Gly at position 248. |
| SEQ ID NO: 107 | X at position 195 is Cys |
| (4385 β chain with wild | |
| type N-terminal signal | |
| peptide) | |
| SEQ ID NO: 74 | X at position 180 is Cys; |
| (4394 α chain with wild | X at position 244 is Ser, Ala, Val, Leu, Ile, Pro, Phe, Met, or Trp; |
| type N-terminal signal | preferably wherein X at position 244 is Leu, Ile, or Val; |
| peptide) | especially preferably wherein X at position 244 is Leu; |
| | X at position 246 is Met, Ala, Val, Leu, Ile, Pro, Phe, or Trp; |
| | preferably wherein X at position 246 is Leu, Ile, or Val; |
| | especially preferably wherein X at position 246 is Ile; and |
| | X at position 247 is Gly, Ala, Val, Leu, Ile, Pro, Phe, Met, or Trp; |
| | preferably wherein X at position 247 is Leu, Ile, or Val; and |
| | especially preferably wherein X at position 247 is Val, |
| | wherein SEQ ID NO: 74 does not simultaneously comprise all of Ser at |
| | position 244, Met at position 246, and Gly at position 247. |
| SEQ ID NO: 75 | X at position 187 is Cys |
| (4394 β chain with | |
| variant N-terminal signal | |
| peptide) | |
| SEQ ID NO: 136 | X at position 180 is Cys; |
| (4394 α chain with | X at position 244 is Ser, Ala, Val, Leu, Ile, Pro, Phe, Met, or Trp; |
| variant N-terminal signal | preferably wherein X at position 244 is Leu, Ile, or Val; |
| peptide) | especially preferably wherein X at position 244 is Leu; |
| | X at position 246 is Met, Ala, Val, Leu, Ile, Pro, Phe, or Trp; |
| | preferably wherein X at position 246 is Leu, Ile, or Val; |
| | especially preferably wherein X at position 246 is Ile; and |
| | X at position 247 is Gly, Ala, Val, Leu, Ile, Pro, Phe, Met, or Trp; |
| | preferably wherein X at position 247 is Leu, Ile, or Val; and |
| | especially preferably wherein X at position 247 is Val, |
| | wherein SEQ ID NO: 74 does not simultaneously comprise all of Ser at |
| | position 244, Met at position 246, and Gly at position 247. |
| SEQ ID NO: 137 | X at position 187 is Cys |
| (4394 β chain with wild | |
| type N-terminal signal | |
| peptide) | |
| SEQ ID NO: 55 | X at position 160 is Cys; |
| (4391 α chain predicted | X at position 224 is Ser, Ala, Val, Leu, Ile, Pro, Phe, Met, or Trp; |
| sequence using IMGT | preferably wherein X at position 224 is Leu, Ile, or Val; |
| without N-terminal | especially preferably wherein X at position 224 is Leu; |
| signal peptide) | X at position 226 is Met, Ala, Val, Leu, Ile, Pro, Phe, or Trp; |
| | preferably wherein X at position 226 is Leu, Ile, or Val; |
| | especially preferably wherein X at position 226 is Ile; and |
| | X at position 227 is Gly, Ala, Val, Leu, Ile, Pro, Phe, Met, or Trp; |
| | preferably wherein X at position 227 is Leu, Ile, or Val; and |
| | especially preferably wherein X at position 227 is Val, |
| | wherein SEQ ID NO: 21 does not simultaneously comprise all of Ser at |
| | position 224, Met at position 226, and Gly at position 227. |
| SEQ ID NO: 56 | X at position 168 is Cys |
| (4391 β chain predicted | |
| sequence using IMGT | |
| without N-terminal | |
| signal peptide) | |
| SEQ ID NO: 112 | X at position 159 is Cys; |
| (4391 α chain predicted | X at position 223 is Ser, Ala, Val, Leu, Ile, Pro, Phe, Met, or Trp; |
| sequence using SignalP | preferably wherein X at position 224 is Leu, Ile, or Val; |
| without N-terminal | especially preferably wherein X at position 224 is Leu; |
| signal peptide) | X at position 225 is Met, Ala, Val, Leu, Ile, Pro, Phe, or Trp; |
| | preferably wherein X at position 226 is Leu, Ile, or Val; |
| | especially preferably wherein X at position 226 is Ile; and |
| | X at position 226 is Gly, Ala, Val, Leu, Ile, Pro, Phe, Met, or Trp; |
| | preferably wherein X at position 227 is Leu, Ile, or Val; and |
| | especially preferably wherein X at position 227 is Val, |
| | wherein SEQ ID NO: 112 does not simultaneously comprise all of Ser at |
| | position 223, Met at position 225, and Gly at position 226. |
| SEQ ID NO: 113 | X at position 173 is Cys |
| (4391 β chain predicted | |
| sequence using SignalP | |
| without N-terminal | |
| signal peptide) | |
| SEQ ID NO: 57 | X at position 160 is Cys; |
| (4385 α chain predicted | X at position 224 is Ser, Ala, Val, Leu, Ile, Pro, Phe, Met, or Trp; |
| sequence using IMGT | preferably wherein X at position 224 is Leu, Ile, or Val; |
| without N-terminal | especially preferably wherein X at position 224 is Leu; |
| signal peptide) | X at position 226 is Met, Ala, Val, Leu, Ile, Pro, Phe, or Trp; |
| | preferably wherein X at position 226 is Leu, Ile, or Val; |
| | especially preferably wherein X at position 226 is Ile; and |
| | X at position 227 is Gly, Ala, Val, Leu, Ile, Pro, Phe, Met, or Trp; |
| | preferably wherein X at position 227 is Leu, Ile, or Val; and |
| | especially preferably wherein X at position 227 is Val, |
| | wherein SEQ ID NO: 41 does not simultaneously comprise all of Ser at |
| | position 224, Met at position 226, and Gly at position 227. |
| SEQ ID NO: 58 | X at position 173 is Cys |
| (4385 β chain predicted | |
| sequence using IMGT | |
| without N-terminal | |
| signal peptide) | |
| SEQ ID NO: 118 | X at position 161 is Cys; |
| (4385 α chain predicted | X at position 225 is Ser, Ala, Val, Leu, Ile, Pro, Phe, Met, or Trp; |
| sequence using SignalP | preferably wherein X at position 224 is Leu, Ile, or Val; |
| without N-terminal | especially preferably wherein X at position 224 is Leu; |
| signal peptide) | X at position 227 is Met, Ala, Val, Leu, Ile, Pro, Phe, or Trp; |
| | preferably wherein X at position 226 is Leu, Ile, or Val; |
| | especially preferably wherein X at position 226 is Ile; and |
| | X at position 228 is Gly, Ala, Val, Leu, Ile, Pro, Phe, Met, or Trp; |
| | preferably wherein X at position 227 is Leu, Ile, or Val; and |
| | especially preferably wherein X at position 227 is Val, |
| | wherein SEQ ID NO: 118 does not simultaneously comprise all of Ser at |
| | position 225, Met at position 227, and Gly at position 228. |
| SEQ ID NO: 119 | X at position 178 is Cys |
| (4385 β chain predicted | |
| sequence using SignalP | |
| without N-terminal | |
| signal peptide) | |
| SEQ ID NO: 76 | X at position 159 is Cys; |
| (4394 α chain without | X at position 223 is Ser, Ala, Val, Leu, Ile, Pro, Phe, Met, or Trp; |
| N-terminal signal | preferably wherein X at position 223 is Leu, Ile, or Val; |
| peptide) | especially preferably wherein X at position 223 is Leu; |
| | X at position 225 is Met, Ala, Val, Leu, Ile, Pro, Phe, or Trp; |
| | preferably wherein X at position 225 is Leu, Ile, or Val; |
| | especially preferably wherein X at position 225 is Ile; and |
| | X at position 226 is Gly, Ala, Val, Leu, Ile, Pro, Phe, Met, or Trp; |
| | preferably wherein X at position 226 is Leu, Ile, or Val; and |
| | especially preferably wherein X at position 226 is Val, |
| | wherein SEQ ID NO: 76 does not simultaneously comprise all of Ser at |
| | position 224, Met at position 226, and Gly at position 227. |
| SEQ ID NO: 77 | X at position 168 is Cys |
| (4394 β chain without | |
| N-terminal signal | |
| peptide) | |
| SEQ ID NO: 144 | X at position 160 is Cys; |
| (4394 α chain predicted | X at position 224 is Ser, Ala, Val, Leu, Ile, Pro, Phe, Met, or Trp; |
| sequence using SignalP | preferably wherein X at position 223 is Leu, Ile, or Val; |
| without N-terminal | especially preferably wherein X at position 223 is Leu; |
| signal peptide) | X at position 226 is Met, Ala, Val, Leu, Ile, Pro, Phe, or Trp; |
| | preferably wherein X at position 225 is Leu, Ile, or Val; |
| | especially preferably wherein X at position 225 is Ile; and |
| | X at position 227 is Gly, Ala, Val, Leu, Ile, Pro, Phe, Met, or Trp; |
| | preferably wherein X at position 226 is Leu, Ile, or Val; and |
| | especially preferably wherein X at position 226 is Val, |
| | wherein SEQ ID NO: 144 does not simultaneously comprise all of Ser at |
| | position 224, Met at position 226, and Gly at position 227. |
| SEQ ID NO: 145 | X at position 166 is Cys |
| (4394 β chain predicted | |
| sequence using SignalP | |
| without N-terminal | |
| signal peptide) | |

In an embodiment of the invention, the cysteine-substituted, LVL-modified TCR comprises (a) SEQ ID NO: 98 (α chain constant region of cysteine-substituted, LVL-modified TCR); (b) SEQ ID NO: 99 (β chain constant region of cysteine-substituted, LVL-modified TCR); (c) SEQ ID NO: 124 (α chain of cysteine-substituted, LVL-modified 4391 TCR with wild type N-terminal signal sequence); (d) SEQ ID NO: 125 (β chain of cysteine-substituted, LVL-modified 4391 TCR with variant N-terminal signal sequence); (e) SEQ ID NO: 128 (α chain of cysteine-substituted, LVL-modified 4391 TCR without N-terminal signal sequence predicted by IMGT); (0 SEQ ID NO: 129 (β chain of cysteine-substituted, LVL-modified 4391 TCR without N-terminal signal sequence predicted by IMGT); (g) SEQ ID NO: 116 (α chain of cysteine-substituted, LVL-modified 4391 TCR without N-terminal signal sequence predicted by SignalP); (h) SEQ ID NO: 117 (α chain of cysteine-substituted, LVL-modified 4391 TCR without N-terminal signal sequence predicted by SignalP); (i) SEQ ID NO: 104 (α chain of cysteine-substituted, LVL-modified 4391 TCR with variant N-terminal signal sequence); (j) SEQ ID NO: 105 (α chain of cysteine-substituted, LVL-modified 4391 TCR with wild type N-terminal signal sequence); (k) both (a) and (b); (1) both (c) and (d); (m) both (e) and (f); (n) both (g) and (h); or (o) both (i) and (j).

In an embodiment of the invention, the cysteine-substituted, LVL-modified TCR comprises (a) SEQ ID NO: 98 (α chain constant region of cysteine-substituted, LVL-modified TCR); (b) SEQ ID NO: 99 (β chain constant region of cysteine-substituted, LVL-modified TCR); (c) SEQ ID NO: 126 (α chain of cysteine-substituted, LVL-modified 4385 TCR with wild type N-terminal signal sequence); (d) SEQ ID NO: 127 (β chain of cysteine-substituted, LVL-modified 4385 TCR with variant N-terminal signal sequence); (e) SEQ ID NO: 130 (α chain of cysteine-substituted, LVL-modified 4385 TCR without N-terminal signal sequence predicted by IMGT); (0 SEQ ID NO: 131 (β chain of cysteine-substituted, LVL-modified 4385 TCR without N-terminal signal sequence predicted by IMGT); (g) SEQ ID NO: 122 (α chain of cysteine-substituted, LVL-modified 4385 TCR without N-terminal signal sequence predicted by SignalP); (h) SEQ ID NO: 123 (α chain of cysteine-substituted, LVL-modified 4385 TCR without N-terminal signal sequence predicted by SignalP); (i) SEQ ID NO: 110 (α chain of cysteine-substituted, LVL-modified 4385 TCR with variant N-terminal signal sequence); (j) SEQ ID NO: 111 (α chain of cysteine-substituted, LVL-modified 4385 TCR with wild type N-terminal signal sequence); (k) both (a) and (b); (1) both (c) and (d); (m) both (e) and (0; (n) both (g) and (h); or (o) both (i) and (j).

In an embodiment of the invention, the cysteine-substituted, LVL-modified TCR comprises (a) SEQ ID NO: 98 (α chain constant region of cysteine-substituted, LVL-modified TCR); (b) SEQ ID NO: 99 (β chain constant region of cysteine-substituted, LVL-modified TCR); (c) SEQ ID NO: 134 (α chain of cysteine-substituted, LVL-modified 4394 TCR with wild type N-terminal signal sequence); (d) SEQ ID NO: 135 (β chain of cysteine-substituted, LVL-modified 4394 TCR with variant N-terminal signal sequence); (e) SEQ ID NO: 142 (α chain of cysteine-substituted, LVL-modified 4394 TCR without N-terminal signal sequence predicted by IMGT); (f) SEQ ID NO: 143 (β chain of cysteine-substituted, LVL-modified 4394 TCR without N-terminal signal sequence predicted by IMGT); (g) SEQ ID NO: 148 (α chain of cysteine-substituted, LVL-modified 4394 TCR without N-terminal signal sequence predicted by SignalP); (h) SEQ ID NO: 149 (α chain of cysteine-substituted, LVL-modified 4394 TCR without N-terminal signal sequence predicted by SignalP); (i) SEQ ID NO: 140 (α chain of cysteine-substituted, LVL-modified 4394 TCR with variant N-terminal signal sequence); (j) SEQ ID NO: 141 (β chain of cysteine-substituted, LVL-modified 4394 TCR with wild type N-terminal signal sequence); (k) both (a) and (b); (1) both (c) and (d); (m) both (e) and (0; (n) both (g) and (h); or (o) both (i) and (j).

Also provided by an embodiment of the invention is a polypeptide comprising a functional portion of any of the TCRs described herein. The term “polypeptide,” as used herein, includes oligopeptides and refers to a single chain of amino acids connected by one or more peptide bonds.

With respect to the inventive polypeptides, the functional portion can be any portion comprising contiguous amino acids of the TCR of which it is a part, provided that the functional portion specifically binds to mutated RAS. The term “functional portion,” when used in reference to a TCR, refers to any part or fragment of the TCR of the invention, which part or fragment retains the biological activity of the TCR of which it is a part (the parent TCR). Functional portions encompass, for example, those parts of a TCR that retain the ability to specifically bind to mutated RAS (e.g., within the context of an HLA-C*01:02 molecule), or detect, treat, or prevent cancer, to a similar extent, the same extent, or to a higher extent, as the parent TCR. In reference to the parent TCR, the functional portion can comprise, for instance, about 10%, about 25%, about 30%, about 50%, about 70%, about 80%, about 90%, about 95%, or more, of the parent TCR.

The functional portion can comprise additional amino acids at the amino or carboxy terminus of the portion, or at both termini, which additional amino acids are not found in the amino acid sequence of the parent TCR. Desirably, the additional amino acids do not interfere with the biological function of the functional portion, e.g., specifically binding to mutated RAS; and/or having the ability to detect cancer, treat or prevent cancer, etc. More desirably, the additional amino acids enhance the biological activity, as compared to the biological activity of the parent TCR.

The polypeptide can comprise a functional portion of either or both of the a and 13 chains of the TCRs of the invention, such as a functional portion comprising one or more of the CDR1, CDR2, and CDR3 of the variable region(s) of the α chain and/or 13 chain of a TCR of the invention. In an embodiment of the invention, the polypeptide can comprise the amino acid sequence of SEQ ID NO: 1 (CDR1 of α chain), SEQ ID NO: 2 (CDR2 of α chain), SEQ ID NO: 3 (CDR3 of α chain), SEQ ID NO: 4 (CDR1 of β chain), SEQ ID NO: 5 (CDR2 of β chain), SEQ ID NO: 6 (CDR3 of β chain), or a combination thereof. In another embodiment of the invention, the polypeptide can comprise the amino acid sequence of SEQ ID NO: 31 (CDR1 of α chain), SEQ ID NO: 32 (CDR2 of α chain), SEQ ID NO: 33 (CDR3 of α chain), SEQ ID NO: 34 (CDR1 of β chain), SEQ ID NO: 35 (CDR2 of β chain), SEQ ID NO: 36 (CDR3 of β chain), or a combination thereof. In another embodiment of the invention, the polypeptide can comprise the amino acid sequence of SEQ ID NO: 64 (CDR1 of α chain), SEQ ID NO: 65 (CDR2 of α chain), SEQ ID NO: 66 (CDR3 of α chain), SEQ ID NO: 67 (CDR1 of β chain), SEQ ID NO: 68 (CDR2 of β chain), SEQ ID NO: 69 (CDR3 of β chain), or a combination thereof.

In this regard, the inventive polypeptide can comprise any one or more of the amino acid sequences selected from SEQ ID NOs: 1-6 and 31-36. In an embodiment of the invention, the TCR comprises the amino acid sequences of: (a) all of SEQ ID NOs: 1-3, (b) all of SEQ ID NOs: 4-6, (c) all of SEQ ID NOs: 31-33, (d) all of SEQ ID NOs: 34-36, (e) all of SEQ ID NOs: 64-66, (0 all of SEQ ID NOs: 67-69, (g) all of SEQ ID NOs: 1-6, (h) all of SEQ ID NOs: 31-36, or (i) all of SEQ ID NOs: 64-69. In an especially preferred embodiment, the TCR comprises the amino acid sequences of: (i) all of SEQ ID NOs: 1-6, (ii) all of SEQ ID NOs: 31-36, or (iii) all of SEQ ID NOs: 64-69. The CDR3 of any one or more of SEQ ID NOS: 3, 6, 33, 36, 66, and 69, i.e., of the α chain or 13 chain or both, may further comprise a cysteine immediately N-terminal to the first amino acid of the CDR or a phenylalanine immediately C-terminal to the final amino acid or both.

In an embodiment of the invention, the inventive polypeptide can comprise, for instance, the variable region of the inventive TCR comprising a combination of the CDR regions set forth above. In this regard, the polypeptide can comprise the amino acid sequence of SEQ ID NO: 7 (variable region of α chain of 4391 TCR with wild type N-terminal signal peptide); SEQ ID NO: 90 (variable region of α chain of 4391 TCR with variant N-terminal signal peptide); SEQ ID NO: 8 (variable region of β chain of 4391 TCR with variant N-terminal signal peptide); SEQ ID NO: 91 (variable region of β chain of 4391 TCR with wild type N-terminal signal peptide); SEQ ID NO: 37 (variable region of α chain of 4385 TCR with wild type N-terminal signal peptide); SEQ ID NO: 92 (variable region of α chain of 4385 TCR with variant N-terminal signal peptide); SEQ ID NO: 38 (variable region of β chain of 4385 TCR with variant N-terminal signal peptide); SEQ ID NO: 93 (variable region of β chain of 4385 TCR with wild type N-terminal signal peptide); SEQ ID NO: 70 (variable region of α chain of 4394 TCR with wild type N-terminal signal peptide); SEQ ID NO: 132 (variable region of α chain of 4394 TCR with variant N-terminal signal peptide); SEQ ID NO: 71 (variable region of β chain of 4394 TCR with variant N-terminal signal peptide); SEQ ID NO: 133 (variable region of β chain of 4394 TCR with wild type N-terminal signal peptide); SEQ ID NO: 47 (variable region of α chain of 4391 TCR without N-terminal signal peptide predicted with IMGT); SEQ ID NO: 94 (variable region of α chain of 4391 TCR without N-terminal signal peptide predicted with SignalP); SEQ ID NO: 48 (variable region of β chain of 4391 TCR without N-terminal signal peptide predicted with IMGT); SEQ ID NO: 95 (variable region of β chain of 4391 TCR without N-terminal signal sequence predicted with SignalP); SEQ ID NO: 49 (variable region of α chain of 4385 TCR without N-terminal signal peptide predicted with IMGT); SEQ ID NO: 96 (variable region of α chain of 4385 TCR without N-terminal signal peptide predicted with SignalP); SEQ ID NO: 50 (variable region of β chain of 4385 TCR without N-terminal signal peptide predicted with IMGT); SEQ ID NO: 97 (variable region of β chain of 4385 TCR without N-terminal signal peptide predicted with SignalP); SEQ ID NO: 72 (variable region of α chain of 4394 TCR without N-terminal signal peptide predicted with IMGT); SEQ ID NO: 88 (variable region of α chain of 4394 TCR without N-terminal signal peptide predicted with SignalP); SEQ ID NO: 73 (variable region of β chain of 4394 TCR without N-terminal signal peptide predicted with IMGT); SEQ ID NO: 89 (variable region of β chain of 4394 TCR without N-terminal signal sequence predicted with SignalP); both of SEQ ID NOs: 7 and 8; both of SEQ ID NOs: 7 and 91; both of SEQ ID NOs: 90 and 8; both of SEQ ID NOs: 90 and 91; both of SEQ ID NOs: 37 and 38; both of SEQ ID NOs: 37 and 93; both of SEQ ID NOs: 92 and 38; both of SEQ ID NOs: 92 and 93; both of SEQ ID NOs: 70 and 71; both of SEQ ID NOs: 70 and 133; both of SEQ ID NOs: 132 and 71; both of SEQ ID NOs: 132 and 133; both of SEQ ID NOs: 47 and 48; both of SEQ ID NOs: 94 and 95; both of SEQ ID NOs: 49 and 50; both of SEQ ID NOs: 96 and 97; both of SEQ ID NOs: 72 and 73; or both of SEQ ID NOs: 88 and 89.

In embodiments of the invention, the inventive polypeptide can further comprise the constant region of the inventive TCR set forth above. In this regard, the polypeptide can further comprise the amino acid sequence of SEQ ID NO: 19 (WT murine constant region of α chain), SEQ ID NO: 20 (WT murine constant region of β chain), SEQ ID NO: 17, (substituted murine constant region of α chain), SEQ ID NO: 18 (substituted murine constant region of β chain), both SEQ ID NOs: 19 and 20, or both SEQ ID NOs: 17 and 18. Preferably, the polypeptide further comprises the amino acid sequences of both of SEQ ID NOs: 19 and 20 or both of SEQ ID NO: 17 and 18 in combination with any of the CDR regions or variable regions described herein with respect to other aspects of the invention.

In embodiments of the invention, the polypeptide comprises: (a) the amino acid sequence of SEQ ID NO: 17, wherein: (i) X at position 48 of SEQ ID NO: 17 is Thr or Cys; (ii) X at position 112 of SEQ ID NO: 17 is Ser, Ala, Val, Leu, Ile, Pro, Phe, Met, or Trp; (iii) X at position 114 of SEQ ID NO: 17 is Met, Ala, Val, Leu, Ile, Pro, Phe, or Trp; and (iv) X at position 115 of SEQ ID NO: 17 is Gly, Ala, Val, Leu, Ile, Pro, Phe, Met, or Trp; (b) the amino acid sequence of SEQ ID NO: 18, wherein X at position 57 of SEQ ID NO: 18 is Ser or Cys; or (c) both (a) and (b). In embodiments of the invention, one or both of SEQ ID NOs: 17 and 18 of the polypeptide are as defined in any one of Tables 2-4. The α chain constant regions provided herein are shown with an N-terminal asparagine. In some embodiments, the N-terminal amino acid of the α chain constant regions described herein is aspartic acid.

In embodiments of the invention, the inventive polypeptide can comprise the entire length of an a or 13 chain of the TCR described herein. In this regard, the inventive polypeptide can comprise the amino acid sequence of SEQ ID NO: 21, SEQ ID NO: 100, SEQ ID NO: 22, SEQ ID NO: 101, SEQ ID NO: 23, SEQ ID NO: 102, SEQ ID NO: 24, SEQ ID NO: 103, SEQ ID NO: 124, SEQ ID NO: 104, SEQ ID NO: 125, SEQ ID NO: 105, both of SEQ ID NOs: 21 and 22, both of SEQ ID NOs: 100 and 101, both of SEQ ID NOs: 23 and 24, both of SEQ ID NOs: 102 and 103, both of SEQ ID NOs: 124 and 125, both of SEQ ID NOs: 104 and 105, SEQ ID NO: 55, SEQ ID NO: 112, SEQ ID NO: 56, SEQ ID NO: 113, SEQ ID NO: 51, SEQ ID NO: 114, SEQ ID NO: 52, SEQ ID NO: 115, SEQ ID NO: 128, SEQ ID NO: 116, SEQ ID NO: 129, SEQ ID NO: 117, both of SEQ ID NO: 55 and 56, both of SEQ ID NO: 112 and 113, both of SEQ ID NO: 51 and 52, both of SEQ ID NO: 114 and 115, both of SEQ ID NO: 128 and 129, or both of SEQ ID NO: 116 and 117. In this regard, the inventive polypeptide can also comprise the amino acid sequence of SEQ ID NO: 41, SEQ ID NO: 106, SEQ ID NO: 42, SEQ ID NO: 107, SEQ ID NO: 39, SEQ ID NO: 108, SEQ ID NO: 40, SEQ ID NO: 109, SEQ ID NO: 126, SEQ ID NO: 110, SEQ ID NO: 127, SEQ ID NO: 111, both of SEQ ID NOs: 41 and 42, both of SEQ ID NOs: 106 and 107, both of SEQ ID NOs: 39 and 40, both of SEQ ID NOs: 108 and 109, both of SEQ ID NOs: 126 and 127, both of SEQ ID NOs: 110 and 111, SEQ ID NO: 57, SEQ ID NO: 118, SEQ ID NO: 58, SEQ ID NO: 119, SEQ ID NO: 53, SEQ ID NO: 120, SEQ ID NO: 54, SEQ ID NO: 121, SEQ ID NO: 130, SEQ ID NO: 122, SEQ ID NO: 131, SEQ ID NO: 123, both of SEQ ID NO: 57 and 58, both of SEQ ID NO: 118 and 119, both of SEQ ID NO: 53 and 54, both of SEQ ID NO: 120 and 121, both of SEQ ID NO: 130 and 131, or both of SEQ ID NO: 122 and 123. In this regard, the inventive polypeptide can also comprise the amino acid sequence of SEQ ID NO: 74, SEQ ID NO: 136, SEQ ID NO: 75, SEQ ID NO: 137, SEQ ID NO: 78, SEQ ID NO: 138, SEQ ID NO: 79, SEQ ID NO: 139, SEQ ID NO: 134, SEQ ID NO: 140, SEQ ID NO: 135, SEQ ID NO: 141, both of SEQ ID NOs: 74 and 75, both of SEQ ID NOs: 136 and 137, both of SEQ ID NOs: 78 and 79, both of SEQ ID NOs: 138 and 139, both of SEQ ID NOs: 134 and 135, both of SEQ ID NOs: 140 and 141, SEQ ID NO: 76, SEQ ID NO: 144, SEQ ID NO: 77, SEQ ID NO: 145, SEQ ID NO: 80, SEQ ID NO: 146, SEQ ID NO: 81, SEQ ID NO: 147, SEQ ID NO: 142, SEQ ID NO: 148, SEQ ID NO: 143, SEQ ID NO: 149, both of SEQ ID NO: 76 and 77, both of SEQ ID NO: 144 and 145, both of SEQ ID NO: 80 and 81, both of SEQ ID NO: 146 and 147, both of SEQ ID NO: 142 and 143, or both of SEQ ID NO: 148 and 149. Alternatively, the polypeptide of the invention can comprise both chains of the TCRs described herein.

In embodiments of the invention, the polypeptide comprises: (a) an α chain comprising the amino acid sequence of SEQ ID NO: 21 (α chain of 4391 TCR with a wild type N-terminal signal peptide), wherein: (i) X at position 180 of SEQ ID NO: 21 is Thr or Cys; (ii) X at position 244 of SEQ ID NO: 21 is Ser, Ala, Val, Leu, Ile, Pro, Phe, Met, or Trp; (iii) X at position 246 of SEQ ID NO: 21 is Met, Ala, Val, Leu, Ile, Pro, Phe, or Trp; and (iv) X at position 247 of SEQ ID NO: 21 is Gly, Ala, Val, Leu, Ile, Pro, Phe, Met, or Trp; (b) an α chain comprising the amino acid sequence of SEQ ID NO: 100 (α chain of 4391 TCR with a variant N-terminal signal peptide), werein: (i) X at position 180 of SEQ ID NO: 100 is Thr or Cys; (ii) X at position 244 of SEQ ID NO: 100 is Ser, Ala, Val, Leu, Ile, Pro, Phe, Met, or Trp; (iii) X at position 246 of SEQ ID NO: 100 is Met, Ala, Val, Leu, Ile, Pro, Phe, or Trp; and (iv) X at position 247 of SEQ ID NO: 100 is Gly, Ala, Val, Leu, Ile, Pro, Phe, Met, or Trp; (c) a β chain comprising the amino acid sequence of SEQ ID NO: 22 (β chain of 4391 TCR with a variant N-terminal signal peptide), wherein X at position 190 of SEQ ID NO: 22 is Ser or Cys; (d) a β chain comprising the amino acid sequence of SEQ ID NO: 101 (β chain of 4391 TCR with a wild type N-terminal signal peptide), wherein X at position 190 of SEQ ID NO: 101 is Ser or Cys; (e) an α chain comprising the amino acid sequence of SEQ ID NO: 41 (α chain of 4385 TCR with a wild type N-terminal signal peptide), wherein: (i) X at position 181 of SEQ ID NO: 41 is Thr or Cys; (ii) X at position 245 of SEQ ID NO: 41 is Ser, Ala, Val, Leu, Ile, Pro, Phe, Met, or Trp; (iii) X at position 247 of SEQ ID NO: 41 is Met, Ala, Val, Leu, Ile, Pro, Phe, or Trp; and (iv) X at position 248 of SEQ ID NO: 41 is Gly, Ala, Val, Leu, Ile, Pro, Phe, Met, or Trp; (0 an α chain comprising the amino acid sequence of SEQ ID NO: 106 (α chain of 4385 TCR with a variant N-terminal signal peptide), wherein: (i) X at position 181 of SEQ ID NO: 106 is Thr or Cys; (ii) X at position 245 of SEQ ID NO: 106 is Ser, Ala, Val, Leu, Ile, Pro, Phe, Met, or Trp; (iii) X at position 247 of SEQ ID NO: 106 is Met, Ala, Val, Leu, Ile, Pro, Phe, or Trp; and (iv) X at position 248 of SEQ ID NO: 106 is Gly, Ala, Val, Leu, Ile, Pro, Phe, Met, or Trp; (g) a β chain comprising the amino acid sequence of SEQ ID NO: 42 (β chain of 4385 TCR with a variant N-terminal signal peptide), wherein X at position 195 of SEQ ID NO: 42 is Ser or Cys; (h) a β chain comprising the amino acid sequence of SEQ ID NO: 107 (β chain of 4385 TCR with a wild type N-terminal signal peptide), wherein X at position 195 of SEQ ID NO: 107 is Ser or Cys; (i) an α chain comprising the amino acid sequence of SEQ ID NO: 74 (α chain of 4394 TCR with a wild type N-terminal signal peptide), wherein: (i) X at position 180 of SEQ ID NO: 74 is Thr or Cys; (ii) X at position 244 of SEQ ID NO: 74 is Ser, Ala, Val, Leu, Ile, Pro, Phe, Met, or Trp; (iii) X at position 246 of SEQ ID NO: 74 is Met, Ala, Val, Leu, Ile, Pro, Phe, or Trp; and (iv) X at position 247 of SEQ ID NO: 74 is Gly, Ala, Val, Leu, Ile, Pro, Phe, Met, or Trp; (j) an α chain comprising the amino acid sequence of SEQ ID NO: 136 (α chain of 4394 TCR with a variant N-terminal signal peptide), werein: (i) X at position 180 of SEQ ID NO: 136 is Thr or Cys; (ii) X at position 244 of SEQ ID NO: 136 is Ser, Ala, Val, Leu, Ile, Pro, Phe, Met, or Trp; (iii) X at position 246 of SEQ ID NO: 136 is Met, Ala, Val, Leu, Ile, Pro, Phe, or Trp; and (iv) X at position 247 of SEQ ID NO: 136 is Gly, Ala, Val, Leu, Ile, Pro, Phe, Met, or Trp; (k) a β chain comprising the amino acid sequence of SEQ ID NO: 75 (β chain of 4394 TCR with a variant N-terminal signal peptide), wherein X at position 187 of SEQ ID NO: 75 is Ser or Cys; (1) a β chain comprising the amino acid sequence of SEQ ID NO: 137 (α chain of 4394 TCR with a wild type N-terminal signal peptide), wherein X at position 187 of SEQ ID NO: 137 is Ser or Cys; (m) both (a) and (c); (n) both (b) and (d); (o) both (e) and (g); (p) both (0 and (h); (q) both (i) and (k); (r) both (j) and (1); (s) an α chain comprising the amino acid sequence of SEQ ID NO: 55 (α chain of 4391 TCR without N-terminal signal peptide predicted by IMGT), wherein: (i) X at position 160 of SEQ ID NO: 55 is Thr or Cys; (ii) X at position 224 of SEQ ID NO: 55 is Ser, Ala, Val, Leu, Ile, Pro, Phe, Met, or Trp; (iii) X at position 226 of SEQ ID NO: 55 is Met, Ala, Val, Leu, Ile, Pro, Phe, or Trp; and (iv) X at position 227 of SEQ ID NO: 55 is Gly, Ala, Val, Leu, Ile, Pro, Phe, Met, or Trp; (t) an a chain comprising the amino acid sequence of SEQ ID NO: 112 (α chain of 4391 TCR without N-terminal signal peptide predicted by SignalP), wherein: (i) X at position 159 of SEQ ID NO: 112 is Thr or Cys; (ii) X at position 223 of SEQ ID NO: 112 is Ser, Ala, Val, Leu, Ile, Pro, Phe, Met, or Trp; (iii) X at position 225 of SEQ ID NO: 112 is Met, Ala, Val, Leu, Ile, Pro, Phe, or Trp; and (iv) X at position 226 of SEQ ID NO: 112 is Gly, Ala, Val, Leu, Ile, Pro, Phe, Met, or Trp; (u) a β chain comprising the amino acid sequence of SEQ ID NO: 56 03 chain of 4391 TCR without N-terminal signal peptide predicted by IMGT), wherein X at position 168 of SEQ ID NO: 56 is Ser or Cys; (v) a β chain comprising the amino acid sequence of SEQ ID NO: 113 (β chain of 4391 TCR without N-terminal signal peptide predicted by SignalP), wherein X at position 173 of SEQ ID NO: 113 is Ser or Cys; (w) an a chain comprising the amino acid sequence of SEQ ID NO: 57 (α chain of 4385 TCR without N-terminal signal peptide predicted by IMGT), wherein: (i) X at position 160 of SEQ ID NO: 57 is Thr or Cys; (ii) X at position 224 of SEQ ID NO: 57 is Ser, Ala, Val, Leu, Ile, Pro, Phe, Met, or Trp; (iii) X at position 226 of SEQ ID NO: 57 is Met, Ala, Val, Leu, Ile, Pro, Phe, or Trp; and (iv) X at position 227 of SEQ ID NO: 57 is Gly, Ala, Val, Leu, Ile, Pro, Phe, Met, or Trp; (x) an α chain comprising the amino acid sequence of SEQ ID NO: 118 (α chain of 4385 TCR without N-terminal signal peptide predicted by SignalP), wherein: (i) X at position 161 of SEQ ID NO: 118 is Thr or Cys; (ii) X at position 225 of SEQ ID NO: 118 is Ser, Ala, Val, Leu, Ile, Pro, Phe, Met, or Trp; (iii) X at position 227 of SEQ ID NO: 118 is Met, Ala, Val, Leu, Ile, Pro, Phe, or Trp; and (iv) X at position 228 of SEQ ID NO: 118 is Gly, Ala, Val, Leu, Ile, Pro, Phe, Met, or Trp; (y) a β chain comprising the amino acid sequence of SEQ ID NO: 58 (α chain of 4385 TCR without N-terminal signal peptide predicted by IMGT), wherein X at position 173 of SEQ ID NO: 58 is Ser or Cys; (z) a β chain comprising the amino acid sequence of SEQ ID NO: 119 (α chain of 4385 TCR without N-terminal signal peptide predicted by SignalP), wherein X at position 178 of SEQ ID NO: 119 is Ser or Cys; (aa) an α chain comprising the amino acid sequence of SEQ ID NO: 76 (α chain of 4394 TCR without N-terminal signal peptide predicted by IMGT), wherein: (i) X at position 159 of SEQ ID NO: 76 is Thr or Cys; (ii) X at position 223 of SEQ ID NO: 76 is Ser, Ala, Val, Leu, Ile, Pro, Phe, Met, or Trp; (iii) X at position 225 of SEQ ID NO: 76 is Met, Ala, Val, Leu, Ile, Pro, Phe, or Trp; and (iv) X at position 226 of SEQ ID NO: 76 is Gly, Ala, Val, Leu, Ile, Pro, Phe, Met, or Trp; (bb) an α chain comprising the amino acid sequence of SEQ ID NO: 144 (a chain of 4394 TCR without N-terminal signal peptide predicted by SignalP), wherein: (i) X at position 160 of SEQ ID NO: 144 is Thr or Cys; (ii) X at position 224 of SEQ ID NO: 144 is Ser, Ala, Val, Leu, Ile, Pro, Phe, Met, or Trp; (iii) X at position 226 of SEQ ID NO: 144 is Met, Ala, Val, Leu, Ile, Pro, Phe, or Trp; and (iv) X at position 227 of SEQ ID NO: 144 is Gly, Ala, Val, Leu, Ile, Pro, Phe, Met, or Trp; (cc) a β chain comprising the amino acid sequence of SEQ ID NO: 77 (β chain of 4394 TCR without N-terminal signal peptide predicted by IMGT), wherein X at position 168 of SEQ ID NO: 77 is Ser or Cys; (dd) a (3 chain comprising the amino acid sequence of SEQ ID NO: 145 (β chain of 4394 TCR without N-terminal signal peptide predicted by SignalP), wherein X at position 166 of SEQ ID NO: 145 is Ser or Cys; (ee) both (s) and (u); (ff) both (t) and (v); (gg) both (w) and (y); (hh) both (x)) and (z); (hh) both (aa) and (cc); or (ii) both (bb) and (dd). In an embodiment of the invention, any one or more of SEQ ID NOs: 21, 22, 41, 42, 55-58, 74-77, 100, 101, 106, 107, 112, 113, 118, 119, 136, 137, 144 and 145 of the polypeptide are as defined in any one of Tables 2-4.

An embodiment of the invention further provides a protein comprising at least one of the polypeptides described herein. By “protein” is meant a molecule comprising one or more polypeptide chains.

In an embodiment, the protein of the invention can comprise (a) a first polypeptide chain comprising the amino acid sequences of SEQ ID NOs: 1-3 and a second polypeptide chain comprising the amino acid sequence of SEQ ID NOs: 4-6; or (b) a first polypeptide chain comprising the amino acid sequences of SEQ ID NOs: 31-33 and a second polypeptide chain comprising the amino acid sequences of SEQ ID NOs: 34-36; or (c) a first polypeptide chain comprising the amino acid sequences of SEQ ID NOs: 64-66 and a second polypeptide chain comprising the amino acid sequences of SEQ ID NOs: 67-69. The CDR3 of SEQ ID NO: 3, 6, 33, 36, 66, and 69, i.e., of the α chain or β chain or both, may further comprise a cysteine immediately N-terminal to the first amino acid of the CDR or a phenylalanine immediately C-terminal to the final amino acid or both.

In another embodiment of the invention, the protein may comprise (i) a first polypeptide chain comprising the amino acid sequence of SEQ ID NO: 7 and a second polypeptide chain comprising the amino acid sequence of SEQ ID NO: 8; (ii) a first polypeptide chain comprising the amino acid sequence of SEQ ID NO: 90 and a second polypeptide chain comprising the amino acid sequence of SEQ ID NO: 91; (iii) a first polypeptide chain comprising the amino acid sequence of SEQ ID NO: 7 and a second polypeptide chain comprising the amino acid sequence of SEQ ID NO: 91; (iv) a first polypeptide chain comprising the amino acid sequence of SEQ ID NO: 90 and a second polypeptide chain comprising the amino acid sequence of SEQ ID NO: 8; (v) a first polypeptide chain comprising the amino acid sequence of SEQ ID NO: 37 and a second polypeptide chain comprising the amino acid sequence of SEQ ID NO: 38; (vi) a first polypeptide chain comprising the amino acid sequence of SEQ ID NO: 92 and a second polypeptide chain comprising the amino acid sequence of SEQ ID NO: 93; (vii) a first polypeptide chain comprising the amino acid sequence of SEQ ID NO: 37 and a second polypeptide chain comprising the amino acid sequence of SEQ ID NO: 93; (viii) a first polypeptide chain comprising the amino acid sequence of SEQ ID NO: 92 and a second polypeptide chain comprising the amino acid sequence of SEQ ID NO: 38; (i) a first polypeptide chain comprising the amino acid sequence of SEQ ID NO: 70 and a second polypeptide chain comprising the amino acid sequence of SEQ ID NO: 71; (ii) a first polypeptide chain comprising the amino acid sequence of SEQ ID NO: 132 and a second polypeptide chain comprising the amino acid sequence of SEQ ID NO: 133; (iii) a first polypeptide chain comprising the amino acid sequence of SEQ ID NO: 70 and a second polypeptide chain comprising the amino acid sequence of SEQ ID NO: 133; (iv) a first polypeptide chain comprising the amino acid sequence of SEQ ID NO: 132 and a second polypeptide chain comprising the amino acid sequence of SEQ ID NO: 71; (ix) a first polypeptide chain comprising the amino acid sequence of SEQ ID NO: 47 and a second polypeptide chain comprising the amino acid sequence of SEQ ID NO: 48; (x) a first polypeptide chain comprising the amino acid sequence of SEQ ID NO: 94 and a second polypeptide chain comprising the amino acid sequence of SEQ ID NO: 95; (xi) a first polypeptide chain comprising the amino acid sequence of SEQ ID NO: 49 and a second polypeptide chain comprising the amino acid sequence of SEQ ID NO: 50; (xii) a first polypeptide chain comprising the amino acid sequence of SEQ ID NO: 96 and a second polypeptide chain comprising the amino acid sequence of SEQ ID NO: 97; (ix) a first polypeptide chain comprising the amino acid sequence of SEQ ID NO: 72 and a second polypeptide chain comprising the amino acid sequence of SEQ ID NO: 73; or (x) a first polypeptide chain comprising the amino acid sequence of SEQ ID NO: 88 and a second polypeptide chain comprising the amino acid sequence of SEQ ID NO: 89.

The inventive protein may further comprise any of the constant regions described herein with respect to other aspects of the invention. In this regard, in embodiments of the invention, the first polypeptide chain may further comprise the amino acid sequence of SEQ ID NO: 17 and the second polypeptide chain may further comprise the amino acid sequence of SEQ ID NO: 18. In embodiments of the invention, the first polypeptide chain may further comprise the amino acid sequence of SEQ ID NO: 19 and the second polypeptide chain may further comprise the amino acid sequence of SEQ ID NO: 20.

In embodiments of the invention, the protein comprises: (a) a first polypeptide chain comprising the amino acid sequence of SEQ ID NO: 17, wherein: (i) X at position 48 of SEQ ID NO: 17 is Thr or Cys; (ii) X at position 112 of SEQ ID NO: 17 is Ser, Ala, Val, Leu, Ile, Pro, Phe, Met, or Trp; (iii) X at position 114 of SEQ ID NO: 17 is Met, Ala, Val, Leu, Ile, Pro, Phe, or Trp; and (iv) X at position 115 of SEQ ID NO: 17 is Gly, Ala, Val, Leu, Ile, Pro, Phe, Met, or Trp; (b) a second polypeptide chain comprising the amino acid sequence of SEQ ID NO: 18, wherein X at position 57 of SEQ ID NO: 18 is Ser or Cys; or (c) both (a) and (b). In embodiments of the invention, one or both of SEQ ID NOs: 17 and 18 of the protein are as defined in any one of Tables 2-4.

Alternatively or additionally, the protein of an embodiment of the invention can comprise (a) an α chain comprising the amino acid sequence of SEQ ID NO: 21 (α chain of 4391 TCR with a wild type N-terminal signal peptide), wherein: (i) X at position 180 of SEQ ID NO: 21 is Thr or Cys; (ii) X at position 244 of SEQ ID NO: 21 is Ser, Ala, Val, Leu, Ile, Pro, Phe, Met, or Trp; (iii) X at position 246 of SEQ ID NO: 21 is Met, Ala, Val, Leu, Ile, Pro, Phe, or Trp; and (iv) X at position 247 of SEQ ID NO: 21 is Gly, Ala, Val, Leu, Ile, Pro, Phe, Met, or Trp; (b) an α chain comprising the amino acid sequence of SEQ ID NO: 100 (a chain of 4391 TCR with a variant N-terminal signal peptide), werein: (i) X at position 180 of SEQ ID NO: 100 is Thr or Cys; (ii) X at position 244 of SEQ ID NO: 100 is Ser, Ala, Val, Leu, Ile, Pro, Phe, Met, or Trp; (iii) X at position 246 of SEQ ID NO: 100 is Met, Ala, Val, Leu, Ile, Pro, Phe, or Trp; and (iv) X at position 247 of SEQ ID NO: 100 is Gly, Ala, Val, Leu, Ile, Pro, Phe, Met, or Trp; (c) a β chain comprising the amino acid sequence of SEQ ID NO: 22 (β chain of 4391 TCR with a variant N-terminal signal peptide), wherein X at position 190 of SEQ ID NO: 22 is Ser or Cys; (d) a β chain comprising the amino acid sequence of SEQ ID NO: 101 (β chain of 4391 TCR with a wild type N-terminal signal peptide), wherein X at position 190 of SEQ ID NO: 101 is Ser or Cys; (e) an α chain comprising the amino acid sequence of SEQ ID NO: 41 (α chain of 4385 TCR with a wild type N-terminal signal peptide), wherein: (i) X at position 181 of SEQ ID NO: 41 is Thr or Cys; (ii) X at position 245 of SEQ ID NO: 41 is Ser, Ala, Val, Leu, Ile, Pro, Phe, Met, or Trp; (iii) X at position 247 of SEQ ID NO: 41 is Met, Ala, Val, Leu, Ile, Pro, Phe, or Trp; and (iv) X at position 248 of SEQ ID NO: 41 is Gly, Ala, Val, Leu, Ile, Pro, Phe, Met, or Trp; (0 an a chain comprising the amino acid sequence of SEQ ID NO: 106 (α chain of 4385 TCR with a variant N-terminal signal peptide), wherein: (i) X at position 181 of SEQ ID NO: 106 is Thr or Cys; (ii) X at position 245 of SEQ ID NO: 106 is Ser, Ala, Val, Leu, Ile, Pro, Phe, Met, or Trp; (iii) X at position 247 of SEQ ID NO: 106 is Met, Ala, Val, Leu, Ile, Pro, Phe, or Trp; and (iv) X at position 248 of SEQ ID NO: 106 is Gly, Ala, Val, Leu, Ile, Pro, Phe, Met, or Trp; (g) a β chain comprising the amino acid sequence of SEQ ID NO: 42 (β chain of 4385 TCR with a variant N-terminal signal peptide), wherein X at position 195 of SEQ ID NO: 42 is Ser or Cys; (h) a β chain comprising the amino acid sequence of SEQ ID NO: 107 (β chain of 4385 TCR with a wild type N-terminal signal peptide), wherein X at position 195 of SEQ ID NO: 107 is Ser or Cys; (i) an α chain comprising the amino acid sequence of SEQ ID NO: 74 (α chain of 4394 TCR with a wild type N-terminal signal peptide), wherein: (i) X at position 180 of SEQ ID NO: 74 is Thr or Cys; (ii) X at position 244 of SEQ ID NO: 74 is Ser, Ala, Val, Leu, Ile, Pro, Phe, Met, or Trp; (iii) X at position 246 of SEQ ID NO: 74 is Met, Ala, Val, Leu, Ile, Pro, Phe, or Trp; and (iv) X at position 247 of SEQ ID NO: 74 is Gly, Ala, Val, Leu, Ile, Pro, Phe, Met, or Trp; (j) an α chain comprising the amino acid sequence of SEQ ID NO: 136 (α chain of 4394 TCR with a variant N-terminal signal peptide), werein: (i) X at position 180 of SEQ ID NO: 136 is Thr or Cys; (ii) X at position 244 of SEQ ID NO: 136 is Ser, Ala, Val, Leu, Ile, Pro, Phe, Met, or Trp; (iii) X at position 246 of SEQ ID NO: 136 is Met, Ala, Val, Leu, Ile, Pro, Phe, or Trp; and (iv) X at position 247 of SEQ ID NO: 136 is Gly, Ala, Val, Leu, Ile, Pro, Phe, Met, or Trp; (k) a β chain comprising the amino acid sequence of SEQ ID NO: 75 (β chain of 4394 TCR with a variant N-terminal signal peptide), wherein X at position 187 of SEQ ID NO: 75 is Ser or Cys; (1) a β chain comprising the amino acid sequence of SEQ ID NO: 137 (β chain of 4394 TCR with a wild type N-terminal signal peptide), wherein X at position 187 of SEQ ID NO: 137 is Ser or Cys; (m) both (a) and (c); (n) both (b) and (d); (o) both (e) and (g); (p) both (0 and (h); (q) both (i) and (k); (r) both (j) and (1); (s) an α chain comprising the amino acid sequence of SEQ ID NO: 55 (α chain of 4391 TCR without N-terminal signal peptide predicted by IMGT), wherein: (i) X at position 160 of SEQ ID NO: 55 is Thr or Cys; (ii) X at position 224 of SEQ ID NO: 55 is Ser, Ala, Val, Leu, Ile, Pro, Phe, Met, or Trp; (iii) X at position 226 of SEQ ID NO: 55 is Met, Ala, Val, Leu, Ile, Pro, Phe, or Trp; and (iv) X at position 227 of SEQ ID NO: 55 is Gly, Ala, Val, Leu, Ile, Pro, Phe, Met, or Trp; (t) an α chain comprising the amino acid sequence of SEQ ID NO: 112 (α chain of 4391 TCR without N-terminal signal peptide predicted by SignalP), wherein: (i) X at position 159 of SEQ ID NO: 112 is Thr or Cys; (ii) X at position 223 of SEQ ID NO: 112 is Ser, Ala, Val, Leu, Ile, Pro, Phe, Met, or Trp; (iii) X at position 225 of SEQ ID NO: 112 is Met, Ala, Val, Leu, Ile, Pro, Phe, or Trp; and (iv) X at position 226 of SEQ ID NO: 112 is Gly, Ala, Val, Leu, Ile, Pro, Phe, Met, or Trp; (u) a β chain comprising the amino acid sequence of SEQ ID NO: 56 (β chain of 4391 TCR without N-terminal signal peptide predicted by IMGT), wherein X at position 168 of SEQ ID NO: 56 is Ser or Cys; (v) a β chain comprising the amino acid sequence of SEQ ID NO: 113 (β chain of 4391 TCR without N-terminal signal peptide predicted by SignalP), wherein X at position 173 of SEQ ID NO: 113 is Ser or Cys; (w) an α chain comprising the amino acid sequence of SEQ ID NO: 57 (α chain of 4385 TCR without N-terminal signal peptide predicted by IMGT), wherein: (i) X at position 160 of SEQ ID NO: 57 is Thr or Cys; (ii) X at position 224 of SEQ ID NO: 57 is Ser, Ala, Val, Leu, Ile, Pro, Phe, Met, or Trp; (iii) X at position 226 of SEQ ID NO: 57 is Met, Ala, Val, Leu, Ile, Pro, Phe, or Trp; and (iv) X at position 227 of SEQ ID NO: 57 is Gly, Ala, Val, Leu, Ile, Pro, Phe, Met, or Trp; (x) an α chain comprising the amino acid sequence of SEQ ID NO: 118 (α chain of 4385 TCR without N-terminal signal peptide predicted by SignalP), wherein: (i) X at position 161 of SEQ ID NO: 118 is Thr or Cys; (ii) X at position 225 of SEQ ID NO: 118 is Ser, Ala, Val, Leu, Ile, Pro, Phe, Met, or Trp; (iii) X at position 227 of SEQ ID NO: 118 is Met, Ala, Val, Leu, Ile, Pro, Phe, or Trp; and (iv) X at position 228 of SEQ ID NO: 118 is Gly, Ala, Val, Leu, Ile, Pro, Phe, Met, or Trp; (y) a 13 chain comprising the amino acid sequence of SEQ ID NO: 58 (β chain of 4385 TCR without N-terminal signal peptide predicted by IMGT), wherein X at position 173 of SEQ ID NO: 58 is Ser or Cys; (z) a 13 chain comprising the amino acid sequence of SEQ ID NO: 119 (α chain of 4385 TCR without N-terminal signal peptide predicted by SignalP), wherein X at position 178 of SEQ ID NO: 119 is Ser or Cys; (aa) an α chain comprising the amino acid sequence of SEQ ID NO: 76 (α chain of 4394 TCR without N-terminal signal peptide predicted by IMGT), wherein: (i) X at position 159 of SEQ ID NO: 76 is Thr or Cys; (ii) X at position 223 of SEQ ID NO: 76 is Ser, Ala, Val, Leu, Ile, Pro, Phe, Met, or Trp; (iii) X at position 225 of SEQ ID NO: 76 is Met, Ala, Val, Leu, Ile, Pro, Phe, or Trp; and (iv) X at position 226 of SEQ ID NO: 76 is Gly, Ala, Val, Leu, Ile, Pro, Phe, Met, or Trp; (bb) an α chain comprising the amino acid sequence of SEQ ID NO: 144 (α chain of 4394 TCR without N-terminal signal peptide predicted by SignalP), wherein: (i) X at position 160 of SEQ ID NO: 144 is Thr or Cys; (ii) X at position 224 of SEQ ID NO: 144 is Ser, Ala, Val, Leu, Ile, Pro, Phe, Met, or Trp; (iii) X at position 226 of SEQ ID NO: 144 is Met, Ala, Val, Leu, Ile, Pro, Phe, or Trp; and (iv) X at position 227 of SEQ ID NO: 144 is Gly, Ala, Val, Leu, Ile, Pro, Phe, Met, or Trp; (cc) a 13 chain comprising the amino acid sequence of SEQ ID NO: 77 (β chain of 4394 TCR without N-terminal signal peptide predicted by IMGT), wherein X at position 168 of SEQ ID NO: 77 is Ser or Cys; (dd) a 13 chain comprising the amino acid sequence of SEQ ID NO: 145 (β chain of 4394 TCR without N-terminal signal peptide predicted by SignalP), wherein X at position 166 of SEQ ID NO: 145 is Ser or Cys; (ee) both (s) and (u); (ff) both (t) and (v); (gg) both (w) and (y); (hh) both (x)) and (z); (hh) both (aa) and (cc); or (ii) both (bb) and (dd). In an embodiment of the invention, one or more of SEQ ID NOs: 21, 22, 41, 42, 55-58, 100, 101, 106, 107, 112, 113, 118, and 119 are as defined in any one of Tables 2-4.

The protein of the invention can be a TCR. Alternatively, if, for example, the protein comprises a single polypeptide chain comprising the amino acid sequences of both SEQ ID NOs: 21 and 22, both SEQ ID NOs: 23 and 24, both SEQ ID NOs: 124 and 125, both SEQ ID NOs: 100 and 101, both SEQ ID NOs: 102 and 103, both SEQ ID NOs: 104 and 105, both SEQ ID NOs: 41 and 42, both SEQ ID NOs: 39 and 40, both SEQ ID NOs: 126 and 127, both SEQ ID NOs: 106 and 107, both SEQ ID NOs: 108 and 109, both SEQ ID NOs: 110 and 85, SEQ ID NOs: 74 and 75, both SEQ ID NOs: 78 and 79, both SEQ ID NOs: 134 and 135, both SEQ ID NOs: 136 and 137, both SEQ ID NOs: 138 and 139, both SEQ ID NOs: 140 and 141, or if the first and/or second polypeptide chain(s) of the protein further comprise(s) other amino acid sequences, e.g., an amino acid sequence encoding an immunoglobulin or a portion thereof, then the inventive protein can be a fusion protein. In this regard, an embodiment of the invention also provides a fusion protein comprising at least one of the inventive polypeptides described herein along with at least one other polypeptide. The other polypeptide can exist as a separate polypeptide of the fusion protein, or can exist as a polypeptide, which is expressed in frame (in tandem) with one of the inventive polypeptides described herein. The other polypeptide can encode any peptidic or proteinaceous molecule, or a portion thereof, including, but not limited to an immunoglobulin, CD3, CD4, CD8, an MHC molecule, a CD1 molecule, e.g., CD1a, CD1b, CD1c, CD1d, etc.

The fusion protein can comprise one or more copies of the inventive polypeptide and/or one or more copies of the other polypeptide. For instance, the fusion protein can comprise 1, 2, 3, 4, 5, or more, copies of the inventive polypeptide and/or of the other polypeptide. Suitable methods of making fusion proteins are known in the art, and include, for example, recombinant methods.

In some embodiments of the invention, the TCRs, polypeptides, and proteins of the invention may be expressed as a single protein comprising a linker peptide linking the a chain and the α chain. In this regard, the TCRs, polypeptides, and proteins of the invention may further comprise a linker peptide. The linker peptide may advantageously facilitate the expression of a recombinant TCR, polypeptide, and/or protein in a host cell. The linker peptide may comprise any suitable amino acid sequence. For example, the linker peptide may be a furin-SGSG-P2A linker comprising the amino acid sequence of SEQ ID NO: 25. Upon expression of the construct including the linker peptide by a host cell, the linker peptide may be cleaved, resulting in separated a and α chains. In embodiments of the invention, the TCR, polypeptide, or protein may comprise an amino acid sequence comprising a full-length α chain, a full-length α chain, and a linker peptide positioned between the a and α chains, for example α chain—linker-0 chain or α chain—linker—α chain.

In an embodiment of the invention, the TCR, polypeptide, or protein may comprise an amino acid sequence as set forth in SEQ ID NO: 161 comprising from N-terminus to C-terminus, a β chain, a linker (SEQ ID NO:25) and an α chain. The variant comprises a β chain variable region (with a variant signal peptide) as set forth in SEQ ID NO: 8 and a modified β constant domain as set forth in SEQ ID NO: 99. The full-length β chain of the variant is set forth in SEQ ID NO: 125. The variant also comprises an α chain variable region (with a wild type signal peptide) as set forth in SEQ ID NO: 7 and a modified α constant domain as set forth in SEQ ID NO: 98. The full-length α chain of the variant is set forth in SEQ ID NO: 124.

In another embodiment of the invention, the TCR, polypeptide, or protein may comprise an amino acid sequence as set forth in SEQ ID NO: 162 comprising from N-terminus to C-terminus, an α chain, a linker (SEQ ID NO:25) and a β chain. The variant comprises an α chain variable region (with a variant signal peptide) as set forth in SEQ ID NO: 90 and a modified a constant domain as set forth in SEQ ID NO: 98. The full-length α chain of the variant is set forth in SEQ ID NO: 104. The variant also comprises a β chain variable region (with a wild type signal peptide) as set forth in SEQ ID NO: 91 and a modified β constant domain as set forth in SEQ ID NO: 99. The full-length 13 chain of the variant is set forth in SEQ ID NO: 105.

In an embodiment of the invention, the TCR, polypeptide, or protein may comprise an amino acid sequence as set forth in SEQ ID NO: 163 comprising from N-terminus to C-terminus, a β chain, a linker (SEQ ID NO:25) and an α chain. The variant comprises a β chain variable region (with a variant signal peptide) as set forth in SEQ ID NO: 38 and a modified β constant domain as set forth in SEQ ID NO: 99. The full-length β chain of the variant is set forth in SEQ ID NO: 127. The variant also comprises an α chain variable region (with a wild type signal peptide) as set forth in SEQ ID NO: 37 and a modified a constant domain as set forth in SEQ ID NO: 98. The full-length α chain of the variant is set forth in SEQ ID NO: 126.

In another embodiment of the invention, the TCR, polypeptide, or protein may comprise an amino acid sequence as set forth in SEQ ID NO: 164 comprising from N-terminus to C-terminus, an α chain, a linker (SEQ ID NO:25) and a β chain. The variant comprises an α chain variable region (with a variant signal peptide) as set forth in SEQ ID NO: 92 and a modified a constant domain as set forth in SEQ ID NO: 98. The full-length a chain of the variant is set forth in SEQ ID NO: 110. The variant also comprises a β chain variable region (with a wild type signal peptide) as set forth in SEQ ID NO: 93 and a modified β constant domain as set forth in SEQ ID NO: 99. The full-length 13 chain of the variant is set forth in SEQ ID NO: 111.

In an embodiment of the invention, the TCR, polypeptide, or protein may comprise an amino acid sequence as set forth in SEQ ID NO: 165 comprising from N-terminus to C-terminus, a β chain, a linker (SEQ ID NO:25) and an α chain. The variant comprises a β chain variable region (with a variant signal peptide) as set forth in SEQ ID NO: 71 and a modified β constant domain as set forth in SEQ ID NO: 99. The full-length β chain of the variant is set forth in SEQ ID NO: 135. The variant also comprises an α chain variable region (with a wild type signal peptide) as set forth in SEQ ID NO: 70 and a modified a constant domain as set forth in SEQ ID NO: 98. The full-length α chain of the variant is set forth in SEQ ID NO: 134.

In another embodiment of the invention, the TCR, polypeptide, or protein may comprise an amino acid sequence as set forth in SEQ ID NO: 166 comprising from N-terminus to C-terminus, an α chain, a linker (SEQ ID NO:25) and a β chain. The variant comprises an α chain variable region (with a variant signal peptide) as set forth in SEQ ID NO: 132 and a modified a constant domain as set forth in SEQ ID NO: 98. The full-length a chain of the variant is set forth in SEQ ID NO: 140. The variant also comprises a β chain variable region (with a wild type signal peptide) as set forth in SEQ ID NO: 133 and a modified β constant domain as set forth in SEQ ID NO: 99. The full-length 13 chain of the variant is set forth in SEQ ID NO: 141.

In some embodiments, the TCR, polypeptide or protein disclosed herein comprises an α chain and/or a β chain, as disclosed herein, comprising a signal peptide. In some embodiments, the sequence of the signal peptide of any of the α chains and/or β chains disclosed herein comprises an alanine or histidine residue substituted for the wild-type residue at position 2.

In some embodiments, the TCR, polypeptide or protein disclosed herein comprises a mature version of an α chain and/or a β chain, as disclosed herein, that lacks a signal peptide. The sequence of the signal peptide or mature form of the α chain and/or a (3 chain can be performed according to any method known in the art including IMGT and SignalP.

The protein of the invention can be a recombinant antibody, or an antigen binding portion thereof, comprising at least one of the inventive polypeptides described herein. As used herein, “recombinant antibody” refers to a recombinant (e.g., genetically engineered) protein comprising at least one of the polypeptides of the invention and a polypeptide chain of an antibody, or an antigen binding portion thereof. The polypeptide of an antibody, or antigen binding portion thereof, can be a heavy chain, a light chain, a variable or constant region of a heavy or light chain, a single chain variable fragment (scFv), or an Fc, Fab, or F(ab)2′ fragment of an antibody, etc. The polypeptide chain of an antibody, or an antigen binding portion thereof, can exist as a separate polypeptide of the recombinant antibody. Alternatively, the polypeptide chain of an antibody, or an antigen binding portion thereof, can exist as a polypeptide, which is expressed in frame (in tandem) with the polypeptide of the invention. The polypeptide of an antibody, or an antigen binding portion thereof, can be a polypeptide of any antibody or any antibody fragment, including any of the antibodies and antibody fragments described herein.

Included in the scope of the invention are functional variants of the inventive TCRs, polypeptides, or proteins described herein. The term “functional variant,” as used herein, refers to a TCR, polypeptide, or protein having substantial or significant sequence identity or similarity to a parent TCR, polypeptide, or protein, which functional variant retains the biological activity of the TCR, polypeptide, or protein of which it is a variant. Functional variants encompass, for example, those variants of the TCR, polypeptide, or protein described herein (the parent TCR, polypeptide, or protein) that retain the ability to specifically bind to mutated RAS for which the parent TCR has antigenic specificity or to which the parent polypeptide or protein specifically binds, to a similar extent, the same extent, or to a higher extent, as the parent TCR, polypeptide, or protein. In reference to the parent TCR, polypeptide, or protein, the functional variant can, for instance, be at least about 30%, about 50%, about 75%, about 80%, about 90%, about 95%, about 96%, about 97%, about 98%, about 99% or more identical in amino acid sequence to the parent TCR, polypeptide, or protein, respectively.

The functional variant can, for example, comprise the amino acid sequence of the parent TCR, polypeptide, or protein with at least one conservative amino acid substitution. Conservative amino acid substitutions are known in the art, and include amino acid substitutions in which one amino acid having certain physical and/or chemical properties is exchanged for another amino acid that has the same chemical or physical properties. For instance, the conservative amino acid substitution can be an acidic amino acid substituted for another acidic amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Val, etc.), a basic amino acid substituted for another basic amino acid (Lys, Arg, etc.), an amino acid with a polar side chain substituted for another amino acid with a polar side chain (Asn, Cys, Gln, Ser, Thr, Tyr, etc.), etc.

Alternatively or additionally, the functional variants can comprise the amino acid sequence of the parent TCR, polypeptide, or protein with at least one non-conservative amino acid substitution. In this case, it is preferable for the non-conservative amino acid substitution to not interfere with or inhibit the biological activity of the functional variant. Preferably, the non-conservative amino acid substitution enhances the biological activity of the functional variant, such that the biological activity of the functional variant is increased as compared to the parent TCR, polypeptide, or protein.

Each signal peptide of the TCRs, polypeptides, proteins, functional variants, and functional portions described herein, when present, can be any suitable TCR signal peptide, so long as the TCR, polypeptide, protein, or functional variant is expressed and has antigenic specificity for a mutated human RAS amino acid sequence with a substitution of glycine at position 12 with valine presented by an HLA Class I molecule.

The TCR, polypeptide, or protein can consist essentially of the specified amino acid sequence or sequences described herein, such that other components of the TCR, polypeptide, or protein, e.g., other amino acids, do not materially change the biological activity of the TCR, polypeptide, or protein. In this regard, the inventive TCR, polypeptide, or protein can, for example, consist essentially of the amino acid sequence of SEQ ID NO: 21, SEQ ID NO: 100, SEQ ID NO: 22, SEQ ID NO: 101, SEQ ID NO: 23, SEQ ID NO: 102, SEQ ID NO: 24, SEQ ID NO: 103, SEQ ID NO: 124, SEQ ID NO: 104, SEQ ID NO: 125, SEQ ID NO: 105, both of SEQ ID NOs: 21 and 22, both of SEQ ID NOs: 100 and 101, both of SEQ ID NOs: 23 and 24, both of SEQ ID NOs: 102 and 103, both of SEQ ID NOs: 124 and 125, both of SEQ ID NOs: 104 and 105, SEQ ID NO: 55, SEQ ID NO: 112, SEQ ID NO: 56, SEQ ID NO: 113, SEQ ID NO: 51, SEQ ID NO: 114, SEQ ID NO: 52, SEQ ID NO: 115, SEQ ID NO: 128, SEQ ID NO: 116, SEQ ID NO: 129, SEQ ID NO: 117, both of SEQ ID NO: 55 and 56, both of SEQ ID NO: 112 and 113, both of SEQ ID NO: 51 and 52, both of SEQ ID NO: 114 and 115, both of SEQ ID NO: 128 and 129, or both of SEQ ID NO: 116 and 117, SEQ ID NO: 41, SEQ ID NO: 106, SEQ ID NO: 42, SEQ ID NO: 107, SEQ ID NO: 39, SEQ ID NO: 108, SEQ ID NO: 40, SEQ ID NO: 109, SEQ ID NO: 126, SEQ ID NO: 110, SEQ ID NO: 127, SEQ ID NO: 111, both of SEQ ID NOs: 41 and 42, both of SEQ ID NOs: 106 and 107, both of SEQ ID NOs: 39 and 40, both of SEQ ID NOs: 108 and 109, both of SEQ ID NOs: 126 and 127, both of SEQ ID NOs: 110 and 111, SEQ ID NO: 57, SEQ ID NO: 118, SEQ ID NO: 58, SEQ ID NO: 119, SEQ ID NO: 53, SEQ ID NO: 120, SEQ ID NO: 54, SEQ ID NO: 121, SEQ ID NO: 130, SEQ ID NO: 122, SEQ ID NO: 131, SEQ ID NO: 123, both of SEQ ID NO: 57 and 58, both of SEQ ID NO: 118 and 119, both of SEQ ID NO: 53 and 54, both of SEQ ID NO: 120 and 121, both of SEQ ID NO: 130 and 131, or both of SEQ ID NO: 122 and 123, SEQ ID NO: 74, SEQ ID NO: 136, SEQ ID NO: 75, SEQ ID NO: 137, SEQ ID NO: 78, SEQ ID NO: 138, SEQ ID NO: 79, SEQ ID NO: 139, SEQ ID NO: 134, SEQ ID NO: 140, SEQ ID NO: 135, SEQ ID NO: 141, both of SEQ ID NOs: 74 and 75, both of SEQ ID NOs: 136 and 137, both of SEQ ID NOs: 78 and 79, both of SEQ ID NOs: 138 and 139, both of SEQ ID NOs: 134 and 135, both of SEQ ID NOs: 140 and 141, SEQ ID NO: 76, SEQ ID NO: 144, SEQ ID NO: 77, SEQ ID NO: 145, SEQ ID NO: 80, SEQ ID NO: 146, SEQ ID NO: 81, SEQ ID NO: 147, SEQ ID NO: 142, SEQ ID NO: 148, SEQ ID NO: 143, SEQ ID NO: 149, both of SEQ ID NO: 76 and 77, both of SEQ ID NO: 144 and 145, both of SEQ ID NO: 80 and 81, both of SEQ ID NO: 146 and 147, both of SEQ ID NO: 142 and 143, or both of SEQ ID NO: 148 and 149. . Also, for instance, the inventive TCRs, polypeptides, or proteins can consist essentially of the amino acid sequence(s) of (i) SEQ ID NO: 7, (ii) SEQ ID NO: 90, (iii) SEQ ID NO: 8, (iv) SEQ ID NO: 91, (v) SEQ ID NO: 37, (vi) SEQ ID NO: 92, (vii) SEQ ID NO: 38, (viii) SEQ ID NO: 105N3, (ix) SEQ ID NO: 70, (x) SEQ ID NO: 132, (xi) SEQ ID NO: 71, (xii) SEQ ID NO: 133, (xiii) both of SEQ ID NOs: 7 and 8, (xiv) both of SEQ ID NOs: 7 and 91, (xv) both of SEQ ID NOs: 90 and 8, (xvi) both of SEQ ID NOs: 90 and 91, (xvii) both of SEQ ID NOs: 37 and 38, (xviii) both of SEQ ID NOs: 92 and 38, (xix) both of SEQ ID NOs: 37 and 93, (xx) both of SEQ ID NOs: 92 and 93; (xxi) both of SEQ ID NOs: 70 and 71, (xxii) both of SEQ ID NOs: 70 and 133, (xxiii) both of SEQ ID NOs: 132 and 71; or (xxiv) both of SEQ ID NOs: 132 and 133. Furthermore, the inventive TCRs, polypeptides, or proteins can consist essentially of the amino acid sequence of (a) any one or more of SEQ ID NOs: 1-6, 31-36 and 64-69; (b) all of SEQ ID NO: 1-3; (c) all of SEQ ID NO: 4-6; (d) all of SEQ ID NO: 31-33; (e) all of SEQ ID NOs: 34-36; (0 all of SEQ ID NOs: 64-66, (g) all of SEQ ID NOs: 67-69, (h) all of SEQ ID NOs: 1-6, (i) all of SEQ ID NOs: 31-36, or (j) all of SEQ ID NOs: 64-69.

The TCRs, polypeptides, and proteins of the invention can be of any length, i.e., can comprise any number of amino acids, provided that the TCRs, polypeptides, or proteins retain their biological activity, e.g., the ability to specifically bind to mutated RAS; detect cancer in a mammal; or treat or prevent cancer in a mammal, etc. For example, the polypeptide can be in the range of from about 50 to about 5000 amino acids long, such as about 50, about 70, about 75, about 100, about 125, about 150, about 175, about 200, about 300, about 400, about 500, about 600, about 700, about 800, about 900, about 1000 or more amino acids in length. In this regard, the polypeptides of the invention also include oligopeptides.

The TCRs, polypeptides, and proteins of the invention can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, a,y-diaminobutyric acid, a, β-diaminopropionic acid, homophenylalanine, and α-tert-butylglycine.

The TCRs, polypeptides, and proteins of the invention can be, e.g., glycosylated, amidated, carboxylated, phosphorylated, esterified, N-acylated, cyclized via, e.g., a disulfide bridge, or converted into an acid addition salt and/or optionally dimerized or polymerized, or conjugated.

The TCR, polypeptide, and/or protein of the invention can be obtained by methods known in the art such as, for example, de novo synthesis. Also, polypeptides and proteins can be recombinantly produced using the nucleic acids described herein using standard recombinant methods. See, for instance, Green and Sambrook, Molecular Cloning: A Laboratory Manual, 4th ed., Cold Spring Harbor Press, Cold Spring Harbor, N.Y. (2012). Alternatively, the TCRs, polypeptides, and/or proteins described herein can be commercially synthesized by any of a variety of commercial entities. In this respect, the inventive TCRs, polypeptides, and proteins can be synthetic, recombinant, isolated, and/or purified. An embodiment of the invention provides an isolated or purified TCR, polypeptide, or protein encoded by any of the nucleic acids or vectors described herein with respect to other aspects of the invention. Another embodiment of the invention provides an isolated or purified TCR, polypeptide, or protein that results from expression of any of the nucleic acids or vectors described herein with respect to other aspects of the invention in a cell. Still another embodiment of the invention provides a method of producing any of the TCRs, polypeptides, or proteins described herein, the method comprising culturing any of the host cells or populations of host cells described herein so that the TCR, polypeptide, or protein is produced.

Included in the scope of the invention are conjugates, e.g., bioconjugates, comprising any of the inventive TCRs, polypeptides, or proteins (including any of the functional portions or variants thereof), nucleic acids, recombinant expression vectors, host cells, populations of host cells, or antibodies, or antigen binding portions thereof. Conjugates, as well as methods of synthesizing conjugates in general, are known in the art.

An embodiment of the invention provides a nucleic acid comprising a nucleotide sequence encoding any of the TCRs, polypeptides, or proteins described herein. “Nucleic acid,” as used herein, includes “polynucleotide,” “oligonucleotide,” and “nucleic acid molecule,” and generally means a polymer of DNA or RNA, which can be single-stranded or double-stranded, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. In embodiments, the nucleic acid comprises complementary DNA (cDNA). It is generally preferred that the nucleic acid does not comprise any insertions, deletions, inversions, and/or substitutions. However, it may be suitable in some instances, as discussed herein, for the nucleic acid to comprise one or more insertions, deletions, inversions, and/or substitutions.

Preferably, the nucleic acids of the invention are recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

The nucleic acids can be constructed based on chemical synthesis and/or enzymatic ligation reactions using procedures known in the art. See, for example, Green and Sambrook et al., supra. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N⁶-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil oxyacetic acid methylester, 3-(3-amino-3-N-2-carboxypropyl) uracil, and 2,6-diaminopurine. Alternatively, one or more of the nucleic acids of the invention can be purchased from any of a variety of commercial entities.

The nucleic acid can comprise any nucleotide sequence which encodes any of the TCRs, polypeptides, or proteins described herein. In embodiments of the invention, the nucleic acid may comprise the nucleotide sequence of any one of SEQ ID NOs: 43-46 (Table 5). In embodiments of the invention, the nucleic acid comprises the nucleotide sequences of both of SEQ ID NOs: 43-44 or both of SEQ ID NOs: 45-46.

TABLE 5

| TCR chain | Nucleotide sequence |
|---|---|
| 4391 Alpha | SEQ ID NO: 43 |
| 4391 Beta | SEQ ID NO: 44 |
| 4385 Alpha | SEQ ID NO: 45 |
| 4385 Beta | SEQ ID NO: 46 |
| 4394 Alpha | SEQ ID NO: 82 |
| 4394 Beta | SEQ ID NO: 83 |

In embodiments of the invention, the nucleic acid comprises a codon-optimized nucleotide sequence encoding any of the TCRs, polypeptides, or proteins described herein. Without being bound to any particular theory or mechanism, it is believed that codon optimization of the nucleotide sequence increases the translation efficiency of the mRNA transcripts. Codon optimization of the nucleotide sequence may involve substituting a native codon for another codon that encodes the same amino acid, but can be translated by tRNA that is more readily available within a cell, thus increasing translation efficiency. Optimization of the nucleotide sequence may also reduce secondary mRNA structures that would interfere with translation, thus increasing translation efficiency.

The invention also provides a nucleic acid comprising a nucleotide sequence which is complementary to the nucleotide sequence of any of the nucleic acids described herein or a nucleotide sequence which hybridizes under stringent conditions to the nucleotide sequence of any of the nucleic acids described herein.

The nucleotide sequence which hybridizes under stringent conditions preferably hybridizes under high stringency conditions. By “high stringency conditions” is meant that the nucleotide sequence specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-10 bases) that matched the nucleotide sequence. Such small regions of complementarily are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the nucleotide sequence and the template or target strand, and are particularly suitable for detecting expression of any of the inventive TCRs. It is generally appreciated that conditions can be rendered more stringent by the addition of increasing amounts of formamide.

The invention also provides a nucleic acid comprising a nucleotide sequence that is at least about 70% or more, e.g., about 80%, about 90%, about 91%, about 92%, about 93%, about 94%, about 95%, about 96%, about 97%, about 98%, or about 99% identical to any of the nucleic acids described herein. In this regard, the nucleic acid may consist essentially of any of the nucleotide sequences described herein.

An embodiment of the invention provides an isolated or purified nucleic acid comprising, from 5′ to 3′, a first nucleic acid sequence and a second nucleotide sequence, wherein the first and second nucleotide sequence, respectively, encode the amino sequences of SEQ ID NOs: 7 and 8; 7 and 91; 90 and 8; 90 and 91; 8 and 7; 91 and 7; 8 and 90; 91 and 90; 37 and 38; 37 and 93; 92 and 38; 92 and 93; 38 and 37; 93 and 37; 38 and 92; 93 and 92; 70 and 71; 70 and 133; 132 and 71; 132 and 133; 71 and 70; 133 and 70; 71 and 132; 133 and 132; 23 and 24; 23 and 103; 102 and 24; 102 and 103; 24 and 23; 103 and 23; 24 and 102; 103 and 102; 39 and 40; 39 and 109; 108 and 40; 108 and 109; 40 and 39; 109 and 39; 40 and 108; 109 and 108; 78 and 79; 78 and 139; 138 and 79; 138 and 139; 79 and 78; 139 and 78; 79 and 138; 139 and 138; 21 and 22; 21 and 101; 100 and 22; 100 and 101; 22 and 21; 101 and 21; 22 and 100; 101 and 100; 41 and 42; 41 and 107; 106 and 42; 106 and 107; 42 and 41; 107 and 41; 42 and 106; 107 and 106; 74 and 75; 74 and 101; 100 and 75, 100 and 101; 75 and 74; 101 and 74; 75 and 100; 101 and 100; 124 and 125; 124 and 105; 104 and 125; 104 and 105; 125 and 124; 105 and 124; 125 and 104; 105 and 104; 126 and 127; 126 and 111; 110 and 127; 110 and 111; 127 and 126; 111 and 126; 127 and 110; 111 and 110; 134 and 135; 140 and 135; 134 and 141; 140 and 141; 135 and 134; 135 and 140; 141 and 134;

141 and 140; 47 and 48; 48 and 47; 49 and 50; 50 and 49; 72 and 73; 73 and 72; 94 and 95; 95 and 94; 94 and 85; 85 and 94; 96 and 97; 97 and 96; 96 and 87; 87 and 96; 88 and 89; 89 and 88; 51 and 52; 52 and 51; 53 and 54; 54 and 53; 80 and 81; 81 and 80; 55 and 56; 56 and 55; 57 and 58; 58 and 57; 76 and 77; 77 and 76; 128 and 129; 129 and 128; 130 and 131; 131 and 130; 142 and 143; 143 and 142; 112 and 113; 113 and 112; 118 and 119; 119 and 118; 144 and 145; 145 and 144; 114 and 115; 115 and 114; 120 and 121; 121 and 120; 146 and 147; 147 and 146; 116 and 117; 117 and 116; 122 and 123; 123 and 122; 148 and 149; 149 and 148; 150 and 151; 151 and 150; 154 and 155; 155 and 154; 152 and 153; 153 and 152; 156 and 157; 157 and 156; 160 and 161; 161 and 160; 158 and 159; or 159 and 158.

In an embodiment of the invention, the isolated or purified nucleic acid further comprises a third nucleotide sequence interposed between the first and second nucleotide sequence, wherein the third nucleotide sequence encodes a cleavable linker peptide. In an embodiment of the invention, the cleavable linker peptide comprises the amino acid sequence of SEQ ID NO: 25.

The nucleic acids of the invention can be incorporated into a recombinant expression vector. In this regard, the invention provides a recombinant expression vector comprising any of the nucleic acids of the invention. In embodiments of the invention, the recombinant expression vector comprises a nucleotide sequence encoding the α chain, the β chain, and linker peptide.

For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors of the invention are not naturally-occurring as a whole. However, parts of the vectors can be naturally-occurring. The inventive recombinant expression vectors can comprise any type of nucleotide, including, but not limited to DNA and RNA, which can be single-stranded or double-stranded, synthesized or obtained in part from natural sources, and which can contain natural, non-natural or altered nucleotides. The recombinant expression vectors can comprise naturally-occurring, non-naturally-occurring internucleotide linkages, or both types of linkages. Preferably, the non-naturally occurring or altered nucleotides or internucleotide linkages do not hinder the transcription or replication of the vector.

The recombinant expression vector of the invention can be any suitable recombinant expression vector, and can be used to transform or transfect any suitable host cell. Suitable vectors include those designed for propagation and expansion or for expression or both, such as plasmids and viruses. The vector can be selected from the pUC series (Fermentas Life Sciences), the pBluescript series (Stratagene, LaJolla, CA), the pET series (Novagen, Madison, WI), the pGEX series (Pharmacia Biotech, Uppsala, Sweden), and the pEX series (Clontech, Palo Alto, CA). Bacteriophage vectors, such as λGT10, λGT11, λZapII (Stratagene), λEMBL4, and λNM1149, also can be used. Examples of plant expression vectors include pBI01, pBI101.2, pBI101.3, pBI121 and pBIN19 (Clontech). Examples of animal expression vectors include pEUK-Cl, pMAM and pMAMneo (Clontech). Preferably, the recombinant expression vector is a viral vector, e.g., a retroviral vector. In an especially preferred embodiment, the recombinant expression vector is an MSGV1 vector. In an embodiment of the invention, the recombinant expression vector is a transposon or a lentiviral vector.

The recombinant expression vectors of the invention can be prepared using standard recombinant DNA techniques described in, for example, Green and Sambrook et al., supra. Constructs of expression vectors, which are circular or linear, can be prepared to contain a replication system functional in a prokaryotic or eukaryotic host cell. Replication systems can be derived, e.g., from ColEl, 2 μ plasmid, λ, SV40, bovine papillomavirus, and the like.

Desirably, the recombinant expression vector comprises regulatory sequences, such as transcription and translation initiation and termination codons, which are specific to the type of host cell (e.g., bacterium, fungus, plant, or animal) into which the vector is to be introduced, as appropriate and taking into consideration whether the vector is DNA- or RNA-based.

The recombinant expression vector can include one or more marker genes, which allow for selection of transformed or transfected host cells. Marker genes include biocide resistance, e.g., resistance to antibiotics, heavy metals, etc., complementation in an auxotrophic host cell to provide prototrophy, and the like. Suitable marker genes for the inventive expression vectors include, for instance, neomycin/G418 resistance genes, hygromycin resistance genes, histidinol resistance genes, tetracycline resistance genes, and ampicillin resistance genes.

The recombinant expression vector can comprise a native or nonnative promoter operably linked to the nucleotide sequence encoding the TCR, polypeptide, or protein, or to the nucleotide sequence which is complementary to or which hybridizes to the nucleotide sequence encoding the TCR, polypeptide, or protein. The selection of promoters, e.g., strong, weak, inducible, tissue-specific and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an SV40 promoter, an RSV promoter, and a promoter found in the long-terminal repeat of the murine stem cell virus.

The inventive recombinant expression vectors can be designed for either transient expression, for stable expression, or for both. Also, the recombinant expression vectors can be made for constitutive expression or for inducible expression.

Further, the recombinant expression vectors can be made to include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art and include, for example, the Herpes Simplex Virus (HSV) thymidine kinase (TK) gene, cytosine deaminase, purine nucleoside phosphorylase, nitroreductase, and the inducible caspase 9 gene system.

Another embodiment of the invention further provides a host cell comprising any of the recombinant expression vectors described herein. As used herein, the term “host cell” refers to any type of cell that can contain the inventive recombinant expression vector. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human or mouse. The host cell can be an adherent cell or a suspended cell, i.e., a cell that grows in suspension. Suitable host cells are known in the art and include, for instance, DH5a E. coli cells, Chinese hamster ovarian cells, monkey VERO cells, COS cells, HEK293 cells, and the like. For purposes of amplifying or replicating the recombinant expression vector, the host cell is preferably a prokaryotic cell, e.g., a DH5a cell. For purposes of producing a recombinant TCR, polypeptide, or protein, the host cell is preferably a mammalian cell. Most preferably, the host cell is a human cell. While the host cell can be of any cell type, can originate from any type of tissue, and can be of any developmental stage, the host cell preferably is a peripheral blood lymphocyte (PBL) or a peripheral blood mononuclear cell (PBMC). More preferably, the host cell is a T cell. In an embodiment of the invention, the host cell is a human lymphocyte. In another embodiment of the invention, the host cell is selected from a T cell, a natural killer T (NKT) cell, an invariant natural killer T (iNKT) cell, and a natural killer (NK) cell. Still another embodiment of the invention provides a method of producing a host cell expressing a TCR that has antigenic specificity for the peptide of SEQ ID NO: 29 or 30, the method comprising contacting a cell with any of the vectors described herein under conditions that allow introduction of the vector into the cell.

For purposes herein, the T cell can be any T cell, such as a cultured T cell, e.g., a primary T cell, or a T cell from a cultured T cell line, e.g., Jurkat, SupT1, etc., or a T cell obtained from a mammal. If obtained from a mammal, the T cell can be obtained from numerous sources, including but not limited to blood, bone marrow, lymph node, the thymus, or other tissues or fluids. T cells can also be enriched for or purified. Preferably, the T cell is a human T cell. The T cell can be any type of T cell and can be of any developmental stage, including but not limited to, CD4⁺/CD8+ double positive T cells, CD4⁺ helper T cells, e.g., Th₁ and Th₂ cells, CD4⁺ T cells, CD8⁺ T cells (e.g., cytotoxic T cells), tumor infiltrating lymphocytes (TILs), memory T cells (e.g., central memory T cells and effector memory T cells), naive T cells, and the like.

Also provided by the invention is a population of cells comprising at least one host cell described herein. The population of cells can be a heterogeneous population comprising the host cell comprising any of the recombinant expression vectors described, in addition to at least one other cell, e.g., a host cell (e.g., a T cell), which does not comprise any of the recombinant expression vectors, or a cell other than a T cell, e.g., a B cell, a macrophage, a neutrophil, an erythrocyte, a hepatocyte, an endothelial cell, an epithelial cell, a muscle cell, a brain cell, etc. Alternatively, the population of cells can be a substantially homogeneous population, in which the population comprises mainly of host cells (e.g., consisting essentially of) comprising the recombinant expression vector. The population also can be a clonal population of cells, in which all cells of the population are clones of a single host cell comprising a recombinant expression vector, such that all cells of the population comprise the recombinant expression vector. In one embodiment of the invention, the population of cells is a clonal population comprising host cells comprising a recombinant expression vector as described herein.

In embodiments of the invention, the numbers of cells in the population may be rapidly expanded. Expansion of the numbers of T cells can be accomplished by any of a number of methods as are known in the art as described in, for example, U.S. Pat. Nos. 8,034,334; 8,383,099; U.S. Patent Application Publication No. 2012/0244133; Dudley et al., J. Immunother., 26:332-42 (2003); and Riddell et al., J. Immunol. Methods, 128:189-201 (1990). In embodiments, expansion of the numbers of T cells is carried out by culturing the T cells with OKT3 antibody, IL-2, and feeder PBMC (e.g., irradiated allogeneic PBMC).

The inventive TCRs, polypeptides, proteins, nucleic acids, recombinant expression vectors, and host cells (including populations thereof), can be isolated and/or purified. The term “isolated,” as used herein, means having been removed from its natural environment. The term “purified,” as used herein, means having been increased in purity, wherein “purity” is a relative term, and not to be necessarily construed as absolute purity. For example, the purity can be at least about 50%, can be greater than about 60%, about 70%, about 80%, about 90%, about 95%, or can be about 100%.

The inventive TCRs, polypeptides, proteins, nucleic acids, recombinant expression vectors, and host cells (including populations thereof), all of which are collectively referred to as “inventive TCR materials” hereinafter, can be formulated into a composition, such as a pharmaceutical composition. In this regard, the invention provides a pharmaceutical composition comprising any of the TCRs, polypeptides, proteins, nucleic acids, expression vectors, and host cells (including populations thereof), described herein, and a pharmaceutically acceptable carrier. The inventive pharmaceutical compositions containing any of the inventive TCR materials can comprise more than one inventive TCR material, e.g., a polypeptide and a nucleic acid, or two or more different TCRs. Alternatively, the pharmaceutical composition can comprise an inventive TCR material in combination with another pharmaceutically active agent(s) or drug(s), such as a chemotherapeutic agent, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc.

Preferably, the carrier is a pharmaceutically acceptable carrier. With respect to pharmaceutical compositions, the carrier can be any of those conventionally used for the particular inventive TCR material under consideration. Methods for preparing administrable compositions are known or apparent to those skilled in the art and are described in more detail in, for example, Remington: The Science and Practice of Pharmacy, 22^{nd} Ed., Pharmaceutical Press (2012). It is preferred that the pharmaceutically acceptable carrier be one which has no detrimental side effects or toxicity under the conditions of use.

The choice of carrier will be determined in part by the particular inventive TCR material, as well as by the particular method used to administer the inventive TCR material. Accordingly, there are a variety of suitable formulations of the pharmaceutical composition of the invention. Suitable formulations may include any of those for parenteral, subcutaneous, intravenous, intramuscular, intraarterial, intrathecal, intratumoral, or interperitoneal administration. More than one route can be used to administer the inventive TCR materials, and in certain instances, a particular route can provide a more immediate and more effective response than another route.

Preferably, the inventive TCR material is administered by injection, e.g., intravenously. When the inventive TCR material is a host cell (or population thereof) expressing the inventive TCR, the pharmaceutically acceptable carrier for the cells for injection may include any isotonic carrier such as, for example, normal saline (about 0.90% w/v of NaCl in water, about 300 mOsm/L NaCl in water, or about 9.0 g NaCl per liter of water), NORMOSOL R electrolyte solution (Abbott, Chicago, Ill.), PLASMA-LYTE A (Baxter, Deerfield, Ill.), about 5% dextrose in water, or Ringer's lactate. In embodiments, the pharmaceutically acceptable carrier is supplemented with human serum albumin.

For purposes of the invention, the amount or dose (e.g., numbers of cells when the inventive TCR material is one or more cells) of the inventive TCR material administered should be sufficient to effect, e.g., a therapeutic or prophylactic response, in the subject or animal over a reasonable time frame. For example, the dose of the inventive TCR material should be sufficient to bind to a cancer antigen (e.g., mutated RAS), or detect, treat or prevent cancer in a period of from about 2 hours or longer, e.g., 12 to 24 or more hours, from the time of administration. In certain embodiments, the time period could be even longer. The dose will be determined by the efficacy of the particular inventive TCR material and the condition of the animal (e.g., human), as well as the body weight of the animal (e.g., human) to be treated.

Many assays for determining an administered dose are known in the art. For purposes of the invention, an assay, which comprises comparing the extent to which target cells are lysed or IFN-γ is secreted by T cells expressing the inventive TCR, polypeptide, or protein upon administration of a given dose of such T cells to a mammal among a set of mammals of which each is given a different dose of the T cells, could be used to determine a starting dose to be administered to a mammal. The extent to which target cells are lysed or IFN-γ is secreted upon administration of a certain dose can be assayed by methods known in the art.

The dose of the inventive TCR material also will be determined by the existence, nature and extent of any adverse side effects that might accompany the administration of a particular inventive TCR material. Typically, the attending physician will decide the dosage of the inventive TCR material with which to treat each individual patient, taking into consideration a variety of factors, such as age, body weight, general health, diet, sex, inventive TCR material to be administered, route of administration, and the severity of the cancer being treated. In embodiments in which the inventive TCR material is a population of cells, the number of cells administered per infusion may vary, e.g., from about 1×10⁶ to about 1×10¹² cells or more. In certain embodiments, fewer than 1×10⁶ cells may be administered.

One of ordinary skill in the art will readily appreciate that the inventive TCR materials of the invention can be modified in any number of ways, such that the therapeutic or prophylactic efficacy of the inventive TCR materials is increased through the modification. For instance, the inventive TCR materials can be conjugated either directly or indirectly through a bridge to a chemotherapeutic agent. The practice of conjugating compounds to a chemotherapeutic agent is known in the art. One of ordinary skill in the art recognizes that sites on the inventive TCR materials, which are not necessary for the function of the inventive TCR materials, are suitable sites for attaching a bridge and/or a chemotherapeutic agent, provided that the bridge and/or chemotherapeutic agent, once attached to the inventive TCR materials, do(es) not interfere with the function of the inventive TCR materials, i.e., the ability to bind to mutated RAS or to detect, treat, or prevent cancer.

It is contemplated that the inventive pharmaceutical compositions, TCRs, polypeptides, proteins, nucleic acids, recombinant expression vectors, host cells, and populations of cells can be used in methods of treating or preventing cancer. Without being bound to a particular theory, the inventive TCRs are believed to bind specifically to mutated RAS, such that the TCR (or related inventive polypeptide or protein), when expressed by a cell, is able to mediate an immune response against a target cell expressing mutated RAS. In this regard, the invention provides a method of treating or preventing cancer in a mammal, comprising administering to the mammal any of the pharmaceutical compositions, TCRs, polypeptides, or proteins described herein, any nucleic acid or recombinant expression vector comprising a nucleotide sequence encoding any of the TCRs, polypeptides, proteins described herein, or any host cell or population of cells comprising a recombinant vector which encodes any of the TCRs, polypeptides, or proteins described herein, in an amount effective to treat or prevent cancer in the mammal.

An embodiment of the invention provides a method of inducing an immune response against a cancer in a mammal, comprising administering to the mammal any of the pharmaceutical compositions, TCRs, polypeptides, or proteins described herein, any nucleic acid or recombinant expression vector comprising a nucleotide sequence encoding any of the TCRs, polypeptides, or proteins described herein, or any host cell or population of cells comprising a recombinant vector which encodes any of the TCRs, polypeptides, or proteins described herein, in an amount effective to induce an immune response against the cancer in the mammal.

An embodiment of the invention provides any of the pharmaceutical compositions, TCRs, polypeptides, or proteins described herein, any nucleic acid or recombinant expression vector comprising a nucleotide sequence encoding any of the TCRs, polypeptides, proteins described herein, or any host cell or population of cells comprising a recombinant vector which encodes any of the TCRs, polypeptides, or proteins described herein, for use in the treatment or prevention of cancer in a mammal.

An embodiment of the invention provides any of the pharmaceutical compositions, TCRs, polypeptides, or proteins described herein, any nucleic acid or recombinant expression vector comprising a nucleotide sequence encoding any of the TCRs, polypeptides, or proteins described herein, or any host cell or population of cells comprising a recombinant vector which encodes any of the TCRs, polypeptides, or proteins described herein, for use in inducing an immune response against a cancer in a mammal.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of cancer in a mammal. Furthermore, the treatment or prevention provided by the inventive method can include treatment or prevention of one or more conditions or symptoms of the cancer being treated or prevented. For example, treatment or prevention can include promoting the regression of a tumor. Also, for purposes herein, “prevention” can encompass delaying the onset of the cancer, or a symptom or condition thereof. Alternatively or additionally, “prevention” may encompass preventing or delaying the recurrence of cancer, or a symptom or condition thereof.

Also provided is a method of detecting the presence of cancer in a mammal. The method comprises (i) contacting a sample comprising one or more cells from the mammal with any of the inventive TCRs, polypeptides, proteins, nucleic acids, recombinant expression vectors, host cells, populations of cells, or pharmaceutical compositions described herein, thereby forming a complex, and (ii) detecting the complex, wherein detection of the complex is indicative of the presence of cancer in the mammal.

With respect to the inventive method of detecting cancer in a mammal, the sample of cells can be a sample comprising whole cells, lysates thereof, or a fraction of the whole cell lysates, e.g., a nuclear or cytoplasmic fraction, a whole protein fraction, or a nucleic acid fraction.

For purposes of the inventive method of detecting cancer, the contacting can take place in vitro or in vivo with respect to the mammal. Preferably, the contacting is in vitro.

Also, detection of the complex can occur through any number of ways known in the art. For instance, the inventive TCRs, polypeptides, proteins, nucleic acids, recombinant expression vectors, host cells, or populations of cells, described herein, can be labeled with a detectable label such as, for instance, a radioisotope, a fluorophore (e.g., fluorescein isothiocyanate (FITC), phycoerythrin (PE)), an enzyme (e.g., alkaline phosphatase, horseradish peroxidase), and element particles (e.g., gold particles).

For purposes of the inventive methods, wherein host cells or populations of cells are administered, the cells can be cells that are allogeneic or autologous to the mammal. Preferably, the cells are autologous to the mammal.

With respect to the inventive methods, the cancer can be any cancer, including any of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vagina, cancer of the vulva, chronic lymphocytic leukemia, chronic myeloid cancer, colon cancer, colorectal cancer, endometrial cancer, esophageal cancer, uterine cervical cancer, gastrointestinal carcinoid tumor, glioma, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, cancer of the oropharynx, ovarian cancer, cancer of the penis, pancreatic cancer, peritoneum, omentum, and mesentery cancer, pharynx cancer, prostate cancer, rectal cancer, renal cancer, skin cancer, small intestine cancer, soft tissue cancer, stomach cancer, testicular cancer, thyroid cancer, cancer of the uterus, ureter cancer, and urinary bladder cancer. A preferred cancer is pancreatic, colorectal, lung, endometrial, ovarian, or prostate cancer. Preferably, the lung cancer is lung adenocarcinoma, the ovarian cancer is epithelial ovarian cancer, and the pancreatic cancer is pancreatic adenocarcinoma. In embodiments of the invention, the cancer expresses a mutated human RAS amino acid sequence, wherein the mutated human RAS amino acid sequence is a mutated human KRAS, a mutated human HRAS, or a mutated human NRAS amino acid sequence. The mutated human KRAS, mutated human HRAS, and mutated human NRAS expressed by the cancer may be as described herein with respect to other aspects of the invention.

The mammal referred to in the inventive methods can be any mammal. As used herein, the term “mammal” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

It shall be noted that the preceding are merely examples of embodiments. Other exemplary embodiments are apparent from the entirety of the description herein. It will also be understood by one of ordinary skill in the art that each of these embodiments may be used in various combinations with the other embodiments provided herein.

The following examples further illustrate the invention but, of course, should not be construed as in any way limiting its scope.

### EXAMPLE 1

This example demonstrates the identification of TIL reactive to RAS G12V from the tumor fragments of colorectal cancer Patient 4391. This example also demonstrates the identification of the minimal epitope recognized by TIL from tumor fragment F1 of Patient 4391.

TIL were screened for reactivity, tested by a 41BB+/OX40+ flow cytometry assay against dendritic cells (DC) loaded with 5μg/ml of Long Peptide (LP) (WT, which is SEQ ID NO: 30, or G12V, which is SEQ ID NO: 27) or transfected with a tandem mini-gene (TMG) encoding RAS WT or RAS G12V.

TILs were isolated from tumor fragments Fl, F13, and a combination of tumor fragments F2, F3, and F5 from Patient 4391. Reactivity was measured by detecting upregulation of OX40 and 4-1BB by FACS. The TIL were gated live/CD3+/CD8+.

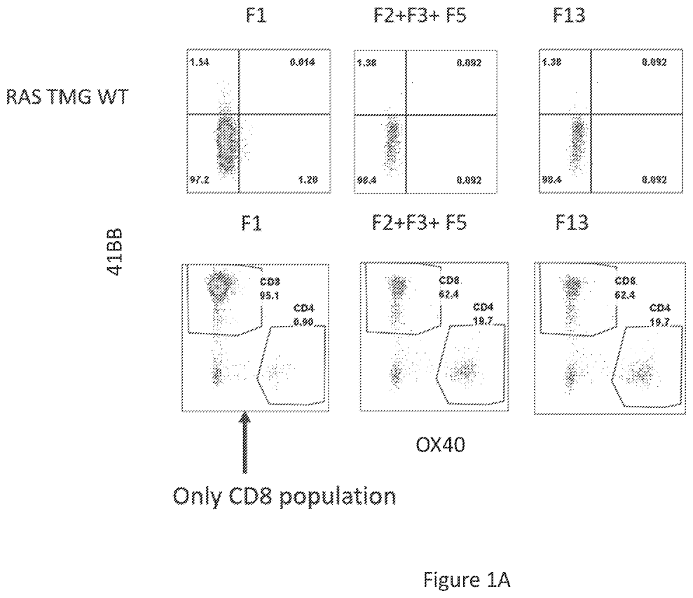

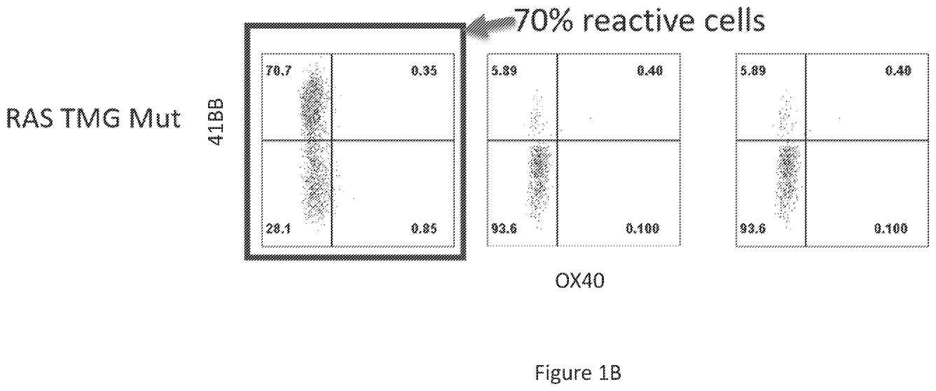

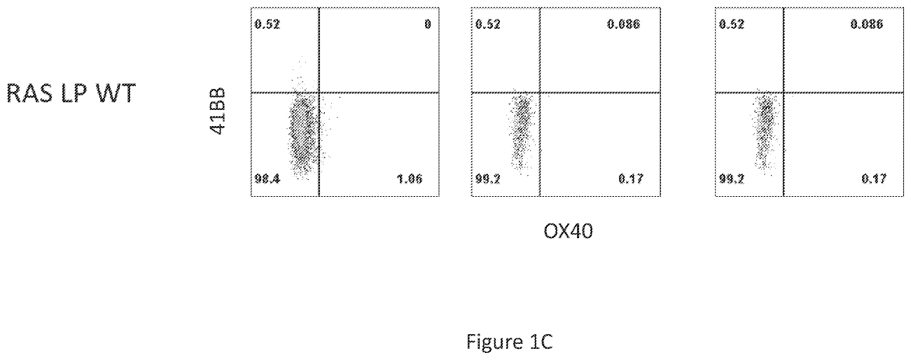

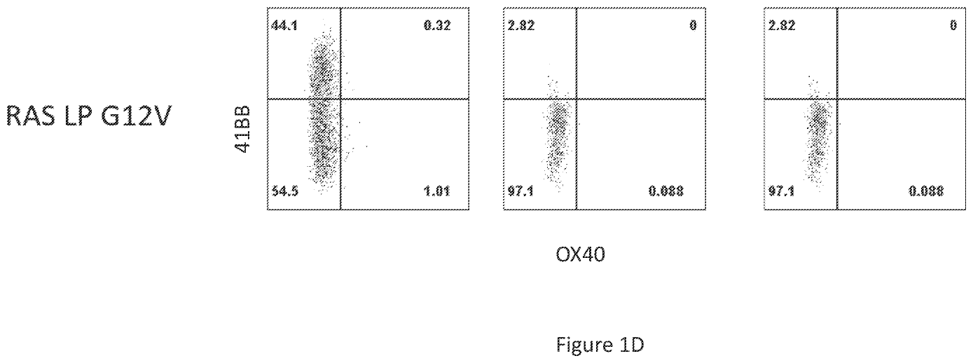

The results are in FIGS. 1A-1D. There was reactivity in fragment 1 (F1; 70% of the cells were reactive when tested with G12V TMG) and also in some other fragments (at a lower frequency).

TIL from tumor fragment F1 were co-cultured with autologous DC, COS7 cells stably express HLA-A02:01, or COS7 cells stably express HLA-A03:01 loaded with 5 μg/ml RAS minimal epitopes (ME) or RAS WT LP as a negative control or RAS G12V LP as a positive control. TIL cultured with dimethyl sulfoxide (DMSO) also served as a negative control. The minimal epitopes are listed in Table 6.

TABLE 6

| Minimal Epitope (ME) | Peptide |
|---|---|
| ME 4 | KLVVVGAVGV |
| | (SEQ ID NO: 59) |
| ME 5 | VVGAVGVGK |
| | (SEQ ID NO: 60) |
| ME 6 | VVVGAVGVGK |
| | (SEQ ID NO: 61) |
| ME 7 | VVVGAVGVG |
| | (SEQ ID NO: 62) |
| ME 8 | AVGVGKSAL |
| | (SEQ ID NO: 63) |

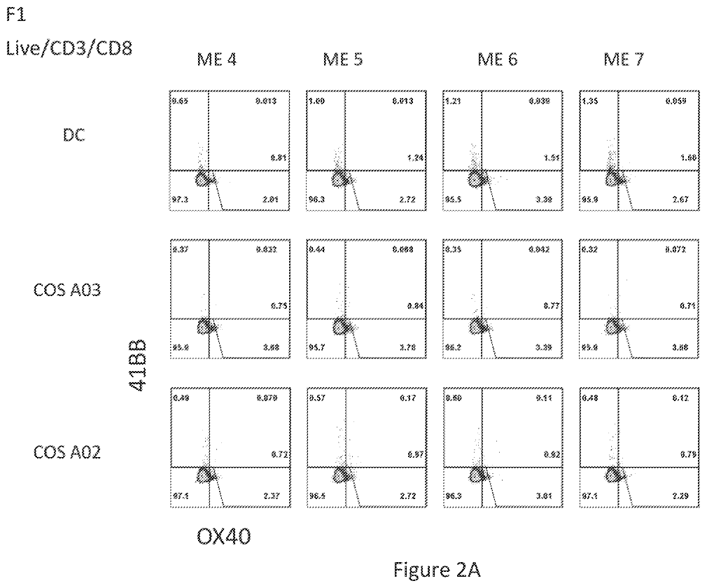

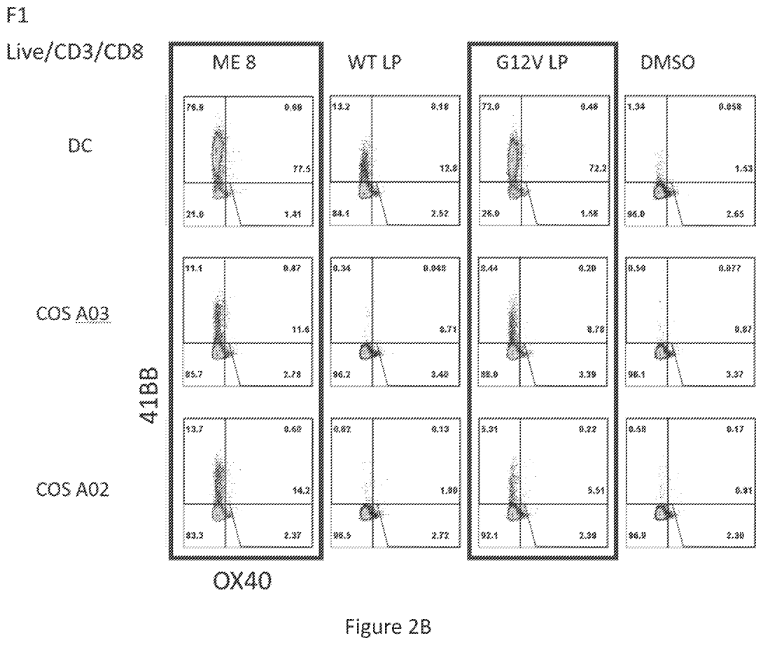

The results are in FIGS. 2A and 2B. The reactivity was tested by 41BB/OX40 flow cytometry assay. The greatest reactivity against the MEs was found against ME 8.

### EXAMPLE 2

This example demonstrates that TIL isolated from tumor fragment F1 of Patient 4391 specifically recognize RAS G12V.

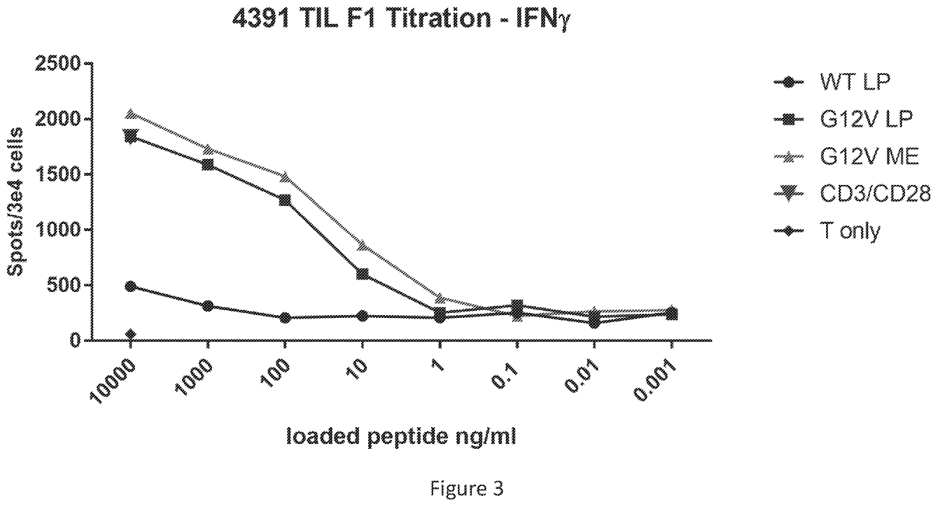

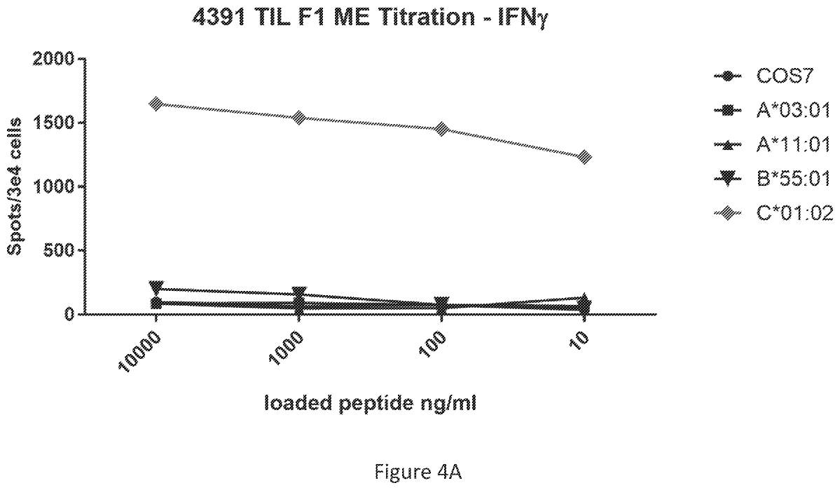

TILs from tumor fragment F1 of Patient 4391 were co-cultured with autologous DC loaded with RAS G12V ME 8 or RAS LP (G12V or WT) in different concentrations. TIL cultured alone served as a negative control. TIL non-specifically stimulated by co-culture with anti-CD3/anti-CD28 antibodies served as a positive control. The reactivity was tested by IFNγ ELISPOT. The results are in FIG. 3.

### EXAMPLE 3

This example demonstrates that TIL isolated from tumor fragment F1 of Patient 4391 specifically recognize RAS G12V presented by HLA-C*01:02.

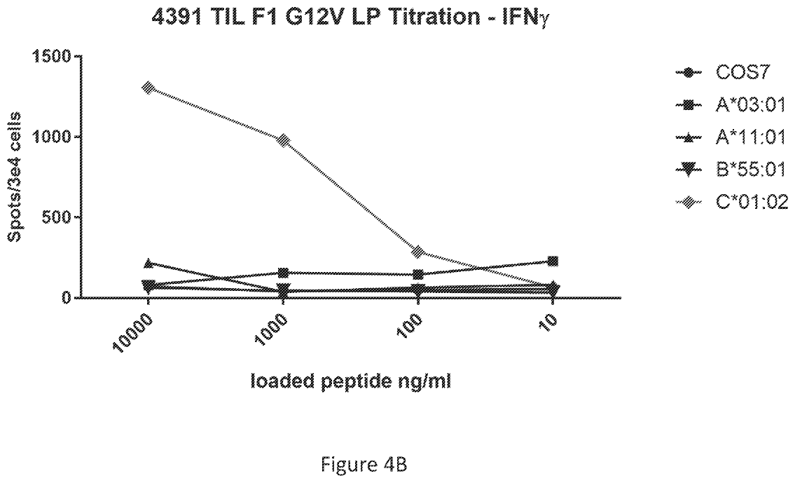

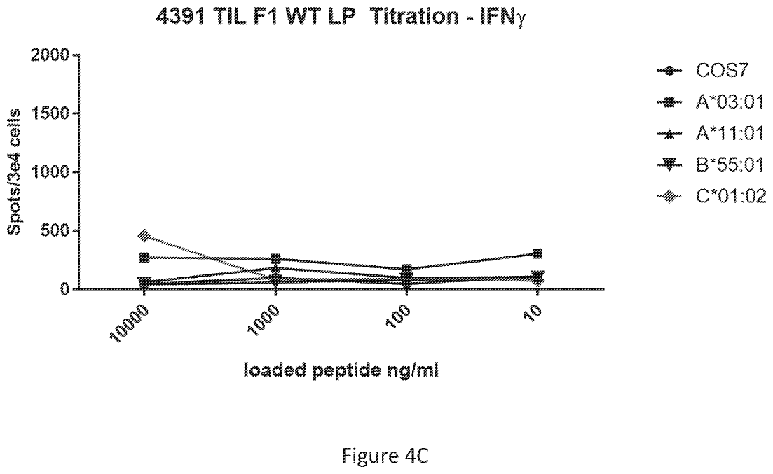

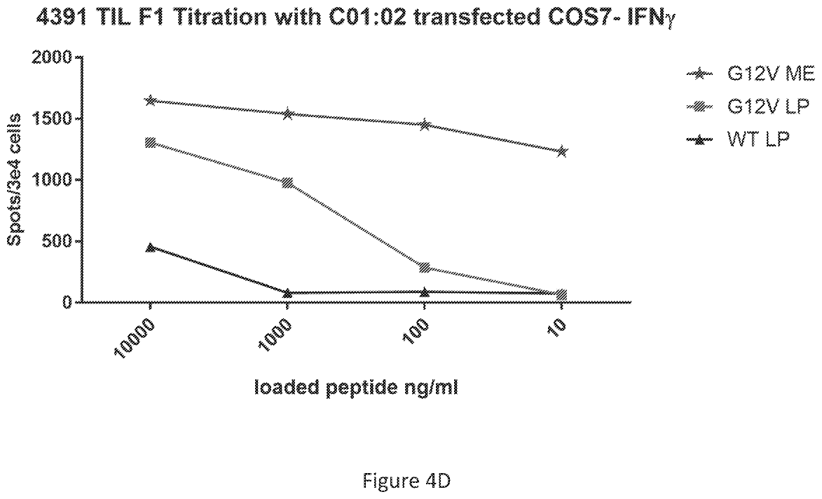

To find the MHC-I restricted element, TIL isolated from tumor fragment F1 of Patient 4391 were co-cultured with COS7 cell line transfected with a plasmid encoding one of four different HLA alleles expressed by Patient 4391 (HLA-A*03:01, HLA-A*11:01, HLA-B*55:01, or HLA-C*01:02). The cells were loaded with RAS G12V ME 8 (FIG. 4A), RAS G12V LP (FIG. 4B), or RAS WT LP (FIG. 4C) in different concentrations. The reactivity was tested by IFNγ ELISPOT. Included are the results of F1 against COS7 transfected with HLA-C*01:02 (FIG. 4D).

### EXAMPLE 4

This example demonstrates the isolation of a TCR having antigenic specificity for human RAS with the G12V mutation presented by HLA-C*01:02 from the TIL of Patient 4391.

The TILs from tumor fragment F1 of Patient 4391 were determined to recognize RAS G12V and not WT RAS in Examples 1-3.

To sequence the reactive TCR, the reactive TILs were sorted by fluorescence-activated cell sorting (FACS) based on the upregulation of the T cell activation markers, 4-1BB/OX40. Subsequently, the cells were lysed, and the TCR transcripts were Sanger sequenced.

### EXAMPLE 5

This example demonstrates a method of preparing a retroviral vector comprising a nucleotide sequence encoding the human anti-G12V TCR of Example 4 with modified murine constant regions.

A nucleic acid sequence encoding the human G12V RAS-reactive 4391 TCR of Example 4 and including a cysteine substituted, LVL-modified murine constant region was cloned into a retroviral expression vector. The α chain murine constant region comprised the amino acid sequence of SEQ ID NO: 17 wherein X at position 48 is Cys, X at position 112 is Leu, X at position 114 is Ile, and X at position 115 is Val. The resulting full-length α chain comprised the amino acid sequence of SEQ ID NO: 23. The 13 chain constant region comprised the amino acid sequence of SEQ ID NO: 18, wherein X at position 57 is Cys. The resulting full-length 13 chain comprised the amino acid sequence of SEQ ID NO: 24. A linker comprising the amino acid sequence of RAKRSGSGATNFSLLKQAGDVEENPGP (SEQ ID NO: 25) was positioned between the α chain constant region and the 13 chain variable region. The sequences as provided are codon optimized.

To allow cloning the second amino acid within the N-terminal signal peptide was changed to an alanine (A). This example describes a synthesis of bicistronic vector in 5′TCRβ to TCRα 3′ orientation, but the order of TCRβ to TCRβ can be reversed.

The amino acid sequences of the TCR alpha and betα chain variable regions are shown in Table 7. The CDRs are underlined.

TABLE 7

| TCR Name | TCR chain | Amino acid sequence |
|---|---|---|
| 4391 TCR | Alpha | MAGIRALFMYLWLQLDWVSRGESVGLHLPTLSVQEGDNSIINCA |
| | (TRAV13-2*01 + | YSNSASDYFIWYKQESGKGPQFIIDIRSNMDKRQGQRVTVLLNK |
| | TRAJ54*01) | TVKHLSLQIAATQPGDSAVYFCAEYIQGAQKLVFGQGTRLTINP |
| | (with wild type N- | (SEQ ID NO: 7) |
| | terminal signal | |
| | peptide) | |
| | Beta | MACRLLCCAVLCLLGAGELVPMETGVTQTPRHLVMGMTNKKSL |
| | (TRBV4-3*01 + | KCEQHLGHNAMYWYKQSAKKPLELMFVYSLEERVENNSVPSR |
| | TRBJ2-1*01 + | FSPECPNSSHLFLHLHTLQPEDSALYLCASSQEDNEQFFGPGT |
| | TRBD1*01) | RLTVL |
| | (with variant N- | (SEQ ID NO: 8) |
| | terminal signal | |
| | peptide) | |
| | Alpha | GESVGLHLPTLSVQEGDNSIINCAYSNSASDYFIWYKQESGKGP |
| | (TRAV13-2*01 + | QFIIDIRSNMDKRQGQRVTVLLNKTVKHLSLQIAATQPGDSAVYF |
| | TRAJ54*01) | CAEYIQGAQKLVFGQGTRLTINP |
| | (without N-terminal | (SEQ ID NO: 47) |
| | signal peptide | |
| | predicted with | |
| | IMGT) | |
| | Beta | ETGVTQTPRHLVMGMTNKKSLKCEQHLGHNAMYWYKQSAKKP |
| | (TRBV4-3*01 + | LELMFVYSLEERVENNSVPSRFSPECPNSSHLFLHLHTLQPEDS |
| | TRBJ2-1*01 + | ALYLCASSQEDNEQFFGPGTRLTVL |
| | TRBDP01) | (SEQ ID NO: 48) |
| | (without N-terminal | |
| | signal peptide | |
| | predicted with | |
| | IMGT) | |
| | Alpha | ESVGLHLPTLSVQEGDNSIINCAYSNSASDYFIWYKQESGKGPQ |
| | (TRAV13-2*01 + | FIIDIRSNMDKRQGQRVTVLLNKTVKHLSLQIAATQPGDSAVYFC |
| | TRAJ54*01) | AEYIQGAQKLVFGQGTRLTINP |
| | (without N-terminal | (SEQ ID NO: 94) |
| | signal peptide | |
| | predicted with | |
| | SignalP) | |
| | Beta | ELVPMETGVTQTPRHLVMGMTNKKSLKCEQHLGHNAMYWYKQ |
| | (TRBV4-3*01 + | SAKKPLELMFVYSLEERVENNSVPSRFSPECPNSSHLFLHLHTL |
| | TRBJ2-1*01 + | QPEDSALYLCASSQEDNEQFFGPGTRLTVL |
| | TRBD1*01) | (SEQ ID NO: 95) |
| | (without N-terminal | |
| | signal peptide | |
| | predicted with | |
| | SignalP) | |
| | Alpha | MAGIRALFMYLWLQLDWVSRGESVGLHLPTLSVQEGDNSIINCA |
| | (TRAV13-2*01 + | YSNSASDYFIWYKQESGKGPQFIIDIRSNMDKRQGQRVTVLLNK |
| | TRAJ54*01) | TVKHLS LQIAATQPGDSAVYFCAEYIQGAQKLVFGQGTRLTINP |
| | (with variant type | (SEQ ID NO: 90) |
| | N-terminal signal | |
| | peptide) | |
| | Beta | MGCRLLCCAVLCLLGAGELVPMETGVTQTPRHLVMGMTNKKSL |
| | (TRBV4-3*01 + | KCEQHLGHNAMYWYKQSAKKPLELMFVYSLEERVENNSVPSR |
| | TRBJ2-1*01 + | FSPECPNSSHLFLHLHTLQPEDSALYLCASSQEDNEQFFGPGT |
| | TRBD1*01) | RLTVL |
| | (with wild type N- | (SEQ ID NO: 91) |
| | terminal signal | |
| | peptide) | |
| | Beta | GVTQTPRHLVMGMTNKKSLKCEQHLGHNAMYWYKQSAKKPLE |
| | (TRBV4-3*01 + | LMFVYSLEERVENNSVPSRFSPECPNSSHLFLHLHTLQPEDSAL |
| | TRBJ2-1*01 + | YLCASSQEDNEQFFGPGTRLTVL |
| | TRBD1*01) | (SEQ ID NO: 85) |
| | (without N-terminal | |
| | signal peptide, | |
| | alternate) | |

### EXAMPLE 6

This example demonstrates that PBL transduced with the retroviral vector of Example 5 specifically recognize RAS G12V.

Healthy donor PBL were transduced with 4391 TCR. The transduced PBL were co-cultured with autologous DC loaded with RAS G12V ME8 or ME WT 4 (WT sequence of ME 8; AGGVGKSAL (SEQ ID NO: 26)) in different concentrations.

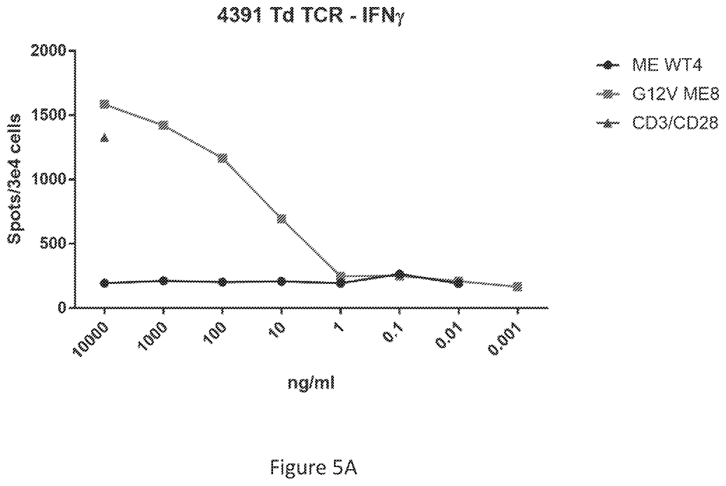

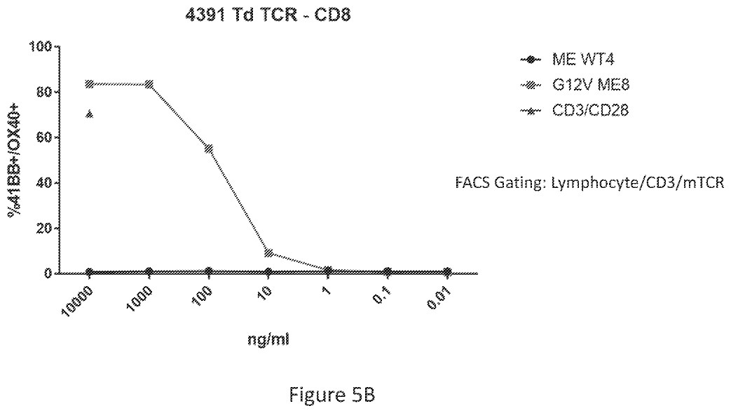

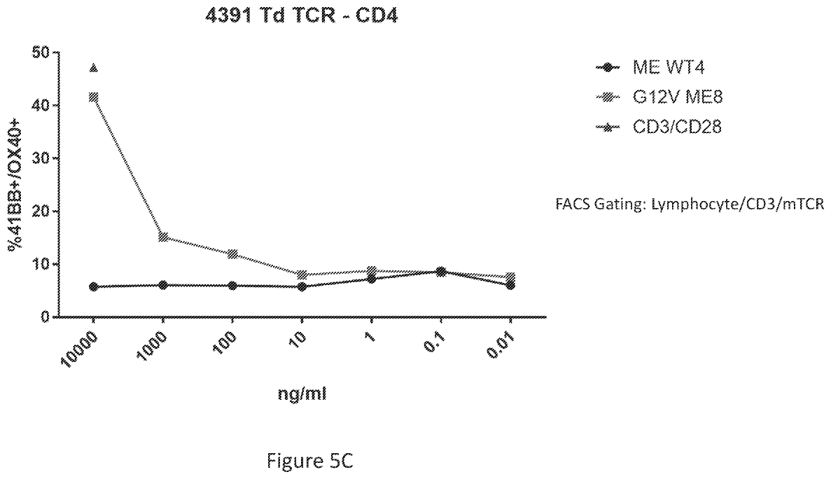

The reactivity was tested by IFNγ ELISPOT (FIG. 5A) and 41BB/OX40 flow cytometry gating to CD8+ (FIG. 5B) and gating to CD4 (FIG. 5C).

### EXAMPLE 7

This example demonstrates the identification of TIL reactive to RAS G12V from the tumor fragments of colorectal cancer Patient 4385.

TIL isolated from tumor fragment F11 from Patient 4385 were screened for reactivity using the IFNγ ELISPOT assay against DC loaded with Peptide pools (PP) or transfected with tandem mini-gene (TMG) mRNA.

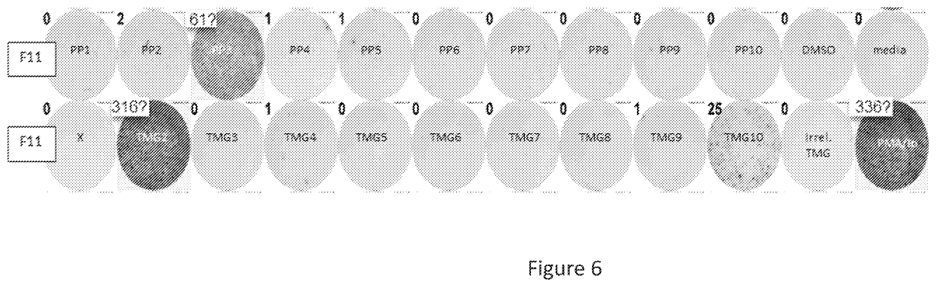

Reactive TILs were found in tumor fragment F11 against TMG2 and PP3, both containing the RAS G12V antigen. The results are in FIG. 6.

Testing of the specific peptides reveals that the reactivity is against RAS G12V.

### EXAMPLE 8

This example demonstrates the isolation of a TCR having antigenic specificity for human RAS G12V mutation presented by HLA-C*01:02 from the TIL of Patient 4385.

The TIL from tumor fragment F11 of Patient 4385 were determined to recognize RAS G12V and not RAS WT.

To sequence the reactive TCR, the reactive TILs were sorted by fluorescence-activated cell sorting (FACS) based on the upregulation of the T cell activation markers, 4-1BB/OX40. Subsequently, the cells were lysed, and the TCR transcripts were Sanger sequenced.

### EXAMPLE 9

This example demonstrates a method of preparing a retroviral vector comprising a nucleotide sequence encoding the human anti-G12V TCR of Example 8 with modified murine constant regions.

A nucleic acid sequence encoding the human RAS G12V -reactive 4385 TCR and including a cysteine substituted, LVL-modified murine constant region was cloned into a retroviral expression vector. The α chain murine constant region comprised the amino acid sequence of SEQ ID NO: 17 wherein X at position 48 is Cys, X at position 112 is Leu, X at position 114 is Ile, and X at position 115 is Val. The resulting full-length α chain comprised the amino acid sequence of SEQ ID NO: 39. The 13 chain constant region comprised the amino acid sequence of SEQ ID NO: 18, wherein X at position 57 is Cys. The resulting full-length β chain comprised the amino acid sequence of SEQ ID NO: 40. A linker comprising the amino acid sequence of RAKRSGSGATNFSLLKQAGDVEENPGP (SEQ ID NO: 25) was positioned between the α chain constant region and the 13 chain variable region. The sequences as provided are codon optimized.

To allow cloning the second amino acid within the N-terminal signal peptide was changed to an alanine (A). This example describes a synthesis of bicistronic vector in 5′TCRβ to TCRα 3′ orientation, but the order of TCRβ to TCRα can be reversed.

The amino acid sequences of the TCR alpha and betα chain variable regions are shown in Table 8. The CDRs are underlined.

TABLE 8

| TCR Name | TCR chain | Amino acid sequence |
|---|---|---|
| 4385 TCR | Alpha | MISLRVLLVILWLQLSWVWSQRKEVEQDPGPFNVPEGATVAFN |
| | (TRAV12-1*01 + | CTYSNSASQSFFWYRQDCRKEPKLLMSVYSSGNEDGRFTAHL |
| | TRAJ9*01) | NRASQYISLLIRDSKLSDSATYLCAVNPKYTGGFKTIFGAGTRLF |
| | (with wild type N- | VKA |
| | terminal signal | (SEQ ID NO: 37) |
| | peptide) | |
| | Beta | MACRLLCCAVLCLLGAGELVPMETGVTQTPRHLVMGMTNKKSL |
| | (TRBV4-3 + TRBJ1- | KCEQHLGHNAMYWYKQSAKKPLELMFVYSLEERVENNSVPSR |
| | 3*01) (with variant | FSPECPNSSHLFLHLHTLQPEDSALYLCASSQDVTSEWVDTIYF |
| | N-terminal signal | GEGSWLTVV |
| | peptide) | (SEQ ID NO: 38) |
| | Alpha | RKEVEQDPGPFNVPEGATVAFNCTYSNSASQSFFWYRQDCRK |
| | (TRAV12-1*01 + | EPKLLMSVYSSGNEDGRFTAHLNRASQYISLLIRDSKLSDSATYL |
| | TRAJ9*01) (without | CAVNPKYTGGFKTIFGAGTRLFVKA |
| | N-terminal signal | (SEQ ID NO: 49) |
| | peptide predicted | |
| | with IMGT) | |
| | Beta | ETGVTQTPRHLVMGMTNKKSLKCEQHLGHNAMYWYKQSAKKP |
| | (TRBV4-3 + TRBJ1- | LELMFVYSLEERVENNSVPSRFSPECPNSSHLFLHLHTLQPEDS |
| | 3*01) (without N- | ALYLCASSQDVTSEWVDTIYFGEGSWLTVV |
| | terminal signal | (SEQ ID NO: 50) |
| | peptide predicted | |
| | with IMGT) | |
| | Alpha | QRKEVEQDPGPFNVPEGATVAFNCTYSNSASQSFFWYRQDCR |
| | (TRAV12-1*01 + | KEPKLLMSVYSSGNEDGRFTAHLNRASQYISLLIRDSKLSDSATY |
| | TRAJ9*01) (without | LCAVNPKYTGGFKTIFGAGTRLFVKA |
| | N-terminal signal | (SEQ ID NO: 96) |
| | peptide predicted | |
| | with SignalP) | |
| | Beta | ELVPMETGVTQTPRHLVMGMTNKKSLKCEQHLGHNAMYWYKQ |
| | (TRBV4-3 + TRBJ1- | SAKKPLELMFVYSLEERVENNSVPSRFSPECPNSSHLFLHLHTL |
| | 3*01) (without N- | QPEDSALYLCASSQDVTSEWVDTIYFGEGSWLTVV |
| | terminal signal | (SEQ ID NO: 97) |
| | peptide predicted | |
| | with SignalP) | |
| | Alpha | MASLRVLLVILWLQLSWVWSQRKEVEQDPGPFNVPEGATVAFN |
| | (TRAV12-1*01 + | CTYSNSASQSFFWYRQDCRKEPKLLMSVYSSGNEDGRFTAHL |
| | TRAJ9*01) | NRASQYISLLIRDSKLSDSATYLCAVNPKYTGGFKTIFGAGTRLF |
| | (with variant type | VKA |
| | N-terminal signal | (SEQ ID NO: 92) |
| | peptide) | |
| | Beta | MGCRLLCCAVLCLLGAGELVPMETGVTQTPRHLVMGMTNKKSL |
| | (TRBV4-3 + TRBJ1- | KCEQHLGHNAMYWYKQSAKKPLELMFVYSLEERVENNSVPSR |
| | 3*01) | FSPECPNSSHLFLHLHTLQPEDSALYLCASSQDVTSEWVDTIYF |
| | (with wild type N- | GEGSWLTVV |
| | terminal signal | (SEQ ID NO: 93) |
| | peptide) | |
| | Beta | GVTQTPRHLVMGMTNKKSLKCEQHLGHNAMYWYKQSAKKPLE |
| | (TRBV4-3 + TRBJ1- | LMFVYSLEERVENNSVPSRFSPECPNSSHLFLHLHTLQPEDSAL |
| | 3*01) (without N- | YLCASSQDVTSEWVDTIYFGEGSWLTVV |
| | terminal signal | (SEQ ID NO: 87) |
| | peptide, alternate) | |

### EXAMPLE 10

This example demonstrates the reactivity of the TIL of tumor fragment F11 against RAS G12V in comparison to TCR4 originally isolated from F11.

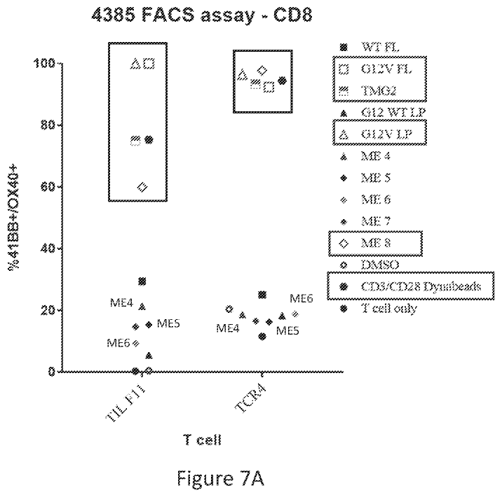

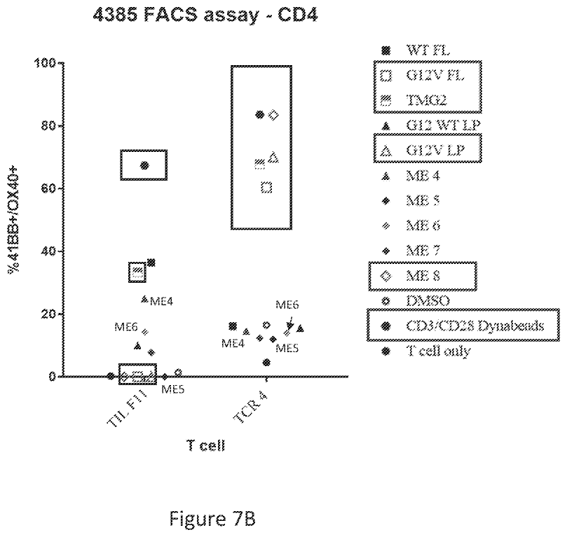

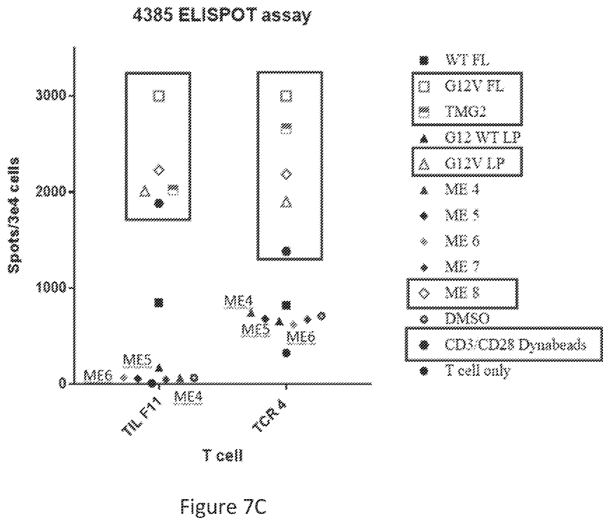

4385 (TCR4) was virally transduced into 4385 PBLs. Transduced cells and TIL F11 were co-cultured with DC loaded with the indicated peptides as LP/ME or mRNA transfected with the indicated genes as TMG/full-length (FL). Reactivity was tested using 41BB/OX40 flow cytometry assay gated to CD8+ (FIG. 7A) and CD4 (FIG. 7B) and IFNγ ELISPOT (FIG. 7C).

PBLs transduced with TCR4 and TIL F11 were reactive against ras G12V FL and TMG2 (containing the RAS G12V antigen) genes and against ras G12V LP. Among the ME peptides listed in Table 6, the greatest reactivity was detected against ME8.

### EXAMPLE 11

This example demonstrates that TIL isolated from tumor fragment F11 of Patient 4385 specifically recognize RAS G12V presented by HLA-C*01:02.

TIL F11- and TCR4-transduced cells were co-cultured with MHC-I transfected COS7 cells (30,000 Cos7 transfected with 10Ong of HLA pulsed+TMG, co-cultured with 20,000 T-cells). Reactivity to C*01:02 was observed.

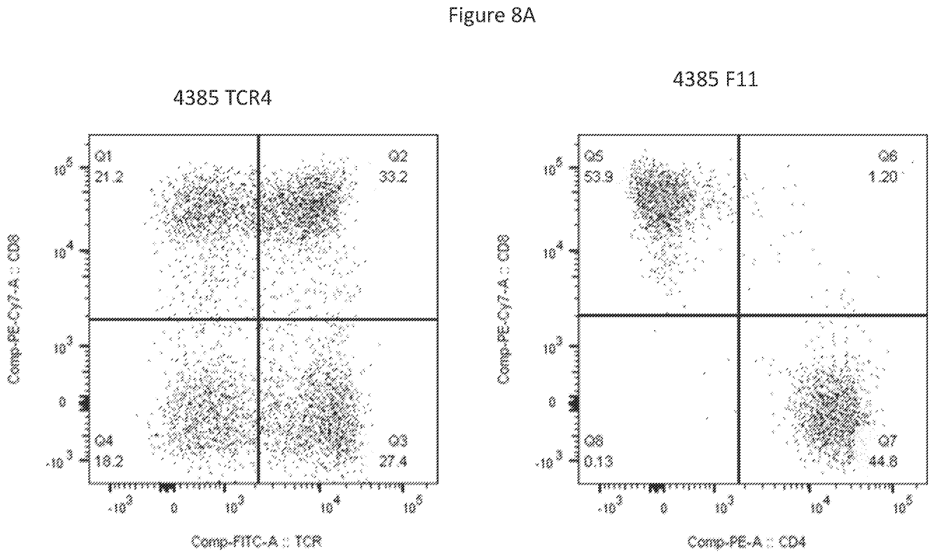

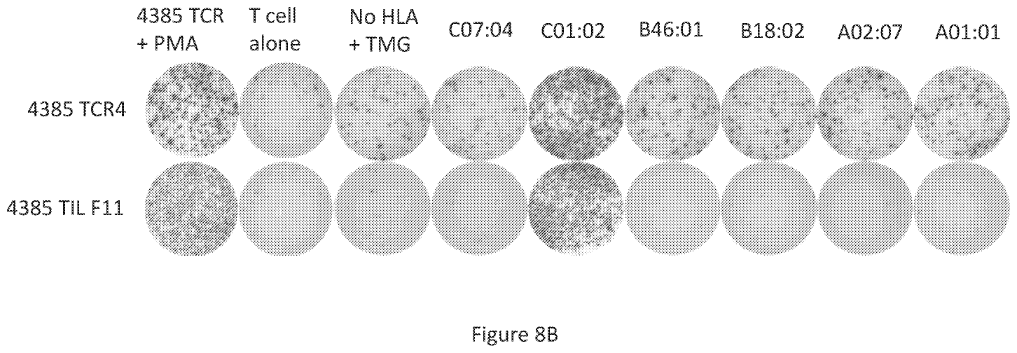

FIG. 8A presents dot plots showing TCR transduction efficacy into 4385 PBLs and the CD8/CD4 population distribution of the cells used in this experiment.

Results are IFNγ ELISPOT (FIG. 8B) assays and from 41BB/OX40 flow cytometry (Table 9). Table 9 presents the percentage of 4-1BB+/OX40+ cells in CD8+gated cells, as a response to co-culture of 4385 PBL transduced with TCR4 or TIL fragment 11 (F11) with COS7 cell line DNA transfected with TMG encoding mutated (Mut) RAS minigenes (including G12V) and one of six different HLA alleles expressed by Patient 4385 (HLA-A*01:01, HLA-A*02:07, HLA-B*18:02, HLA-B*46:01, HLA-C*01:02 or HLA-C*07:04)

TABLE 9

| | 4385 TCR4 | 4385 TIL F11 |
|---|---|---|
| A01:01 | 2.26 | 0.82 |
| A02:07 | 2.11 | 1.65 |
| B18:02 | 2.37 | 1.51 |
| B46:01 | 2.55 | 1.05 |
| C01:02 | 20.5 | 46.8 |
| C07:04 | 2.04 | 1.22 |
| No HLA + TMG | 2.71 | 1.12 |
| T cell alone | 3.03 | 0 |
| 4385 TCR + PMA | 4.76 | 6.12 |

### EXAMPLE 12

This example presents titration assays using FACS and ELISPOT (4385 TIL F11).

4385 TIL F11 were co-cultured with autologous DC loaded with RAS G12V ME8 or RAS LP (G12V/WT) in different concentrations.

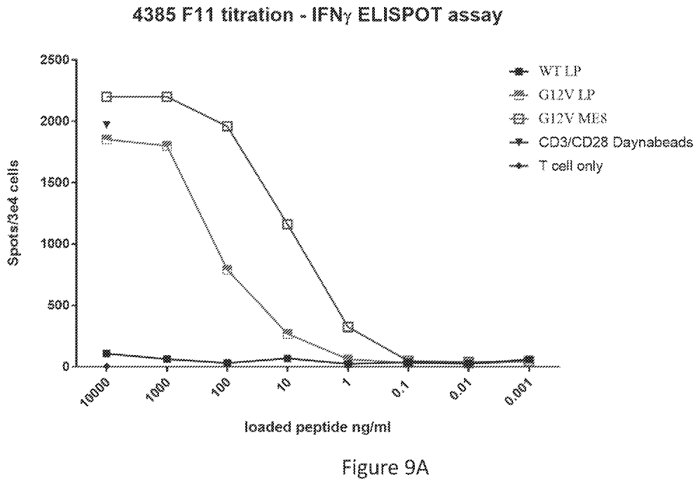

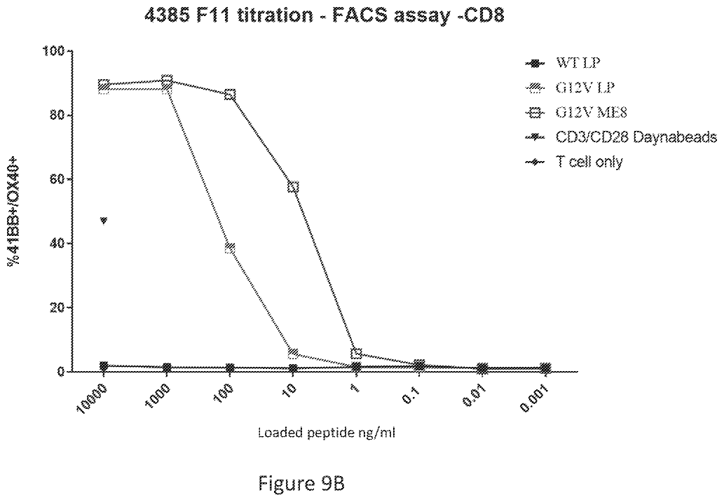

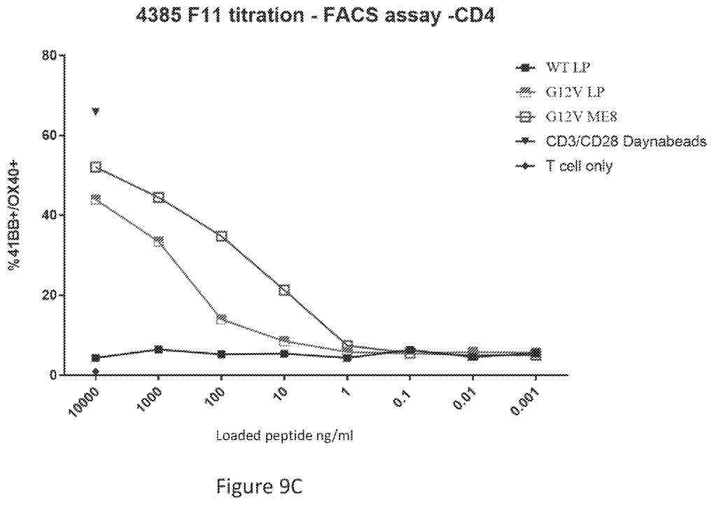

The reactivity was tested by IFNγ ELISPOT (FIG. 9A), and 41BB/OX40 flow cytometry with gating to CD8 (FIG. 9B) and gating to CD4 (FIG. 9C).

### EXAMPLE 13

This example presents titration assays using FACS and ELISPOT (4385 Transduced (Td) TCR4).

4385 TCR4-transduced PBL were co-cultured with autologous DC loaded with RAS G12V ME8 or RAS LP (G12V/WT) in different concentrations.

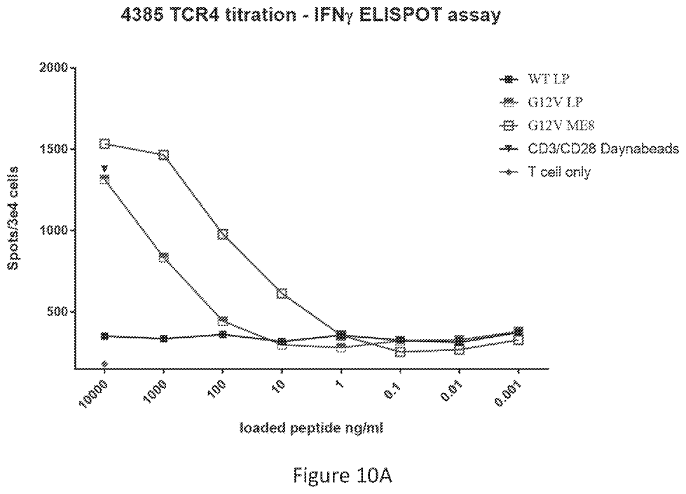

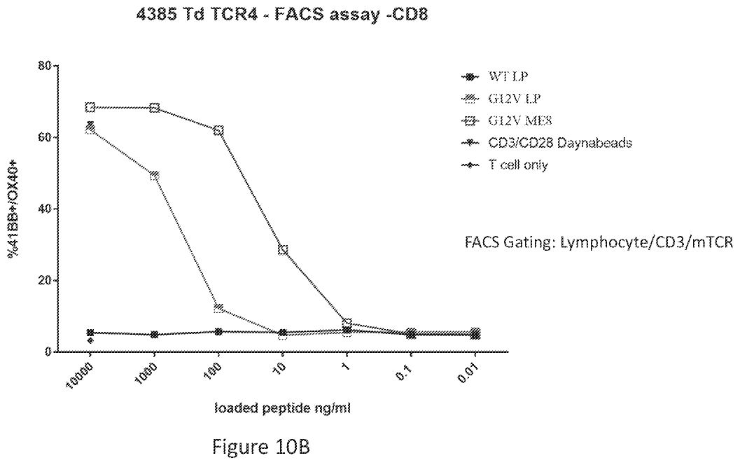

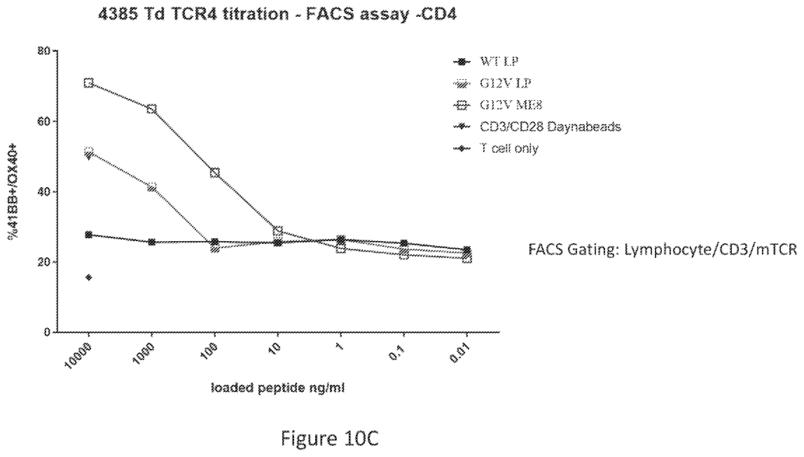

The reactivity was tested by IFNγ ELISPOT (FIG. 10A), and 41BB/OX40 flow cytometry with gating to CD8 (FIG. 10B) and gating to CD4 (FIG. 10C).

### EXAMPLE 14

This example presents titration assays using FACS and ELISPOT (healthy donor PBLs Transduced (Td) with 4385 TCR4).

4385 TCR4-transduced PBL were co-cultured with 4385 DC loaded with RAS G12V ME8 or RAS ME WT4 (WT sequence of ME8) in different concentrations.

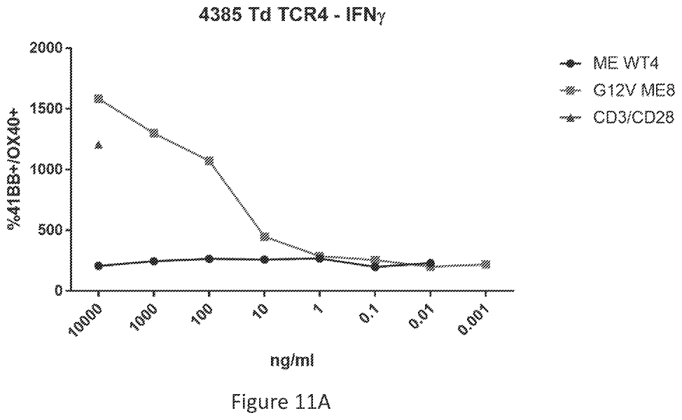

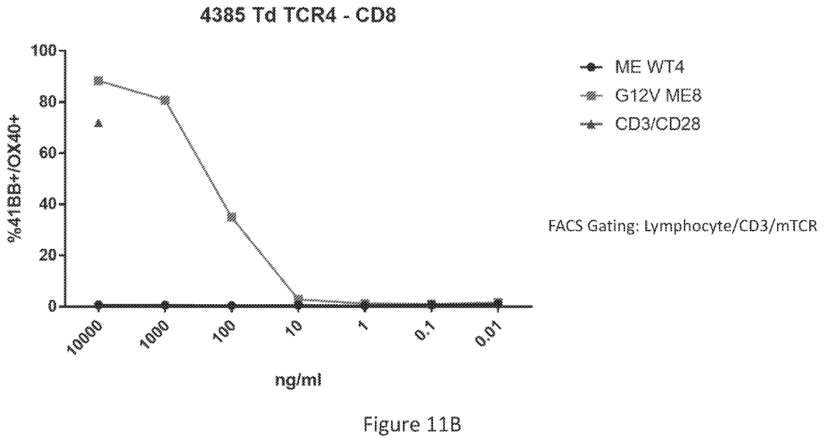

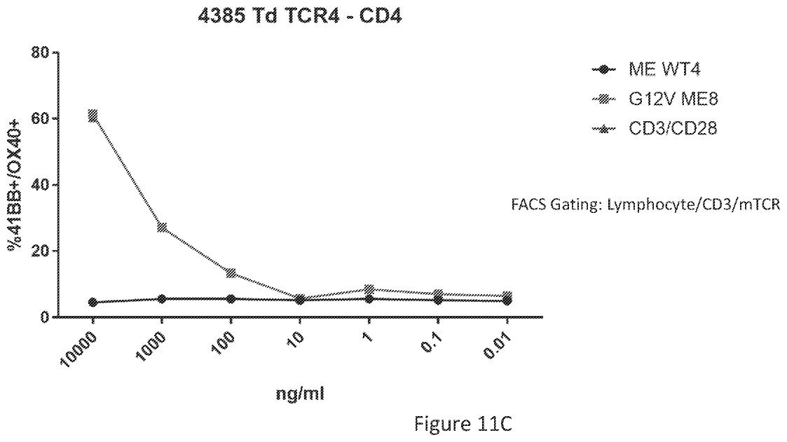

The reactivity was tested by IFNγ ELISPOT (FIG. 11A), and 41BB/OX40 flow cytometry with gating to CD8 (FIG. 11B) and gating to CD4 (FIG. 11C).

### EXAMPLE 15

This example demonstrates the identification of TIL reactive to RAS G12V from the tumor fragments of colorectal cancer Patient 4394.

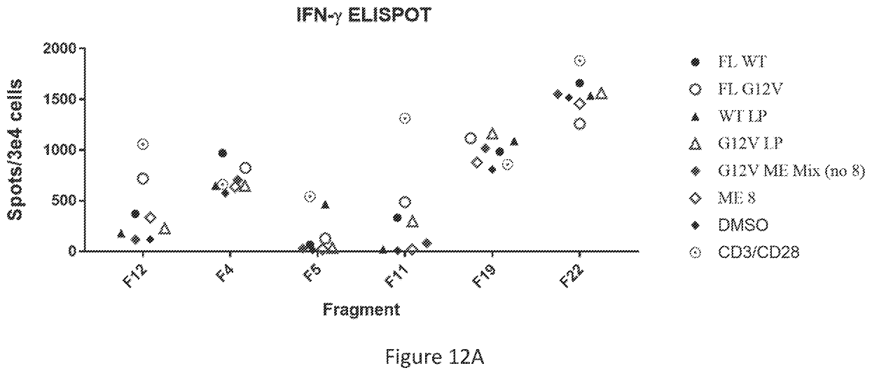

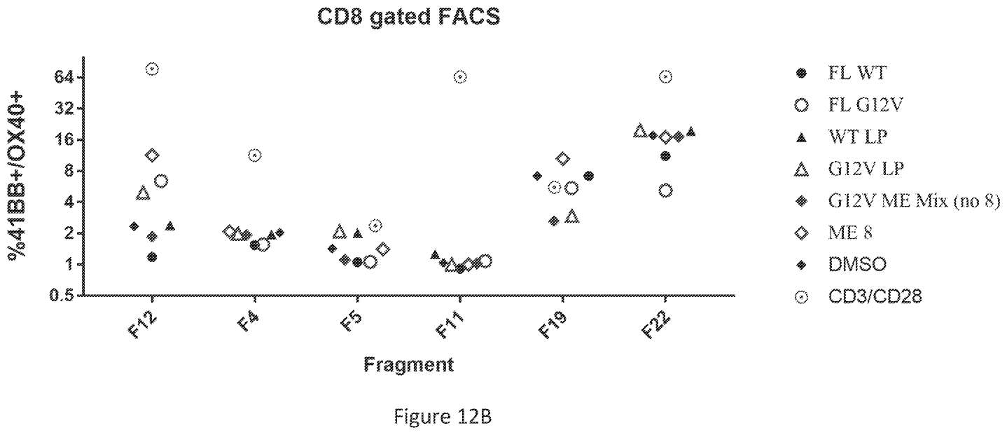

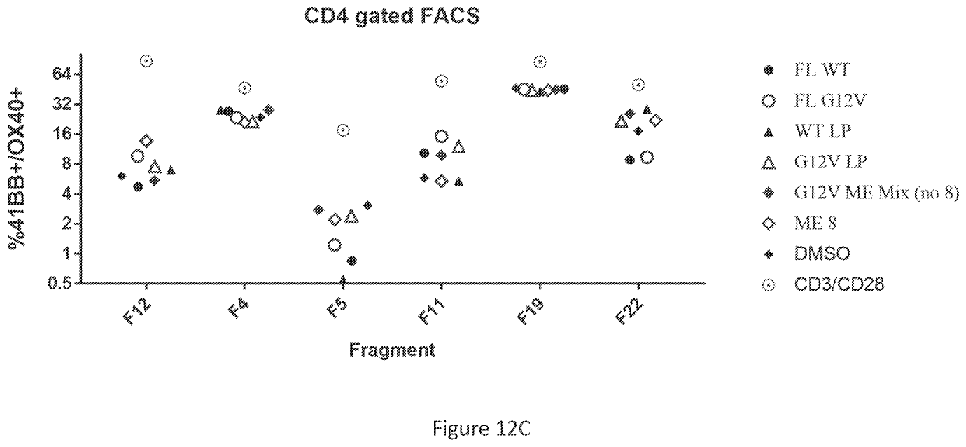

TIL from tumor fragments of Patient 4394 were co-cultured with autologous DC loaded with RAS G12V ME8 or RAS LP (G12V or WT) or transfected with a RAS FL (G12V or WT) genes. TIL co-cultured with DC treated with DMSO served as negative controls. TIL non-specifically stimulated with anti-CD3/anti-CD28 Dynabeads material served as a positive control. The reactivity was tested by IFNγ ELISPOT (FIG. 12A), and 41BB/OX40 flow cytometry with gating to CD8 (FIG. 12B) and gating to CD4 (FIG. 12C).

### EXAMPLE 16

This example demonstrates that TIL isolated from tumor fragment F12 of Patient 4394 specifically recognize RAS G12V.

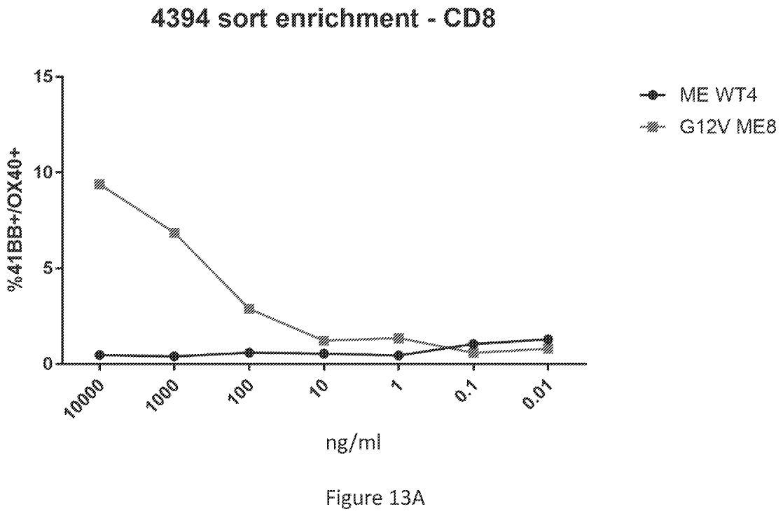

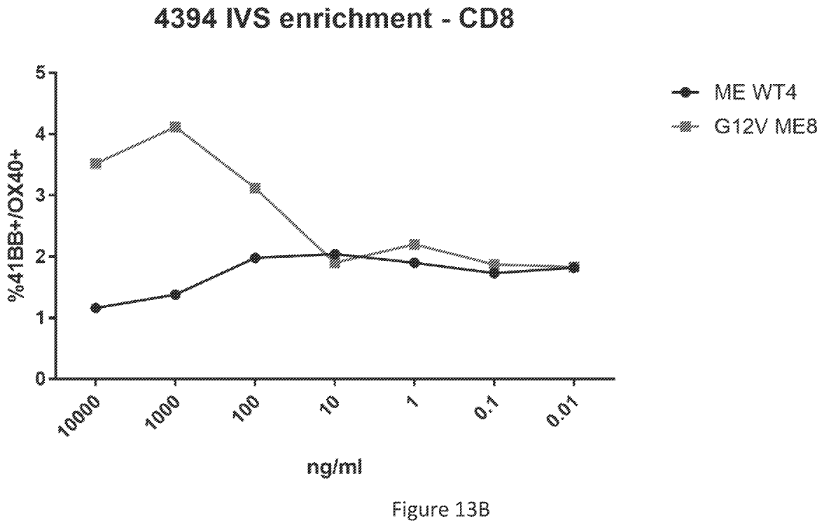

TILs from tumor fragment F12 of Patient 4394 were co-cultured with autologous DC loaded with RAS G12V ME8 or ME WT4 (WT sequence of ME8) in different concentrations, after enrichment of the TIL. The reactivity was tested using 41BB/OX40 flow cytometry assay gated to CD8 after sort enrichment (FIG. 13). The reactivity was tested by 41BB/OX40 flow cytometry with gating to CD8 (FIG. 13A) and by IFNy ELISPOT after sort enrichment (FIG. 13B) where TIL non-specifically stimulated by co-culture with anti-CD3/anti-CD28 Dynabeads material served as a positive control.

Two TCRs (termed TCRA and TCRB) from fragment F12 were further studied.

TABLE 10

| | Alpha chain | Beta chain |
|---|---|---|
| TCRA | TRAV12-1*01 + TRAJ36*01 | TRBV6-5*01 + TRBJ2-3*01 |
| TCRB | TRAV25*01 + TRAJ53*01 | TRBV4-1*01 + TRBJ2-7*01 |

TCRA and TCRB were found to be restricted by C*01:02.

### EXAMPLE 17

This example demonstrates the isolation of a TCR having antigenic specificity for human RAS G12V mutation presented by HLA-C*01:02 from the TIL of Patient 4394.

The TIL from tumor fragment F12 of Patient 4394 were determined to recognize RAS G12V and not WT RAS in Examples 15 and 16.

To identify the TCR sequence, the reactive TILs were sorted by fluorescence-activated cell sorting (FACS) based on the upregulation of the T cell activation markers, 4-1BB/OX40. Subsequently, the cells were lysed, and the TCR transcripts were Sanger sequenced.

### EXAMPLE 18

This example demonstrates a method of preparing a retroviral vector comprising a nucleotide sequence encoding the human anti-RAS G12V TCR of Example 17 with modified murine constant regions.

A nucleic acid sequence encoding the human RAS G12V reactive 4394 TCRA of Example 17 and including a cysteine substituted, LVL-modified murine constant region was cloned into a retroviral expression vector. The α chain murine constant region comprised the amino acid sequence of SEQ ID NO: 17 wherein X at position 48 is Cys, X at position 112 is Leu, X at position 114 is Ile, and X at position 115 is Val. The resulting full-length α chain comprised the amino acid sequence of SEQ ID NO: 74. The β chain constant region comprised the amino acid sequence of SEQ ID NO: 18, wherein X at position 57 is Cys. The resulting full-length β chain comprised the amino acid sequence of SEQ ID NO: 75. A linker comprising the amino acid sequence of RAKRSGSGATNFSLLKQAGDVEENPGP (SEQ ID NO: 25) was positioned between the α chain constant region and the 13 chain variable region. The sequences as provided are codon optimized.

To allow cloning the second amino acid within the N-terminal signal peptide was changed to an alanine (A). This example describes a synthesis of bicistronic vector in 5′TCRβ to TCRα 3′ orientation, but the order of TCRβ to TCRα can be reversed.

The amino acid sequences of the TCR alpha and betα chain variable regions are shown in Table 11. The CDRs are underlined.

TABLE 11

| TCR Name | TCR chain | Amino acid sequence |
|---|---|---|
| 4394 TCR | Alpha | MISLRVLLVILWLQLSWVWSQRKEVEQDPGPFNVPEGATVAFN |
| (TCRA) | (TRAV12-1*01 + | CTYSNSASQSFFWYRQDCRKEPKLLMSVYSSGNEDGRFTAQL |
| | TRAJ36*01) | NRASQYISLLIRDSKLSDSATYLCVVDDQTGANNLFFGTGTRLTV |
| | (with wild type N- | IP |
| | terminal signal | (SEQ ID NO: 70) |
| | peptide) | |
| | Beta | MAIGLLCCAALSLLWAGPVNAGVTQTPKFQVLKTGQSMTLQCA |
| | (TRBV6-5*01 + | QDMNHEYMSWYRQDPGMGLRLIHYSVGAGITDQGEVPNGYNV |
| | TRBJ2-3*01 + | SRSTTEDFPLRLLSAAPSQTSVYFCASRNLGDTQYFGPGTRLTV |
| | TRBDP01) | L |
| | (with variant N- | (SEQ ID NO: 71) |
| | terminal signal | |
| | peptide) | |
| | Alpha | RKEVEQDPGPFNVPEGATVAFNCTYSNSASQSFFWYRQDCRK |
| | (TRAV12-1*01 + | EPKLLMSVYSSGNEDGRFTAQLNRASQYISLLIRDSKLSDSATYL |
| | TRAJ36*01) | CVVDDQTGANNLFFGTGTRLTVIP |
| | (without N-terminal | (SEQ ID NO: 72) |
| | signal peptide | |
| | predicted by IMGT) | |
| | Beta | NAGVTQTPKFQVLKTGQSMTLQCAQDMNHEYMSWYRQDPGM |
| | (TRBV6-5*01 + | GLRLIHYSVGAGITDQGEVPNGYNVSRSTTEDFPLRLLSAAPSQ |
| | TRBJ2-3*01 + | TSVYFCASRNLGDTQYFGPGTRLTVL |
| | TRBD1*01) | (SEQ ID NO: 73) |
| | (without N-terminal | |
| | signal peptide | |
| | predicted by IMGT) | |
| | Alpha | MASLRVLLVILWLQLSWVWSQRKEVEQDPGPFNVPEGATVAFN |
| | (TRAV12-1*01 + | CTYSNSASQSFFWYRQDCRKEPKLLMSVYSSGNEDGRFTAQL |
| | TRAJ36*01) | NRASQYISLLIRDSKLSDSATYLCVVDDQTGANNLFFGTGTRLTV |
| | (with variant N- | IP |
| | terminal signal | (SEQ ID NO: 132) |
| | peptide) | |
| | Beta | MSIGLLCCAALSLLWAGPVNAGVTQTPKFQVLKTGQSMTLQCA |
| | (TRBV6-5*01 + | QDMNHEYMSWYRQDPGMGLRLIHYSVGAGITDQGEVPNGYNV |
| | TRBJ2-3*01 + | SRSTTEDFPLRLLSAAPSQTSVYFCASRNLGDTQYFGPGTRLTV |
| | TRBDP01) | L |
| | (with wild type N- | (SEQ ID NO: 133) |
| | terminal signal | |
| | peptide) | |
| | Alpha | QRKEVEQDPGPFNVPEGATVAFNCTYSNSASQSFFWYRQDCR |
| | (TRAV12-1*01 + | KEPKLLMSVYSSGNEDGRFTAQLNRASQYISLLIRDSKLSDSAT |
| | TRAJ36*01) | YLCVVDDQTGANNLFFGTGTRLTVIP |
| | (without N-terminal | (SEQ ID NO: 88) |
| | signal peptide | |
| | predicted by | |
| | SignalP) | |
| | Beta | GVTQTPKFQVLKTGQSMTLQCAQDMNHEYMSWYRQDPGMGL |
| | (TRBV6-5*01 + | RLIHYSVGAGITDQGEVPNGYNVSRSTTEDFPLRLLSAAPSQTS |
| | TRBJ2-3*01 + | VYFCASRNLGDTQYFGPGTRLTVL |
| | TRBD1*01) | (SEQ ID NO: 89) |
| | (without N-terminal | |
| | signal peptide | |
| | predicted by | |
| | SignalP) | |

### EXAMPLE 19

This example presents assays using FACS and ELISPOT (4394 Transduced (Td) TCRA of Example 18 and TCRB).

4394 TCRA- and 4394 TCRB-transduced PBL were co-cultured with autologous DC loaded with RAS G12V ME8, RAS ME WT4 (WT sequence of ME8), RAS LP (G12V or WT) or transfected with a RAS WT FL or RAS G12V FL mRNA. Cells co-cultured with DC treated with DMSO served as negative controls. PBL non-specifically stimulated by co-culture with anti-CD3/anti-CD28 Dynabeads material served as a positive control.

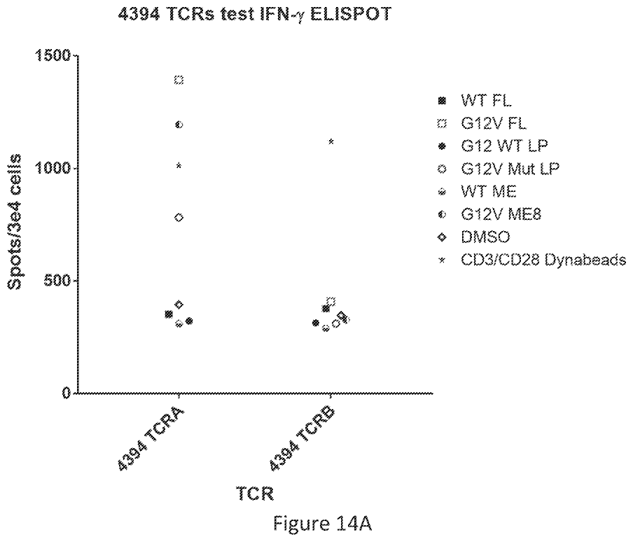

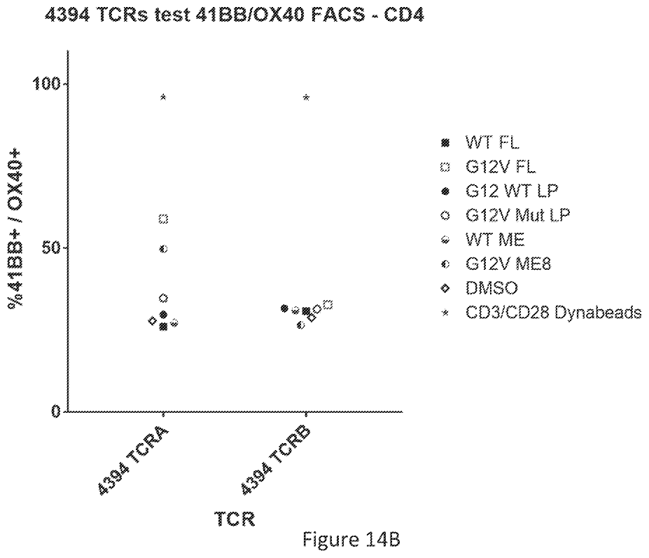

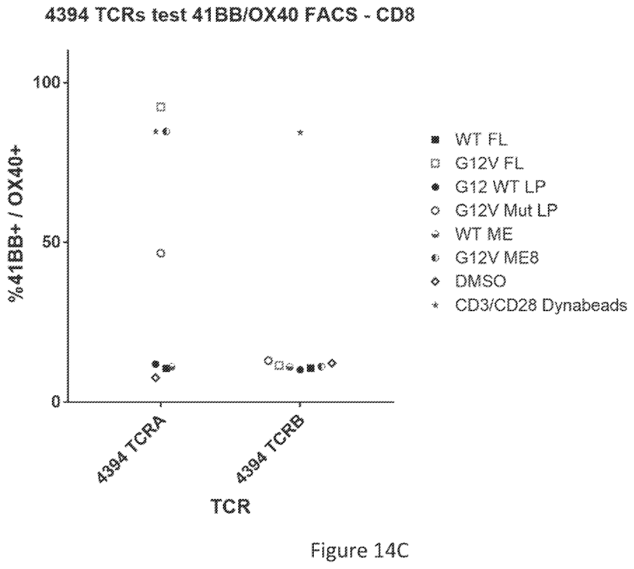

The reactivity was tested by IFNγ ELISPOT (FIG. 14A) and 41BB/OX40 flow cytometry with gating to CD4 (FIG. 14B) and gating to CD8 (FIG. 14C).

### EXAMPLE 20

This example presents titration assays using FACS and ELISPOT (healthy donor PBLs Transduced (Td) with 4394 TCRA of Example 18).

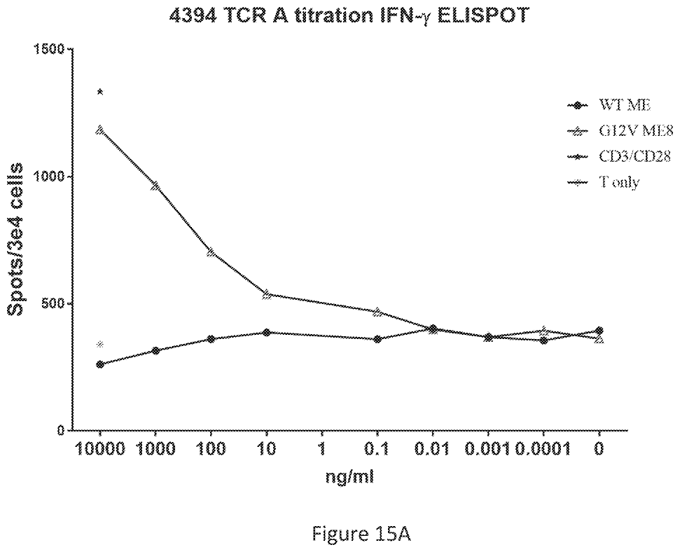

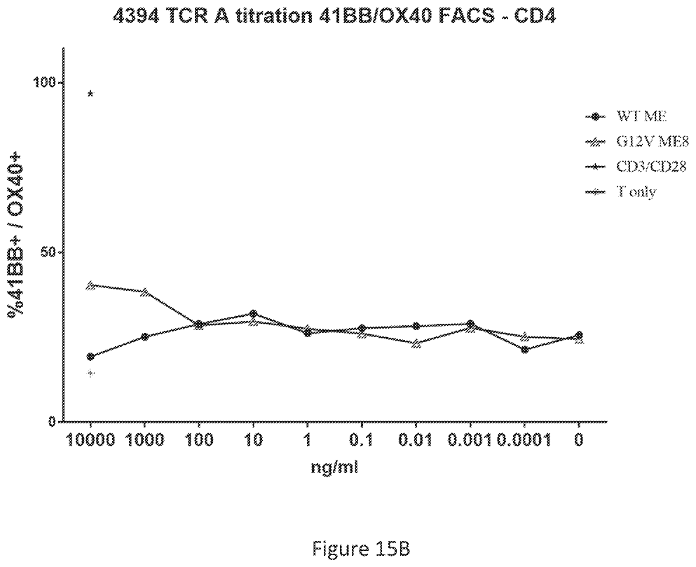

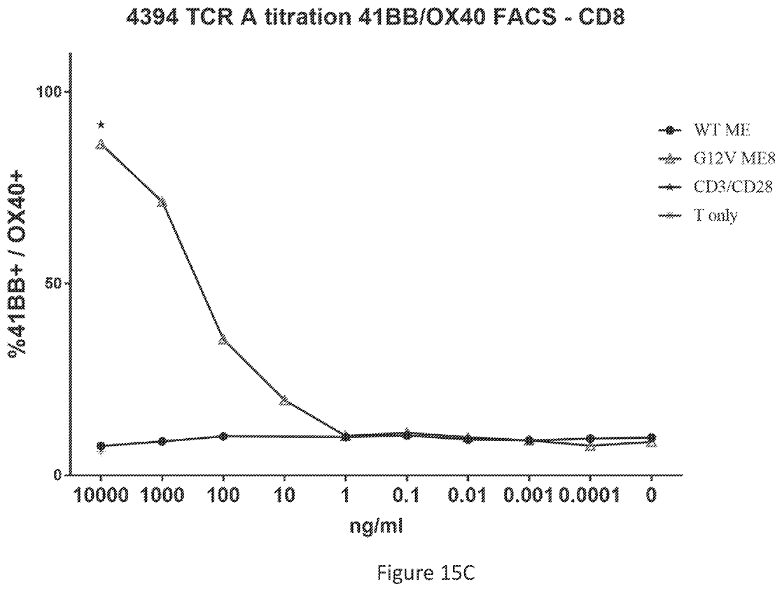

4394 TCRA-transduced PBL were co-cultured with 4394 DC loaded with RAS G12V ME8 or RAS ME WT4 (WT sequence of ME8) in different concentrations. PBL cultured alone in DMSO served as negative controls. PBL non-specifically stimulated by co-culture with anti-CD3/anti-CD28 Dynabeads material served as a positive control. The reactivity was tested by IFNγ ELISPOT (FIG. 15A), and 41BB/OX40 flow cytometry with gating to CD4 (FIG. 15B) and gating to CD8 (FIG. 15C).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An isolated or purified T-cell receptor (TCR) comprising the amino acid sequences of
(a) all of SEQ ID NOs: 1-3,
(b) all of SEQ ID NOs: 4-6,
(c) all of SEQ ID NOs: 31-33,
(d) all of SEQ ID NOs: 34-36,
(e) all of SEQ ID NOs: 64-66,
(f) all of SEQ ID NOs: 67-69,
(g) all of SEQ ID NOs: 1-6,
(h) all of SEQ ID NOs: 31-36, or
(i) all of SEQ ID NOs: 64-69,
wherein the TCR has antigenic specificity for a mutated human RAS amino acid sequence presented by a human leukocyte antigen (HLA) Class I molecule, and
wherein the mutated human RAS amino acid sequence is a mutated human Kirsten rat sarcoma viral oncogene homolog (KRAS), a mutated human Harvey rat sarcoma viral oncogene homolog (HRAS), or a mutated human Neuroblastoma rat sarcoma viral oncogene homolog (NRAS) amino acid sequence.

2. The isolated or purified TCR according to claim 1, wherein the mutated human RAS amino acid sequence is SEQ ID NO: 29 or SEQ ID NO: 30.

3. The isolated or purified TCR according to claim 1, wherein the TCR does not have antigenic specificity for the wild-type human RAS amino acid sequence of one or both of SEQ ID NO: 26 and SEQ ID NO: 27.

4. The isolated or purified TCR according to claim 1, wherein the HLA Class I molecule is an HLA-C molecule.

5. The isolated or purified TCR according to claim 1, wherein the HLA Class I molecule is an HLA-C*01 molecule.

6. The isolated or purified TCR according to claim 1, wherein the HLA Class I molecule is an HLA-C*01:02 molecule.

7. The isolated or purified TCR according to claim 1, comprising:
(i) an amino acid sequence at least 99% identical to the amino acid sequence of SEQ ID NO: 7;
(ii) an amino acid sequence at least 99% identical to the amino acid sequence of SEQ ID NO: 8;
(iii) an amino acid sequence at least 99% identical to the amino acid sequence of SEQ ID NO: 90;
(iv) an amino acid sequence at least 99% identical to the amino acid sequence of SEQ ID NO: 91;
(v) an amino acid sequence at least 99% identical to the amino acid sequence of SEQ ID NO: 37;
(vi) an amino acid sequence at least 99% identical to the amino acid sequence of SEQ ID NO: 38;
(vii) an amino acid sequence at least 99% identical to the amino acid sequence of SEQ ID NO: 92;
(viii) an amino acid sequence at least 99% identical to the amino acid sequence of SEQ ID NO: 93;
(ix) an amino acid sequence at least 99% identical to the amino acid sequence of SEQ ID NO: 70;
(x) an amino acid sequence at least 99% identical to the amino acid sequence of SEQ ID NO: 71;
(xi) an amino acid sequence at least 99% identical to the amino acid sequence of SEQ ID NO: 132;
(xii) an amino acid sequence at least 99% identical to the amino acid sequence of SEQ ID NO: 133;
(xiii) an amino acid sequence at least 99% identical to the amino acid sequence of SEQ ID NO: 47;
(xiv) an amino acid sequence at least 99% identical to the amino acid sequence of SEQ ID NO: 48;
(xv) an amino acid sequence at least 99% identical to the amino acid sequence of SEQ ID NO: 94;
(xvi) an amino acid sequence at least 99% identical to the amino acid sequence of SEQ ID NO: 85;
(xvii) an amino acid sequence at least 99% identical to the amino acid sequence of SEQ ID NO: 95;
(xviii) an amino acid sequence at least 99% identical to the amino acid sequence of SEQ ID NO: 49;
(xix) an amino acid sequence at least 99% identical to the amino acid sequence of SEQ ID NO: 50;
(xx) an amino acid sequence at least 99% identical to the amino acid sequence of SEQ ID NO: 96;
(xxi) an amino acid sequence at least 99% identical to the amino acid sequence of SEQ ID NO: 87;
(xxii) an amino acid sequence at least 99% identical to the amino acid sequence of SEQ ID NO: 97;
(xxiii) an amino acid sequence at least 99% identical to the amino acid sequence of SEQ ID NO: 72;
(xxvi) an amino acid sequence at least 99% identical to the amino acid sequence of SEQ ID NO: 73;
(xxv) an amino acid sequence at least 99% identical to the amino acid sequence of SEQ ID NO: 88;
(xxvi) an amino acid sequence at least 99% identical to the amino acid sequence of SEQ ID NO: 89;
(xxvii) both (i) and (ii), both (i) and (iv), both (ii) and (iii), both (iii) and (iv), both (v) and (vi), both (v) and (viii), both (vi) and (vii), both (vii) and (viii), both (ix) and (x), both (ix) and (xii), both (x) and (xi), both (xi) and (xii), both (xiii) and (xiv), both (xiii) and (xvi), both (xiii) and (xvii), both (xiv) and (xv), both (xv) and (xvi), both (xv) and (xvii), both (xviii) and (xix), both (xviii) and (xxi), both (xviii) and (xxii), both (xix) and (xx), both (xx) and (xxi), both (xx) and (xxii), both (xxiii) and (xxiv), both (xxiii) and (xxvi), both (xxiv) and (xxv), or both (xxv) and (xxvi).

8. The isolated or purified TCR according to claim 1, comprising:
(i) the amino acid sequence of SEQ ID NO: 7;
(ii) the amino acid sequence of SEQ ID NO: 8;
(iii) the amino acid sequence of SEQ ID NO: 90;
(iv) the amino acid sequence of SEQ ID NO: 91;
(v) the amino acid sequence of SEQ ID NO: 37;
(vi) the amino acid sequence of SEQ ID NO: 38;
(vii) the amino acid sequence of SEQ ID NO: 92;
(viii) the amino acid sequence of SEQ ID NO: 93;
(ix) the amino acid sequence of SEQ ID NO: 70;
(x) the amino acid sequence of SEQ ID NO: 71;
(xi) the amino acid sequence of SEQ ID NO: 132;
(xii) the amino acid sequence of SEQ ID NO: 133;
(xiii) the amino acid sequence of SEQ ID NO: 47;
(xiv) the amino acid sequence of SEQ ID NO: 48;
(xv) the amino acid sequence of SEQ ID NO: 94;
(xvi) the amino acid sequence of SEQ ID NO: 85;
(xvii) the amino acid sequence of SEQ ID NO: 95;
(xviii) the amino acid sequence of SEQ ID NO: 49;
(xix) the amino acid sequence of SEQ ID NO: 50;
(xx) the amino acid sequence of SEQ ID NO: 96;
(xxi) the amino acid sequence of SEQ ID NO: 87;
(xxii) the amino acid sequence of SEQ ID NO: 97;
(xxiii) the amino acid sequence of SEQ ID NO: 72;
(xxvi) the amino acid sequence of SEQ ID NO: 73;
(xxv) the amino acid sequence of SEQ ID NO: 88;
(xxvi) the amino acid sequence of SEQ ID NO: 89;
(xxvii) both (i) and (ii), both (i) and (iv), both (ii) and (iii), both (iii) and (iv), both (v) and (vi), both (v) and (viii), both (vi) and (vii), both (vii) and (viii), both (ix) and (x), both (ix) and (xii), both (x) and (xi), both (xi) and (xii), both (xiii) and (xiv), both (xiii) and (xvi), both (xiii) and (xvii), both (xiv) and (xv), both (xv) and (xvi), both (xv) and (xvii), both (xviii) and (xix), both (xviii) and (xxi), both (xviii) and (xxii), both (xix) and (xx), both (xx) and (xxi), both (xx) and (xxii), both (xxiii) and (xxiv), both (xxiii) and (xxvi), both (xxiv) and (xxv), or both (xxv) and (xxvi).

9. The isolated or purified TCR according to claim 1, further comprising:
(a) an α chain constant region comprising an amino acid sequence at least 99% identical to the amino acid sequence of SEQ ID NO: 17, wherein:
(i) X at position 48 of SEQ ID NO: 17 is Thr or Cys;
(ii) X at position 112 of SEQ ID NO: 17 is Ser, Ala, Val, Leu, Ile, Pro, Phe, Met, or Trp;
(iii) X at position 114 of SEQ ID NO: 17 is Met, Ala, Val, Leu, Ile, Pro, Phe, or Trp; and
(iv) X at position 115 of SEQ ID NO: 17 is Gly, Ala, Val, Leu, Ile, Pro, Phe, Met, or Trp;
(b) a β chain constant region comprising an amino acid sequence at least 99% identical to the amino acid sequence of SEQ ID NO: 18, wherein X at position 57 of SEQ ID NO: 18 is Ser or Cys; or
(c) both (a) and (b) SEQ ID NO

10. The isolated or purified TCR according to claim 1, further comprising:
(a) an α chain constant region comprising the amino acid sequence of SEQ ID NO: 17, wherein:
(i) X at position 48 of SEQ ID NO: 17 is Thr or Cys;
(ii) X at position 112 of SEQ ID NO: 17 is Ser, Ala, Val, Leu, Ile, Pro, Phe, Met, or Trp;
(iii) X at position 114 of SEQ ID NO: 17 is Met, Ala, Val, Leu, Ile, Pro, Phe, or Trp; and
(iv) X at position 115 of SEQ ID NO: 17 is Gly, Ala, Val, Leu, Ile, Pro, Phe, Met, or Trp;
(b) a β chain constant region comprising the amino acid sequence of SEQ ID NO: 18, wherein X at position 57 of SEQ ID NO: 18 is Ser or Cys; or
(c) both (a) and (b) SEQ ID NO

11. The isolated or purified TCR according to claim 1, comprising:
(a) an α chain comprising an amino acid sequence at least 99% identical to the amino acid sequence of SEQ ID NO: 21, wherein:
(i) X at position 180 of SEQ ID NO: 21 is Thr or Cys;
(ii) X at position 244 of SEQ ID NO: 21 is Ser, Ala, Val, Leu, Ile, Pro, Phe, Met, or Trp;
(iii) X at position 246 of SEQ ID NO: 21 is Met, Ala, Val, Leu, Ile, Pro, Phe, or Trp; and
(iv) X at position 247 of SEQ ID NO: 21 is Gly, Ala, Val, Leu, Ile, Pro, Phe, Met, or Trp;
(b) a β chain comprising an amino acid sequence at least 99% identical to the amino acid sequence of SEQ ID NO: 22, wherein X at position 190 of SEQ ID NO: 22 is Ser or Cys;
(c) an α chain comprising an amino acid sequence at least 99% identical to the amino acid sequence of SEQ ID NO: 100, wherein:
(i) X at position 180 of SEQ ID NO: 100 is Thr or Cys;
(ii) X at position 244 of SEQ ID NO: 100 is Ser, Ala, Val, Leu, Ile, Pro, Phe, Met, or Trp;
(iii) X at position 246 of SEQ ID NO: 100 is Met, Ala, Val, Leu, Ile, Pro, Phe, or Trp; and
(iv) X at position 247 of SEQ ID NO: 100 is Gly, Ala, Val, Leu, Ile, Pro, Phe, Met, Trp;
(d) a β chain comprising an amino acid sequence at least 99% identical to the amino acid sequence of SEQ ID NO: 101, wherein X at position 190 of SEQ ID NO: 101 is Ser or Cys;
(e) an α chain comprising an amino acid sequence at least 99% identical to the amino acid sequence of SEQ ID NO: 41, wherein:
(i) X at position 181 of SEQ ID NO: 41 is Thr or Cys;
(ii) X at position 245 of SEQ ID NO: 41 is Ser, Ala, Val, Leu, Ile, Pro, Phe, Met, or Trp;
(iii) X at position 247 of SEQ ID NO: 41 is Met, Ala, Val, Leu, Ile, Pro, Phe, or Trp; and
(iv) X at position 248 of SEQ ID NO: 41 is Gly, Ala, Val, Leu, Ile, Pro, Phe, Met, or Trp;
(f) a β chain comprising an amino acid sequence at least 99% identical to the amino acid sequence of SEQ ID NO: 42, wherein X at position 195 of SEQ ID NO: 42 is Ser or Cys;
(g) an α chain comprising an amino acid sequence at least 99% identical to the amino acid sequence of SEQ ID NO: 106, wherein:
(i) X at position 181 of SEQ ID NO: 106 is Thr or Cys;
(ii) X at position 245 of SEQ ID NO: 106 is Ser, Ala, Val, Leu, Ile, Pro, Phe, Met, or Trp;
(iii) X at position 247 of SEQ ID NO: 106 is Met, Ala, Val, Leu, Ile, Pro, Phe, or Trp; and
(iv) X at position 248 of SEQ ID NO: 106 is Gly, Ala, Val, Leu, Ile, Pro, Phe, Met, or Trp;
(h) a β chain comprising an amino acid sequence at least 99% identical to the amino acid sequence of SEQ ID NO: 107, wherein X at position 195 of SEQ ID NO: 107 is Ser or Cys;
(i) an α chain comprising an amino acid sequence at least 99% identical to the amino acid sequence of SEQ ID NO: 74, wherein:
(i) X at position 180 of SEQ ID NO: 74 is Thr or Cys;
(ii) X at position 244 of SEQ ID NO: 74 is Ser, Ala, Val, Leu, Ile, Pro, Phe, Met, or Trp;
(iii) X at position 246 of SEQ ID NO: 74 is Met, Ala, Val, Leu, Ile, Pro, Phe, or Trp; and
(iv) X at position 247 of SEQ ID NO: 74 is Gly, Ala, Val, Leu, Ile, Pro, Phe, Met, or Trp;
(j) a β chain comprising an amino acid sequence at least 99% identical to the amino acid sequence of SEQ ID NO: 75, wherein X at position 187 of SEQ ID NO: 75 is Ser or Cys;
(k) an α chain comprising an amino acid sequence at least 99% identical to the amino acid sequence of SEQ ID NO: 136, wherein:
(i) X at position 180 of SEQ ID NO: 136 is Thr or Cys;
(ii) X at position 244 of SEQ ID NO: 136 is Ser, Ala, Val, Leu, Ile, Pro, Phe, Met, or Trp;
(iii) X at position 246 of SEQ ID NO: 136 is Met, Ala, Val, Leu, Ile, Pro, Phe, or Trp; and
(iv) X at position 247 of SEQ ID NO: 136 is Gly, Ala, Val, Leu, Ile, Pro, Phe, Met, or Trp;
(l) a β chain comprising an amino acid sequence at least 99% identical to the amino acid sequence of SEQ ID NO: 137, wherein X at position 187 of SEQ ID NO: 75 is Ser or Cys;
(m) both (a) and (b); both (a) and (d); both (b) and (c); or both (c) and (d);
(n) both (e) and (o); both (e) and h); both (f) and (g); or both (g) and (h);
(n) both (i) and (j); both (i) and 1); both (j) and (k); or both (k) and (l);
(o) an α chain comprising an amino acid sequence at least 99% identical to the amino acid sequence of SEQ ID NO: 55, wherein:
(i) X at position 160 of SEQ ID NO: 55 is Thr or Cys;
(ii) X at position 224 of SEQ ID NO: 55 is Ser, Ala, Val, Leu, Ile, Pro, Phe, Met, or Trp;
(iii) X at position 226 of SEQ ID NO: 55 is Met, Ala, Val, Leu, Ile, Pro, Phe, or Trp; and <(iv) X at position 227 of SEQ ID NO: 55 is Gly, Ala, Val, Leu, Ile, Pro, Phe, Met, or Trp;
(p) a β chain comprising an amino acid sequence at least 99% identical to the amino acid sequence of SEQ ID NO: 56, wherein X at position 168 of SEQ ID NO: 56 is Ser or Cys;
(q) an α chain comprising an amino acid sequence at least 99% identical to the amino acid sequence of SEQ ID NO: 112, wherein:
(i) X at position 159 of SEQ ID NO: 112 is Thr or Cys;
(ii) X at position 223 of SEQ ID NO: 112 is Ser, Ala, Val, Leu, Ile, Pro, Phe, Met, or Trp;
(iii) X at position 225 of SEQ ID NO: 112 is Met, Ala, Val, Leu, Ile, Pro, Phe, or Trp; and
(iv) X at position 226 of SEQ ID NO: 112 is Gly, Ala, Val, Leu, Ile, Pro, Phe, Met, or Trp;
(r) a β chain comprising an amino acid sequence at least 99% identical to the amino acid sequence of SEQ ID NO: 113, wherein X at position 173 of SEQ ID NO: 113 is Ser or Cys;
(s) an α chain comprising an amino acid sequence at least 99% identical to the amino acid sequence of SEQ ID NO: 57, wherein:
(i) X at position 160 of SEQ ID NO: 57 is Thr or Cys;
(ii) X at position 224 of SEQ ID NO: 57 is Ser, Ala, Val, Leu, Ile, Pro, Phe, Met, or Trp;
(iii) X at position 226 of SEQ ID NO: 57 is Met, Ala, Val, Leu, Ile, Pro, Phe, or Trp; and
(iv) X at position 227 of SEQ ID NO: 57 is Gly, Ala, Val, Leu, Ile, Pro, Phe, Met, or Trp;
(t) a β chain comprising an amino acid sequence at least 99% identical to the amino acid sequence of SEQ ID NO: 58, wherein X at position 173 of SEQ ID NO: 58 is Ser or Cys;
(u) an α chain comprising an amino acid sequence at least 99% identical to the amino acid sequence of SEQ ID NO: 118, wherein:
(i) X at position 161 of SEQ ID NO: 118 is Thr or Cys;
(ii) X at position 225 of SEQ ID NO: 118 is Ser, Ala, Val, Leu, Ile, Pro, Phe, Met, or Trp;
(iii) X at position 227 of SEQ ID NO: 118 is Met, Ala, Val, Leu, Ile, Pro, Phe, or Trp; and
(iv) X at position 228 of SEQ ID NO: 118 is Gly, Ala, Val, Leu, Ile, Pro, Phe, Met, or Trp;
(v) a β chain comprising an amino acid sequence at least 99% identical to the amino acid sequence of SEQ ID NO: 119, wherein X at position 103178 of SEQ ID NO: 119 is Ser or Cys;
(w) an α chain comprising an amino acid sequence at least 99% identical to the amino acid sequence of SEQ ID NO: 76, wherein:
(i) X at position 159 of SEQ ID NO: 76 is Thr or Cys;
(ii) X at position 223 of SEQ ID NO: 76 is Ser, Ala, Val, Leu, Ile, Pro, Phe, Met, or Trp;
(iii) X at position 225 of SEQ ID NO: 76 is Met, Ala, Val, Leu, Ile, Pro, Phe, or Trp; and
(iv) X at position 226 of SEQ ID NO: 76 is Gly, Ala, Val, Leu, Ile, Pro, Phe, Met, or Trp;
(x) a β chain comprising an amino acid sequence at least 99% identical to the amino acid sequence of SEQ ID NO: 77, wherein X at position 168 of SEQ ID NO: 77 is Ser or Cys;
(y) an α chain comprising an amino acid sequence at least 99% identical to the amino acid sequence of SEQ ID NO: 144, wherein:
(i) X at position 160 of SEQ ID NO: 144 is Thr or Cys;
(ii) X at position 224 of SEQ ID NO: 144 is Ser, Ala, Val, Leu, Ile, Pro, Phe, Met, or Trp;
(iii) X at position 226 of SEQ ID NO: 144 is Met, Ala, Val, Leu, Ile, Pro, Phe, or Trp; and
(iv) X at position 227 of SEQ ID NO: 144 is Gly, Ala, Val, Leu, Ile, Pro, Phe, Met, or Trp;
(z) a β chain comprising an amino acid sequence at least 99% identical to the amino acid sequence of SEQ ID NO: 145, wherein X at position 166 of SEQ ID NO: 145 is Ser or Cys;
(aa) both (o) and (p); or both (q) and (r);
(bb) both (s) and (t); or both (u) and (v);
(cc) both (w) and (x); or both (y) and (z);
(dd) an α chain comprising an amino acid sequence at least 99% identical to the amino acid sequence of SEQ ID NO: 51;
(ee) a β chain comprising an amino acid sequence at least 99% identical to the amino acid sequence of SEQ ID NO: 52;
(ff) an α chain comprising an amino acid sequence at least 99% identical to the amino acid sequence of SEQ ID NO: 114;
(gg) a β chain comprising an amino acid sequence at least 99% identical to the amino acid sequence of SEQ ID NO: 115;
(hh) an α chain comprising an amino acid sequence at least 99% identical to the amino acid sequence of SEQ ID NO: 128;
(ii) a β chain comprising an amino acid sequence at least 99% identical to the amino acid sequence of SEQ ID NO: 129;
(jj) an α chain comprising an amino acid sequence at least 99% identical to the amino acid sequence of SEQ ID NO: 116;
(kk) a β chain comprising an amino acid sequence at least 99% identical to the amino acid sequence of SEQ ID NO: 117;
(ll) an α chain comprising an amino acid sequence at least 99% identical to the amino acid sequence of SEQ ID NO: 53;
(mm) a β chain comprising an amino acid sequence at least 99% identical to the amino acid sequence of SEQ ID NO: 54;
(nn) an α chain comprising an amino acid sequence at least 99% identical to the amino acid sequence of SEQ ID NO: 120;
(oo) a β chain comprising an amino acid sequence at least 99% identical to the amino acid sequence of SEQ ID NO: 121;
(pp) an α chain comprising an amino acid sequence at least 99% identical to the amino acid sequence of SEQ ID NO: 130;
(qq) a β chain comprising an amino acid sequence at least 99% identical to the amino acid sequence of SEQ ID NO: 131;
(rr) an α chain comprising an amino acid sequence at least 99% identical to the amino acid sequence of SEQ ID NO: 122;
(ss) a β chain comprising an amino acid sequence at least 99% identical to the amino acid sequence of SEQ ID NO: 123;
(tt) an α chain comprising an amino acid sequence at least 99% identical to the amino acid sequence of SEQ ID NO: 80;
(uu) a β chain comprising an amino acid sequence at least 99% identical to the amino acid sequence of SEQ ID NO: 81;
(vv) an α chain comprising an amino acid sequence at least 99% identical to the amino acid sequence of SEQ ID NO: 146;
(ww) a β chain comprising an amino acid sequence at least 99% identical to the amino acid sequence of SEQ ID NO: 146;
(xx) an α chain comprising an amino acid sequence at least 99% identical to the amino acid sequence of SEQ ID NO: 142;
(yy) a β chain comprising an amino acid sequence at least 99% identical to the amino acid sequence of SEQ ID NO: 143;
(zz) an α chain comprising an amino acid sequence at least 99% identical to the amino acid sequence of SEQ ID NO: 148;
(aaa) a β chain comprising an amino acid sequence at least 99% identical to the amino acid sequence of SEQ ID NO: 149;
(bbb) both (dd) and (ee); both (ff) and (gg); both (hh) and (ii); or both (jj) and (kk);
(ccc) both (ll) and (mm); both (nn) and (oo); both (pp) and (qq); or both (rr) and (ss);
(ddd) both (tt) and (uu); both (vv) and (ww); both (xx) and (yy); or both (zz) and (aaa);
(eee) an α chain comprising an amino acid sequence at least 99% identical to the amino acid sequence of SEQ ID NO: 23;
(fff) a β chain comprising an amino acid sequence at least 99% identical to the amino acid sequence of SEQ ID NO: 24;
(ggg) an α chain comprising an amino acid sequence at least 99% identical to the amino acid sequence of SEQ ID NO: 102;
(hhh) a β chain comprising an amino acid sequence at least 99% identical to the amino acid sequence of SEQ ID NO: 103;
(iii) an α chain comprising an amino acid sequence at least 99% identical to the amino acid sequence of SEQ ID NO: 124;
(jjj) a β chain comprising an amino acid sequence at least 99% identical to the amino acid sequence of SEQ ID NO: 125;
(kkk) an α chain comprising an amino acid sequence at least 99% identical to the amino acid sequence of SEQ ID NO: 104;
(lll) a β chain comprising an amino acid sequence at least 99% identical to the amino acid sequence of SEQ ID NO: 105;
(mmm) an α chain comprising an amino acid sequence at least 99% identical to the amino acid sequence of SEQ ID NO: 39;
(nnn) a β chain comprising an amino acid sequence at least 99% identical to the amino acid sequence of SEQ ID NO: 40;
(ooo) an α chain comprising an amino acid sequence at least 99% identical to the amino acid sequence of SEQ ID NO: 108;
(ppp) a β chain comprising an amino acid sequence at least 99% identical to the amino acid sequence of SEQ ID NO: 109;
(qqq) an α chain comprising an amino acid sequence at least 99% identical to the amino acid sequence of SEQ ID NO: 126;
(rrr) a β chain comprising an amino acid sequence at least 99% identical to the amino acid sequence of SEQ ID NO: 127;
(sss) an α chain comprising an amino acid sequence at least 99% identical to the amino acid sequence of SEQ ID NO: 110;
(ttt) a β chain comprising an amino acid sequence at least 99% identical to the amino acid sequence of SEQ ID NO: 111;
(uuu) an α chain comprising an amino acid sequence at least 99% identical to the amino acid sequence of SEQ ID NO: 78;
(vvv) a β chain comprising an amino acid sequence at least 99% identical to the amino acid sequence of SEQ ID NO: 79;
(www) an α chain comprising an amino acid sequence at least 99% identical to the amino acid sequence of SEQ ID NO: 138;
(xxx) a β chain comprising an amino acid sequence at least 99% identical to the amino acid sequence of SEQ ID NO: 139;
(yyy) an α chain comprising an amino acid sequence at least 99% identical to the amino acid sequence of SEQ ID NO: 134;
(zzz) a β chain comprising an amino acid sequence at least 99% identical to the amino acid sequence of SEQ ID NO: 135;
(aaaa) an α chain comprising an amino acid sequence at least 99% identical to the amino acid sequence of SEQ ID NO: 140;
(bbbb) a β chain comprising an amino acid sequence at least 99% identical to the amino acid sequence of SEQ ID NO: 141;
(dddd) both (eee) and (fff); both (eee) and (hhh); both (fff) and (ggg); both (ggg) and (hhh); both (iii) and (jjj); both (iii) and (lll); both (jjj) and (kkk); or both (kkk) and (lll);
(eeee) both (mmm) and (nnn); both (mmm) and (ppp); both (nnn) and (ooo); both (ooo) and (ppp); both (qqq) and (rrr); both (qqq) and (ttt); both (rrr) and (sss); or both (sss) and (ttt); or
(ffff) both (uuu) and (vvv); both (uuu) and (xxx); both (vvv) and (www); both (www) and (xxx); both (yyy) and (zzz); both (yyy) and (bbbb); both (yyy) and (aaaa); or both (aaaa) and (bbbb).

12. The isolated or purified TCR according to claim 1, comprising:
(a) an α chain comprising the amino acid sequence of SEQ ID NO: 21, wherein:
(i) X at position 180 of SEQ ID NO: 21 is Thr or Cys;
(ii) X at position 244 of SEQ ID NO: 21 is Ser, Ala, Val, Leu, Ile, Pro, Phe, Met, or Trp;
(iii) X at position 246 of SEQ ID NO: 21 is Met, Ala, Val, Leu, Ile, Pro, Phe, or Trp; and
(iv) X at position 247 of SEQ ID NO: 21 is Gly, Ala, Val, Leu, Ile, Pro, Phe, Met, or Trp;
(b) a β chain comprising the amino acid sequence of SEQ ID NO: 22, wherein X at position 190 of SEQ ID NO: 22 is Ser or Cys;
(c) an α chain comprising the amino acid sequence of SEQ ID NO: 100, wherein:
(i) X at position 180 of SEQ ID NO: 100 is Thr or Cys;
(ii) X at position 244 of SEQ ID NO: 100 is Ser, Ala, Val, Leu, Ile, Pro, Phe,
(iii) X at position 246 of SEQ ID NO: 100 is Met, Ala, Val, Leu, Ile, Pro, Phe, or Trp; and
(iv) X at position 247 of SEQ ID NO: 100 is Gly, Ala, Val, Leu, Ile, Pro, Phe, Met, or Trp;
(d) a β chain comprising the amino acid sequence of SEQ ID NO: 101, wherein X at position 190 of SEQ ID NO: 101 is Ser or Cys;
(e) an α chain comprising the amino acid sequence of SEQ ID NO: 41, wherein:
(i) X at position 181 of SEQ ID NO: 41 is Thr or Cys;
(ii) X at position 245 of SEQ ID NO: 41 is Ser, Ala, Val, Leu, Ile, Pro, Phe, Met, or Trp;
(iii) X at position 247 of SEQ ID NO: 41 is Met, Ala, Val, Leu, Ile, Pro, Phe, or Trp; and
(iv) X at position 248 of SEQ ID NO: 41 is Gly, Ala, Val, Leu, Ile, Pro, Phe, Met, or Trp;
(f) a β chain comprising the amino acid sequence of SEQ ID NO: 42, wherein X at position 195 of SEQ ID NO: 42 is Ser or Cys;
(g) an α chain comprising the amino acid sequence of SEQ ID NO: 106, wherein:
(i) X at position 181 of SEQ ID NO: 106 is Thr or Cys;
(ii) X at position 245 of SEQ ID NO: 106 is Ser, Ala, Val, Leu, Ile, Pro, Phe, Met, or Trp;
(iii) X at position 247 of SEQ ID NO: 106 is Met, Ala, Val, Leu, Ile, Pro, Phe, or Trp; and
(iv) X at position 248 of SEQ ID NO: 106 is Gly, Ala, Val, Leu, Ile, Pro, Phe, Met, or Trp;
(h) a β chain comprising the amino acid sequence of SEQ ID NO: 107, wherein X at position 195 of SEQ ID NO: 107 is Ser or Cys;
(i) an α chain comprising the amino acid sequence of SEQ ID NO: 74, wherein:
(i) X at position 180 of SEQ ID NO: 74 is Thr or Cys;
(ii) X at position 244 of SEQ ID NO: 74 is Ser, Ala, Val, Leu, Ile, Pro, Phe, Met, or Trp;
(iii) X at position 246 of SEQ ID NO: 74 is Met, Ala, Val, Leu, Ile, Pro, Phe, or Trp; and
(iv) X at position 247 of SEQ ID NO: 74 is Gly, Ala, Val, Leu, Ile, Pro, Phe, Met, or Trp;
(j) a β chain comprising the amino acid sequence of SEQ ID NO: 75, wherein X at position 187 of SEQ ID NO: 75 is Ser or Cys;
(k) an α chain comprising the amino acid sequence of SEQ ID NO: 136, wherein:
(i) X at position 180 of SEQ ID NO: 136 is Thr or Cys;
(ii) X at position 244 of SEQ ID NO: 136 is Ser, Ala, Val, Leu, Ile, Pro, Phe, Met, or Trp;
(iii) X at position 246 of SEQ ID NO: 136 is Met, Ala, Val, Leu, Ile, Pro, Phe, or Trp; and
(iv) X at position 247 of SEQ ID NO: 136 is Gly, Ala, Val, Leu, Ile, Pro, Phe, Met, or Trp;
(l) a β chain comprising the amino acid sequence of SEQ ID NO: 137, wherein X at position 187 of SEQ ID NO: 75 is Ser or Cys;
(m) both (a) and (b); both (a) and (d); both (b) and (c); or both (c) and (d);
(n) both (e) and (o); both (e) and h); both (f) and (g); or both (g) and (h);
(n) both (i) and (j); both (i) and 1); both (j) and (k); or both (k) and (l);
(o) an α chain comprising the amino acid sequence of SEQ ID NO: 55, wherein:
(i) X at position 160 of SEQ ID NO: 55 is Thr or Cys;
(ii) X at position 224 of SEQ ID NO: 55 is Ser, Ala, Val, Leu, Ile, Pro, Phe, Met, or Trp;
(iii) X at position 226 of SEQ ID NO: 55 is Met, Ala, Val, Leu, Ile, Pro, Phe, or Trp; and
(iv) X at position 227 of SEQ ID NO: 55 is Gly, Ala, Val, Leu, Ile, Pro, Phe, Met, or Trp;
(p) a β chain comprising the amino acid sequence of SEQ ID NO: 56, wherein X at position 168 of SEQ ID NO: 56 is Ser or Cys;
(q) an α chain comprising the amino acid sequence of SEQ ID NO: 112, wherein:
(i) X at position 159 of SEQ ID NO: 112 is Thr or Cys;
(ii) X at position 223 of SEQ ID NO: 112 is Ser, Ala, Val, Leu, Ile, Pro, Phe,
(iii) X at position 225 of SEQ ID NO: 112 is Met, Ala, Val, Leu, Ile, Pro, Phe, or Trp; and
(iv) X at position 226 of SEQ ID NO: 112 is Gly, Ala, Val, Leu, Ile, Pro, Phe, Met, or Trp;
(r) a β chain comprising the amino acid sequence of SEQ ID NO: 113, wherein X at position 173 of SEQ ID NO: 113 is Ser or Cys;
(s) an α chain comprising the amino acid sequence of SEQ ID NO: 57, wherein:
(i) X at position 160 of SEQ ID NO: 57 is Thr or Cys;
(ii) X at position 224 of SEQ ID NO: 57 is Ser, Ala, Val, Leu, Ile, Pro, Phe, Met, or Trp;
(iii) X at position 226 of SEQ ID NO: 57 is Met, Ala, Val, Leu, Ile, Pro, Phe, or Trp; and
(iv) X at position 227 of SEQ ID NO: 57 is Gly, Ala, Val, Leu, Ile, Pro, Phe, Met, or Trp;
(t) a β chain comprising the amino acid sequence of SEQ ID NO: 58, wherein X at position 173 of SEQ ID NO: 58 is Ser or Cys;
(u) an α chain comprising the amino acid sequence of SEQ ID NO: 118, wherein:
(i) X at position 161 of SEQ ID NO: 118 is Thr or Cys;
(ii) X at position 225 of SEQ ID NO: 118 is Ser, Ala, Val, Leu, Ile, Pro, Phe, Met, or Trp;
(iii) X at position 227 of SEQ ID NO: 118 is Met, Ala, Val, Leu, Ile, Pro, Phe, or Trp; and
(iv) X at position 228 of SEQ ID NO: 118 is Gly, Ala, Val, Leu, Ile, Pro, Phe, Met, or Trp;
(v) a β chain comprising the amino acid sequence of SEQ ID NO: 119, wherein X at position 103178 of SEQ ID NO: 119 is Ser or Cys;
(w) an α chain comprising the amino acid sequence of SEQ ID NO: 76, wherein:
(i) X at position 159 of SEQ ID NO: 76 is Thr or Cys;
(ii) X at position 223 of SEQ ID NO: 76 is Ser, Ala, Val, Leu, Ile, Pro, Phe, Met, or Trp;
(iii) X at position 225 of SEQ ID NO: 76 is Met, Ala, Val, Leu, Ile, Pro, Phe, or Trp; and
(iv) X at position 226 of SEQ ID NO: 76 is Gly, Ala, Val, Leu, Ile, Pro, Phe, Met, or Trp;
(x) a β chain comprising the amino acid sequence of SEQ ID NO: 77, wherein X at position 168 of SEQ ID NO: 77 is Ser or Cys;
(y) an α chain comprising the amino acid sequence of SEQ ID NO: 144, wherein:
(i) X at position 160 of SEQ ID NO: 144 is Thr or Cys;
(ii) X at position 224 of SEQ ID NO: 144 is Ser, Ala, Val, Leu, Ile, Pro, Phe, Met, or Trp;
(iii) X at position 226 of SEQ ID NO: 144 is Met, Ala, Val, Leu, Ile, Pro, Phe, or Trp; and
(iv) X at position 227 of SEQ ID NO: 144 is Gly, Ala, Val, Leu, Ile, Pro, Phe, Met, or Trp;
(z) a β chain comprising the amino acid sequence of SEQ ID NO: 145, wherein X at position 166 of SEQ ID NO: 145 is Ser or Cys;
(aa) both (o) and (p); or both (q) and (r);
(bb) both (s) and (t); or both (u) and (v);
(cc) both (w) and (x); or both (y) and (z);
(dd) an α chain comprising the amino acid sequence of SEQ ID NO: 51;
(ee) a β chain comprising the amino acid sequence of SEQ ID NO: 52;
(ff) an α chain comprising the amino acid sequence of SEQ ID NO: 114;
(gg) a β chain comprising the amino acid sequence of SEQ ID NO: 115;
(hh) an α chain comprising the amino acid sequence of SEQ ID NO: 128;
(ii) a β chain comprising the amino acid sequence of SEQ ID NO: 129;
(jj) an α chain comprising the amino acid sequence of SEQ ID NO: 116;
(kk) a β chain comprising the amino acid sequence of SEQ ID NO: 117;
(ll) an α chain comprising the amino acid sequence of SEQ ID NO: 53;
(mm) a β chain comprising the amino acid sequence of SEQ ID NO: 54;
(nn) an α chain comprising the amino acid sequence of SEQ ID NO: 120;
(oo) a β chain comprising the amino acid sequence of SEQ ID NO: 121;
(pp) an α chain comprising the amino acid sequence of SEQ ID NO: 130;
(qq) a β chain comprising the amino acid sequence of SEQ ID NO: 131;
(rr) an α chain comprising the amino acid sequence of SEQ ID NO: 122;
(ss) a β chain comprising the amino acid sequence of SEQ ID NO: 123;
(tt) an α chain comprising the amino acid sequence of SEQ ID NO: 80;
(uu) a β chain comprising the amino acid sequence of SEQ ID NO: 81;
(vv) an α chain comprising the amino acid sequence of SEQ ID NO: 146;
(ww) a β chain comprising the amino acid sequence of SEQ ID NO: 146;
(xx) an α chain comprising the amino acid sequence of SEQ ID NO: 142;
(yy) a β chain comprising the amino acid sequence of SEQ ID NO: 143;
(zz) an α chain comprising the amino acid sequence of SEQ ID NO: 148;
(aaa) a β chain comprising the amino acid sequence of SEQ ID NO: 149;
(bbb) both (dd) and (ee); both (ff) and (gg); both (hh) and (ii); or both (jj) and (kk);
(ccc) both (ll) and (mm); both (nn) and (oo); both (pp) and (qq); or both (rr) and (ss);
(ddd) both (tt) and (uu); both (vv) and (ww); both (xx) and (yy); or both (zz) and (aaa);
(eee) an α chain comprising the amino acid sequence of SEQ ID NO: 23;
(fff) a β chain comprising the amino acid sequence of SEQ ID NO: 24;
(ggg) an α chain comprising the amino acid sequence of SEQ ID NO: 102;
(hhh) a β chain comprising the amino acid sequence of SEQ ID NO: 103;
(iii) an α chain comprising the amino acid sequence of SEQ ID NO: 124;
(jjj) a β chain comprising the amino acid sequence of SEQ ID NO: 125;
(kkk) an α chain comprising the amino acid sequence of SEQ ID NO: 104;
(lll) a β chain comprising the amino acid sequence of SEQ ID NO: 105;
(mmm) an α chain comprising the amino acid sequence of SEQ ID NO: 39;
(nnn) a β chain comprising the amino acid sequence of SEQ ID NO: 40;
(ooo) an α chain comprising the amino acid sequence of SEQ ID NO: 108;
(ppp) a β chain comprising the amino acid sequence of SEQ ID NO: 109;
(qqq) an α chain comprising the amino acid sequence of SEQ ID NO: 126;
(rrr) a β chain comprising the amino acid sequence of SEQ ID NO: 127;
(sss) an α chain comprising the amino acid sequence of SEQ ID NO: 110;
(ttt) a β chain comprising the amino acid sequence of SEQ ID NO: 111;
(uuu) an α chain comprising the amino acid sequence of SEQ ID NO: 78;
(vvv) a β chain comprising the amino acid sequence of SEQ ID NO: 79;
(www) an α chain comprising the amino acid sequence of SEQ ID NO: 138;
(xxx) a β chain comprising the amino acid sequence of SEQ ID NO: 139;
(yyy) an α chain comprising the amino acid sequence of SEQ ID NO: 134;
(zzz) a β chain comprising the amino acid sequence of SEQ ID NO: 135;
(aaaa) an α chain comprising the amino acid sequence of SEQ ID NO: 140;
(bbbb) a β chain comprising the amino acid sequence of SEQ ID NO: 141;
(dddd) both (eee) and (fff); both (eee) and (hhh); both (fff) and (ggg); both (ggg) and
(hhh); both (iii) and (jjj); both (iii) and (lll); both (jjj) and (kkk); or both (kkk) and (lll);
(eeee) both (mmm) and (nnn); both (mmm) and (ppp); both (nnn) and (ooo); both
(ooo) and (ppp); both (qqq) and (rrr); both (qqq) and (ttt); both (rrr) and (sss); or both (sss) and (ttt); or
(ffff) both (uuu) and (vvv); both (uuu) and (xxx); both (vvv) and (www); both (www) and (xxx); both (yyy) and (zzz); both (yyy) and (bbbb); both (yyy) and (aaaa); or both (aaaa) and (bbbb).

13. An isolated or purified polypeptide comprising a functional portion of the TCR according to claim 1, wherein the functional portion comprises the amino acid sequences of:
(a) all of SEQ ID NOs: 1-3,
(b) all of SEQ ID NOs: 4-6,
(c) all of SEQ ID NOs: 31-33,
(d) all of SEQ ID NOs: 34-36,
(e) all of SEQ ID NOs: 64-66,
(f) all of SEQ ID NOs: 67-69
(g) all of SEQ ID NOs: 1-6,
(h) all of SEQ ID NOs: 31-36, or
(i) all of SEQ ID NOs: 64-69.

14. The isolated or purified polypeptide according to claim 13, wherein the functional portion comprises:
(i) an amino acid sequence at least 99% identical to the amino acid sequence of SEQ ID NO: 7;
(ii) an amino acid sequence at least 99% identical to the amino acid sequence of SEQ ID NO: 8;
(iii) an amino acid sequence at least 99% identical to the amino acid sequence of SEQ ID NO: 90;
(iv) an amino acid sequence at least 99% identical to the amino acid sequence of SEQ ID NO: 91;
(v) an amino acid sequence at least 99% identical to the amino acid sequence of SEQ ID NO: 37;
(vi) an amino acid sequence at least 99% identical to the amino acid sequence of SEQ ID NO: 38;
(vii) an amino acid sequence at least 99% identical to the amino acid sequence of SEQ ID NO: 92;
(viii) an amino acid sequence at least 99% identical to the amino acid sequence of SEQ ID NO: 93;
(ix) an amino acid sequence at least 99% identical to the amino acid sequence of SEQ ID NO: 70;
(x) an amino acid sequence at least 99% identical to the amino acid sequence of SEQ ID NO: 71;
(xi) an amino acid sequence at least 99% identical to the amino acid sequence of SEQ ID NO: 132;
(xii) an amino acid sequence at least 99% identical to the amino acid sequence of SEQ ID NO: 133;
(xiii) an amino acid sequence at least 99% identical to the amino acid sequence of SEQ ID NO: 47;
(xiv) an amino acid sequence at least 99% identical to the amino acid sequence of SEQ ID NO: 48;
(xv) an amino acid sequence at least 99% identical to the amino acid sequence of SEQ ID NO: 94;
(xvi) an amino acid sequence at least 99% identical to the amino acid sequence of SEQ ID NO: 85;
(xvii) an amino acid sequence at least 99% identical to the amino acid sequence of SEQ ID NO: 95;
(xviii) an amino acid sequence at least 99% identical to the amino acid sequence of SEQ ID NO: 49;
(xix) an amino acid sequence at least 99% identical to the amino acid sequence of SEQ ID NO: 50;
(xx) an amino acid sequence at least 99% identical to the amino acid sequence of SEQ ID NO: 96;
(xxi) an amino acid sequence at least 99% identical to the amino acid sequence of SEQ ID NO: 87;
(xxii) an amino acid sequence at least 99% identical to the amino acid sequence of SEQ ID NO: 97;
(xxiii) an amino acid sequence at least 99% identical to the amino acid sequence of SEQ ID NO: 72;
(xxvi) an amino acid sequence at least 99% identical to the amino acid sequence of SEQ ID NO: 73;
(xxv) an amino acid sequence at least 99% identical to the amino acid sequence of SEQ ID NO: 88;
(xxvi) an amino acid sequence at least 99% identical to the amino acid sequence of SEQ ID NO: 89;
(xxvii) both (i) and (ii), both (i) and (iv), both (ii) and (iii), both (iii) and (iv), both (v) and (vi), both (v) and (viii), both (vi) and (vii), both (vii) and (viii), both (ix) and (x), both (ix) and (xii), both (x) and (xi), both (xi) and (xii), both (xiii) and (xiv), both (xiii) and (xvi), both (xiii) and (xvii), both (xiv) and (xv), both (xv) and (xvi), both (xv) and (xvii), both (xviii) and (xix), both (xviii) and (xxi), both (xviii) and (xxii), both (xix) and (xx), both (xx) and (xxi), both (xx) and (xxii), both (xxiii) and (xxiv), both (xxiii) and (xxvi), both (xxiv) and (xxv), or both (xxv) and (xxvi).

15. The isolated or purified polypeptide according to claim 1, wherein the functional portion comprises:
(i) the amino acid sequence of SEQ ID NO: 7;
(ii) the amino acid sequence of SEQ ID NO: 8;
(iii) the amino acid sequence of SEQ ID NO: 90;
(iv) the amino acid sequence of SEQ ID NO: 91;
(v) the amino acid sequence of SEQ ID NO: 37;
(vi) the amino acid sequence of SEQ ID NO: 38;
(vii) the amino acid sequence of SEQ ID NO: 92;
(viii) the amino acid sequence of SEQ ID NO: 93;
(ix) the amino acid sequence of SEQ ID NO: 70;
(x) the amino acid sequence of SEQ ID NO: 71;
(xi) the amino acid sequence of SEQ ID NO: 132;
(xii) the amino acid sequence of SEQ ID NO: 133;
(xiii) the amino acid sequence of SEQ ID NO: 47;
(xiv) the amino acid sequence of SEQ ID NO: 48;
(xv) the amino acid sequence of SEQ ID NO: 94;
(xvi) the amino acid sequence of SEQ ID NO: 85;
(xvii) the amino acid sequence of SEQ ID NO: 95;
(xviii) the amino acid sequence of SEQ ID NO: 49;
(xix) the amino acid sequence of SEQ ID NO: 50;
(xx) the amino acid sequence of SEQ ID NO: 96;
(xxi) the amino acid sequence of SEQ ID NO: 87;
(xxii) the amino acid sequence of SEQ ID NO: 97;
(xxiii) the amino acid sequence of SEQ ID NO: 72;
(xxvi) the amino acid sequence of SEQ ID NO: 73;
(xxv) the amino acid sequence of SEQ ID NO: 88;
(xxvi) the amino acid sequence of SEQ ID NO: 89;
(xxvii) both (i) and (ii), both (i) and (iv), both (ii) and (iii), both (iii) and (iv), both (v) and (vi), both (v) and (viii), both (vi) and (vii), both (vii) and (viii), both (ix) and (x), both (ix) and (xii), both (x) and (xi), both (xi) and (xii), both (xiii) and (xiv), both (xiii) and (xvi), both (xiii) and (xvii), both (xiv) and (xv), both (xv) and (xvi), both (xv) and (xvii), both (xviii) and (xix), both (xviii) and (xxi), both (xviii) and (xxii), both (xix) and (xx), both (xx) and (xxi), both (xx) and (xxii), both (xxiii) and (xxiv), both (xxiii) and (xxvi), both (xxiv) and (xxv), or both (xxv) and (xxvi).

16. The isolated or purified polypeptide according to claim 1, further comprising:
(a) an amino acid sequence at least 99% identical to the amino acid sequence of SEQ ID NO: 17, wherein:
(i) X at position 48 of SEQ ID NO: 17 is Thr or Cys;
(ii) X at position 112 of SEQ ID NO: 17 is Ser, Ala, Val, Leu, Ile, Pro, Phe, Met, or Trp;
(iii) X at position 114 of SEQ ID NO: 17 is Met, Ala, Val, Leu, Ile, Pro, Phe, or Trp; and
(iv) X at position 115 of SEQ ID NO: 17 is Gly, Ala, Val, Leu, Ile, Pro, Phe, Met, or Trp;
(b) an amino acid sequence at least 99% identical to the amino acid sequence of SEQ ID NO: 18, wherein X at position 57 of SEQ ID NO: 18 is Ser or Cys; or
(c) both (a) and (b).

17. The isolated or purified polypeptide according to claim 1, further comprising:
(a) the amino acid sequence of SEQ ID NO: 17, wherein:
(i) X at position 48 of SEQ ID NO: 17 is Thr or Cys;
(ii) X at position 112 of SEQ ID NO: 17 is Ser, Ala, Val, Leu, Ile, Pro, Phe, Met, or Trp;
(iii) X at position 114 of SEQ ID NO: 17 is Met, Ala, Val, Leu, Ile, Pro, Phe, or Trp; and
(iv) X at position 115 of SEQ ID NO: 17 is Gly, Ala, Val, Leu, Ile, Pro, Phe, Met, or Trp;
(b) the amino acid sequence of SEQ ID NO: 18, wherein X at position 57 of SEQ ID NO: 18 is Ser or Cys; or (c) both (a) and (b).

18. The isolated or purified polypeptide according to claim 1, comprising:
(a) an α chain comprising an amino acid sequence at least 99% identical to the amino acid sequence of SEQ ID NO: 21, wherein:
(i) X at position 180 of SEQ ID NO: 21 is Thr or Cys;
(ii) X at position 244 of SEQ ID NO: 21 is Ser, Ala, Val, Leu, Ile, Pro, Phe, Met, or Trp;
(iii) X at position 246 of SEQ ID NO: 21 is Met, Ala, Val, Leu, Ile, Pro, Phe, or Trp; and
(iv) X at position 247 of SEQ ID NO: 21 is Gly, Ala, Val, Leu, Ile, Pro, Phe, Met, or Trp;
(b) a β chain comprising an amino acid sequence at least 99% identical to the amino acid sequence of SEQ ID NO: 22, wherein X at position 190 of SEQ ID NO: 22 is Ser or Cys;
(c) an α chain comprising an amino acid sequence at least 99% identical to the amino acid sequence of SEQ ID NO: 100, wherein:
(i) X at position 180 of SEQ ID NO: 100 is Thr or Cys;
(ii) X at position 244 of SEQ ID NO: 100 is Ser, Ala, Val, Leu, Ile, Pro, Phe, Met, or Trp;
(iii) X at position 246 of SEQ ID NO: 100 is Met, Ala, Val, Leu, Ile, Pro, Phe, or Trp; and
(iv) X at position 247 of SEQ ID NO: 100 is Gly, Ala, Val, Leu, Ile, Pro, Phe, Met, or Trp;
(d) a β chain comprising an amino acid sequence at least 99% identical to the amino acid sequence of SEQ ID NO: 101, wherein X at position 190 of SEQ ID NO: 101 is Ser or Cys;
(e) an α chain comprising an amino acid sequence at least 99% identical to the amino acid sequence of SEQ ID NO: 41, wherein:
(i) X at position 181 of SEQ ID NO: 41 is Thr or Cys;
(ii) X at position 245 of SEQ ID NO: 41 is Ser, Ala, Val, Leu, Ile, Pro, Phe, Met, or Trp;
(iii) X at position 247 of SEQ ID NO: 41 is Met, Ala, Val, Leu, Ile, Pro, Phe, or Trp; and
(iv) X at position 248 of SEQ ID NO: 41 is Gly, Ala, Val, Leu, Ile, Pro, Phe,
(f) a β chain comprising an amino acid sequence at least 99% identical to the amino acid sequence of SEQ ID NO: 42, wherein X at position 195 of SEQ ID NO: 42 is Ser or Cys;
(g) an α chain comprising an amino acid sequence at least 99% identical to the amino acid sequence of SEQ ID NO: 106, wherein:
(i) X at position 181 of SEQ ID NO: 106 is Thr or Cys;
(ii) X at position 245 of SEQ ID NO: 106 is Ser, Ala, Val, Leu, Ile, Pro, Phe, Met, or Trp;
(iii) X at position 247 of SEQ ID NO: 106 is Met, Ala, Val, Leu, Ile, Pro, Phe, or Trp; and
(iv) X at position 248 of SEQ ID NO: 106 is Gly, Ala, Val, Leu, Ile, Pro, Phe, Met, or Trp;
(h) a β chain comprising an amino acid sequence at least 99% identical to the amino acid sequence of SEQ ID NO: 107, wherein X at position 195 of SEQ ID NO: 107 is Ser or Cys;
(i) an α chain comprising an amino acid sequence at least 99% identical to the amino acid sequence of SEQ ID NO: 74, wherein:
(i) X at position 180 of SEQ ID NO: 74 is Thr or Cys;
(ii) X at position 244 of SEQ ID NO: 74 is Ser, Ala, Val, Leu, Ile, Pro, Phe, Met, or Trp;
(iii) X at position 246 of SEQ ID NO: 74 is Met, Ala, Val, Leu, Ile, Pro, Phe, or Trp; and
(iv) X at position 247 of SEQ ID NO: 74 is Gly, Ala, Val, Leu, Ile, Pro, Phe, Met, or Trp;
(j) a β chain comprising an amino acid sequence at least 99% identical to the amino acid sequence of SEQ ID NO: 75, wherein X at position 187 of SEQ ID NO: 75 is Ser or Cys;
(k) an α chain comprising an amino acid sequence at least 99% identical to the amino acid sequence of SEQ ID NO: 136, wherein:
(i) X at position 180 of SEQ ID NO: 136 is Thr or Cys;
(ii) X at position 244 of SEQ ID NO: 136 is Ser, Ala, Val, Leu, Ile, Pro, Phe,
(iii) X at position 246 of SEQ ID NO: 136 is Met, Ala, Val, Leu, Ile, Pro, Phe, or Trp; and
(iv) X at position 247 of SEQ ID NO: 136 is Gly, Ala, Val, Leu, Ile, Pro, Phe, Met, or Trp;
(l) a β chain comprising an amino acid sequence at least 99% identical to the amino acid sequence of SEQ ID NO: 137, wherein X at position 187 of SEQ ID NO: 75 is Ser or Cys;
(m) both (a) and (b); both (a) and (d); both (b) and (c); or both (c) and (d);
(n) both (e) and (o); both (e) and h); both (f) and (g); or both (g) and (h);
(n) both (i) and (j); both (i) and 1); both (j) and (k); or both (k) and (l);
(o) an α chain comprising an amino acid sequence at least 99% identical to the amino acid sequence of SEQ ID NO: 55, wherein:
(i) X at position 160 of SEQ ID NO: 55 is Thr or Cys;
(ii) X at position 224 of SEQ ID NO: 55 is Ser, Ala, Val, Leu, Ile, Pro, Phe, Met, or Trp;
(iii) X at position 226 of SEQ ID NO: 55 is Met, Ala, Val, Leu, Ile, Pro, Phe, or Trp; and
(iv) X at position 227 of SEQ ID NO: 55 is Gly, Ala, Val, Leu, Ile, Pro, Phe, Met, or Trp;
(p) a β chain comprising an amino acid sequence at least 99% identical to the amino acid sequence of SEQ ID NO: 56, wherein X at position 168 of SEQ ID NO: 56 is Ser or Cys;
(q) an α chain comprising an amino acid sequence at least 99% identical to the amino acid sequence of SEQ ID NO: 112, wherein:
(i) X at position 159 of SEQ ID NO: 112 is Thr or Cys;
(ii) X at position 223 of SEQ ID NO: 112 is Ser, Ala, Val, Leu, Ile, Pro, Phe, Met, or Trp;
(iii) X at position 225 of SEQ ID NO: 112 is Met, Ala, Val, Leu, Ile, Pro, Phe, or Trp; and
(iv) X at position 226 of SEQ ID NO: 112 is Gly, Ala, Val, Leu, Ile, Pro, Phe,
(r) a β chain comprising an amino acid sequence at least 99% identical to the amino acid sequence of SEQ ID NO: 113, wherein X at position 173 of SEQ ID NO: 113 is Ser or Cys;
(s) an α chain comprising an amino acid sequence at least 99% identical to the amino acid sequence of SEQ ID NO: 57, wherein:
(i) X at position 160 of SEQ ID NO: 57 is Thr or Cys;
(ii) X at position 224 of SEQ ID NO: 57 is Ser, Ala, Val, Leu, Ile, Pro, Phe, Met, or Trp;
(iii) X at position 226 of SEQ ID NO: 57 is Met, Ala, Val, Leu, Ile, Pro, Phe, or Trp; and
(iv) X at position 227 of SEQ ID NO: 57 is Gly, Ala, Val, Leu, Ile, Pro, Phe, Met, or Trp;
(t) a β chain comprising an amino acid sequence at least 99% identical to the amino acid sequence of SEQ ID NO: 58, wherein X at position 173 of SEQ ID NO: 58 is Ser or Cys;
(u) an α chain comprising an amino acid sequence at least 99% identical to the amino acid sequence of SEQ ID NO: 118, wherein:
(i) X at position 161 of SEQ ID NO: 118 is Thr or Cys;
(ii) X at position 225 of SEQ ID NO: 118 is Ser, Ala, Val, Leu, Ile, Pro, Phe, Met, or Trp;
(iii) X at position 227 of SEQ ID NO: 118 is Met, Ala, Val, Leu, Ile, Pro, Phe, or Trp; and
(iv) X at position 228 of SEQ ID NO: 118 is Gly, Ala, Val, Leu, Ile, Pro, Phe, Met, or Trp;
(v) a β chain comprising an amino acid sequence at least 99% identical to the amino acid sequence of SEQ ID NO: 119, wherein X at position 103178 of SEQ ID NO: 119 is Ser or Cys;
(w) an α chain comprising an amino acid sequence at least 99% identical to the amino acid sequence of SEQ ID NO: 76, wherein:
(i) X at position 159 of SEQ ID NO: 76 is Thr or Cys;
(ii) X at position 223 of SEQ ID NO: 76 is Ser, Ala, Val, Leu, Ile, Pro, Phe,
(iii) X at position 225 of SEQ ID NO: 76 is Met, Ala, Val, Leu, Ile, Pro, Phe, or Trp; and
(iv) X at position 226 of SEQ ID NO: 76 is Gly, Ala, Val, Leu, Ile, Pro, Phe, Met, or Trp;
(x) a β chain comprising an amino acid sequence at least 99% identical to the amino acid sequence of SEQ ID NO: 77, wherein X at position 168 of SEQ ID NO: 77 is Ser or Cys;
(y) an α chain comprising an amino acid sequence at least 99% identical to the amino acid sequence of SEQ ID NO: 144, wherein:
(i) X at position 160 of SEQ ID NO: 144 is Thr or Cys;
(ii) X at position 224 of SEQ ID NO: 144 is Ser, Ala, Val, Leu, Ile, Pro, Phe, Met, or Trp;
(iii) X at position 226 of SEQ ID NO: 144 is Met, Ala, Val, Leu, Ile, Pro, Phe, or Trp; and
(iv) X at position 227 of SEQ ID NO: 144 is Gly, Ala, Val, Leu, Ile, Pro, Phe, Met, or Trp;
(z) a β chain comprising an amino acid sequence at least 99% identical to the amino acid sequence of SEQ ID NO: 145, wherein X at position 166 of SEQ ID NO: 145 is Ser or Cys;
(aa) both (o) and (p); or both (q) and (r);
(bb) both (s) and (t); or both (u) and (v);
(cc) both (w) and (x); or both (y) and (z);
(dd) an α chain comprising an amino acid sequence at least 99% identical to the amino acid sequence of SEQ ID NO: 51;
(ee) a β chain comprising an amino acid sequence at least 99% identical to the amino acid sequence of SEQ ID NO: 52;
(ff) an α chain comprising an amino acid sequence at least 99% identical to the amino acid sequence of SEQ ID NO: 114;
(gg) a β chain comprising an amino acid sequence at least 99% identical to the amino acid sequence of SEQ ID NO: 115;
(hh) an α chain comprising an amino acid sequence at least 99% identical to the amino acid sequence of SEQ ID NO: 128;
(ii) a β chain comprising an amino acid sequence at least 99% identical to the amino acid sequence of SEQ ID NO: 129;
(jj) an α chain comprising an amino acid sequence at least 99% identical to the amino acid sequence of SEQ ID NO: 116;
(kk) a β chain comprising an amino acid sequence at least 99% identical to the amino acid sequence of SEQ ID NO: 117;
(ll) an α chain comprising an amino acid sequence at least 99% identical to the amino acid sequence of SEQ ID NO: 53;
(mm) a β chain comprising an amino acid sequence at least 99% identical to the amino acid sequence of SEQ ID NO: 54;
(nn) an α chain comprising an amino acid sequence at least 99% identical to the amino acid sequence of SEQ ID NO: 120;
(oo) a β chain comprising an amino acid sequence at least 99% identical to the amino acid sequence of SEQ ID NO: 121;
(pp) an α chain comprising an amino acid sequence at least 99% identical to the amino acid sequence of SEQ ID NO: 130;
(qq) a β chain comprising an amino acid sequence at least 99% identical to the amino acid sequence of SEQ ID NO: 131;
(rr) an α chain comprising an amino acid sequence at least 99% identical to the amino acid sequence of SEQ ID NO: 122;
(ss) a β chain comprising an amino acid sequence at least 99% identical to the amino acid sequence of SEQ ID NO: 123;
(tt) an α chain comprising an amino acid sequence at least 99% identical to the amino acid sequence of SEQ ID NO: 80;
(uu) a β chain comprising an amino acid sequence at least 99% identical to the amino acid sequence of SEQ ID NO: 81;
(vv) an α chain comprising an amino acid sequence at least 99% identical to the amino acid sequence of SEQ ID NO: 146;
(ww) a β chain comprising an amino acid sequence at least 99% identical to the amino acid sequence of SEQ ID NO: 146;
(xx) an α chain comprising an amino acid sequence at least 99% identical to the amino acid sequence of SEQ ID NO: 142;
(yy) a β chain comprising an amino acid sequence at least 99% identical to the amino acid sequence of SEQ ID NO: 143;
(zz) an α chain comprising an amino acid sequence at least 99% identical to the amino acid sequence of SEQ ID NO: 148;
(aaa) a β chain comprising an amino acid sequence at least 99% identical to the amino acid sequence of SEQ ID NO: 149;
(bbb) both (dd) and (ee); both (ff) and (gg); both (hh) and (ii); or both (jj) and (kk);
(ccc) both (ll) and (mm); both (nn) and (oo); both (pp) and (qq); or both (rr) and (ss);
(ddd) both (tt) and (uu); both (vv) and (ww); both (xx) and (yy); or both (zz) and (aaa);
(eee) an α chain comprising an amino acid sequence at least 99% identical to the amino acid sequence of SEQ ID NO: 23;
(fff) a β chain comprising an amino acid sequence at least 99% identical to the amino acid sequence of SEQ ID NO: 24;
(ggg) an α chain comprising an amino acid sequence at least 99% identical to the amino acid sequence of SEQ ID NO: 102;
(hhh) a β chain comprising an amino acid sequence at least 99% identical to the amino acid sequence of SEQ ID NO: 103;
(iii) an α chain comprising an amino acid sequence at least 99% identical to the amino acid sequence of SEQ ID NO: 124;
(jjj) a β chain comprising an amino acid sequence at least 99% identical to the amino acid sequence of SEQ ID NO: 125;
(kkk) an α chain comprising an amino acid sequence at least 99% identical to the amino acid sequence of SEQ ID NO: 104;
(lll) a β chain comprising an amino acid sequence at least 99% identical to the amino acid sequence of SEQ ID NO: 105;
(mmm) an α chain comprising an amino acid sequence at least 99% identical to the amino acid sequence of SEQ ID NO: 39;
(nnn) a β chain comprising an amino acid sequence at least 99% identical to the amino acid sequence of SEQ ID NO: 40;
(ooo) an α chain comprising an amino acid sequence at least 99% identical to the amino acid sequence of SEQ ID NO: 108;
(ppp) a β chain comprising an amino acid sequence at least 99% identical to the amino acid sequence of SEQ ID NO: 109;
(qqq) an α chain comprising an amino acid sequence at least 99% identical to the amino acid sequence of SEQ ID NO: 126;
(rrr) a β chain comprising an amino acid sequence at least 99% identical to the amino acid sequence of SEQ ID NO: 127;
(sss) an α chain comprising an amino acid sequence at least 99% identical to the amino acid sequence of SEQ ID NO: 110;
(ttt) a β chain comprising an amino acid sequence at least 99% identical to the amino acid sequence of SEQ ID NO: 111;
(uuu) an α chain comprising an amino acid sequence at least 99% identical to the amino acid sequence of SEQ ID NO: 78;
(vvv) a β chain comprising an amino acid sequence at least 99% identical to the amino acid sequence of SEQ ID NO: 79;
(www) an α chain comprising an amino acid sequence at least 99% identical to the amino acid sequence of SEQ ID NO: 138;
(xxx) a β chain comprising an amino acid sequence at least 99% identical to the amino acid sequence of SEQ ID NO: 139;
(yyy) an α chain comprising an amino acid sequence at least 99% identical to the amino acid sequence of SEQ ID NO: 134;
(zzz) a β chain comprising an amino acid sequence at least 99% identical to the amino acid sequence of SEQ ID NO: 135;
(aaaa) an α chain comprising an amino acid sequence at least 99% identical to the amino acid sequence of SEQ ID NO: 140;
(bbbb) a β chain comprising an amino acid sequence at least 99% identical to the amino acid sequence of SEQ ID NO: 141;
(dddd) both (eee) and (fff); both (eee) and (hhh); both (fff) and (ggg); both (ggg) and (hhh); both (iii) and (jjj); both (iii) and (lll); both (jjj) and (kkk); or both (kkk) and (lll);
(eeee) both (mmm) and (nnn); both (mmm) and (ppp); both (nnn) and (ooo); both (ooo) and (ppp); both (qqq) and (rrr); both (qqq) and (ttt); both (rrr) and (sss); or both (sss) and (ttt); or (ffff) both (uuu) and (vvv); both (uuu) and (xxx); both (vvv) and (www); both (www) and (xxx); both (yyy) and (zzz); both (yyy) and (bbbb); both (yyy) and (aaaa); or both (aaaa) and (bbbb).

19. The isolated or purified polypeptide according to claim 13, comprising:
(a) an α chain comprising the amino acid sequence of SEQ ID NO: 21, wherein:
(i) X at position 180 of SEQ ID NO: 21 is Thr or Cys;
(ii) X at position 244 of SEQ ID NO: 21 is Ser, Ala, Val, Leu, Ile, Pro, Phe, Met, or Trp;
(iii) X at position 246 of SEQ ID NO: 21 is Met, Ala, Val, Leu, Ile, Pro, Phe, or Trp; and
(iv) X at position 247 of SEQ ID NO: 21 is Gly, Ala, Val, Leu, Ile, Pro, Phe, Met, or Trp;
(b) a β chain comprising the amino acid sequence of SEQ ID NO: 22, wherein X at position 190 of SEQ ID NO: 22 is Ser or Cys;
(c) an α chain comprising the amino acid sequence of SEQ ID NO: 100, wherein:
(i) X at position 180 of SEQ ID NO: 100 is Thr or Cys;
(ii) X at position 244 of SEQ ID NO: 100 is Ser, Ala, Val, Leu, Ile, Pro, Phe, Met, or Trp;
(iii) X at position 246 of SEQ ID NO: 100 is Met, Ala, Val, Leu, Ile, Pro, Phe, or Trp; and
(iv) X at position 247 of SEQ ID NO: 100 is Gly, Ala, Val, Leu, Ile, Pro, Phe, Met, or Trp;
(d) a β chain comprising the amino acid sequence of SEQ ID NO: 101, wherein X at position 190 of SEQ ID NO: 101 is Ser or Cys;
(e) an α chain comprising the amino acid sequence of SEQ ID NO: 41, wherein:
(i) X at position 181 of SEQ ID NO: 41 is Thr or Cys;
(ii) X at position 245 of SEQ ID NO: 41 is Ser, Ala, Val, Leu, Ile, Pro, Phe, Met, or Trp;
(iii) X at position 247 of SEQ ID NO: 41 is Met, Ala, Val, Leu, Ile, Pro, Phe, or Trp; and
(iv) X at position 248 of SEQ ID NO: 41 is Gly, Ala, Val, Leu, Ile, Pro, Phe, Met, or Trp;
(f) a β chain comprising the amino acid sequence of SEQ ID NO: 42, wherein X at position 195 of SEQ ID NO: 42 is Ser or Cys;
(g) an α chain comprising the amino acid sequence of SEQ ID NO: 106, wherein:
(i) X at position 181 of SEQ ID NO: 106 is Thr or Cys;
(ii) X at position 245 of SEQ ID NO: 106 is Ser, Ala, Val, Leu, Ile, Pro, Phe, Met, or Trp;
(iii) X at position 247 of SEQ ID NO: 106 is Met, Ala, Val, Leu, Ile, Pro, Phe, or Trp; and
(iv) X at position 248 of SEQ ID NO: 106 is Gly, Ala, Val, Leu, Ile, Pro, Phe, Met, or Trp;
(h) a β chain comprising the amino acid sequence of SEQ ID NO: 107, wherein X at position 195 of SEQ ID NO: 107 is Ser or Cys;
(i) an α chain comprising the amino acid sequence of SEQ ID NO: 74, wherein:
(i) X at position 180 of SEQ ID NO: 74 is Thr or Cys;
(ii) X at position 244 of SEQ ID NO: 74 is Ser, Ala, Val, Leu, Ile, Pro, Phe, Met, or Trp;
(iii) X at position 246 of SEQ ID NO: 74 is Met, Ala, Val, Leu, Ile, Pro, Phe, or Trp; and
(iv) X at position 247 of SEQ ID NO: 74 is Gly, Ala, Val, Leu, Ile, Pro, Phe, Met, or Trp;
(j) a β chain comprising the amino acid sequence of SEQ ID NO: 75, wherein X at position 187 of SEQ ID NO: 75 is Ser or Cys;
(k) an α chain comprising the amino acid sequence of SEQ ID NO: 136, wherein:
(i) X at position 180 of SEQ ID NO: 136 is Thr or Cys;
(ii) X at position 244 of SEQ ID NO: 136 is Ser, Ala, Val, Leu, Ile, Pro, Phe, Met, or Trp;
(iii) X at position 246 of SEQ ID NO: 136 is Met, Ala, Val, Leu, Ile, Pro, Phe, or Trp; and
(iv) X at position 247 of SEQ ID NO: 136 is Gly, Ala, Val, Leu, Ile, Pro, Phe,
(l) a β chain comprising the amino acid sequence of SEQ ID NO: 137, wherein X at position 187 of SEQ ID NO: 75 is Ser or Cys;
(m) both (a) and (b); both (a) and (d); both (b) and (c); or both (c) and (d);
(n) both (e) and (o); both (e) and h); both (f) and (g); or both (g) and (h);
(n) both (i) and (j); both (i) and (l); both (j) and (k); or both (k) and (l);
(o) an α chain comprising the amino acid sequence of SEQ ID NO: 55, wherein:
(i) X at position 160 of SEQ ID NO: 55 is Thr or Cys;
(ii) X at position 224 of SEQ ID NO: 55 is Ser, Ala, Val, Leu, Ile, Pro, Phe, Met, or Trp;
(iii) X at position 226 of SEQ ID NO: 55 is Met, Ala, Val, Leu, Ile, Pro, Phe, or Trp; and
(iv) X at position 227 of SEQ ID NO: 55 is Gly, Ala, Val, Leu, Ile, Pro, Phe, Met, or Trp;
(p) a β chain comprising the amino acid sequence of SEQ ID NO: 56, wherein X at position 168 of SEQ ID NO: 56 is Ser or Cys;
(q) an α chain comprising the amino acid sequence of SEQ ID NO: 112, wherein:
(i) X at position 159 of SEQ ID NO: 112 is Thr or Cys;
(ii) X at position 223 of SEQ ID NO: 112 is Ser, Ala, Val, Leu, Ile, Pro, Phe, Met, or Trp;
(iii) X at position 225 of SEQ ID NO: 112 is Met, Ala, Val, Leu, Ile, Pro, Phe, or Trp; and
(iv) X at position 226 of SEQ ID NO: 112 is Gly, Ala, Val, Leu, Ile, Pro, Phe, Met, or Trp;
(r) a β chain comprising the amino acid sequence of SEQ ID NO: 113, wherein X at position 173 of SEQ ID NO: 113 is Ser or Cys;
(s) an α chain comprising the amino acid sequence of SEQ ID NO: 57, wherein:
(i) X at position 160 of SEQ ID NO: 57 is Thr or Cys;
(ii) X at position 224 of SEQ ID NO: 57 is Ser, Ala, Val, Leu, Ile, Pro, Phe, Met, or Trp;
(iii) X at position 226 of SEQ ID NO: 57 is Met, Ala, Val, Leu, Ile, Pro, Phe, or Trp; and
(iv) X at position 227 of SEQ ID NO: 57 is Gly, Ala, Val, Leu, Ile, Pro, Phe, Met, or Trp;
(t) a β chain comprising the amino acid sequence of SEQ ID NO: 58, wherein X at position 173 of SEQ ID NO: 58 is Ser or Cys;
(u) an α chain comprising the amino acid sequence of SEQ ID NO: 118, wherein:
(i) X at position 161 of SEQ ID NO: 118 is Thr or Cys;
(ii) X at position 225 of SEQ ID NO: 118 is Ser, Ala, Val, Leu, Ile, Pro, Phe, Met, or Trp;
(iii) X at position 227 of SEQ ID NO: 118 is Met, Ala, Val, Leu, Ile, Pro, Phe, or Trp; and
(iv) X at position 228 of SEQ ID NO: 118 is Gly, Ala, Val, Leu, Ile, Pro, Phe, Met, or Trp;
(v) a β chain comprising the amino acid sequence of SEQ ID NO: 119, wherein X at position 103178 of SEQ ID NO: 119 is Ser or Cys;
(w) an α chain comprising the amino acid sequence of SEQ ID NO: 76, wherein:
(i) X at position 159 of SEQ ID NO: 76 is Thr or Cys;
(ii) X at position 223 of SEQ ID NO: 76 is Ser, Ala, Val, Leu, Ile, Pro, Phe, Met, or Trp;
(iii) X at position 225 of SEQ ID NO: 76 is Met, Ala, Val, Leu, Ile, Pro, Phe, or Trp; and
(iv) X at position 226 of SEQ ID NO: 76 is Gly, Ala, Val, Leu, Ile, Pro, Phe, Met, or Trp;
(x) a β chain comprising the amino acid sequence of SEQ ID NO: 77, wherein X at position 168 of SEQ ID NO: 77 is Ser or Cys;
(y) an α chain comprising the amino acid sequence of SEQ ID NO: 144, wherein:
(i) X at position 160 of SEQ ID NO: 144 is Thr or Cys;
(ii) X at position 224 of SEQ ID NO: 144 is Ser, Ala, Val, Leu, Ile, Pro, Phe, Met, or Trp;
(iii) X at position 226 of SEQ ID NO: 144 is Met, Ala, Val, Leu, Ile, Pro, Phe, or Trp; and
(iv) X at position 227 of SEQ ID NO: 144 is Gly, Ala, Val, Leu, Ile, Pro, Phe,
(z) a β chain comprising the amino acid sequence of SEQ ID NO: 145, wherein X at position 166 of SEQ ID NO: 145 is Ser or Cys;
(aa) both (o) and (p); or both (q) and (r);
(bb) both (s) and (t); or both (u) and (v);
(cc) both (w) and (x); or both (y) and (z);
(dd) an α chain comprising the amino acid sequence of SEQ ID NO: 51;
(ee) a β chain comprising the amino acid sequence of SEQ ID NO: 52;
(ff) an α chain comprising the amino acid sequence of SEQ ID NO: 114;
(gg) a β chain comprising the amino acid sequence of SEQ ID NO: 115;
(hh) an α chain comprising the amino acid sequence of SEQ ID NO: 128;
(ii) a β chain comprising the amino acid sequence of SEQ ID NO: 129;
(jj) an α chain comprising the amino acid sequence of SEQ ID NO: 116;
(kk) a β chain comprising the amino acid sequence of SEQ ID NO: 117;
(ll) an α chain comprising the amino acid sequence of SEQ ID NO: 53;
(mm) a β chain comprising the amino acid sequence of SEQ ID NO: 54;
(nn) an α chain comprising the amino acid sequence of SEQ ID NO: 120;
(oo) a β chain comprising the amino acid sequence of SEQ ID NO: 121;
(pp) an α chain comprising the amino acid sequence of SEQ ID NO: 130;
(qq) a β chain comprising the amino acid sequence of SEQ ID NO: 131;
(rr) an α chain comprising the amino acid sequence of SEQ ID NO: 122;
(ss) a β chain comprising the amino acid sequence of SEQ ID NO: 123;
(tt) an α chain comprising the amino acid sequence of SEQ ID NO: 80;
(uu) a β chain comprising the amino acid sequence of SEQ ID NO: 81;
(vv) an α chain comprising the amino acid sequence of SEQ ID NO: 146;
(ww) a β chain comprising the amino acid sequence of SEQ ID NO: 146;
(xx) an α chain comprising the amino acid sequence of SEQ ID NO: 142;
(yy) a β chain comprising the amino acid sequence of SEQ ID NO: 143;
(zz) an α chain comprising the amino acid sequence of SEQ ID NO: 148;
(aaa) a β chain comprising the amino acid sequence of SEQ ID NO: 149;
(bbb) both (dd) and (ee); both (ff) and (gg); both (hh) and (ii); or both (jj) and (kk);
(ccc) both (ll) and (mm); both (nn) and (oo); both (pp) and (qq); or both (rr) and (ss);
(ddd) both (tt) and (uu); both (vv) and (ww); both (xx) and (yy); or both (zz) and (aaa);
(eee) an α chain comprising the amino acid sequence of SEQ ID NO: 23;
(fff) a β chain comprising the amino acid sequence of SEQ ID NO: 24;
(ggg) an α chain comprising the amino acid sequence of SEQ ID NO: 102;
(hhh) a β chain comprising the amino acid sequence of SEQ ID NO: 103;
(iii) an α chain comprising the amino acid sequence of SEQ ID NO: 124;
(jjj) a β chain comprising the amino acid sequence of SEQ ID NO: 125;
(kkk) an α chain comprising the amino acid sequence of SEQ ID NO: 104;
(lll) a β chain comprising the amino acid sequence of SEQ ID NO: 105;
(mmm) an α chain comprising the amino acid sequence of SEQ ID NO: 39;
(nnn) a β chain comprising the amino acid sequence of SEQ ID NO: 40;
(ooo) an α chain comprising the amino acid sequence of SEQ ID NO: 108;
(ppp) a β chain comprising the amino acid sequence of SEQ ID NO: 109;
(qqq) an α chain comprising the amino acid sequence of SEQ ID NO: 126;
(rrr) a β chain comprising the amino acid sequence of SEQ ID NO: 127;
(sss) an α chain comprising the amino acid sequence of SEQ ID NO: 110;
(ttt) a β chain comprising the amino acid sequence of SEQ ID NO: 111;
(uuu) an α chain comprising the amino acid sequence of SEQ ID NO: 78;
(vvv) a β chain comprising the amino acid sequence of SEQ ID NO: 79;
(www) an α chain comprising the amino acid sequence of SEQ ID NO: 138;
(xxx) a β chain comprising the amino acid sequence of SEQ ID NO: 139;
(yyy) an α chain comprising the amino acid sequence of SEQ ID NO: 134;
(zzz) a β chain comprising the amino acid sequence of SEQ ID NO: 135;
(aaaa) an α chain comprising the amino acid sequence of SEQ ID NO: 140;
(bbbb) a β chain comprising the amino acid sequence of SEQ ID NO: 141;
(dddd) both (eee) and (fff); both (eee) and (hhh); both (fff) and (ggg); both (ggg) and
(hhh); both (iii) and (jjj); both (iii) and (lll); both (jjj) and (kkk); or both (kkk) and (lll);
(eeee) both (mmm) and (nnn); both (mmm) and (ppp); both (nnn) and (ooo); both (ooo) and (ppp); both (qqq) and (rrr); both (qqq) and (ttt); both (rrr) and (sss); or both (sss) and (ttt); or
(ffff) both (uuu) and (vvv); both (uuu) and (xxx); both (vvv) and (www); both (www) and (xxx); both (yyy) and (zzz); both (yyy) and (bbbb); both (yyy) and (aaaa); or both (aaaa) and (bbbb).

20. An isolated or purified protein comprising at least one of the polypeptides according to claim 13.

21. The isolated or purified protein according to claim 20, comprising:
(a) a first polypeptide chain comprising the amino acid sequences of SEQ ID NOs: 1-3 and a second polypeptide chain comprising the amino acid sequences of SEQ ID NOs: 4-6;
(b) a first polypeptide chain comprising the amino acid sequences of SEQ ID NOs: 31-33 and a second polypeptide chain comprising the amino acid sequences of SEQ ID NOs: 34-36; or
(c) a first polypeptide chain comprising the amino acid sequences of SEQ ID NOs: 64-66 and a second polypeptide chain comprising the amino acid sequences of SEQ ID NOs: 67-69.

22. The isolated or purified protein according to claim 20, comprising:
(i) a first polypeptide chain comprising an amino acid sequence at least 99% identical to the amino acid sequence of SEQ ID NO: 7;
(ii) a second polypeptide chain comprising an amino acid sequence at least 99% identical to the amino acid sequence of SEQ ID NO: 8;
(iii) a first polypeptide chain comprising an amino acid sequence at least 99% identical to the amino acid sequence of SEQ ID NO: 90;
(iv) a second polypeptide chain comprising an amino acid sequence at least 99% identical to the amino acid sequence of SEQ ID NO: 91;
(v) a first polypeptide chain comprising an amino acid sequence at least 99% identical to the amino acid sequence of SEQ ID NO: 37;
(vi) a second polypeptide chain comprising an amino acid sequence at least 99% identical to the amino acid sequence of SEQ ID NO: 38;
(vii) a first polypeptide chain comprising an amino acid sequence at least 99% identical to the amino acid sequence of SEQ ID NO: 92;
(viii) a second polypeptide chain comprising an amino acid sequence at least 99% identical to the amino acid sequence of SEQ ID NO: 93;
(ix) a first polypeptide chain comprising an amino acid sequence at least 99% identical to the amino acid sequence of SEQ ID NO: 70;
(x) a second polypeptide chain comprising an amino acid sequence at least 99% identical to the amino acid sequence of SEQ ID NO: 71;
(xi) a first polypeptide chain comprising an amino acid sequence at least 99% identical to the amino acid sequence of SEQ ID NO: 132;
(xii) a second polypeptide chain comprising an amino acid sequence at least 99% identical to the amino acid sequence of SEQ ID NO: 133;
(xiii) a first polypeptide chain comprising an amino acid sequence at least 99% identical to the amino acid sequence of SEQ ID NO: 47;
(xiv) a second polypeptide chain comprising an amino acid sequence at least 99% identical to the amino acid sequence of SEQ ID NO: 48;
(xv) a first polypeptide chain comprising an amino acid sequence at least 99% identical to the amino acid sequence of SEQ ID NO: 94;
(xvi) a second polypeptide chain comprising an amino acid sequence at least 99% identical to the amino acid sequence of SEQ ID NO: 85;
(xvii) a second polypeptide chain comprising an amino acid sequence at least 99% identical to the amino acid sequence of SEQ ID NO: 95;
(xviii) a first polypeptide chain comprising an amino acid sequence at least 99% identical to the amino acid sequence of SEQ ID NO: 49;
(xix) a second polypeptide chain comprising an amino acid sequence at least 99% identical to the amino acid sequence of SEQ ID NO: 50;
(xx) a first polypeptide chain comprising an amino acid sequence at least 99% identical to the amino acid sequence of SEQ ID NO: 96;
(xxi) a second polypeptide chain comprising an amino acid sequence at least 99% identical to the amino acid sequence of SEQ ID NO: 87;
(xxii) a second polypeptide chain comprising an amino acid sequence at least 99% identical to the amino acid sequence of SEQ ID NO: 97;
(xxiii) a first polypeptide chain comprising an amino acid sequence at least 99% identical to the amino acid sequence of SEQ ID NO: 72;
(xxvi) a second polypeptide chain comprising an amino acid sequence at least 99% identical to the amino acid sequence of SEQ ID NO: 73;
(xxv) a first polypeptide chain comprising an amino acid sequence at least 99% identical to the amino acid sequence of SEQ ID NO: 88;
(xxvi) a second polypeptide chain comprising an amino acid sequence at least 99% identical to the amino acid sequence of SEQ ID NO: 89;
(xxvii) both (i) and (ii), both (i) and (iv), both (ii) and (iii), both (iii) and (iv), both (v) and (vi), both (v) and (viii), both (vi) and (vii), both (vii) and (viii), both (ix) and (x), both (ix) and (xii), both (x) and (xi), both (xi) and (xii), both (xiii) and (xiv), both (xiii) and (xvi), both (xiii) and (xvii), both (xiv) and (xv), both (xv) and (xvi), both (xv) and (xvii), both (xviii) and (xix), both (xviii) and (xxi), both (xviii) and (xxii), both (xix) and (xx), both (xx) and (xxi), both (xx) and (xxii), both (xxiii) and (xxiv), both (xxiii) and (xxvi), both (xxiv) and (xxv), or both (xxv) and (xxvi).

23. The isolated or purified protein according to claim 20, comprising:
(i) a first polypeptide chain comprising the amino acid sequence of SEQ ID NO: 7;
(ii) a second polypeptide chain comprising the amino acid sequence of SEQ ID NO: 8;
(iii) a first polypeptide chain comprising the amino acid sequence of SEQ ID NO: 90;
(iv) a second polypeptide chain comprising the amino acid sequence of SEQ ID NO: 91;
(v) a first polypeptide chain comprising the amino acid sequence of SEQ ID NO: 37;
(vi) a second polypeptide chain comprising the amino acid sequence of SEQ ID NO: 38;
(vii) a first polypeptide chain comprising the amino acid sequence of SEQ ID NO: 92;
(viii) a second polypeptide chain comprising the amino acid sequence of SEQ ID NO: 93;
(ix) a first polypeptide chain comprising the amino acid sequence of SEQ ID NO: 70;
(x) a second polypeptide chain comprising the amino acid sequence of SEQ ID NO: 71;
(xi) a first polypeptide chain comprising the amino acid sequence of SEQ ID NO: 132;
(xii) a second polypeptide chain comprising the amino acid sequence of SEQ ID NO: 133;
(xiii) a first polypeptide chain comprising the amino acid sequence of SEQ ID NO: 47;
(xiv) a second polypeptide chain comprising the amino acid sequence of SEQ ID NO: 48;
(xv) a first polypeptide chain comprising the amino acid sequence of SEQ ID NO: 94;
(xvi) a second polypeptide chain comprising the amino acid sequence of SEQ ID NO: 85;
(xvii) a second polypeptide chain comprising the amino acid sequence of SEQ ID NO: 95;
(xviii) a first polypeptide chain comprising the amino acid sequence of SEQ ID NO: 49;
(xix) a second polypeptide chain comprising the amino acid sequence of SEQ ID NO: 50;
(xx) a first polypeptide chain comprising the amino acid sequence of SEQ ID NO: 96;
(xxi) a second polypeptide chain comprising the amino acid sequence of SEQ ID NO: 87;
(xxii) a second polypeptide chain comprising the amino acid sequence of SEQ ID NO: 97;
(xxiii) a first polypeptide chain comprising the amino acid sequence of SEQ ID NO: 72;
(xxvi) a second polypeptide chain comprising the amino acid sequence of SEQ ID NO: 73;
(xxv) a first polypeptide chain comprising the amino acid sequence of SEQ ID NO: 88;
(xxvi) a second polypeptide chain comprising the amino acid sequence of SEQ ID NO: 89;
(xxvii) both (i) and (ii), both (i) and (iv), both (ii) and (iii), both (iii) and (iv), both (v) and (vi), both (v) and (viii), both (vi) and (vii), both (vii) and (viii), both (ix) and (x), both (ix) and (xii), both (x) and (xi), both (xi) and (xii), both (xiii) and (xiv), both (xiii) and (xvi), both (xiii) and (xvii), both (xiv) and (xv), both (xv) and (xvi), both (xv) and (xvii), both (xviii) and (xix), both (xviii) and (xxi), both (xviii) and (xxii), both (xix) and (xx), both (xx) and (xxi), both (xx) and (xxii), both (xxiii) and (xxiv), both (xxiii) and (xxvi), both (xxiv) and (xxv), or both (xxv) and (xxvi).

24. The isolated or purified protein according to claim 20, further comprising:
(a) a first polypeptide chain comprising an amino acid sequence at least 99% identical to the amino acid sequence of SEQ ID NO: 17, wherein:
(i) X at position 48 of SEQ ID NO: 17 is Thr or Cys;
(ii) X at position 112 of SEQ ID NO: 17 is Ser, Ala, Val, Leu, Ile, Pro, Phe, Met, or Trp;
(iii) X at position 114 of SEQ ID NO: 17 is Met, Ala, Val, Leu, Ile, Pro, Phe, or Trp; and
(iv) X at position 115 of SEQ ID NO: 17 is Gly, Ala, Val, Leu, Ile, Pro, Phe, Met, or Trp;
(b) a second polypeptide chain comprising an amino acid sequence at least 99% identical to the amino acid sequence of SEQ ID NO: 18, wherein X at position 57 of SEQ ID NO: 18 is Ser or Cys; or
(c) both (a) and (b).

25. The isolated or purified protein according to claim 20, further comprising:
(a) a first polypeptide chain comprising the amino acid sequence of SEQ ID NO: 17, wherein:
(i) X at position 48 of SEQ ID NO: 17 is Thr or Cys;
(ii) X at position 112 of SEQ ID NO: 17 is Ser, Ala, Val, Leu, Ile, Pro, Phe,
(iii) X at position 114 of SEQ ID NO: 17 is Met, Ala, Val, Leu, Ile, Pro, Phe, or Trp; and
(iv) X at position 115 of SEQ ID NO: 17 is Gly, Ala, Val, Leu, Ile, Pro, Phe, Met, or Trp;
(b) a second polypeptide chain comprising the amino acid sequence of SEQ ID NO: 18, wherein X at position 57 of SEQ ID NO: 18 is Ser or Cys; or
(c) both (a) and (b).

26. The isolated or purified protein according to claim 20, comprising:
(a) a first polypeptide chain comprising an amino acid sequence at least 99% identical to the amino acid sequence of SEQ ID NO: 21, wherein:
(i) X at position 180 of SEQ ID NO: 21 is Thr or Cys;
(ii) X at position 244 of SEQ ID NO: 21 is Ser, Ala, Val, Leu, Ile, Pro, Phe, Met, or Trp;
(iii) X at position 246 of SEQ ID NO: 21 is Met, Ala, Val, Leu, Ile, Pro, Phe, or Trp; and
(iv) X at position 247 of SEQ ID NO: 21 is Gly, Ala, Val, Leu, Ile, Pro, Phe, Met, or Trp;
(b) a second polypeptide chain comprising an amino acid sequence at least 99% identical to the amino acid sequence of SEQ ID NO: 22, wherein X at position 190 of SEQ ID NO: 22 is Ser or Cys;
(c) a first polypeptide chain comprising an amino acid sequence at least 99% identical to the amino acid sequence of SEQ ID NO: 100, wherein:
(i) X at position 180 of SEQ ID NO: 100 is Thr or Cys;
(ii) X at position 244 of SEQ ID NO: 100 is Ser, Ala, Val, Leu, Ile, Pro, Phe, Met, or Trp;
(iii) X at position 246 of SEQ ID NO: 100 is Met, Ala, Val, Leu, Ile, Pro, Phe, or Trp; and
(iv) X at position 247 of SEQ ID NO: 100 is Gly, Ala, Val, Leu, Ile, Pro, Phe,
(d) a second polypeptide chain comprising an amino acid sequence at least 99% identical to the amino acid sequence of SEQ ID NO: 101, wherein X at position 190 of SEQ ID NO: 101 is Ser or Cys;
(e) a first polypeptide chain comprising an amino acid sequence at least 99% identical to the amino acid sequence of SEQ ID NO: 41, wherein:
(i) X at position 181 of SEQ ID NO: 41 is Thr or Cys;
(ii) X at position 245 of SEQ ID NO: 41 is Ser, Ala, Val, Leu, Ile, Pro, Phe, Met, or Trp;
(iii) X at position 247 of SEQ ID NO: 41 is Met, Ala, Val, Leu, Ile, Pro, Phe, or Trp; and
(iv) X at position 248 of SEQ ID NO: 41 is Gly, Ala, Val, Leu, Ile, Pro, Phe, Met, or Trp;
(f) a second polypeptide chain comprising an amino acid sequence at least 99% identical to the amino acid sequence of SEQ ID NO: 42, wherein X at position 195 of SEQ ID NO: 42 is Ser or Cys;
(g) a first polypeptide chain comprising an amino acid sequence at least 99% identical to the amino acid sequence of SEQ ID NO: 106, wherein:
(i) X at position 181 of SEQ ID NO: 106 is Thr or Cys;
(ii) X at position 245 of SEQ ID NO: 106 is Ser, Ala, Val, Leu, Ile, Pro, Phe, Met, or Trp;
(iii) X at position 247 of SEQ ID NO: 106 is Met, Ala, Val, Leu, Ile, Pro, Phe, or Trp; and
(iv) X at position 248 of SEQ ID NO: 106 is Gly, Ala, Val, Leu, Ile, Pro, Phe, Met, or Trp;
(h) a second polypeptide chain comprising an amino acid sequence at least 99% identical to the amino acid sequence of SEQ ID NO: 107, wherein X at position 195 of SEQ ID NO: 107 is Ser or Cys;
(i) a first polypeptide chain comprising an amino acid sequence at least 99% identical to the amino acid sequence of SEQ ID NO: 74, wherein:
(i) X at position 180 of SEQ ID NO: 74 is Thr or Cys;
(ii) X at position 244 of SEQ ID NO: 74 is Ser, Ala, Val, Leu, Ile, Pro, Phe,
(iii) X at position 246 of SEQ ID NO: 74 is Met, Ala, Val, Leu, Ile, Pro, Phe, or Trp; and
(iv) X at position 247 of SEQ ID NO: 74 is Gly, Ala, Val, Leu, Ile, Pro, Phe, Met, or Trp;
(j) a second polypeptide chain comprising an amino acid sequence at least 99% identical to the amino acid sequence of SEQ ID NO: 75, wherein X at position 187 of SEQ ID NO: 75 is Ser or Cys;
(k) a first polypeptide chain comprising an amino acid sequence at least 99% identical to the amino acid sequence of SEQ ID NO: 136, wherein:
(i) X at position 180 of SEQ ID NO: 136 is Thr or Cys;
(ii) X at position 244 of SEQ ID NO: 136 is Ser, Ala, Val, Leu, Ile, Pro, Phe, Met, or Trp;
(iii) X at position 246 of SEQ ID NO: 136 is Met, Ala, Val, Leu, Ile, Pro, Phe, or Trp; and
(iv) X at position 247 of SEQ ID NO: 136 is Gly, Ala, Val, Leu, Ile, Pro, Phe, Met, or Trp;
(l) a second polypeptide chain comprising an amino acid sequence at least 99% identical to the amino acid sequence of SEQ ID NO: 137, wherein X at position 187 of SEQ ID NO: 75 is Ser or Cys;
(m) both (a) and (b); both (a) and (d); both (b) and (c); or both (c) and (d);
(n) both (e) and (o); both (e) and h); both (f) and (g); or both (g) and (h);
(n) both (i) and (j); both (i) and 1); both (j) and (k); or both (k) and (l);
(o) a first polypeptide chain comprising an amino acid sequence at least 99% identical to the amino acid sequence of SEQ ID NO: 55, wherein:
(i) X at position 160 of SEQ ID NO: 55 is Thr or Cys;
(ii) X at position 224 of SEQ ID NO: 55 is Ser, Ala, Val, Leu, Ile, Pro, Phe, Met, or Trp;
(iii) X at position 226 of SEQ ID NO: 55 is Met, Ala, Val, Leu, Ile, Pro, Phe, or Trp; and
(iv) X at position 227 of SEQ ID NO: 55 is Gly, Ala, Val, Leu, Ile, Pro, Phe,
(p) a second polypeptide chain comprising an amino acid sequence at least 99% identical to the amino acid sequence of SEQ ID NO: 56, wherein X at position 168 of SEQ ID NO: 56 is Ser or Cys;
(q) a first polypeptide chain comprising an amino acid sequence at least 99% identical to the amino acid sequence of SEQ ID NO: 112, wherein:
(i) X at position 159 of SEQ ID NO: 112 is Thr or Cys;
(ii) X at position 223 of SEQ ID NO: 112 is Ser, Ala, Val, Leu, Ile, Pro, Phe, Met, or Trp;
(iii) X at position 225 of SEQ ID NO: 112 is Met, Ala, Val, Leu, Ile, Pro, Phe, or Trp; and
(iv) X at position 226 of SEQ ID NO: 112 is Gly, Ala, Val, Leu, Ile, Pro, Phe, Met, or Trp;
(r) a second polypeptide chain comprising an amino acid sequence at least 99% identical to the amino acid sequence of SEQ ID NO: 113, wherein X at position 173 of SEQ ID NO: 113 is Ser or Cys;
(s) a first polypeptide chain comprising an amino acid sequence at least 99% identical to the amino acid sequence of SEQ ID NO: 57, wherein:
(i) X at position 160 of SEQ ID NO: 57 is Thr or Cys;
(ii) X at position 224 of SEQ ID NO: 57 is Ser, Ala, Val, Leu, Ile, Pro, Phe, Met, or Trp;
(iii) X at position 226 of SEQ ID NO: 57 is Met, Ala, Val, Leu, Ile, Pro, Phe, or Trp; and
(iv) X at position 227 of SEQ ID NO: 57 is Gly, Ala, Val, Leu, Ile, Pro, Phe, Met, or Trp;
(t) a second polypeptide chain comprising an amino acid sequence at least 99% identical to the amino acid sequence of SEQ ID NO: 58, wherein X at position 173 of SEQ ID NO: 58 is Ser or Cys;
(u) a first polypeptide chain comprising an amino acid sequence at least 99% identical to the amino acid sequence of SEQ ID NO: 118, wherein:
(i) X at position 161 of SEQ ID NO: 118 is Thr or Cys;
(ii) X at position 225 of SEQ ID NO: 118 is Ser, Ala, Val, Leu, Ile, Pro, Phe,
(iii) X at position 227 of SEQ ID NO: 118 is Met, Ala, Val, Leu, Ile, Pro, Phe, or Trp; and
(iv) X at position 228 of SEQ ID NO: 118 is Gly, Ala, Val, Leu, Ile, Pro, Phe, Met, or Trp;
(v) a second polypeptide chain comprising an amino acid sequence at least 99% identical to the amino acid sequence of SEQ ID NO: 119, wherein X at position 103178 of SEQ ID NO: 119 is Ser or Cys;
(w) a first polypeptide chain comprising an amino acid sequence at least 99% identical to the amino acid sequence of SEQ ID NO: 76, wherein:
(i) X at position 159 of SEQ ID NO: 76 is Thr or Cys;
(ii) X at position 223 of SEQ ID NO: 76 is Ser, Ala, Val, Leu, Ile, Pro, Phe, Met, or Trp;
(iii) X at position 225 of SEQ ID NO: 76 is Met, Ala, Val, Leu, Ile, Pro, Phe, or Trp; and
(iv) X at position 226 of SEQ ID NO: 76 is Gly, Ala, Val, Leu, Ile, Pro, Phe, Met, or Trp;
(x) a second polypeptide chain comprising an amino acid sequence at least 99% identical to the amino acid sequence of SEQ ID NO: 77, wherein X at position 168 of SEQ ID NO: 77 is Ser or Cys;
(y) a first polypeptide chain comprising an amino acid sequence at least 99% identical to the amino acid sequence of SEQ ID NO: 144, wherein:
(i) X at position 160 of SEQ ID NO: 144 is Thr or Cys;
(ii) X at position 224 of SEQ ID NO: 144 is Ser, Ala, Val, Leu, Ile, Pro, Phe, Met, or Trp;
(iii) X at position 226 of SEQ ID NO: 144 is Met, Ala, Val, Leu, Ile, Pro, Phe, or Trp; and
(iv) X at position 227 of SEQ ID NO: 144 is Gly, Ala, Val, Leu, Ile, Pro, Phe, Met, or Trp;
(z) a second polypeptide chain comprising an amino acid sequence at least 99% identical to the amino acid sequence of SEQ ID NO: 145, wherein X at position 166 of SEQ ID NO: 145 is Ser or Cys;
(aa) both (o) and (p); or both (q) and (r);
(bb) both (s) and (t); or both (u) and (v);
(cc) both (w) and (x); or both (y) and (z);
(dd) a first polypeptide chain comprising an amino acid sequence at least 99% identical to the amino acid sequence of SEQ ID NO: 51;
(ee) a second polypeptide chain comprising an amino acid sequence at least 99% identical to the amino acid sequence of SEQ ID NO: 52;
(ff) a first polypeptide chain comprising an amino acid sequence at least 99% identical to the amino acid sequence of SEQ ID NO: 114;
(gg) a second polypeptide chain comprising an amino acid sequence at least 99% identical to the amino acid sequence of SEQ ID NO: 115;
(hh) a first polypeptide chain comprising an amino acid sequence at least 99% identical to the amino acid sequence of SEQ ID NO: 128;
(ii) a second polypeptide chain comprising an amino acid sequence at least 99% identical to the amino acid sequence of SEQ ID NO: 129;
(jj) a first polypeptide chain comprising an amino acid sequence at least 99% identical to the amino acid sequence of SEQ ID NO: 116;
(kk) a second polypeptide chain comprising an amino acid sequence at least 99% identical to the amino acid sequence of SEQ ID NO: 117;
(ll) a first polypeptide chain comprising an amino acid sequence at least 99% identical to the amino acid sequence of SEQ ID NO: 53;
(mm) a second polypeptide chain comprising an amino acid sequence at least 99% identical to the amino acid sequence of SEQ ID NO: 54;
(nn) a first polypeptide chain comprising an amino acid sequence at least 99% identical to the amino acid sequence of SEQ ID NO: 120;
(oo) a second polypeptide chain comprising an amino acid sequence at least 99% identical to the amino acid sequence of SEQ ID NO: 121;
(pp) a first polypeptide chain comprising an amino acid sequence at least 99% identical to the amino acid sequence of SEQ ID NO: 130;
(qq) a second polypeptide chain comprising an amino acid sequence at least 99% identical to the amino acid sequence of SEQ ID NO: 131;
(rr) a first polypeptide chain comprising an amino acid sequence at least 99% identical to the amino acid sequence of SEQ ID NO: 122;
(ss) a second polypeptide chain comprising an amino acid sequence at least 99% identical to the amino acid sequence of SEQ ID NO: 123;
(tt) a first polypeptide chain comprising an amino acid sequence at least 99% identical to the amino acid sequence of SEQ ID NO: 80;
(uu) a second polypeptide chain comprising an amino acid sequence at least 99% identical to the amino acid sequence of SEQ ID NO: 81;
(vv) a first polypeptide chain comprising an amino acid sequence at least 99% identical to the amino acid sequence of SEQ ID NO: 146;
(ww) a second polypeptide chain comprising an amino acid sequence at least 99% identical to the amino acid sequence of SEQ ID NO: 146;
(xx) a first polypeptide chain comprising an amino acid sequence at least 99% identical to the amino acid sequence of SEQ ID NO: 142;
(yy) a second polypeptide chain comprising an amino acid sequence at least 99% identical to the amino acid sequence of SEQ ID NO: 143;
(zz) a first polypeptide chain comprising an amino acid sequence at least 99% identical to the amino acid sequence of SEQ ID NO: 148;
(aaa) a second polypeptide chain comprising an amino acid sequence at least 99% identical to the amino acid sequence of SEQ ID NO: 149;
(bbb) both (dd) and (ee); both (ff) and (gg); both (hh) and (ii); or both (jj) and (kk);
(ccc) both (ll) and (mm); both (nn) and (oo); both (pp) and (qq); or both (rr) and (ss);
(ddd) both (tt) and (uu); both (vv) and (ww); both (xx) and (yy); or both (zz) and (aaa);
(eee) a first polypeptide chain comprising an amino acid sequence at least 99% identical to the amino acid sequence of SEQ ID NO: 23;
(fff) a second polypeptide chain comprising an amino acid sequence at least 99% identical to the amino acid sequence of SEQ ID NO: 24;
(ggg) a first polypeptide chain comprising an amino acid sequence at least 99% identical to the amino acid sequence of SEQ ID NO: 102;
(hhh) a second polypeptide chain comprising an amino acid sequence at least 99% identical to the amino acid sequence of SEQ ID NO: 103;
(iii) a first polypeptide chain comprising an amino acid sequence at least 99% identical to the amino acid sequence of SEQ ID NO: 124;
(jjj) a second polypeptide chain comprising an amino acid sequence at least 99% identical to the amino acid sequence of SEQ ID NO: 125;
(kkk) a first polypeptide chain comprising an amino acid sequence at least 99% identical to the amino acid sequence of SEQ ID NO: 104;
(lll) a second polypeptide chain comprising an amino acid sequence at least 99% identical to the amino acid sequence of SEQ ID NO: 105;
(mmm) a first polypeptide chain comprising an amino acid sequence at least 99% identical to the amino acid sequence of SEQ ID NO: 39;
(nnn) a second polypeptide chain comprising an amino acid sequence at least 99% identical to the amino acid sequence of SEQ ID NO: 40;
(ooo) a first polypeptide chain comprising an amino acid sequence at least 99% identical to the amino acid sequence of SEQ ID NO: 108;
(ppp) a second polypeptide chain comprising an amino acid sequence at least 99% identical to the amino acid sequence of SEQ ID NO: 109;
(qqq) a first polypeptide chain comprising an amino acid sequence at least 99% identical to the amino acid sequence of SEQ ID NO: 126;
(rrr) a second polypeptide chain comprising an amino acid sequence at least 99% identical to the amino acid sequence of SEQ ID NO: 127;
(sss) a first polypeptide chain comprising an amino acid sequence at least 99% identical to the amino acid sequence of SEQ ID NO: 110;
(ttt) a second polypeptide chain comprising an amino acid sequence at least 99% identical to the amino acid sequence of SEQ ID NO: 111;
(uuu) a first polypeptide chain comprising an amino acid sequence at least 99% identical to the amino acid sequence of SEQ ID NO: 78;
(vvv) a second polypeptide chain comprising an amino acid sequence at least 99% identical to the amino acid sequence of SEQ ID NO: 79;
(www) a first polypeptide chain comprising an amino acid sequence at least 99% identical to the amino acid sequence of SEQ ID NO: 138;
(xxx) a second polypeptide chain comprising an amino acid sequence at least 99% identical to the amino acid sequence of SEQ ID NO: 139;
(yyy) a first polypeptide chain comprising an amino acid sequence at least 99% identical to the amino acid sequence of SEQ ID NO: 134;
(zzz) a second polypeptide chain comprising an amino acid sequence at least 99% identical to the amino acid sequence of SEQ ID NO: 135;
(aaaa) a first polypeptide chain comprising an amino acid sequence at least 99% identical to the amino acid sequence of SEQ ID NO: 140;
(bbbb) a second polypeptide chain comprising an amino acid sequence at least 99% identical to the amino acid sequence of SEQ ID NO: 141;
(dddd) both (eee) and (fff); both (eee) and (hhh); both (fff) and (ggg); both (ggg) and (hhh); both (iii) and (jjj); both (iii) and (lll); both (jjj) and (kkk); or both (kkk) and (lll);
(eeee) both (mmm) and (nnn); both (mmm) and (ppp); both (nnn) and (ooo); both (ooo) and (ppp); both (qqq) and (rrr); both (qqq) and (ttt); both (rrr) and (sss); or both (sss) and (ttt); or
(ffff) both (uuu) and (vvv); both (uuu) and (xxx); both (vvv) and (www); both (www) and (xxx); both (yyy) and (zzz); both (yyy) and (bbbb); both (yyy) and (aaaa); or both (aaaa) and (bbbb).

27. The isolated or purified protein according to claim 20, comprising:
(a) a first polypeptide chain comprising the amino acid sequence of SEQ ID NO: 21, wherein:
(i) X at position 180 of SEQ ID NO: 21 is Thr or Cys;
(ii) X at position 244 of SEQ ID NO: 21 is Ser, Ala, Val, Leu, Ile, Pro, Phe, Met, or Trp;
(iii) X at position 246 of SEQ ID NO: 21 is Met, Ala, Val, Leu, Ile, Pro, Phe, or Trp; and
(iv) X at position 247 of SEQ ID NO: 21 is Gly, Ala, Val, Leu, Ile, Pro, Phe, Met, or Trp;
(b) a second polypeptide chain comprising the amino acid sequence of SEQ ID NO: 22, wherein X at position 190 of SEQ ID NO: 22 is Ser or Cys;
(c) a first polypeptide chain comprising the amino acid sequence of SEQ ID NO: 100, wherein:
(i) X at position 180 of SEQ ID NO: 100 is Thr or Cys;
(ii) X at position 244 of SEQ ID NO: 100 is Ser, Ala, Val, Leu, Ile, Pro, Phe, Met, or Trp;
(iii) X at position 246 of SEQ ID NO: 100 is Met, Ala, Val, Leu, Ile, Pro, Phe, or Trp; and
(iv) X at position 247 of SEQ ID NO: 100 is Gly, Ala, Val, Leu, Ile, Pro, Phe, Met, or Trp;
(d) a second polypeptide chain comprising the amino acid sequence of SEQ ID NO: 101, wherein X at position 190 of SEQ ID NO: 101 is Ser or Cys;
(e) a first polypeptide chain comprising the amino acid sequence of SEQ ID NO: 41, wherein:
(i) X at position 181 of SEQ ID NO: 41 is Thr or Cys;
(ii) X at position 245 of SEQ ID NO: 41 is Ser, Ala, Val, Leu, Ile, Pro, Phe, Met, or Trp;
(iii) X at position 247 of SEQ ID NO: 41 is Met, Ala, Val, Leu, Ile, Pro, Phe, or Trp; and
(iv) X at position 248 of SEQ ID NO: 41 is Gly, Ala, Val, Leu, Ile, Pro, Phe, Met, or Trp;
(f) a second polypeptide chain comprising the amino acid sequence of SEQ ID NO: 42, wherein X at position 195 of SEQ ID NO: 42 is Ser or Cys;
(g) a first polypeptide chain comprising the amino acid sequence of SEQ ID NO: 106, wherein:
(i) X at position 181 of SEQ ID NO: 106 is Thr or Cys;
(ii) X at position 245 of SEQ ID NO: 106 is Ser, Ala, Val, Leu, Ile, Pro, Phe, Met, or Trp;
(iii) X at position 247 of SEQ ID NO: 106 is Met, Ala, Val, Leu, Ile, Pro, Phe, or Trp; and
(iv) X at position 248 of SEQ ID NO: 106 is Gly, Ala, Val, Leu, Ile, Pro, Phe, Met, or Trp;
(h) a second polypeptide chain comprising the amino acid sequence of SEQ ID NO: 107, wherein X at position 195 of SEQ ID NO: 107 is Ser or Cys;
(i) a first polypeptide chain comprising the amino acid sequence of SEQ ID NO: 74, wherein:
(i) X at position 180 of SEQ ID NO: 74 is Thr or Cys;
(ii) X at position 244 of SEQ ID NO: 74 is Ser, Ala, Val, Leu, Ile, Pro, Phe, Met, or Trp;
(iii) X at position 246 of SEQ ID NO: 74 is Met, Ala, Val, Leu, Ile, Pro, Phe, or Trp; and
(iv) X at position 247 of SEQ ID NO: 74 is Gly, Ala, Val, Leu, Ile, Pro, Phe, Met, or Trp;
(j) a second polypeptide chain comprising the amino acid sequence of SEQ ID NO: 75, wherein X at position 187 of SEQ ID NO: 75 is Ser or Cys;
(k) a first polypeptide chain comprising the amino acid sequence of SEQ ID NO: 136, wherein:
(i) X at position 180 of SEQ ID NO: 136 is Thr or Cys;
(ii) X at position 244 of SEQ ID NO: 136 is Ser, Ala, Val, Leu, Ile, Pro, Phe, Met, or Trp;
(iii) X at position 246 of SEQ ID NO: 136 is Met, Ala, Val, Leu, Ile, Pro, Phe, or Trp; and
(iv) X at position 247 of SEQ ID NO: 136 is Gly, Ala, Val, Leu, Ile, Pro, Phe, Met, or Trp;
(l) a second polypeptide chain comprising the amino acid sequence of SEQ ID NO: 137, wherein X at position 187 of SEQ ID NO: 75 is Ser or Cys;
(m) both (a) and (b); both (a) and (d); both (b) and (c); or both (c) and (d);
(n) both (e) and (o); both (e) and h); both (f) and (g); or both (g) and (h);
(n) both (i) and (j); both (i) and 1); both (j) and (k); or both (k) and (l);
(o) a first polypeptide chain comprising the amino acid sequence of SEQ ID NO: 55, wherein:
(i) X at position 160 of SEQ ID NO: 55 is Thr or Cys;
(ii) X at position 224 of SEQ ID NO: 55 is Ser, Ala, Val, Leu, Ile, Pro, Phe, Met, or Trp;
(iii) X at position 226 of SEQ ID NO: 55 is Met, Ala, Val, Leu, Ile, Pro, Phe, or Trp; and
(iv) X at position 227 of SEQ ID NO: 55 is Gly, Ala, Val, Leu, Ile, Pro, Phe,
(p) a second polypeptide chain comprising the amino acid sequence of SEQ ID NO: 56, wherein X at position 168 of SEQ ID NO: 56 is Ser or Cys;
(q) a first polypeptide chain comprising the amino acid sequence of SEQ ID NO: 112, wherein:
(i) X at position 159 of SEQ ID NO: 112 is Thr or Cys;
(ii) X at position 223 of SEQ ID NO: 112 is Ser, Ala, Val, Leu, Ile, Pro, Phe, Met, or Trp;
(iii) X at position 225 of SEQ ID NO: 112 is Met, Ala, Val, Leu, Ile, Pro, Phe, or Trp; and
(iv) X at position 226 of SEQ ID NO: 112 is Gly, Ala, Val, Leu, Ile, Pro, Phe, Met, or Trp;
(r) a second polypeptide chain comprising the amino acid sequence of SEQ ID NO: 113, wherein X at position 173 of SEQ ID NO: 113 is Ser or Cys;
(s) a first polypeptide chain comprising the amino acid sequence of SEQ ID NO: 57, wherein:
(i) X at position 160 of SEQ ID NO: 57 is Thr or Cys;
(ii) X at position 224 of SEQ ID NO: 57 is Ser, Ala, Val, Leu, Ile, Pro, Phe, Met, or Trp;
(iii) X at position 226 of SEQ ID NO: 57 is Met, Ala, Val, Leu, Ile, Pro, Phe, or Trp; and
(iv) X at position 227 of SEQ ID NO: 57 is Gly, Ala, Val, Leu, Ile, Pro, Phe, Met, or Trp;
(t) a second polypeptide chain comprising the amino acid sequence of SEQ ID NO: 58, wherein X at position 173 of SEQ ID NO: 58 is Ser or Cys;
(u) a first polypeptide chain comprising the amino acid sequence of SEQ ID NO: 118, wherein:
(i) X at position 161 of SEQ ID NO: 118 is Thr or Cys;
(ii) X at position 225 of SEQ ID NO: 118 is Ser, Ala, Val, Leu, Ile, Pro, Phe, Met, or Trp;
(iii) X at position 227 of SEQ ID NO: 118 is Met, Ala, Val, Leu, Ile, Pro, Phe, or Trp; and
(iv) X at position 228 of SEQ ID NO: 118 is Gly, Ala, Val, Leu, Ile, Pro, Phe, Met, or Trp;
(v) a second polypeptide chain comprising the amino acid sequence of SEQ ID NO: 119, wherein X at position 103178 of SEQ ID NO: 119 is Ser or Cys;
(w) a first polypeptide chain comprising the amino acid sequence of SEQ ID NO: 76, wherein:
(i) X at position 159 of SEQ ID NO: 76 is Thr or Cys;
(ii) X at position 223 of SEQ ID NO: 76 is Ser, Ala, Val, Leu, Ile, Pro, Phe, Met, or Trp;
(iii) X at position 225 of SEQ ID NO: 76 is Met, Ala, Val, Leu, Ile, Pro, Phe, or Trp; and
(iv) X at position 226 of SEQ ID NO: 76 is Gly, Ala, Val, Leu, Ile, Pro, Phe, Met, or Trp;
(x) a second polypeptide chain comprising the amino acid sequence of SEQ ID NO: 77, wherein X at position 168 of SEQ ID NO: 77 is Ser or Cys;
(y) a first polypeptide chain comprising the amino acid sequence of SEQ ID NO: 144, wherein:
(i) X at position 160 of SEQ ID NO: 144 is Thr or Cys;
(ii) X at position 224 of SEQ ID NO: 144 is Ser, Ala, Val, Leu, Ile, Pro, Phe, Met, or Trp;
(iii) X at position 226 of SEQ ID NO: 144 is Met, Ala, Val, Leu, Ile, Pro, Phe, or Trp; and
(iv) X at position 227 of SEQ ID NO: 144 is Gly, Ala, Val, Leu, Ile, Pro, Phe, Met, or Trp;
(z) a second polypeptide chain comprising the amino acid sequence of SEQ ID NO: 145, wherein X at position 166 of SEQ ID NO: 145 is Ser or Cys;
(aa) both (o) and (p); or both (q) and (r);
(bb) both (s) and (t); or both (u) and (v);
(cc) both (w) and (x); or both (y) and (z);
(dd) a first polypeptide chain comprising the amino acid sequence of SEQ ID NO: 51;
(ee) a second polypeptide chain comprising the amino acid sequence of SEQ ID NO: 52;
(ff) a first polypeptide chain comprising the amino acid sequence of SEQ ID NO: 114;
(gg) a second polypeptide chain comprising the amino acid sequence of SEQ ID NO: 115;
(hh) a first polypeptide chain comprising the amino acid sequence of SEQ ID NO: 128;
(ii) a second polypeptide chain comprising the amino acid sequence of SEQ ID NO: 129;
(jj) a first polypeptide chain comprising the amino acid sequence of SEQ ID NO: 116;
(kk) a second polypeptide chain comprising the amino acid sequence of SEQ ID NO: 117;
(ll) a first polypeptide chain comprising the amino acid sequence of SEQ ID NO: 53;
(mm) a second polypeptide chain comprising the amino acid sequence of SEQ ID NO: 54;
(nn) a first polypeptide chain comprising the amino acid sequence of SEQ ID NO: 120;
(oo) a second polypeptide chain comprising the amino acid sequence of SEQ ID NO: 121;
(pp) a first polypeptide chain comprising the amino acid sequence of SEQ ID NO: 130;
(qq) a second polypeptide chain comprising the amino acid sequence of SEQ ID NO: 131;
(rr) a first polypeptide chain comprising the amino acid sequence of SEQ ID NO: 122;
(ss) a second polypeptide chain comprising the amino acid sequence of SEQ ID NO: 123;
(tt) a first polypeptide chain comprising the amino acid sequence of SEQ ID NO: 80;
(uu) a second polypeptide chain comprising the amino acid sequence of SEQ ID NO: 81;
(vv) a first polypeptide chain comprising the amino acid sequence of SEQ ID NO: 146;
(ww) a second polypeptide chain comprising the amino acid sequence of SEQ ID NO: 146;
(xx) a first polypeptide chain comprising the amino acid sequence of SEQ ID NO: 142;
(yy) a second polypeptide chain comprising the amino acid sequence of SEQ ID NO: 143;
(zz) a first polypeptide chain comprising the amino acid sequence of SEQ ID NO: 148;
(aaa) a second polypeptide chain comprising the amino acid sequence of SEQ ID NO: 149;
(bbb) both (dd) and (ee); both (ff) and (gg); both (hh) and (ii); or both (jj) and (kk);
(ccc) both (ll) and (mm); both (nn) and (oo); both (pp) and (qq); or both (rr) and (ss);
(ddd) both (tt) and (uu); both (vv) and (ww); both (xx) and (yy); or both (zz) and (aaa);
(eee) a first polypeptide chain comprising the amino acid sequence of SEQ ID NO: 23;
(fff) a second polypeptide chain comprising the amino acid sequence of SEQ ID NO: 24;
(ggg) a first polypeptide chain comprising the amino acid sequence of SEQ ID NO: 102;
(hhh) a second polypeptide chain comprising the amino acid sequence of SEQ ID NO: 103;
(iii) a first polypeptide chain comprising the amino acid sequence of SEQ ID NO: 124;
(jjj) a second polypeptide chain comprising the amino acid sequence of SEQ ID NO: 125;
(kkk) a first polypeptide chain comprising the amino acid sequence of SEQ ID NO: 104;
(lll) a second polypeptide chain comprising the amino acid sequence of SEQ ID NO: 105;
(mmm) a first polypeptide chain comprising the amino acid sequence of SEQ ID NO: 39;
(nnn) a second polypeptide chain comprising the amino acid sequence of SEQ ID NO: 40;
(ooo) a first polypeptide chain comprising the amino acid sequence of SEQ ID NO: 108;
(ppp) a second polypeptide chain comprising the amino acid sequence of SEQ ID NO: 109;
(qqq) a first polypeptide chain comprising the amino acid sequence of SEQ ID NO: 126;
(rrr) a second polypeptide chain comprising the amino acid sequence of SEQ ID NO: 127;
(sss) a first polypeptide chain comprising the amino acid sequence of SEQ ID NO: 110;
(ttt) a second polypeptide chain comprising the amino acid sequence of SEQ ID NO: 111;
(uuu) a first polypeptide chain comprising the amino acid sequence of SEQ ID NO: 78;
(vvv) a second polypeptide chain comprising the amino acid sequence of SEQ ID NO: 79;
(www) a first polypeptide chain comprising the amino acid sequence of SEQ ID NO: 138;
(xxx) a second polypeptide chain comprising the amino acid sequence of SEQ ID NO: 139;
(yyy) a first polypeptide chain comprising the amino acid sequence of SEQ ID NO: 134;
(zzz) a second polypeptide chain comprising the amino acid sequence of SEQ ID NO: 135;
(aaaa) a first polypeptide chain comprising the amino acid sequence of SEQ ID NO: 140;
(bbbb) a second polypeptide chain comprising the amino acid sequence of SEQ ID NO: 141;
(dddd) both (eee) and (fff); both (eee) and (hhh); both (fff) and (ggg); both (ggg) and (hhh); both (iii) and (jjj); both (iii) and (lll); both (jjj) and (kkk); or both (kkk) and (lll);
(eeee) both (mmm) and (nnn); both (mmm) and (ppp); both (nnn) and (ooo); both (ooo) and (ppp); both (qqq) and (rrr); both (qqq) and (ttt); both (rrr) and (sss); or both (sss) and (ttt); or
(ffff) both (uuu) and (vvv); both (uuu) and (xxx); both (vvv) and (www); both (www) and (xxx); both (yyy) and (zzz); both (yyy) and (bbbb); both (yyy) and (aaaa); or both (aaaa) and (bbbb).

28. An isolated or purified nucleic acid comprising a nucleotide sequence encoding the TCR according to claim 1.

29. An isolated or purified nucleic acid comprising, from 5′ to 3′, a first nucleic acid sequence and a second nucleotide sequence, wherein the first and second nucleotide sequence, respectively, encode the amino sequences of SEQ ID NOs: 7 and 8; 7 and 91; 90 and 8; 90 and 91; 8 and 7; 91 and 7; 8 and 90; 91 and 90; 37 and 38; 37 and 93; 92 and 38; 92 and 93; 38 and 37; 93 and 37; 38 and 92; 93 and 92; 70 and 71; 70 and 133; 132 and 71; 132 and 133; 71 and 70; 133 and 70; 71 and 132; 133 and 132; 23 and 24; 23 and 103; 102 and 24; 102 and 103; 24 and 23; 103 and 23; 24 and 102; 103 and 102; 39 and 40;
39 and 109; 108 and 40; 108 and 109; 40 and 39; 109 and 39; 40 and 108; 109 and 108; 78 and 79; 78 and 139; 138 and 79; 138 and 139; 79 and 78; 139 and 78; 79 and 138; 139 and 138; 21 and 22; 21 and 101; 100 and 22; 100 and 101; 22 and 21; 101 and 21; 22 and 100;
101 and 100; 41 and 42; 41 and 107; 106 and 42; 106 and 107; 42 and 41; 107 and 41; 42 and 106; 107 and 106; 74 and 75; 74 and 101; 100 and 75, 100 and 101; 75 and 74; 101 and 74;
75 and 100; 101 and 100; 124 and 125; 124 and 105; 104 and 125; 104 and 105; 125 and 124;
105 and 124; 125 and 104; 105 and 104; 126 and 127; 126 and 111; 110 and 127; 110 and 111; 127 and 126; 111 and 126; 127 and 110; 111 and 110; 134 and 135; 140 and 135; 134 and 141; 140 and 141; 135 and 134; 135 and 140; 141 and 134; 141 and 140; 47 and 48; 48 and 47; 49 and 50; 50 and 49; 72 and 73; 73 and 72; 94 and 95; 95 and 94; 94 and 85; 85 and 94; 96 and 97; 97 and 96; 96 and 87; 87 and 96; 88 and 89; 89 and 88; 51 and 52; 52 and 51;
53 and 54; 54 and 53; 80 and 81; 81 and 80; 55 and 56; 56 and 55; 57 and 58; 58 and 57; 76 and 77; 77 and 76; 128 and 129; 129 and 128; 130 and 131; 131 and 130; 142 and 143; 143 and 142; 112 and 113; 113 and 112; 118 and 119; 119 and 118; 144 and 145; 145 and 144; 114 and 115; 115 and 114; 120 and 121; 121 and 120; 146 and 147; 147 and 146; 116 and 117; 117 and 116; 122 and 123; 123 and 122; 148 and 149; 149 and 148; 150 and 151; 151 and 150; 154 and 155; 155 and 154; 152 and 153; 153 and 152; 156 and 157; 157 and 156; 160 and 161; 161 and 160; 158 and 159; or 159 and 158.

30. The isolated or purified nucleic acid according to claim 29, further comprising a third nucleotide sequence interposed between the first and second nucleotide sequence, wherein the third nucleotide sequence encodes a cleavable linker peptide.

31. The isolated or purified nucleic acid according to claim 30, wherein the cleavable linker peptide comprises the amino acid sequence of SEQ ID NO: 25.

32. A recombinant expression vector comprising the nucleic acid according to claim 28.

33. The recombinant expression vector according to claim 32, which is a transposon or a lentiviral vector.

34. An isolated or purified TCR, polypeptide, or protein encoded by the nucleic acid according to claim 28.

35. An isolated or purified TCR, polypeptide, or protein that results from expression of the nucleic acid according to claim 28.

36. A method of producing a host cell expressing a TCR that has antigenic specificity for the peptide of SEQ ID NO: 30, the method comprising contacting a cell with the vector according to claim 32 under conditions that allow introduction of the vector into the cell.

37. An isolated or purified host cell comprising the nucleic acid according to the recombinant expression vector according to claim 32.

38. The host cell according to claim 37 wherein the cell is a human lymphocyte.

39. The host cell according to claim 37, wherein the cell is selected from the group consisting of a T cell, a natural killer T (NKT) cell, an invariant natural killer T (iNKT) cell, and a natural killer (NK) cell.

40. An isolated or purified population of cells comprising the host cell according to claim 37.

41. A method of producing a T cell receptor the method comprising culturing the host cell according to claim 37, so that the TCR, polypeptide, or protein is produced.

42. A pharmaceutical composition comprising (a) the TCR according to claim 1 and (b) a pharmaceutically acceptable carrier.

43. A method of detecting the presence of cancer in mammal, the method comprising:
(a) contacting a sample comprising cells of the cancer with the TCR according to claim 1, thereby forming a complex; and
(b) detecting the complex,
wherein detection of the complex is indicative of the presence of cancer in the mammal.

44. A method of inducing an immune response against a cancer in a mammal, the method comprising administering to the mammal an effective amount of the host cell according to claim 37 or a population of cells thereof.

45. A method of treating or preventing cancer in a mammal the method comprising administering to the mammal an effective amount of the host cell according to claim 37 or a population of cells thereof.

46. The method according to claim 45, wherein the cancer expresses a mutated human RAS amino acid sequence with a substitution of glycine at position 12 with valine,
wherein the mutated human RAS amino acid sequence is a mutated human Kirsten rat sarcoma viral oncogene homolog (KRAS), a mutated human Harvey rat sarcoma viral oncogene homolog (HRAS), or a mutated human Neuroblastoma rat sarcoma viral oncogene homolog (NRAS) amino acid sequence, and
wherein position 12 is defined by reference to the wild-type human KRAS, wild-type human HRAS, or wild-type human NRAS protein, respectively.

47. The method according to claim 46, wherein the mutated human RAS amino acid sequence is a mutated human Kirsten rat sarcoma viral oncogene homolog (KRAS) amino acid sequence.

48. The method according to claim 46, wherein the mutated human RAS amino acid sequence is a mutated human neuroblastoma rat sarcoma viral oncogene homolog (NRAS) amino acid sequence.

49. The method according to claim 46, wherein the mutated human RAS amino acid sequence is a mutated human Harvey rat sarcoma viral oncogene homolog (HRAS) amino acid sequence.

50. The method according to claim 46, wherein the cancer is pancreatic, colorectal, lung, endometrial, ovarian, or prostate cancer.
